(19) United States
(12) Patent Application Publication
(10) Pub. No.: US 2013/0130928 A1
(43) Pub. Date: May 23, 2013

(54) METHODS FOR PREDICTING OR MONITORING WHETHER A PATIENT AFFECTED BY A CANCER IS RESPONSIVE TO A TREATMENT WITH A MOLECULE OF THE TAXOID FAMILY

(71) Applicants: Anne Chauchereau, Fontenay-Aux-Roses (FR); Frederic Commo, Rueil Malmaison (FR); Nader Al Nakouzi, Amchit (LB)

(72) Inventors: Anne Chauchereau, Fontenay-Aux-Roses (FR); Frederic Commo, Rueil Malmaison (FR); Nader Al Nakouzi, Amchit (LB)

(73) Assignee: INSTITUT GUSTAVE ROUSSY, VILLEJUIF CEDEX (FR)

(21) Appl. No.: 13/638,053

(22) PCT Filed: Apr. 8, 2011
(86) PCT No.: PCT/EP2011/055482

§ 371 Date: Dec. 4, 2012

(30) Foreign Application Priority Data
Apr. 8, 2010 (EP) 10305361.7

(51) Int. Cl. C12Q 1/02

(52) U.S. Cl. CPC C12Q 1/025
USPC 506/9; 506/17; 506/18

(57) ABSTRACT

The present invention concerns in vitro methods for predicting or monitoring whether a patient affected by a cancer is responsive to a treatment with a molecule of the taxoid family based on a resistance expression signature, kits for performing the methods, and methods for screening or identifying a compound suitable for improving the treatment of a cancer with a molecule of the taxoid family or for reducing the resistance development during the treatment of a cancer with the molecule of the taxoid family.

### FIELD OF THE INVENTION

The present invention relates to method for predicting the response to a treatment with a molecule of the taxoid family, kits and method for screening compounds useful for improve the treatment with the molecule.

### BACKGROUND OF THE INVENTION

Prostate cancer became, based on frequency and in Western countries, the first cancer in men, behind the lung cancer. This disease is the second cause of cancer death in men. Since 2005, more than 60,000 men are touched by prostate cancer (PCa) each year and 10,000 men died of this disease. The efficiency of docetaxel chemotherapy (Taxotere®) in prostate cancer (CaP) has been demonstrated for the first time in 2004 in two clinical trials, i.e. TAX 327 and SWOG 99-16, with an increase in survival. Accordingly, docetaxel became today a treatment of choice of metastatic hormone-refractory prostate cancers and phase III clinical trials are ongoing to assess its efficacy for the treatment of high-risk localized prostate cancer. Taxotere® is currently approved in 5 different cancer types in Europe and the US: Prostate cancer, breast cancer, lung cancer, gastric cancer and head and neck cancer. However, in spite of the survival benefit provided by this molecule, docetaxel has a great toxicity and almost half of the patients treated with docetaxel develop a resistance to the chemotherapy either from the beginning, or in a secondary way. Moreover, docetaxel is not effective on all the types of cancer. For instance, in case of breast cancer, only 30 to 50% of the metastatic tumours respond to docetaxel. Resistance to taxanes is common and there is an increasing need to try and identify those patients who will respond to treatment.

A genomic analysis was performed with two cell lines (PC3 and DU145) resistant to a docetaxel dose of 11 nM (Patterson et al, Oncogene, 2006, 25: 6113-6122). The article discloses an expression signature of 30 genes. The authors also demonstrated the effect of STAT1 and Clusterin in an in vitro model for the docetaxel resistance. However, the validation of the expression of these two genes in the docetaxel-resistance has not been performed on tumours. The authors further demonstrated that resveratrol leads to a decreased expression of clusterin in docetaxel resistant cells and, then to an increase of apoptosis (Sallman et al, Mol. Can. Ther., 2007, 6: 2938-2947). Other groups used docetaxel resistance cell lines (PC3-R) in their research (Lo Nigro et al, BJU Int., 2008, 102: 622-7). Some other groups used prostate cancer cell lines treated during a short period (24-72 h) with docetaxel for studying the role of genes in the docetaxel response.

In addition, a patent application WO 2006/062811 concerns a method for measuring resistance or sensitivity to docetaxel.

Therefore, there is still a strong need of a diagnostic method for predicting responsiveness to docetaxel and avoiding useless treatments. Indeed, before the initiation of the treatment, it is currently impossible to identify the patients who will respond to or who will have a resistance to docetaxel.

### SUMMARY OF THE INVENTION

The present invention provides an expression signature specific of the docetaxel resistance in human prostate cancer. Based on this signature, the present invention provides a method for predicting or monitoring whether a patient affected by a cancer is responsive to a treatment with a molecule of the taxoid family.

Accordingly, the present invention concerns an in vitro method for predicting or monitoring whether a patient affected by a cancer is responsive to a treatment with a molecule of the taxoid family, wherein the method comprises: 1) providing a biological sample from said subject; 2) determining in the biological sample the expression level of the following genes JAM3, PCDH7, DCDC2, KHDRBS2, MFAP5, AUTS2, C2orf55, SLC3A1, AKAP12, ZNF649, RNASET2, NCF2, DLC1, CXCR4, CR594735, TRIM6 and MBNL3.

Preferably, the expression level is compared to a reference expression level, for instance the expression level of the genes in cell-lines or patients sensitive to the treatment by the molecule of the taxoid family. In particular, the over-expression of genes from Tables 1 and 3 and/or the under-expression of genes from Tables 2 and 4 are indicative of a resistance to the treatment by the molecule of the taxoid family. More preferably, the over-expression of genes selected from the group consisting of PCDH7, KHDRBS2, AUTS2, and C2orf55 and/or the under-expression of genes selected from the group consisting of JAM3, DCDC2, MFAP5, SLC3A1, AKAP12, ZNF649, RNASET2, NCF2, DLC1, CXCR4, CR594735, TRIM6, and MBNL3 are indicative of a resistance to the treatment by the molecule of the taxoid family. The expression level of genes can be determined by the quantity of protein or mRNA encoded by said genes. Preferably, the biological sample is a cancer sample.

In a preferred embodiment, the molecule of the taxoid family is selected from the group consisting of docetaxel, larotaxel, cabazitaxel (XRP6258), BMS-184476, BMS-188797, BMS-275183, ortataxel, RPR 109881A, RPR 116258, NBT-287, PG-paclitaxel, ABRAXANE®, Tesetaxel, IDN 5390, Taxoprexin, DHA-paclitaxel, and MAC-321. More preferably, the molecule of the taxoid family is docetaxel.

Optionally, the method further comprises determining the expression level of at least one gene selected from the group consisting of FBN2, HIST2H2AA4, WDR31, FBXO15, THAP2, BF207040, HIST1H2BK, UNC13A, FAM27E3, LOC728613, FAM27E1, NPDC1, HIST1H2BL, UBE2J1, TJP2, HAVCR1, ZBTB24, CDKAL1, COQ3, TMCC3, ZFPM2, SLC3A2, LIMCH1, EPB41L2, B4GALT4, BX281122 and TPD52L1. Alternatively, the method further comprises determining the expression level of the genes FBN2, HIST2H2AA4, WDR31, FBXO15, THAP2, BF207040, HIST1H2BK, UNC13A, FAM27E3, LOC728613, FAM27E1, NPDC1, HIST1H2BL, UBE2J1, TJP2, HAVCR1, ZBTB24, CDKAL1, COQ3, TMCC3, ZFPM2, SLC3A2, LIMCH1, EPB41L2, B4GALT4, BX281122 and TPD52L1.

Optionally, the method further comprises determining the expression level of at least one gene selected from the group consisting of the genes listed in Tables 1-4.

Preferably, the biological sample is a cancer sample.

Preferably, the cancer is selected from the group consisting of the breast cancer, the lung cancer, the prostate cancer, the gastric cancer and the head and neck cancer, more preferably a prostate cancer.

The present invention also concerns kits and DNA chips suitable for this method. Accordingly, the present invention concerns a kit for predicting or monitoring whether a patient affected by a cancer is responsive to a treatment with a molecule of the taxoid family, wherein the kit comprises detection means selected from the group consisting of a pair of primers, a probe and an antibody specific to the genes JAM3, PCDH7, DCDC2, KHDRBS2, MFAP5, AUTS2, C2orf55, SLC3A1, AKAP12, ZNF649, RNASET2, NCF2, DLC1, CXCR4, CR594735, TRIM6 and MBNL3 or a DNA chip comprises a solid support which carries nucleic acids that are specific to the genes JAM3, PCDH7, DCDC2, KHDRBS2, MFAP5, AUTS2, C2orf55, SLC3A1, AKAP12, ZNF649, RNASET2, NCF2, DLC1, CXCR4, CR594735, TRIM6 and MBNL3. Optionally, the kit further comprises detection means selected from the group consisting of a pair of primers, a probe and an antibody specific to at least one gene selected from the group consisting of the genes the kit or DNA chip further comprises detection means for at least one gene selected from the group consisting of FBN2, HIST2H2AA4, WDR31, FBXO15, THAP2, BF207040, HIST1H2BK, UNC13A, FAM27E3, LOC728613, FAM27E1, NPDC1, HIST1H2BL, UBE2J1, TJP2, HAVCR1, ZBTB24, CDKAL1, COQ3, TMCC3, ZFPM2, SLC3A2, LIMCH1, EPB41L2, B4GALT4, BX281122 and TPD52L1. Optionally, the DNA chip further carries nucleic acids that are specific to at least one gene selected from the group consisting of the genes the kit or DNA chip further comprises detection means for at least one gene selected from the group consisting of FBN2, HIST2H2AA4, WDR31, FBXO15, THAP2, BF207040, HIST1H2BK, UNC13A, FAM27E3, LOC728613, FAM27E1, NPDC1, HIST1H2BL, UBE2J1, TJP2, HAVCR1, ZBTB24, CDKAL1, COQ3, TMCC3, ZFPM2, SLC3A2, LIMCH1, EPB41L2, B4GALT4, BX281122 and TPD52L1.

The present invention further concerns methods for screening or identifying a compound suitable for improving the treatment of a cancer with a molecule of the taxoid family or for reducing the resistance development during the treatment of a cancer with a molecule of the taxoid family. In a first embodiment, the method comprises: 1) providing a cell-line with the genes PCDH7, KHDRBS2, AUTS2, and C2orf55 being over-expressed and the genes JAM3, DCDC2, MFAP5, SLC3A1, AKAP12, ZNF649, RNASET2, NCF2, DLC1, CXCR4, CR594735, TRIM6, and MBNL3 being under-expressed; 2) contacting said cell-line with a test compound; 3) determining the expression level of said genes; and, 4) selecting the compound which decreases the expression level of one or several of the over-expressed genes and increases the expression level of one or several of the under-expressed genes. In a second embodiment, the method comprises: 1) providing a cell-line sensitive to the molecule of the taxoid family; 2) contacting said cell-line with a test compound and the molecule of the taxoid family; 3) determining the expression level of the genes JAM3, PCDH7, DCDC2, KHDRBS2, MFAP5, AUTS2, C2orf55, SLC3A1, AKAP12, ZNF649, RNASET2, NCF2, DLC1, CXCR4, CR594735, TRIM6, and MBNL3; and, 4) selecting the compound which inhibits the appearance of an over-expression of the genes PCDH7, KHDRBS2, AUTS2, and C2orf55 and/or an under-expression of the genes JAM3, DCDC2, MFAP5, SLC3A1, AKAP12, ZNF649, RNASET2, NCF2, DLC1, CXCR4, CR594735, TRIM6, and MBNL3.

### DETAILED DESCRIPTION OF THE INVENTION

The present invention provides the identification of protein coding genes involved in the mechanism of docetaxel resistance in prostate cancer treatment.

The inventors prepared in vitro cellular models of docetaxel resistant prostate cancer by selecting cell clones by pharmaceutical pressure from a cellular model of prostate cancer i.e. IGR-CaP1 cell line. Cell lines became resistant to increasing doses of docetaxel (5 nM; 12 nM; 25 nM, 50 nM; 100 nM; 200 nM). The prostate cancer cell line IGR-CaP1 is described in CNCM deposit number I-4126 on Feb. 10, 2009.

A micro-array genomic analysis was performed by comparing sensitive and resistant IGR-CaP1 cell line at six docetaxel concentrations (5; 12; 25, 50, 100 and 200 nM), as detailed in the experimental section. This analysis led to the identification of 1081 genes associated to the resistance to increasing doses of docetaxel (with a P value ≦1e-3 and a fold change between the first and the last doses of docetaxel (between 5 and 200 nM)≧2). In this signature, 772 genes are over-expressed and 309 genes are under-expressed. These genes are presented in Tables 1 to 4.

The inventors realized a second microarray experiment in which the same IGR-CaP1 resistant cell lines have been used. Contrasting with the first analysis, the different resistant cell lines were cultured in the total absence of docetaxel for 2 passages before RNA extraction and microarray analysis, to retain only irreversible resistance mechanisms. To enhance the robustness of bioinformatics analysis, this analysis was generated from biological duplicates.

The second microarray genomic analysis was performed by comparing sensitive and resistant IGR-CaP1 cell line at six docetaxel concentrations (5; 12; 25, 50, 100 and 200 nM), as detailed in the experimental section. This analysis led to the identification of 486 genes associated to the resistance to increasing doses of docetaxel (with a P value ≦1e-5 and a fold change between the first and the last doses of docetaxel (between 5 and 200 nM)≧2). Table 5 showed the 44 genes which were found in common in the two microarray analyses. Among these genes, a final list of 17 genes was selected with 4 genes that were overexpressed in a dose-dependent manner and 13 genes that were under-expressed in a dose-dependent manner (Table 6).

On this basis, the inventors identified a set of genes whose combined expression profiles allow to distinguish patients between responder and non-responder to a treatment with a molecule of the taxoid family. A “responder” or “responsive” patient refers to a patient who shows or will show a clinically significant recovery when treated in the cancer when treated with a molecule of the taxoid family. In particular, the size of the tumor will no more increase, decrease or the tumor will disappear.

Therefore, the present invention discloses an expression signature useful for in vitro method for predicting whether a patient suffering of a cancer would be responsive to a treatment with a molecule of the taxoid family.

In a preferred embodiment of the invention, the method comprises determining the expression level of genes of Table 6, namely JAM3, PCDH7, DCDC2, KHDRBS2, MFAP5, AUTS2, C2orf55, SLC3A1, AKAP12, ZNF649, RNASET2, NCF2, DLC1, CXCR4, CR594735, TRIM6, and MBNL3. In particular, when comparing with the expression level of the genes in cell-lines or patients sensitive to the treatment by the molecule of the taxoid family, the over-expression of genes PCDH7, KHDRBS2, AUTS2, and C2orf55, and/or the under-expression of genes JAM3, DCDC2, MFAP5, SLC3A1, AKAP12, ZNF649, RNASET2, NCF2, DLC1, CXCR4, CR594735, TRIM6, and MBNL3 are indicative of a resistance to the treatment by the molecule of the taxoid family. In addition, the expression level of one or several genes of Table 5 may further be determined, namely genes selected from the group consisting of FBN2, HIST2H2AA4, WDR31, FBXO15, THAP2, BF207040, HIST1H2BK, UNC13A, FAM27E3, LOC728613, FAM27E1, NPDC1, HIST1H2BL, UBE2J1, TJP2, HAVCR1, ZBTB24, CDKAL1, COQ3, TMCC3, ZFPM2, SLC3A2, LIMCH1, EPB41L2, B4GALT4, BX281122 and TPD52L1. More particularly, when comparing with the expression level of the genes in cell-lines or patients sensitive to the treatment by the molecule of the taxoid family, the over-expression of one or several genes selected among FBN2, HIST2H2AA4, WDR31, FBXO15, THAP2, BF207040, HIST1H2BK, UNC13A, FAM27E3, LOC728613, FAM27E1, NPDC1 and HIST1H2BL, and/or the under-expression of one or several genes selected among UBE2J1, TJP2, HAVCR1, ZBTB24, CDKAL1, COQ3, TMCC3, ZFPM2, SLC3A2, LIMCH1, EPB41L2, B4GALT4, BX281122 and TPD52L1 may be indicative of a resistance to the treatment by the molecule of the taxoid family. Optionally, the expression level of at least 6, 7, 8, 9, 10, 15, 20, 25, 30, 40 or all genes of Table 5 are determined. Optionally, the expression level of at least 6, 7, 8, 9, 10, 15, 20, 25, 30, 40, 50, 60, 70, 80, 90, 100, 150, 200, 300, 500 or 1000 additional genes of Tables 1-4 may also be determined. However, in a preferred embodiment, the method does not comprise the determination of the expression level of more than 200, 100 or 50 genes, more preferably no more than 50, 40, 30 or 20 genes.

Alternatively, the method comprises determining the expression level of genes from the present expression signature (see Tables 1 to 4 or Tables 1-6) in a biological sample of said patient. In particular, the method comprises determining the expression level of at least 5 genes selected from the group consisting of the genes listed in Tables 1-6 or in Tables 1 and 2, in a biological sample of said patient. Preferably, the method comprises determining the expression level of at least 6, 7, 8, 9, 10, 15, 20, 25, 30, 40, 50, 60, 70, 80, 90, 100, 150, 200, 300, 500 or 1000 genes of Tables 1-6 or Tables 1 and 2. Optionally, the method further comprises determining the expression level of at least one gene selected from the group consisting of the genes listed in Tables 3 and 4. Optionally, the method further comprises determining the expression level of at least one gene selected from the group consisting of the genes listed in Tables 5 and 6. Preferably, the expression level of at least 2, 3, 5, 10, 15 or 20 genes selected from the group consisting of the genes listed in Tables 3-6 or Tables 3 and 4, is determined. Preferably, the expression level of at least 2, 3, 5, 10, 15 or 20 genes selected from the group consisting of the genes listed in Tables 5 and 6, is also determined. Alternatively, the method comprises determining the expression level of 5 to 1081 genes of Tables 1 to 4, optionally of 10 to 500, 20 to 300, 30 to 250, 50 to 250, 20 to 250, 30 to 200, 40 to 150, 50 to 100, 60 to 90 or 70 to 80. However, in a preferred embodiment, the method does not comprise the determination of the expression level of more than 200, 100 or 50 genes, more preferably no more than 50, 40, 30 or 20 genes.

By “predicting” or “prediction” is intended herein the likelihood that a patient will respond or not to a molecule of the taxoid family and also the extent of the response. Predictive methods of the invention can be used clinically to make treatment decisions by choosing the most appropriate treatment modalities for any particular patient.

Therefore, the present invention also concerns a method for selecting a patient suffering of a cancer for a treatment with a molecule of the taxoid family, comprising determining the expression level of genes of Table 6, namely JAM3, PCDH7, DCDC2, KHDRBS2, MFAP5, AUTS2, C2orf55, SLC3A1, AKAP12, ZNF649, RNASET2, NCF2, DLC1, CXCR4, CR594735, TRIM6, and MBNL3 in a biological sample of said patient and selecting the patient predicted to be responsive to a treatment with a molecule of the taxoid family. In particular, when comparing with the expression level of the genes in cell-lines or patients sensitive to the treatment by the molecule of the taxoid family, the over-expression of genes PCDH7, KHDRBS2, AUTS2, and C2orf55, and/or the under-expression of genes JAM3, DCDC2, MFAP5, SLC3A1, AKAP12, ZNF649, RNASET2, NCF2, DLC1, CXCR4, CR594735, TRIM6, and MBNL3 are indicative of a resistance to the treatment by the molecule of the taxoid family. Optionally, the method further comprises determining the expression level of at least one gene selected from the group consisting of genes of Table 5, namely FBN2, HIST2H2AA4, WDR31, FBXO15, THAP2, BF207040, HIST1H2BK, UNC13A, FAM27E3, LOC728613, FAM27E1, NPDC1, HIST1H2BL, UBE2J1, TJP2, HAVCR1, ZBTB24, CDKAL1, COQ3, TMCC3, ZFPM2, SLC3A2, LIMCH1, EPB41L2, B4GALT4, BX281122 and TPD52L1. More particularly, when comparing with the expression level of the genes in cell-lines or patients sensitive to the treatment by the molecule of the taxoid family, the over-expression of one or several genes selected among FBN2, HIST2H2AA4, WDR31, FBXO15, THAP2, BF207040, HIST1H2BK, UNC13A, FAM27E3, LOC728613, FAM27E1, NPDC1 and HIST1H2BL, and/or the under-expression of one or several genes selected among UBE2J1, TJP2, HAVCR1, ZBTB24, CDKAL1, COQ3, TMCC3, ZFPM2, SLC3A2, LIMCH1, EPB41L2, B4GALT4, BX281122 and TPD52L1 may be indicative of a resistance to the treatment by the molecule of the taxoid family. Optionally, the expression level of at least 6, 7, 8, 9, 10, 15, 20, 25, 30, 40 or all genes of Table 5 are determined. Optionally, the expression level of at least 6, 7, 8, 9, 10, 15, 20, 25, 30, 40, 50, 60, 70, 80, 90, 100, 150, 200, 300, 500 or 1000 additional genes of Tables 1-4 may also be determined. However, in a preferred embodiment, the method does not comprise the determination of the expression level of more than 200, 100 or 50 genes, more preferably no more than 50, 40, 30 or 20 genes.

In addition, the present invention also concerns a method for selecting a patient suffering of a cancer for a treatment with a molecule of the taxoid family, comprising determining the expression level of at least 5, 6, 7, 8, 9, 10, 15, 20, 25, 30, 40, 50, 60, 70, 80, 90, 100, 150, 200, 300, 500 or 1000 genes selected from the group consisting of the genes listed in Tables 1-6 or Tables 1 and 2, in a biological sample of said patient and selecting the patient predicted to be responsive to a treatment with a molecule of the taxoid family. Optionally, the method further comprises determining the expression level of at least one gene selected from the group consisting of the genes listed in Tables 3-6 or Tables 3 and 4.

In an embodiment, the genes, preferably the additional genes, are selected from Tables 1 and 2 on the criteria of “fold change”. Accordingly, the genes with the greatest fold change (in absolute value) between 5 nM of docetaxel and 200 nM of docetaxel are chosen. For instance, the genes associated with a fold change greater (in absolute value) than 3, preferably than 4, 5, 6, 7, 8, 9 or 10, are selected. In a particular embodiment, the genes are selected from the group consisting of ENST00000399723, BI836406, C10orf79, AK022962, TMTC1, LOC728295, SUSD5, WNT6, BC044624, AY358241, ZNF251, ST6GAL2, LOC643401, NOV, CLGN, PROM1, SPEF2, FLRT2, RGS2, FOXP2, TRIM55, PKD2L1, RP4-692D3.1, CB985069, ARL14, AY831680, XRN1, THAP5, ZNF248, BC016022, PLAG1, THC2724353, THC2488083, C5orf41, BMS1P5, BMS1, THC2627008, PLA2G4A, DPY19L2, VCX2, PPP1R1C, GLT25D2, KIAA1841, IFIT2, ZNF596, TSPAN19, BC029907, C10orf107, ZNF594, AMPD1, C21orf88, THC2694827, HSPC105, IFI44, THC2662262, FAM84A, DNAH7, KHDRBS2, NANP, AK091357, N4BP2L1, FAM105A, CA941346, CCDC68, CASC1, FAM90A12, PBX1, THC2739159, KCNQ2, ANXA1, AL122040, THC2655194, ENST00000342608, DSC2, ENOX1, IL13, BG571904, BX455216, LOC729085, BG188151, LOC729409, C1orf103, PPP1R14C, NAIP, C13orf31, GOLGA8E, AK022848, CXorf22, KIF5C, LRRCC1, FAM81B, ID2, CMYA5, C1orf194, TTC18, tcag7.1314, ZNF385B, ADAMTS6, RHOU, ENST00000378850, C2orf55, GPR83, LRRIQ1, WDR31, DEFB126, ARMETL1, LOC642826, LOC129881, C2orf13, THC2553512, ACVR1C, ZNF207, ANTXR1, CHD9, THC2526838, ABCA12, TncRNA, FKTN, PTPRG, ZNF233, ENST00000370378, FANK1, PCM1, SERPINI1, ARID4B, KIAA1377, FGF7, CV339166, LINCR, DA834198, CFH, SCG2, ARHGEF10, DA093175, GOLGA8A, AK021467, LOC283666, FLJ35767, THC2725553, ZNF430, CCDC141, MAP3K13, CCDC66, THC2727226, THC2528990, THC2718728, THC2507829, AK123972, EDEM3, DB304731, TPD52L1, MFAP5, EHF, NCF2, TRIM6, PERLD1, ATXN1, INHBB, CR627122, JAM3, CXCL14, CR594735, FLJ11235, C15orf52, LIMCH1, LOH11CR2A, BX281122, GPR110, ARNT2, ATP6V0A4, PDGFRB, ELA3B, NEDD9, MYH6, SLC35F2, HAS3, COLEC12, SLC3A2, AW993939, RUNX2, SUSD3, PLAU, SLC22A3, FCRL4, DOCK2, SOX3, THC2616558, RNASET2, LOC100130360, IL1R2, MGAT5B, TCF7L1, AF222857, AHNAK, HOXB8, S100A16, INSIG1 and DCDC2. Preferably, the genes are selected from the group consisting of ENST00000399723, BI836406, C10orf79, AK022962, TMTC1, LOC728295, SUSD5, WNT6, BC044624, AY358241, ZNF251, ST6GAL2, LOC643401, NOV, CLGN, PROM1, SPEF2, FLRT2, RGS2, FOXP2, TRIM55, PKD2L1, RP4-692D3.1, CB985069, ARL14, AY831680, XRN1, THAP5, ZNF248, BC016022, PLAG1, THC2724353, THC2488083, C5orf41, BMS1P5, BMS1, THC2627008, PLA2G4A, DPY19L2, VCX2, PPP1R1C, GLT25D2, KIAA1841, IFIT2, ZNF596, TSPAN19, BC029907, C10orf107, ZNF594, AMPD1, C21orf88, THC2694827, HSPC105, IFI44, THC2662262, FAM84A, DNAH7, KHDRBS2, NANP, AK091357, N4BP2L1, FAM105A, CA941346, CCDC68, CASC1, FAM90A12, PBX1, THC2739159, KCNQ2, ANXA1, AL122040, THC2655194, ENST00000342608, DSC2, ENOX1, IL13, BG571904, BX455216, LOC729085, BG188151, LOC729409, C1orf103, PPP1R14C, NAIP, C13orf31, GOLGA8E, AK022848, CXorf22, KIF5C, TPD52L1, MFAP5, EHF, NCF2, TRIM6, PERLD1, ATXN1, INHBB, CR627122, JAM3, CXCL14, CR594735, FLJ11235, C15orf52, LIMCH1, LOH11CR2A, BX281122, GPR110, ARNT2, ATP6V0A4, PDGFRB, ELA3B, NEDD9, MYH6, SLC35F2, HAS3, COLEC12, SLC3A2, AW993939, RUNX2 and SUSD3. More preferably, the genes are selected from the group consisting of ENST00000399723, BI836406, C10orf79, AK022962, TMTC1, LOC728295, SUSD5, WNT6, BC044624, AY358241, ZNF251, ST6GAL2, LOC643401, NOV, CLGN, PROM1, SPEF2, FLRT2, RGS2, FOXP2, TRIM55, PKD2L1, RP4-692D3.1, TPD52L1, MFAP5, EHF, NCF2, TRIM6, PERLD1, ATXN1, INHBB, CR627122, JAM3, CXCL14 and CR594735. In the most preferred embodiment, the genes are selected from the group consisting of ENST00000399723, BI836406, C10orf79, AK022962, TMTC1, LOC728295, SUSD5, WNT6, BC044624, TPD52L1, MFAP5, EHF, NCF2, TRIM6, PERLD1, ATXN1, INHBB and CR627122.

In another embodiment, the genes, preferably the additional genes, are selected from Tables 1 on the criteria of “fold change”. Accordingly, the over-expressed genes with the greatest fold change (in absolute value) are chosen. For instance, the over-expressed genes associated with a fold change greater (in absolute value) than 3, preferably than 4, 5, 6, 7, 8, 9 or 10, are selected. In a particular embodiment, the genes are selected from the group consisting of ENST00000399723, BI836406, C10orf79, AK022962, TMTC1, LOC728295, SUSD5, WNT6, BC044624, AY358241, ZNF251, ST6GAL2, LOC643401, NOV, CLGN, PROM1, SPEF2, FLRT2, RGS2, FOXP2, TRIM55, PKD2L1, RP4-692D3.1, CB985069, ARL14, AY831680, XRN1, THAP5, ZNF248, BC016022, PLAG1, THC2724353, THC2488083, C5orf41, BMS1P5, BMS1, THC2627008, PLA2G4A, DPY19L2, VCX2, PPP1R1C, GLT25D2, KIAA1841, IFIT2, ZNF596, TSPAN19, BC029907, C10orf107, ZNF594, AMPD1, C21orf88, THC2694827, HSPC105, IFI44, THC2662262, FAM84A, DNAH7, KHDRBS2, NANP, AK091357, N4BP2L1, FAM105A, CA941346, CCDC68, CASC1, FAM90A12, PBX1, THC2739159, KCNQ2, ANXA1, AL122040, THC2655194, ENST00000342608, DSC2, ENOX1, IL13, BG571904, BX455216, LOC729085, BG188151, LOC729409, C1orf103, PPP1R14C, NAIP, C13orf31, GOLGA8E, AK022848, CXorf22, KIF5C, LRRCC1, FAM81B, ID2, CMYA5, C1orf194, TTC18, tcag7.1314, ZNF385B, ADAMTS6, RHOU, ENST00000378850, C2orf55, GPR83, LRRIQ1, WDR31, DEFB126, ARMETL1, LOC642826, LOC129881, C2orf13, THC2553512, ACVR1C, ZNF207, ANTXR1, CHD9, THC2526838, ABCA12, TncRNA, FKTN, PTPRG, ZNF233, ENST00000370378, FANK1, PCM1, SERPINI1, ARID4B, KIAA1377, FGF7, CV339166, L1NCR, DA834198, CFH, SCG2, ARHGEF10, DA093175, GOLGA8A, AK021467, LOC283666, FLJ35767, THC2725553, ZNF430, CCDC141, MAP3K13, CCDC66, THC2727226, THC2528990, THC2718728, THC2507829, AK123972, EDEM3 and DB304731. Preferably, the genes are selected from the group consisting of ENST00000399723, BI836406, C10orf79, AK022962, TMTC1, LOC728295, SUSD5, WNT6, BC044624, AY358241, ZNF251, ST6GAL2, LOC643401, NOV, CLGN, PROM1, SPEF2, FLRT2, RGS2, FOXP2, TRIM55, PKD2L1, RP4-692D3.1, CB985069, ARL14, AY831680, XRN1, THAP5, ZNF248, BC016022, PLAG1, THC2724353, THC2488083, C5orf41, BMS1P5, BMS1, THC2627008, PLA2G4A, DPY19L2, VCX2, PPP1R1C, GLT25D2, KIAA1841, IFIT2, ZNF596, TSPAN19, BC029907, C10orf107, ZNF594, AMPD1, C21orf88, THC2694827, HSPC105, IFI44, THC2662262, FAM84A, DNAH7, KHDRBS2, NANP, AK091357, N4BP2L1, FAM105A, CA941346, CCDC68, CASC1, FAM90A12, PBX1, THC2739159, KCNQ2, ANXA1, AL122040, THC2655194, ENST00000342608, DSC2, ENOX1, IL13, BG571904, BX455216, LOC729085, BG188151, LOC729409, C1orf103, PPP1R14C, NAIP, C13orf31, GOLGA8E, AK022848, CXorf22 and KIF5C. More preferably, the genes are selected from the group consisting of ENST00000399723, BI836406, C10orf79, AK022962, TMTC1, LOC728295, SUSD5, WNT6, BC044624, AY358241, ZNF251, ST6GAL2, LOC643401, NOV, CLGN, PROM1, SPEF2, FLRT2, RGS2, FOXP2, TRIM55, PKD2L1 and RP4-692D3.1. In the most preferred embodiment, the genes are selected from the group consisting of ENST00000399723, BI836406, C10orf79, AK022962, TMTC1, LOC728295, SUSD5, WNT6 and BC044624.

In a further embodiment, the genes, preferably the additional genes are selected from Tables 2 on the criteria of “fold change”. Accordingly, the under-expressed genes with the greatest fold change (in absolute value) are chosen. For instance, the under-expressed genes associated with a fold change greater (in absolute value) than 3, preferably than 4, 5, 6, 7, 8, 9 or 10, are selected. In a particular embodiment, the genes are selected from the group consisting of TPD52L1, MFAP5, EHF, NCF2, TRIM6, PERLD1, ATXN1, INHBB, CR627122, JAM3, CXCL14, CR594735, FLJ11235, C15orf52, LIMCH1, LOH11CR2A, BX281122, GPR110, ARNT2, ATP6V0A4, PDGFRB, ELA3B, NEDD9, MYH6, SLC35F2, HAS3, COLEC12, SLC3A2, AW993939, RUNX2, SUSD3, PLAU, SLC22A3, FCRL4, DOCK2, SOX3, THC2616558, RNASET2, LOC100130360, IL1R2, MGAT5B, TCF7L1, AF222857, AHNAK, HOXB8, S100A16, INSIG1, DCDC2. Preferably, the genes are selected from the group consisting of TPD52L1, MFAP5, EHF, NCF2, TRIM6, PERLD1, ATXN1, INHBB, CR627122, JAM3, CXCL14, CR594735, FLJ11235, C15orf52, LIMCH1, LOH11CR2A, BX281122, GPR110, ARNT2, ATP6V0A4, PDGFRB, ELA3B, NEDD9, MYH6, SLC35F2, HAS3, COLEC12, SLC3A2, AW993939, RUNX2 and SUSD3. More preferably, the genes are selected from the group consisting of TPD52L1, MFAP5, EHF, NCF2, TRIM6, PERLD1, ATXN1, INHBB, CR627122, JAM3, CXCL14 and CR594735. In the most preferred embodiment, the genes are selected from the group consisting of TPD52L1, MFAP5, EHF, NCF2, TRIM6, PERLD1, ATXN1, INHBB and CR627122.

Optionally, the method of the invention further comprises determining the expression level of at least one gene, preferably at least one additional gene, selected from the group consisting of the genes listed in Table 3 and 4, preferably TFPI2, PCDH7, SMAD9, AK090762, RAB39B, BF831953, AL050204, VCX, ITGA2, CXCR4, SLC16A10, PDE1A, MAL, KRT80, FXYD2 and AK3L1, more preferably TFPI2, PCDH7, SMAD9, AK090762, RAB39B, BF831953, AL050204, VCX, CXCR4, SLC16A10, PDE1A, MAL, and the most preferably TFPI2, PCDH7, SMAD9, CXCR4 and SLC16A10. In particular, the expression level of at least 2, 3, 4, 5, 6, 7, 8, 9 or 10 genes was determined. In an embodiment, the method of the invention further comprises determining the expression level of at least one gene, preferably at least one additional gene, selected from the group consisting of TFPI2, PCDH7, SMAD9, AK090762, RAB39B, BF831953, AL050204, VCX, ITGA2, AKAP9, AUTS2, CEP152, SLITRK6, CCPG1, MANEAL, THC2733296, CD55, ANKRD18A, LAT2, BRCA2, LRP2BP, LPHN2 and ITGB8, preferably TFPI2, PCDH7, SMAD9, AK090762, RAB39B, BF831953, AL050204, VCX and ITGA2, more preferably TFPI2, PCDH7, SMAD9, AK090762, RAB39B, BF831953, AL050204 and VCX, the most preferably TFPI2, PCDH7 and SMAD9. In another embodiment, the method of the invention further comprises determining the expression level of at least one gene, preferably at least one additional gene, selected from the group consisting of CXCR4, SLC16A10, PDE1A, MAL, KRT80, FXYD2, AK3L1, LIN7A, GPR177, TNF, WNT2B, CGNL1, RPS6KA2, SUNC1, DIAPH2, AKAP12, NRG1, PDE4DIP, IL1R1, LZTS1, SLC3A1, MGST1, ACOT9, SLC12A3, ASRGL1 and HRG, preferably CXCR4, SLC16A10, PDE1A, MAL, KRT80, FXYD2 and AK3L1, more preferably CXCR4, SLC16A10, PDE1A and MAL, and the most preferably CXCR4 and SLC16A10.

The method can comprise the step of comparing the expression levels of the genes determined in the sample to reference or control expression levels. The reference or control expression levels are determined with a sample of cells, preferably cancer cells, which are sensitive to the molecule of the taxoid family. Alternatively, reference or control expression levels are determined with a sample of patients or subjects sensitive to the treatment with the molecule of the taxoid family. Hence, an over-expressed gene herein refers to a gene having an increased expression in comparison to the expression level of this gene in a sensitive cell, and an under-expressed gene herein refers to a gene having a decreased expression in comparison to the expression level of this gene in a sensitive cell. However, the man skilled in art understands that other references can be used. For instance, the invention also contemplates a reference level corresponding to the expression level in a cell resistant to the molecule of the taxoid family.

In particular, when the genes selected from Table 1 (and optionally from Table 3) are over-expressed, one can predict that the patient would be resistant to a treatment with a molecule of the taxoid family. On the contrary, when the genes selected from Table 1 (and optionally from Table 3) are not over-expressed, one can predict that the patient would be responsive to a treatment with a molecule of the taxoid family. The same rules applied to the over-expressed genes in Tables 5 and 6 (with a positive fold change (FC)). At the opposite, when the genes selected from Table 2 (and optionally from Table 4) are under-expressed, one can predict that the patient would be resistant to a treatment with a molecule of the taxoid family. On the contrary, when the genes selected from Table 2 (and optionally from Table 4) are not under-expressed, one can predict that the patient would be responsive to a treatment with a molecule of the taxoid family. The same rules applied to the under-expressed genes in Tables 5 and 6 (with a negative fold change (FC)).

In addition, the genes can be selected in such a way that they comprise some over-expressed genes and some under-expressed ones. In this embodiment, the selected genes can comprise at least 5, 6, 7, 8, 9, 10, 15, 20, 25, 30, 40, 50, 60, 70, 80, 90, 100, or 150 genes of Tables 1 and 3 and overexpressed genes of Tables 5 and 6, and at least 5, 6, 7, 8, 9, 10, 15, 20, 25, 30, 40, 50, 60, 70, 80, 90, 100, or 150 genes of Tables 2 and 4 and under-expressed genes of Tables 5 and 6. In a particular embodiment, the genes are selected in Tables 1 and 3 for overexpressed genes and in Tables 2 and 4 for the under-expressed genes. Alternatively, they can be selected in such a way that they comprise only over-expressed or under-expressed genes. In a preferred embodiment, the genes are selected among the genes having the greatest fold change.

In addition to the genes selected from Tables 1 to 6, optionally 1 to 4, the method can also comprise the determination of the expression level for control genes. The control genes are chosen among the genes known to have a constant expression level, in particular between sensitive and resistant cells to a molecule of the taxoid family. In addition, the expression level of at least one control gene is determined in order to normalize the result. For instance, the control gene can be GAPDH, 18S RNA, beta-actine or lamin.

The molecule of the taxoid family refers to a class of anti-tumoral drugs belonging to the taxane family. It can be selected from the group consisting of paclitaxel, docetaxel and analogs, prodrugs or formulations thereof. In particular, analogs, prodrugs or formulations thereof can be for instance selected in the group consisting of larotaxel (also called XRP9881; Sanofi-Aventis), cabazitaxel (XRP6258) (Sanofi-Aventis), BMS-184476 (Bristol-Meyer-Squibb), BMS-188797 (Bristol-Meyer-Squibb), BMS-275183 (Bristol-Meyer-Squibb), ortataxel (also called IDN 5109, BAY 59-8862 or SB-T-101131; Bristol-Meyer-Squibb), RPR 109881A (Bristol-Meyer-Squibb), RPR 116258 (Bristol-Meyer-Squibb), NBT-287 (TAPESTRY), PG-paclitaxel (also called CT-2103, PPX, paclitaxel poliglumex, paclitaxel polyglutamate or Xyotax™), ABRAXANE® (also called Nab-Paclitaxel; ABRAXIS BIOSCIENCE), Tesetaxel (also called DJ-927), IDN 5390 (INDENA), Taxoprexin (also called docosahexanoic acid-paclitaxel; PROTARGA), DHA-paclitaxel (also called Taxoprexin®), and MAC-321 (WYETH). Also see the review of Hennenfent & Govindan (2006, Annals of Oncology, 17, 735-749). In a preferred embodiment of the present invention, the molecule of the taxoid family is the docetaxel.

The expression level of the selected genes can be determined by measuring the amounts of RNA, in particular mRNA, DNA, in particular cDNA, or protein using a variety of techniques well-known by the man skilled in art. In a particular embodiment, the under-expression of a gene can be indirectly assessed through the determination of the methylation status of its promoter. Indeed, a methylated promoter is indicative of an expression repression, and therefore of an under-expression. At the opposite, an unmethylated promoter is indicative of a normal expression. The methylation state of a promoter can be assessed by any method known by the one skilled in the art, for instance by the methods disclosed in the following documents: Frommer et al (Proc Natl Acad Sci USA. 1992; 89:1827-31) and Boyd et al (Anal Biochem. 2004; 326:278-80).

The cancer can be selected from the group consisting of the breast cancer, the lung cancer, the prostate cancer, the gastric cancer and the head and neck cancer. In a preferred embodiment, the cancer is the prostate cancer.

The term “biological sample” means any biological sample derived from a patient, preferably a sample which contains nucleic acids or proteins. Examples of such samples include fluids, tissues, cell samples, organs, biopsies, etc. Most preferred samples are cancer tissue samples, in particular breast, lung, prostate, stomach, ovary or head and neck tumor samples. Blood, plasma, saliva, urine, seminal fluid, etc, may also be used. Cancer cells obtain form blood as circulating tumor cells may also be used. The biological sample may be treated prior to its use, e.g. in order to render nucleic acids or proteins available. Techniques of cell lysis, concentration or dilution of nucleic acids or proteins, are known by the skilled person.

Generally, the expression level as determined is a relative expression level (mRNA or protein).

More preferably, the determination comprises contacting the sample with selective reagents such as probes, primers or ligands, and thereby detecting the presence, or measuring the amount, of proteins or nucleic acids of interest originally in the sample. Contacting may be performed in any suitable device, such as a plate, microtiter dish, test tube, well, glass, column, and so forth. In specific embodiments, the contacting is performed on a substrate coated with the reagent, such as a nucleic acid array or chip or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or proteins of the sample.

In a preferred embodiment, the expression level may be determined by determining the quantity of mRNA.

Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous.

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e.g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate). For instance, the probes and primers can be selected from the Taqman Applied ones cited in the present application.

The nucleic acid primers or probes used herein may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, et 2006)

Other methods for determining the expression level of said genes include the determination of the quantity of proteins encoded by said genes.

Such methods comprise contacting a biological sample with a binding partner capable of selectively interacting with a marker protein present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

The invention further provides a tool for implementing said methods, e.g. a DNA chip comprising a solid support which carries nucleic acids that are specific to at least 5, 6, 7, 8, 9, 10, 15, 20, 25, 30, 40, 50, 60, 70, 80, 90, 100, 150, 200, 300, 500 or 1000 genes selected from the group consisting of the genes listed in Tables 1 to 6, optionally Tables 1 and 2. Optionally, the DNA chip further carries nucleic acids that are specific to at least one gene selected from the group consisting of the genes listed in Tables 3 to 6, optionally Tables 3 and 4. In a preferred embodiment, the DNA chip carries nucleic acids that are specific to genes of Table 6, and optionally of one, several or all genes of Table 5. Optionally, the DNA chip may further include nucleic acids specific of additional genes from Tables 1-4. The DNA chip can further comprise nucleic acids for control gene, for instance a positive and negative control or a nucleic acid for an ubiquitous gene in order to normalize the results. In addition, the present invention also provides a kit for implementing said methods comprising detection means that are specific to at least 5, 6, 7, 8, 9, 10, 15, 20, 25, 30, 40, 50, 60, 70, 80, 90, 100, 150, 200, 300, 500 or 1000 genes selected from the group consisting of the genes listed in Tables 1 to 6, optionally Tables 1 and 2. Optionally, the kit further comprises detection means that are specific to at least one gene selected from the group consisting of the genes listed in Tables 3 to 6, optionally Tables 3 and 4. In a preferred embodiment, the kit carries detection means that are specific to genes of Table 6, and optionally of one, several or all genes of Table 5. Optionally, the kit may further include detection means for additional genes from Tables 1-4. In particular, the detection means can be a pair of primers, a probe or an antibody. The kit can further comprise control reagents and other necessary reagents.

In a particular embodiment, the genes, preferably additional genes are selected for the tool or kit as above detailed for the methods of the invention. Preferably, the at least 5 genes, preferably additional genes, are selected from the group consisting of ENST00000399723, BI836406, C10orf79, AK022962, TMTC1, LOC728295, SUSD5, WNT6, BC044624, AY358241, ZNF251, ST6GAL2, LOC643401, NOV, CLGN, PROM1, SPEF2, FLRT2, RGS2, FOXP2, TRIM55, PKD2L1, RP4-692D3.1, CB985069, ARL14, AY831680, XRN1, THAP5, ZNF248, BC016022, PLAG1, THC2724353, THC2488083, C5orf41, BMS1P5, BMS1, THC2627008, PLA2G4A, DPY19L2, VCX2, PPP1R1C, GLT25D2, KIAA1841, IFIT2, ZNF596, TSPAN19, BC029907, C10orf107, ZNF594, AMPD1, C21orf88, THC2694827, HSPC105, IFI44, THC2662262, FAM84A, DNAH7, KHDRBS2, NANP, AK091357, N4BP2L1, FAM105A, CA941346, CCDC68, CASC1, FAM90A12, PBX1, THC2739159, KCNQ2, ANXA1, AL122040, THC2655194, ENST00000342608, DSC2, ENOX1, IL13, BG571904, BX455216, LOC729085, BG188151, LOC729409, C1orf103, PPP1R14C, NAIP, C13orf31, GOLGA8E, AK022848, CXorf22, KIF5C, LRRCC1, FAM81B, ID2, CMYA5, C1orf194, TTC18, tcag7.1314, ZNF385B, ADAMTS6, RHOU, ENST00000378850, C2orf55, GPR83, LRRIQ1, WDR31, DEFB126, ARMETL1, LOC642826, LOC129881, C2orf13, THC2553512, ACVR1C, ZNF207, ANTXR1, CHD9, THC2526838, ABCA12, TncRNA, FKTN, PTPRG, ZNF233, ENST00000370378, FANK1, PCM1, SERPINI1, ARID4B, KIAA1377, FGF7, CV339166, LINCR, DA834198, CFH, SCG2, ARHGEF10, DA093175, GOLGA8A, AK021467, LOC283666, FLJ35767, THC2725553, ZNF430, CCDC141, MAP3K13, CCDC66, THC2727226, THC2528990, THC2718728, THC2507829, AK123972, EDEM3, DB304731, TPD52L1, MFAP5, EHF, NCF2, TRIM6, PERLD1, ATXN1, INHBB, CR627122, JAM3, CXCL14, CR594735, FLJ11235, C15orf52, LIMCH1, LOH11CR2A, BX281122, GPR110, ARNT2, ATP6V0A4, PDGFRB, ELA3B, NEDD9, MYH6, SLC35F2, HAS3, COLEC12, SLC3A2, AW993939, RUNX2, SUSD3, PLAU, SLC22A3, FCRL4, DOCK2, SOX3, THC2616558, RNASET2, LOC100130360, IL1R2, MGAT5B, TCF7L1, AF222857, AHNAK, HOXB8, S100A16, INSIG1 and DCDC2. More preferably, the genes are selected from the group consisting of ENST00000399723, BI836406, C10orf79, AK022962, TMTC1, LOC728295, SUSD5, WNT6, BC044624, AY358241, ZNF251, ST6GAL2, LOC643401, NOV, CLGN, PROM1, SPEF2, FLRT2, RGS2, FOXP2, TRIM55, PKD2L1, RP4-692D3.1, CB985069, ARL14, AY831680, XRN1, THAP5, ZNF248, BC016022, PLAG1, THC2724353, THC2488083, C5orf41, BMS1P5, BMS1, THC2627008, PLA2G4A, DPY19L2, VCX2, PPP1R1C, GLT25D2, KIAA1841, IFIT2, ZNF596, TSPAN19, BC029907, C10orf107, ZNF594, AMPD1, C21orf88, THC2694827, HSPC105, IFI44, THC2662262, FAM84A, DNAH7, KHDRBS2, NANP, AK091357, N4BP2L1, FAM105A, CA941346, CCDC68, CASC1, FAM90A12, PBX1, THC2739159, KCNQ2, ANXA1, AL122040, THC2655194, ENST00000342608, DSC2, ENOX1, IL13, BG571904, BX455216, LOC729085, BG188151, LOC729409, C1orf103, PPP1R14C, NAIP, C13orf31, GOLGA8E, AK022848, CXorf22, KIF5C, TPD52L1, MFAP5, EHF, NCF2, TRIM6, PERLD1, ATXN1, INHBB, CR627122, JAM3, CXCL14, CR594735, FLJ11235, C15orf52, LIMCH1, LOH11CR2A, BX281122, GPR110, ARNT2, ATP6V0A4, PDGFRB, ELA3B, NEDD9, MYH6, SLC35F2, HAS3, COLEC12, SLC3A2, AW993939, RUNX2 and SUSD3. Even more preferably, the genes are selected from the group consisting of ENST00000399723, BI836406, C10orf79, AK022962, TMTC1, LOC728295, SUSD5, WNT6, BC044624, AY358241, ZNF251, ST6GAL2, LOC643401, NOV, CLGN, PROM1, SPEF2, FLRT2, RGS2, FOXP2, TRIM55, PKD2L1, RP4-692D3.1, TPD52L1, MFAP5, EHF, NCF2, TRIM6, PERLD1, ATXN1, INHBB, CR627122, JAM3, CXCL14 and CR594735. In the most preferred embodiment, the genes are selected from the group consisting of ENST00000399723, BI836406, C10orf79, AK022962, TMTC1, LOC728295, SUSD5, WNT6, BC044624, TPD52L1, MFAP5, EHF, NCF2, TRIM6, PERLD1, ATXN1, INHBB and CR627122. Optionally, at least one further gene is selected for the tool or kit, said gene being selected from the group consisting of the genes listed in Tables 3 and 4, preferably TFPI2, PCDH7, SMAD9, AK090762, RAB39B, BF831953, AL050204, VCX, ITGA2, CXCR4, SLC16A10, PDE1A, MAL, KRT80, FXYD2 and AK3L1, more preferably TFPI2, PCDH7, SMAD9, AK090762, RAB39B, BF831953, AL050204, VCX, CXCR4, SLC16A10, PDE1A, MAL, and even more preferably TFPI2, PCDH7, SMAD9, CXCR4 and SLC16A10.

The present invention also relates to the use of a DNA chip or a kit of the invention for preparing a kit for predicting or monitoring whether a patient affected by a cancer is responsive to a treatment with a molecule of the taxoid family. Preferably, the cancer is selected from the group consisting of the breast cancer, the lung cancer, the prostate cancer, the gastric cancer and the head and neck cancer. More preferably the cancer is the prostate cancer. In a preferred embodiment, the molecule of the taxoid family is selected from the group consisting of docetaxel, larotaxel, cabazitaxel (XRP6258), BMS-184476, BMS-188797, BMS-275183, ortataxel, RPR 109881A, RPR 116258, NBT-287, PG-paclitaxel, ABRAXANE®, Tesetaxel, IDN 5390, Taxoprexin, DHA-paclitaxel, and MAC-321. More preferably, the molecule of the taxoid family is docetaxel.

The present invention further concerns methods for screening or identifying a compound suitable for improving the treatment of a cancer with a molecule of the taxoid family or for reducing the resistance development during the treatment of a cancer with a molecule of the taxoid family.

In a first embodiment, the method comprises: 1) providing a cell-line with at least 5, 6, 7, 8, 9, 10, 15, 20, 25, 30, 40, 50, 60, 70, 80, 90, 100, 150, 200, 300, 500 or 1000 genes over-expressed and/or under-expressed respectively selected from the group of over-expressed genes of Tables 1, 3 and 5, optionally of Table 1, and under-expressed genes of Tables 2, 4 and 5, optionally of Table 2; 2) contacting said cell-line with a test compound; 3) determining the expression level of said at least 5, 6, 7, 8, 9, 10, 15, 20, 25, 30, 40, 50, 60, 70, 80, 90, 100, 150, 200, 300, 500 or 1000 genes; and, 4) selecting the compound which decreases the expression level of over-expressed genes and increases the expression level of under-expressed genes. More preferably, the genes are selected from the genes of Tables 5 and 6. Still more preferably, at least the genes of Table 6 are selected, and optionally one, several or all genes of Table 5.

In a second embodiment, the method comprises: 1) providing a cell-line sensitive to the molecule of the taxoid family; 2) contacting said cell-line with a test compound and the molecule of the taxoid family; 3) determining the expression level of said at least 5, 6, 7, 8, 9, 10, 15, 20, 25, 30, 40, 50, 60, 70, 80, 90, 100, 150, 200, 300, 500 or 1000 genes selected from the genes listed in Tables 1 to 6, optionally of Tables 1 and 2; and, 4) selecting the compound which inhibits the appearance of an over-expression and/or an under-expression of at least 5, 6, 7, 8, 9, 10, 15, 20, 25, 30, 40, 50, 60, 70, 80, 90, 100, 150, 200, 300, 500 or 1000 genes respectively selected from the group of genes of over-expressed genes of Tables 1, 3 and 5, optionally of Table 1, and under-expressed genes of Tables 2, 4 and 5, optionally of Table 2. More preferably, the genes are selected from the genes of Tables 5 and 6. Still more preferably, at least the genes of Table 6 are selected, and optionally one, several or all genes of Table 5.

In a third embodiment, the method comprises: 1) providing a cell-line with at least one gene over-expressed and/or under-expressed respectively selected from the group consisting of ENST00000399723, BI836406, C10orf79, AK022962, TMTC1, LOC728295, SUSD5, WNT6, BC044624, AY358241, ZNF251, ST6GAL2, LOC643401, NOV, CLGN, PROM1, SPEF2, FLRT2, RGS2, FOXP2, TRIM55, PKD2L1, RP4-692D3.1, CB985069, ARL14, AY831680, XRN1, THAP5, ZNF248, BC016022, PLAG1, THC2724353, THC2488083, C5orf41, BMS1P5, BMS1, THC2627008, PLA2G4A, DPY19L2, VCX2, PPP1R1C, GLT25D2, KIAA1841, IFIT2, ZNF596, TSPAN19, BC029907, C10orf107, ZNF594, AMPD1, C21orf88, THC2694827, HSPC105, IFI44, THC2662262, FAM84A, DNAH7, KHDRBS2, NANP, AK091357, N4BP2L1, FAM105A, CA941346, CCDC68, CASC1, FAM90A12, PBX1, THC2739159, KCNQ2, ANXA1, AL122040, THC2655194, ENST00000342608, DSC2, ENOX1, IL13, BG571904, BX455216, LOC729085, BG188151, LOC729409, C1orf103, PPP1R14C, NAIP, C13orf31, GOLGA8E, AK022848, CXorf22, KIF5C, LRRCC1, FAM81B, ID2, CMYA5, C1orf194, TTC18, tcag7.1314, ZNF385B, ADAMTS6, RHOU, ENST00000378850, C2orf55, GPR83, LRRIQ1, WDR31, DEFB126, ARMETL1, LOC642826, LOC129881, C2orf13, THC2553512, ACVR1C, ZNF207, ANTXR1, CHD9, THC2526838, ABCA12, TncRNA, FKTN, PTPRG, ZNF233, ENST00000370378, FANK1, PCM1, SERPINI1, ARID4B, KIAA1377, FGF7, CV339166, LINCR, DA834198, CFH, SCG2, ARHGEF10, DA093175, GOLGA8A, AK021467, LOC283666, FLJ35767, THC2725553, ZNF430, CCDC141, MAP3K13, CCDC66, THC2727226, THC2528990, THC2718728, THC2507829, AK123972, EDEM3, DB304731, preferably ENST00000399723, BI836406, C10orf79, AK022962, TMTC1, LOC728295, SUSD5, WNT6, BC044624, AY358241, ZNF251, ST6GAL2, LOC643401, NOV, CLGN, PROM1, SPEF2, FLRT2, RGS2, FOXP2, TRIM55, PKD2L1, RP4-692D3.1, CB985069, ARL14, AY831680, XRN1, THAP5, ZNF248, BC016022, PLAG1, THC2724353, THC2488083, C5orf41, BMS1P5, BMS1, THC2627008, PLA2G4A, DPY19L2, VCX2, PPP1R1C, GLT25D2, KIAA1841, IFIT2, ZNF596, TSPAN19, BC029907, C10orf107, ZNF594, AMPD1, C21orf88, THC2694827, HSPC105, IFI44, THC2662262, FAM84A, DNAH7, KHDRBS2, NANP, AK091357, N4BP2L1, FAM105A, CA941346, CCDC68, CASC1, FAM90A12, PBX1, THC2739159, KCNQ2, ANXA1, AL122040, THC2655194, ENST00000342608, DSC2, ENOX1, IL13, BG571904, BX455216, LOC729085, BG188151, LOC729409, C1orf103, PPP1R14C, NAIP, C13orf31, GOLGA8E, AK022848, CXorf22 and KIF5C, more preferably ENST00000399723, BI836406, C10orf79, AK022962, TMTC1, LOC728295, SUSD5, WNT6, BC044624, AY358241, ZNF251, ST6GAL2, LOC643401, NOV, CLGN, PROM1, SPEF2, FLRT2, RGS2, FOXP2, TRIM55, PKD2L1 and RP4-692D3.1, even more preferably ENST00000399723, BI836406, C10orf79, AK022962, TMTC1, LOC728295, SUSD5, WNT6 and BC044624 for the over-expressed genes, and TPD52L1, MFAP5, EHF, NCF2, TRIM6, PERLD1, ATXN1, INHBB, CR627122, JAM3, CXCL14, CR594735, FLJ11235, C15orf52, LIMCH1, LOH11CR2A, BX281122, GPR110, ARNT2, ATP6V0A4, PDGFRB, ELA3B, NEDD9, MYH6, SLC35F2, HAS3, COLEC12, SLC3A2, AW993939, RUNX2, SUSD3, PLAU, SLC22A3, FCRL4, DOCK2, SOX3, THC2616558, RNASET2, LOC100130360, IL1R2, MGAT5B, TCF7L1, AF222857, AHNAK, HOXB8, S100A16, INSIG1 and DCDC2, preferably TPD52L1, MFAP5, EHF, NCF2, TRIM6, PERLD1, ATXN1, INHBB, CR627122, JAM3, CXCL14, CR594735, FLJ11235, C15orf52, LIMCH1, LOH11CR2A, BX281122, GPR110, ARNT2, ATP6V0A4, PDGFRB, ELA3B, NEDD9, MYH6, SLC35F2, HAS3, COLEC12, SLC3A2, AW993939, RUNX2 and SUSD3, more preferably TPD52L1, MFAP5, EHF, NCF2, TRIM6, PERLD1, ATXN1, INHBB, CR627122, JAM3, CXCL14 and CR594735, even more preferably TPD52L1, MFAP5, EHF, NCF2, TRIM6, PERLD1, ATXN1, INHBB and CR627122 for the under-expressed genes; 2) contacting said cell-line with a test compound; 3) determining the expression level of said at least one gene; and, 4) selecting the compound which decreases the expression level of over-expressed genes and increases the expression level of under-expressed genes.

In a fourth embodiment, the method comprises 1) providing a cell-line with the genes PCDH7, KHDRBS2, AUTS2, and C2orf55 being over-expressed and the genes JAM3, DCDC2, MFAP5, SLC3A1, AKAP12, ZNF649, RNASET2, NCF2, DLC1, CXCR4, CR594735, TRIM6, and MBNL3 being under-expressed; 2) contacting said cell-line with a test compound; 3) determining the expression level of said genes; and, 4) selecting the compound which decreases the expression level of one or several of the over-expressed genes and increases the expression level of one or several of the under-expressed genes.

In a fifth embodiment, the method comprises 1) providing a cell-line sensitive to the molecule of the taxoid family; 2) contacting said cell-line with a test compound and the molecule of the taxoid family; 3) determining the expression level of the genes JAM3, PCDH7, DCDC2, KHDRBS2, MFAP5, AUTS2, C2orf55, SLC3A1, AKAP12, ZNF649, RNASET2, NCF2, DLC1, CXCR4, CR594735, TRIM6, and MBNL3; and, 4) selecting the compound which inhibits the appearance of an over-expression of the genes PCDH7, KHDRBS2, AUTS2, and C2orf55 and/or an under-expression of the genes JAM3, DCDC2, MFAP5, SLC3A1, AKAP12, ZNF649, RNASET2, NCF2, DLC1, CXCR4, CR594735, TRIM6, and MBNL3.

Preferably, the cell-line is a cancer cell-line. In particular, the cancer cell-line is specific of the targeted cancer. For instance, if the prostate cancer is to be treated, then the cell-line is a prostate cancer cell-line.

In a preferred embodiment, the molecule of the taxoid family is selected from the group consisting of docetaxel, larotaxel, cabazitaxel (XRP6258), BMS-184476, BMS-188797, BMS-275183, ortataxel, RPR 109881A, RPR 116258, NBT-287, PG-paclitaxel, ABRAXANE®, Tesetaxel, IDN 5390, Taxoprexin, DHA-paclitaxel, and MAC-321. More preferably, the molecule of the taxoid family is docetaxel. Preferably, the cancer is selected from the group consisting of the breast cancer, the lung cancer, the prostate cancer, the gastric cancer and the head and neck cancer. More preferably the cancer is the prostate cancer.

The example illustrates the invention without limiting its scope.

### Example

Methods

Cell Culture and Selection of Docetaxel-Resistant Clones

The human androgen-independent IGR-CaP1 cell line recently obtained for a localized prostate cancer was maintained in RPMI medium complemented with 10% FBS and antibiotics. Docetaxel-resistant clones were selected by culturing the cells in docetaxel in a dose-escalation manner. Initial culture was done in 5 nM docetaxel. Cellular clones surviving in the presence of 5 nM docetaxel were maintained in culture during four passages, and then the concentration of docetaxel in the medium was increased to 12 nM, 25 nM, 50 nM, 100 nM and 200 nM. The same selection methodology was followed with each increase in docetaxel concentration. Once cells were freely dividing in each dose of docetaxel mediums, they were considered as resistant and labelled IGR-CaP1-R. Cell cultures were maintained at 70% confluency and medium was changed every 48 h.

Total RNA Preparation and Reverse Transcription

Total RNA from parental and docetaxel-resistant IGR-CaP1 cells was isolated using TriReagent (Sigma-Aldrich) and purified with RNeasy Micro Kit (Qiagen) according to manufacturer's protocols. Quality of RNA preparation, based on the RNA Integrity Number (RIN), was assessed using the Agilent RNA 6000 Nano Kit as developed on the Agilent 2100 Bioanalyzer device (Agilent Technologies, Palo Alto, Calif.). All specimens included in this study displayed a RIN of 10. RNA samples were frozen in nuclease-free water (Qiagen).

Oligo Microarray Technology

Parental and resistant-cell line total RNAs were directly compared by using Agilent oligonucleotide dual-color technology, running dye-swap and duplicate experiments. Total RNA from the parental IGR-CaP1 cell line without treatment was used as the RNA reference. Total RNA from IGR-CaP1 cells resistant to treatment with 5 nM, 12 nM, 25 nM, 50 nM, 100 nM and 200 nM of docetaxel respectively, were used as samples. Probe synthesis and labeling were performed by Agilent's Low Fluorescent Low input Linear Amplification Kit. Hybridization was performed on the Agilent 4×44K Human 1A (G4112F) long (60-bp) oligonucleotide microarrays (Agilent Technologies) by using reagents and protocols provided by the manufacturer. Feature extraction software provided by Agilent (Version A.9.5.3.1) was used to quantify the intensity of fluorescent images and to normalize results using the linear and lowess subtraction method.

The methodology described below is based on a dose-dependent gene expression changes:

Under the hypothesis of a clone enrichment, and/or a biological effect due to drug increasing, monotonically increasing or decreasing expression profiles were identified by using a 5-parameters logistic regression model:

$${y}_{g} = B + \frac{( T - B )}{{[ 1 + {10}^{{( {x}_{c} - x )}^{*} s} ]}^{p}}$$

where y_{g} is the log.ratio of treatment vs. reference for the gene g, x is the drug-dose in Log₁₀[nM], and B, T, x_{c}, p are, respectively, the estimated minimal value, the estimated maximal value, the slope at the inflexion point, and the asymmetric parameter.
For each probe, parameters were first initialized with the observed values, and then optimized by an iterative method of gradient (the Newton-Raphson method). The aim of this iterative algorithm is to minimize the weighted quadratic sum of residuals:

$$S = {\sum }_{i} {{w}_{i} ( {y}_{i} \cdot fit - {y}_{i} \cdot obs )}^{2}$$

$$where {w}_{i} = \frac{1}{\vert {y}_{i} \cdot fit - {y}_{i} \cdot obs \vert}$$

The performance of the fitting was measured, for each probe, by a robust linear regression (RLR) of the fitted values against the observed values.
Probes potentially associated with the drug increasing were selected on the 2 following criterion:

RLRp-value ≦1e-5, and |fold change|≧2 between the first and the last dose (resp: 5 and 200 nM), considering the fold change estimated by the 5-parameters logistic regression model. Calculations and graphic visualizations were performed in R (free software version 2.6.2), by using the package “MASS” (version 7.2-40), and supplemental scripts, in R language, written in the lab (F. Commo).

Results

Generation of Acquired Resistance to Docetaxel In Vitro.

Prostate cancer IGR-CaP1 cells were used to generate successive docetaxel-resistant cell lines. The addition of docetaxel induced a selection process, whereby a large majority of cells initially underwent cell death until the ability to proliferate was regained. The inventors obtained IGR-CaP1 resistant (IGR-CaP1-R) clones which survived in medium containing respectively 5 nM, 12 nM, 25 nM, 50 nM, 100 nM and 200 nM of docetaxel. Cell cycle analysis was done to show acquired resistance to drug. The resistant cell lines showed cell cycle similar to the parental IGR-CaP1 cells, suggesting that acquired resistance had been gained (not shown).

Genome-Wide Analysis of IGR-CaP1 Docetaxel-Resistant Lines Using Microarray.

Human genome-wide analysis of gene expression changes was realized in order to stringently identify human genes that might represent the molecular signature of resistance or sensitivity to docetaxel in prostate cancer. Untreated IGR-CaP1 parental cell lines were used as baseline. Such genes were those for which expression changes (at least one probe in case of multiple probe sets per gene) appeared as drug-dependent, in the sense of criterion described above.

In the first analysis, 772 genes were over-expressed (Tables 1 and 3) and 309 were down-regulated (Tables 2 and 4) in docetaxel-resistant cells. These genes were sorted out by the mean of the fold change observed between the first and the last doses of docetaxel (between 5 and 200 nM).

A second analysis was performed from biological duplicates to confirm the first data set. In the second analysis, only the irreversible resistance mechanisms were retained by using resistant cells cultured in the absence of drug during two passages before the microarray analysis. The second analysis generated a list of 486 genes in which 44 genes were already observed in the first analysis. In the list of 44 genes commons in the two analyses, 17 genes were over-expressed and 27 genes were down-regulated in docetaxel-resistance cells (Table 5). These genes were sorted out by the mean of the fold change observed between the first and the last doses of docetaxel (between 5 and 200 nM).

Among these genes, a subset of 17 genes was selected containing 4 over-expressed genes and 13 under-expressed genes in docetaxel-resistance cells (Table 6). This set of genes has been selected by the following method.

TABLE 1
First list of the over-expressed genes

| | Genbank | | | 5 | 12 | 25 | 50 | 100 | 200 | Fold | |
|---|---|---|---|---|---|---|---|---|---|---|---|
| Gene Symbol | Accessions | UniGeneID | Cytoband | nM | nM | nM | nM | nM | nM | Change | p.value |
| ENST00000399723 | AK090412 | Hs.656011 | hs\|1q12 | −0.532 | 0.134 | −0.173 | −0.222 | 0.451 | 0.773 | 20.197 | 2.20E−06 |
| BI836406 | BI836406 | Hs.130203 | hs\|4q22.1 | −0.285 | 0.130 | 0.601 | 0.857 | 1.070 | 0.984 | 18.566 | 1.61E−10 |
| C10orf79 | NM_025145 | Hs.288927 | hs\|10q25.1 | −0.498 | 0.030 | 0.049 | 0.449 | 0.347 | 0.766 | 18.347 | 5.60E−04 |
| AK022962 | AK022962 | Hs.654412 | hs\|1q23.3 | 0.000 | 0.046 | 0.780 | 1.186 | 1.188 | 1.118 | 15.444 | 2.64E−12 |
| TMTC1 | NM_175861 | Hs.401954 | hs\|12p11.22 | −0.321 | −0.077 | 0.643 | 0.839 | 0.981 | 0.820 | 15.296 | 7.16E−12 |
| LOC728295 | XR_015377 | Hs.636711 | hs\|1q12 | −0.490 | 0.017 | −0.233 | −0.459 | 0.354 | 0.680 | 14.803 | 8.14E−08 |
| SUSD5 | AB011099 | Hs.196647 | hs\|3p22.3 | −0.568 | −0.260 | −0.364 | −0.179 | 0.398 | 0.554 | 13.302 | 7.36E−04 |
| WNT6 | NM_006522 | Hs.29764 | hs\|2q35 | 0.165 | 0.593 | 0.914 | 1.045 | 1.170 | 1.304 | 12.542 | 1.08E−05 |
| BC044624 | BC044624 | Hs.654412 | hs\|1q23.3 | 0.325 | 0.756 | 1.134 | 1.226 | 1.389 | 1.334 | 10.428 | 5.01E−05 |
| AY358241 | AY358241 | Hs.626042 | hs\|12q23.3 | −0.170 | 0.637 | 0.980 | 0.826 | 0.827 | 0.876 | 9.965 | 7.40E−07 |
| ZNF251 | BC006258 | Hs.534516 | hs\|8q24.3 | −0.365 | 0.089 | 0.042 | 0.278 | 0.077 | 0.624 | 9.730 | 2.11E−05 |
| ST6GAL2 | AB058780 | Hs.98265 | hs\|2q12.3 | −0.599 | −0.541 | −0.040 | −0.067 | 0.403 | 0.393 | 9.586 | 7.95E−05 |
| LOC643401 | BC039509 | Hs.533212 | hs\|5p14.1 | 0.111 | 0.333 | 0.137 | 0.165 | 0.981 | 1.118 | 9.578 | 6.61E−14 |
| NOV | NM_002514 | Hs.235935 | hs\|8q24.12 | −0.304 | 0.024 | 0.568 | 0.436 | 0.590 | 0.677 | 9.304 | 2.21E−05 |
| CLGN | NM_004362 | Hs.86368 | hs\|4q31.1 | −0.214 | 0.064 | 0.256 | 0.241 | 0.765 | 0.743 | 9.045 | 7.64E−04 |
| PROM1 | NM_006017 | Hs.614734 | hs\|4p15.32 | −0.640 | −0.574 | 0.286 | 0.333 | 0.225 | 0.311 | 8.952 | 2.30E−15 |
| SPEF2 | NM_024867 | Hs.298863 | hs\|5p13.2 | −0.304 | 0.000 | 0.156 | 0.337 | 0.551 | 0.644 | 8.861 | 4.78E−05 |
| FLRT2 | NM_013231 | Hs.533710 | hs\|14q31.3 | −0.690 | −0.382 | 0.040 | 0.180 | 0.113 | 0.284 | 8.564 | 3.90E−05 |
| RGS2 | NM_002923 | Hs.78944 | hs\|1q31.2 | −0.032 | 0.048 | 0.417 | 0.368 | 0.723 | 0.893 | 8.426 | 2.12E−04 |
| FOXP2 | | | hs\|7q31.1 | 0.063 | 0.265 | 0.717 | 0.781 | 0.546 | 0.987 | 8.291 | 7.83E−06 |
| TRIM55 | NM_184086 | Hs.85524 | hs\|8q13.1 | −0.382 | −0.104 | 0.582 | 0.576 | 0.147 | 0.523 | 8.181 | 5.17E−04 |
| PKD2L1 | NM_033215 | Hs.433652 | hs\|Xp11.23 | 0.353 | 0.056 | 0.071 | 0.409 | 0.461 | 0.965 | 8.134 | 1.80E−05 |
| RP4-692D3.1 | NM_001080850 | Hs.473495 | hs\|1p34.2 | 0.235 | 0.481 | 0.692 | 0.649 | 0.892 | 1.272 | 8.036 | 4.13E−04 |
| CB985069 | | | hs\|4q22.1 | −0.976 | −0.365 | −0.860 | −0.627 | −0.356 | −0.077 | 7.853 | 4.41E−06 |
| ARL14 | NM_025047 | Hs.287702 | hs\|3q26.1 | −0.437 | −0.545 | −0.408 | −0.525 | −0.009 | 0.452 | 7.749 | 3.74E−04 |
| AY831680 | AY831680 | Hs.526752 | hs\|3q13.12 | 0.181 | 0.386 | 0.913 | 1.091 | 1.033 | 1.058 | 7.541 | 6.17E−10 |
| XRN1 | NM_019001 | Hs.435103 | hs\|3q23 | 0.005 | 0.078 | 0.084 | 0.109 | 0.318 | 0.379 | 7.483 | 2.62E−12 |
| THAP5 | NM_182529 | Hs.650237 | hs\|7q31.1 | 0.103 | 0.133 | 0.117 | 0.131 | 0.387 | 0.499 | 7.368 | 3.83E−05 |
| ZNF248 | NM_021045 | Hs.572001 | hs\|10p11.21 | −0.123 | 0.326 | −0.062 | −0.037 | 0.391 | 0.742 | 7.332 | 8.55E−04 |
| BC016022 | BC016022 | Hs.679496 | | −0.098 | 0.398 | 0.160 | 0.227 | 0.590 | 0.767 | 7.315 | 3.75E−04 |
| PLAG1 | NM_002655 | Hs.14968 | hs\|8q12.1 | −0.353 | −0.429 | −0.074 | −0.175 | 0.268 | 0.505 | 7.207 | 7.04E−04 |
| THC2724353 | | | hs\|15q11.2 | 0.186 | 0.317 | 0.216 | 0.357 | 0.696 | 1.032 | 7.024 | 5.20E−05 |
| THC2488083 | | | hs\|17p11.2 | −0.038 | 0.000 | −0.011 | −0.005 | 0.514 | 0.829 | 6.932 | 8.84E−08 |
| C5orf41 | NM_153607 | Hs.484195 | hs\|5q35.2 | 0.059 | 0.425 | 0.254 | 0.416 | 0.618 | 1.010 | 6.865 | 1.02E−04 |
| BMS1P5 | AL833330 | Hs.652959 | hs\|10q11.22 | −0.013 | 0.270 | 0.525 | 0.675 | 0.793 | 0.824 | 6.850 | 4.34E−08 |
| BMS1 | NM_014753 | Hs.10848 | hs\|10q11.21 | −0.043 | 0.276 | 0.457 | 0.656 | 0.801 | 0.654 | 6.701 | 1.04E−04 |
| THC2627008 | | | hs\|4q24 | −0.500 | −0.162 | −0.038 | 0.227 | 0.267 | 0.324 | 6.668 | 1.34E−05 |
| PLA2G4A | NM_024420 | Hs.497200 | hs\|1q31.1 | −0.526 | −0.241 | −0.434 | −0.267 | 0.158 | 0.375 | 6.580 | 8.10E−12 |
| DPY19L2 | NM_173812 | Hs.533644 | hs\|12q14.2 | 0.157 | 0.309 | 0.303 | 0.659 | 0.708 | 0.787 | 6.543 | 3.18E−05 |
| VCX2 | NM_016378 | Hs.279737 | hs\|Xp22.31 | 0.421 | 0.441 | 0.602 | 0.451 | 0.877 | 1.223 | 6.329 | 1.77E−07 |
| PPP1R1C | NM_001080545 | Hs.10941 | hs\|2q31.3 | −0.431 | −0.367 | −0.349 | −0.704 | 0.096 | 0.364 | 6.244 | 3.36E−05 |
| GLT25D2 | NM_015101 | Hs.387995 | hs\|1q25.3 | −0.557 | −0.383 | 0.233 | 0.284 | 0.112 | 0.143 | 6.207 | 1.85E−04 |
| KIAA1841 | BC039298 | Hs.468653 | hs\|2p15 | −0.347 | 0.132 | −0.135 | −0.012 | 0.311 | 0.445 | 6.191 | 6.70E−04 |
| IFIT2 | NM_001547 | Hs.437609 | hs\|10q23.31 | 0.181 | 0.194 | 0.031 | 0.613 | 0.586 | 0.974 | 6.185 | 6.76E−05 |
| ZNF596 | NM_173539 | Hs.591388 | hs\|8p23.3 | −0.058 | 0.000 | 0.066 | −0.051 | 0.419 | 0.781 | 6.175 | 1.94E−04 |
| TSPAN19 | NM_001924 | Hs.80409 | hs\|1p31.3 | 0.000 | 0.068 | 0.405 | 0.491 | 0.607 | 0.789 | 6.155 | 1.68E−04 |
| BC029907 | BC029907 | Hs.405427 | hs\|1p22.1 | 0.022 | 0.174 | 0.251 | 0.478 | 0.628 | 0.811 | 6.131 | 5.50E−06 |
| C10orf107 | NM_173554 | Hs.673160 | hs\|10q21.2 | 0.508 | 0.660 | 1.258 | 1.312 | 1.232 | 1.294 | 6.106 | 3.87E−12 |
| ZNF594 | AB058774 | Hs.658402 | hs\|17p13.2 | −0.050 | 0.212 | −0.099 | −0.037 | 0.391 | 0.732 | 6.100 | 4.14E−06 |
| AMPD1 | NM_000036 | Hs.89570 | hs\|1p13.2 | −0.338 | 0.000 | −0.180 | −0.261 | 0.060 | 0.668 | 6.082 | 9.22E−04 |
| C21orf88 | BC080530 | Hs.375120 | hs\|21q22.2 | 0.077 | 0.271 | 0.783 | 0.940 | 0.855 | 0.642 | 6.007 | 3.23E−12 |
| THC2694827 | | | hs\|Xq22.1 | −0.035 | 0.284 | −0.078 | 0.202 | 0.440 | 0.742 | 5.999 | 1.56E−05 |
| HSPC105 | NM_145168 | Hs.87779 | hs\|16q23.3 | 0.000 | −0.008 | −0.421 | 0.000 | 0.478 | 0.764 | 5.976 | 4.68E−05 |
| IFI44 | NM_006417 | Hs.82316 | hs\|1p31.1 | −0.039 | 0.217 | 0.475 | 0.248 | 0.686 | 0.735 | 5.948 | 2.49E−06 |
| THC2662262 | | | hs\|14q32.32 | −0.646 | −0.243 | −0.458 | −0.263 | −0.178 | 0.130 | 5.940 | 9.66E−04 |
| FAM84A | NM_145175 | Hs.260855 | hs\|2p24.3 | 0.485 | 0.175 | 0.418 | 0.711 | 1.062 | 1.146 | 5.923 | 2.19E−09 |
| DNAH7 | NM_018897 | Hs.97403 | hs\|2q32.3 | −0.145 | 0.079 | 0.243 | 0.356 | 0.373 | 0.653 | 5.856 | 9.23E−05 |
| KHDRBS2 | NM_152688 | Hs.519794 | hs\|6q11.1 | −0.433 | −0.171 | 0.316 | 0.587 | 0.210 | 0.330 | 5.791 | 8.68E−12 |
| NANP | AK074335 | Hs.666255 | hs\|20p11.21 | −0.507 | −0.097 | −0.185 | 0.083 | 0.237 | 0.253 | 5.704 | 3.94E−04 |
| AK091357 | BC036917 | Hs.485528 | hs\|6p12.3 | 0.149 | 0.006 | 0.093 | 0.072 | 0.567 | 0.822 | 5.626 | 4.35E−05 |
| N4BP2L1 | NM_052818 | Hs.161220 | hs\|13q13.1 | 0.041 | 0.204 | 0.110 | 0.144 | 0.492 | 0.795 | 5.548 | 5.93E−05 |
| FAM105A | NM_019018 | Hs.591751 | hs\|5p15.2 | −0.404 | −0.095 | −0.078 | −0.209 | 0.201 | 0.352 | 5.537 | 2.43E−04 |
| CA941346 | CA941346 | | hs\|15q11.2 | −0.666 | 0.120 | −0.415 | −0.207 | −0.126 | 0.110 | 5.522 | 3.90E−04 |
| CCDC68 | NM_025214 | Hs.120790 | hs\|18q21.2 | −0.069 | 0.000 | 0.248 | 0.445 | 0.446 | 0.668 | 5.448 | 1.72E−08 |
| CASC1 | NM_018272 | Hs.407771 | hs\|12p12.1 | 0.073 | 0.384 | 0.774 | 1.070 | 0.808 | 0.760 | 5.439 | 2.61E−07 |
| FAM90A12 | XM_496957 | Hs.694406 | hs\|8p23.1 | 0.277 | 0.349 | 0.420 | 0.186 | 0.718 | 1.013 | 5.435 | 2.07E−04 |
| PBX1 | NM_002585 | Hs.654412 | hs\|1q23.3 | 0.099 | 0.250 | 0.602 | 0.979 | 0.827 | 0.824 | 5.428 | 4.16E−07 |
| THC2739159 | | | hs\|9q12 | −0.115 | 0.034 | 0.573 | 0.587 | 0.482 | 0.574 | 5.381 | 8.89E−05 |
| KCNQ2 | NM_172109 | Hs.161851 | hs\|20q13.33 | −0.345 | −0.015 | 0.466 | 0.327 | 0.341 | 0.385 | 5.348 | 2.52E−06 |
| ANXA1 | NM_000700 | Hs.494173 | hs\|9q21.13 | 0.130 | 0.078 | 0.350 | 0.398 | 0.713 | 0.856 | 5.329 | 4.80E−04 |
| AL122040 | AL122040 | Hs.594784 | hs\|15q21.2 | 0.168 | 0.000 | 0.389 | 0.518 | 0.805 | 0.894 | 5.326 | 6.86E−04 |
| THC2655194 | | | hs\|8q11.23 | −0.122 | 0.320 | 0.129 | 0.290 | 0.291 | 0.604 | 5.321 | 2.72E−05 |
| ENST00000342608 | NM_001013675 | Hs.291198 | hs\|22q11.21 | 0.031 | 0.000 | 0.038 | 0.751 | 0.732 | 0.860 | 5.301 | 1.29E−05 |
| DSC2 | NM_025004 | Hs.287555 | hs\|11q24.2 | −0.956 | −0.615 | −0.148 | −0.188 | −0.302 | −0.235 | 5.248 | 9.74E−04 |
| ENOX1 | NM_017993 | Hs.128258 | hs\|13q14.11 | −0.535 | −0.492 | −0.165 | −0.386 | 0.121 | 0.184 | 5.231 | 2.00E−04 |
| IL13 | NM_002188 | Hs.845 | hs\|5q31.1 | 0.305 | 0.276 | 0.355 | 0.648 | 0.810 | 0.978 | 5.230 | 8.80E−07 |
| BG571904 | BG571904 | Hs.660990 | hs\|10q22.2 | −0.033 | 0.157 | 0.445 | 0.616 | 0.668 | 0.492 | 5.151 | 7.70E−09 |
| BX455216 | N52197 | Hs.300701 | hs\|2q33.3 | −0.514 | −0.306 | 0.029 | 0.486 | 0.195 | 0.135 | 5.141 | 5.12E−08 |
| LOC729085 | AL117530 | Hs.646840 | hs\|3p22.1 | −0.801 | −1.033 | −0.260 | −0.124 | 0.023 | −0.093 | 5.116 | 2.51E−04 |
| BG188151 | BG188151 | Hs.71944 | hs\|5q14.2 | 0.159 | 0.224 | 0.513 | 0.480 | 0.808 | 0.868 | 5.114 | 8.26E−04 |
| LOC729409 | XR_015594 | Hs.587721 | hs\|12q15 | −0.023 | 0.028 | 0.197 | 0.321 | 0.486 | 0.684 | 5.093 | 1.37E−06 |
| C1orf103 | NM_018372 | Hs.25245 | hs\|1p13.3 | 0.018 | 0.155 | 0.213 | 0.273 | 0.619 | 0.725 | 5.077 | 2.08E−04 |
| PPP1R14C | NM_030949 | Hs.486798 | hs\|6q25.1 | −0.212 | 0.256 | 0.054 | −0.074 | 0.378 | 0.492 | 5.061 | 2.52E−04 |
| NAIP | NM_004536 | Hs.654500 | hs\|5q13.2 | −0.017 | 0.370 | 0.145 | 0.231 | 0.518 | 0.687 | 5.056 | 2.09E−05 |
| C13orf31 | NM_153218 | Hs.210586 | hs\|13q14.11 | −0.227 | −0.175 | 0.005 | −0.140 | 0.382 | 0.477 | 5.056 | 6.87E−06 |
| GOLGA8E | NM_001012423 | Hs.454647 | hs\|15q11.2 | 0.009 | 0.360 | 0.119 | 0.144 | 0.453 | 0.712 | 5.051 | 1.35E−10 |
| AK022848 | AK022848 | Hs.112482 | hs\|11q14.3 | 0.027 | 0.099 | −0.026 | −0.109 | 0.207 | 0.850 | 5.049 | 3.49E−04 |
| CXorf22 | NM_152632 | Hs.680415 | hs\|Xp21.1 | −0.667 | −0.354 | −0.432 | −0.776 | −0.077 | 0.035 | 5.033 | 4.89E−04 |
| KIF5C | NM_004522 | Hs.660699 | hs\|2q23.1 | −0.173 | −0.142 | 0.013 | 0.007 | 0.476 | 0.528 | 5.024 | 5.08E−04 |
| LRRCC1 | NM_033402 | Hs.193115 | hs\|8q21.2 | −0.084 | 0.040 | 0.125 | 0.212 | 0.634 | 0.613 | 4.985 | 2.78E−04 |
| FAM81B | NM_152548 | Hs.276287 | hs\|5q15 | 0.486 | 0.685 | 1.083 | 1.506 | 1.152 | 1.125 | 4.929 | 7.09E−04 |
| ID2 | NM_002166 | Hs.180919 | hs\|2p25.1 | −0.446 | −0.253 | −0.153 | −0.356 | 0.100 | 0.279 | 4.928 | 2.33E−04 |
| CMYA5 | NM_153610 | Hs.482625 | hs\|5q14.1 | 0.000 | 0.082 | 0.052 | 0.046 | 0.181 | 0.728 | 4.899 | 1.91E−04 |
| C1orf194 | BC127905 | Hs.446962 | hs\|1p13.3 | 0.145 | 0.337 | 0.549 | 0.971 | 0.626 | 0.833 | 4.874 | 4.56E−10 |
| TTC18 | NM_145170 | Hs.591367 | hs\|10q22.2 | 0.004 | 0.274 | 0.386 | 0.469 | 0.603 | 0.706 | 4.849 | 1.66E−05 |
| tcag7.1314 | AK126364 | Hs.186649 | hs\|7q11.23 | 0.229 | 0.442 | 0.554 | 0.723 | 0.862 | 0.913 | 4.832 | 9.08E−07 |
| ZNF385B | NM_152520 | Hs.655005 | hs\|2q31.2 | −0.525 | −0.088 | 0.315 | 0.147 | 0.299 | 0.406 | 4.799 | 4.16E−04 |
| ADAMTS6 | NM_197941 | Hs.482291 | hs\|5q12.3 | −0.037 | 0.225 | −0.181 | 0.083 | 0.287 | 0.645 | 4.793 | 6.71E−04 |
| RHOU | NM_021205 | Hs.647774 | hs\|1q42.13 | 0.352 | 0.367 | 0.554 | 0.734 | 0.736 | 1.030 | 4.767 | 1.07E−07 |
| ENST00000378850 | BC127739 | Hs.549398 | hs\|4q35.1 | −0.429 | −0.199 | 0.001 | 0.103 | 0.140 | 0.249 | 4.762 | 3.02E−07 |
| C2orf55 | NM_207362 | Hs.658091 | hs\|2q11.2 | 0.459 | 0.649 | 1.123 | 1.227 | 1.137 | 1.030 | 4.756 | 6.90E−08 |
| GPR83 | NM_016540 | Hs.272385 | hs\|11q21 | 0.221 | 0.166 | 0.385 | 0.417 | 0.785 | 0.898 | 4.755 | 2.91E−04 |
| LRRIQ1 | NM_032165 | Hs.402200 | hs\|12q21.31 | 0.008 | 0.144 | 0.331 | 0.463 | 0.718 | 0.683 | 4.736 | 5.64E−09 |
| WDR31 | NM_001012361 | Hs.133331 | hs\|9q32 | −0.436 | −0.054 | 0.222 | 0.311 | 0.229 | 0.142 | 4.726 | 9.33E−04 |
| DEFB126 | NM_178001 | Hs.400740 | hs\|9q34.11 | −0.263 | −0.147 | 0.448 | 0.355 | 0.407 | 0.437 | 4.690 | 4.25E−05 |
| ARMETL1 | NM_001029954 | Hs.559067 | hs\|10p13 | −0.147 | −0.102 | −0.092 | 0.118 | 0.157 | 0.571 | 4.686 | 5.83E−06 |
| LOC642826 | BC019715 | Hs.680765 | hs\|10q22.2 | −0.178 | 0.049 | 0.342 | 0.464 | 0.501 | 0.478 | 4.678 | 8.50E−12 |
| LOC129881 | BC117445 | Hs.370111 | hs\|2q31.1 | 0.000 | 0.000 | 0.369 | 0.867 | 0.670 | 0.590 | 4.677 | 1.44E−12 |
| C2orf13 | NM_173545 | Hs.258941 | hs\|2p14 | −0.172 | −0.113 | 0.180 | 0.121 | 0.305 | 0.495 | 4.646 | 8.01E−11 |
| THC2553512 | | | hs\|1q42.11 | −0.053 | 0.614 | −0.137 | 0.094 | 0.401 | 0.616 | 4.621 | 4.30E−04 |
| ACVR1C | NM_145259 | Hs.352338 | hs\|2q24.1 | −0.390 | −0.198 | −0.182 | −0.211 | −0.009 | 0.070 | 4.611 | 5.20E−06 |
| ZNF207 | AL834501 | Hs.500775 | hs\|17q11.2 | −0.067 | 0.242 | 0.013 | 0.130 | 0.439 | 0.600 | 4.600 | 2.04E−04 |
| ANTXR1 | NM_032208 | Hs.165859 | hs\|2p14 | 0.344 | 0.341 | 0.727 | 0.739 | 0.920 | 1.007 | 4.595 | 8.65E−04 |
| CHD9 | NM_0225134 | Hs.59159 | hs\|16q12.2 | 0.091 | 0.246 | 0.222 | 0.301 | 0.583 | 0.751 | 4.575 | 2.29E−04 |
| THC2526838 | | | hs\|1q23.3 | 0.030 | 0.111 | 0.305 | 0.339 | 0.378 | 0.688 | 4.547 | 2.57E−05 |
| ABCA12 | NM_173076 | Hs.134585 | hs\|2q35 | −0.320 | 0.372 | −0.327 | −0.615 | 0.167 | 0.336 | 4.536 | 4.34E−05 |
| TncRNA | U60873 | Hs.648467 | hs\|11q13.1 | 0.087 | 0.572 | 0.252 | 0.195 | 0.531 | 0.754 | 4.532 | 2.42E−04 |
| FKTN | NM_006731 | Hs.55777 | hs\|9q31.2 | 0.048 | −0.013 | −0.044 | 0.041 | 0.410 | 0.518 | 4.506 | 2.78E−04 |
| PTPRG | BC036018 | Hs.654488 | hs\|3p14.2 | −0.253 | 0.048 | 0.230 | 0.442 | 0.381 | 0.000 | 4.502 | 8.40E−05 |
| ZNF233 | NM_181756 | Hs.466891 | hs\|19q13.31 | 0.117 | 0.093 | 0.261 | 0.229 | 0.395 | 0.784 | 4.492 | 7.07E−05 |
| ENST00000370378 | AB029030 | Hs.21554 | hs\|1p22.1 | −0.318 | 0.222 | −0.180 | −0.196 | 0.138 | 0.396 | 4.450 | 8.81E−04 |
| FANK1 | NM_145235 | Hs.352591 | hs\|10q26.2 | −0.604 | −0.263 | 0.100 | 0.034 | −0.019 | 0.054 | 4.378 | 3.62E−04 |
| PCM1 | NM_006197 | Hs.491148 | hs\|8p22 | −0.047 | −0.007 | 0.122 | 0.209 | 0.390 | 0.481 | 4.371 | 5.67E−04 |
| SERPINI1 | NM_005025 | Hs.478153 | hs\|3q26.1 | −0.040 | 0.236 | 0.123 | 0.088 | 0.463 | 0.599 | 4.362 | 6.27E−04 |
| ARID4B | NM_016374 | Hs.575782 | hs\|1q42.3 | 0.023 | 0.213 | 0.151 | 0.202 | 0.517 | 0.662 | 4.352 | 4.67E−04 |
| KIAA1377 | NM_020802 | Hs.156352 | hs\|11q22.1 | −0.342 | 0.256 | −0.004 | 0.127 | 0.177 | 0.295 | 4.326 | 2.06E−05 |
| FGF7 | NM_014379 | Hs.13285 | hs\|8q23.2 | −0.117 | −0.039 | −0.021 | −0.026 | 0.224 | 0.591 | 4.320 | 8.18E−05 |
| CV339166 | CV339166 | Hs.694226 | hs\|1q41 | 0.078 | 0.183 | 0.131 | 0.162 | 0.507 | 0.713 | 4.300 | 1.10E−04 |
| LINCR | NM_001080535 | Hs.149219 | hs\|2q11.2 | −0.002 | 0.646 | 0.116 | 0.236 | 0.220 | 0.635 | 4.278 | 3.20E−05 |
| DA834198 | DA834198 | Hs.491872 | hs\|8q13.1 | −0.040 | −0.090 | 0.088 | 0.234 | 0.454 | 0.591 | 4.272 | 2.13E−06 |
| CFH | NM_002113 | Hs.363396 | hs\|1q31.3 | 0.050 | −0.092 | 0.080 | 0.173 | 0.369 | 0.677 | 4.271 | 2.32E−07 |
| SCG2 | NM_003469 | Hs.516726 | hs\|2q36.1 | −0.155 | 0.000 | −0.154 | −0.059 | 0.210 | 0.474 | 4.258 | 1.52E−10 |
| ARHGEF10 | BC026965 | Hs.98594 | hs\|8p23.3 | −0.184 | 0.148 | 0.004 | 0.154 | 0.309 | 0.443 | 4.233 | 1.71E−06 |
| DA093175 | | | hs\|9p12 | −0.275 | −0.037 | 0.164 | 0.365 | 0.337 | 0.239 | 4.229 | 3.32E−04 |
| GOLGA8A | NM_032632 | Hs.253726 | hs\|14q32.2 | −0.122 | 0.014 | 0.041 | 0.135 | 0.318 | 0.369 | 4.225 | 8.36E−06 |
| AK021467 | AK021467 | Hs.661311 | hs\|1q23.3 | −0.529 | 0.000 | −0.280 | −0.144 | −0.029 | 0.146 | 4.217 | 2.14E−04 |
| LOC283666 | BC035094 | Hs.655155 | hs\|15q22.2 | 0.103 | 0.222 | 0.508 | 0.328 | 0.582 | 0.817 | 4.217 | 6.08E−04 |
| FLJ35767 | NM_207459 | Hs.231897 | hs\|17q25.3 | −0.165 | 0.068 | 0.148 | 0.365 | 0.365 | 0.462 | 4.209 | 1.84E−04 |
| THC2725553 | | | hs\|21q21.1 | −0.354 | 0.048 | −0.087 | 0.055 | 0.171 | −0.055 | 4.194 | 1.20E−04 |
| ZNF430 | NM_025189 | Hs.466289 | hs\|19p12 | 0.057 | −0.130 | −0.054 | 0.053 | 0.454 | 0.565 | 4.162 | 4.12E−08 |
| CCDC141 | AK096821 | Hs.324341 | hs\|2q31.2 | −1.010 | −0.339 | −0.967 | −0.874 | −0.337 | −0.374 | 4.151 | 2.35E−04 |
| MAP3K13 | NM_004721 | Hs.656069 | hs\|3q27.2 | −0.532 | 0.051 | −0.354 | −0.181 | −0.009 | 0.086 | 4.150 | 3.08E−04 |
| CCDC66 | NM_001012506 | Hs.476399 | hs\|3p14.3 | −0.069 | 0.105 | 0.004 | 0.090 | 0.436 | 0.555 | 4.143 | 2.33E−04 |
| THC2727226 | | | hs\|3q13.31 | −0.061 | 0.163 | 0.511 | 0.757 | 0.558 | 0.529 | 4.140 | 5.58E−06 |
| THC2528990 | | | hs\|10q11.22 | −0.146 | 0.244 | 0.285 | 0.431 | 0.537 | 0.471 | 4.132 | 1.23E−04 |
| THC2718728 | | | hs\|9p12 | −0.279 | −0.002 | 0.186 | 0.335 | 0.319 | 0.203 | 4.103 | 4.60E−09 |
| THC2507829 | | | hs\|5q13.2 | 0.101 | 0.345 | 0.339 | 0.402 | 0.655 | 0.714 | 4.103 | 1.56E−04 |
| AK123972 | AK123972 | Hs.435458 | hs\|18q12.3 | 0.309 | 0.261 | 0.737 | 0.700 | 0.975 | 0.912 | 4.073 | 3.45E−04 |
| EDEM3 | NM_025191 | Hs.523811 | hs\|1q25.3 | 0.091 | 0.088 | 0.227 | 0.235 | 0.554 | 0.694 | 4.023 | 2.59E−04 |
| DB304731 | BX111927 | Hs.659410 | hs\|2q24.2 | −0.066 | 0.131 | 0.287 | 0.150 | 0.505 | 0.538 | 4.015 | 3.14E−09 |
| MNS1 | NM_018365 | Hs.444483 | hs\|15q21.3 | −0.109 | −0.140 | 0.100 | 0.321 | 0.386 | 0.302 | 3.976 | 1.70E−05 |
| AK022443 | AK022443 | Hs.656237 | hs\|3p14.1 | −0.146 | 0.221 | 0.352 | 0.431 | 0.528 | 0.407 | 3.970 | 1.17E−04 |
| PHF21B | NM_138415 | Hs.254097 | hs\|22q13.31 | −0.245 | −0.061 | 0.316 | 0.353 | 0.408 | 0.347 | 3.970 | 1.31E−11 |
| CPE | NM_001873 | Hs.75360 | hs\|4q32.3 | −0.529 | −0.304 | −0.070 | −0.188 | −0.044 | 0.071 | 3.970 | 7.37E−04 |
| BDH2 | NM_020139 | Hs.124696 | hs\|4q24 | −0.159 | −0.209 | −0.049 | −0.065 | 0.184 | 0.439 | 3.964 | 1.56E−04 |
| CP110 | NM_014711 | Hs.279912 | hs\|16p12.3 | 0.023 | 0.007 | 0.054 | 0.105 | 0.356 | 0.382 | 3.952 | 9.22E−06 |
| TRIP11 | NM_004239 | Hs.654511 | hs\|14q32.12 | −0.066 | 0.171 | −0.028 | −0.013 | 0.363 | 0.567 | 3.948 | 1.51E−07 |
| DMXL2 | NM_015263 | Hs.511386 | hs\|15q21.2 | −0.092 | 0.039 | 0.118 | 0.262 | 0.424 | 0.499 | 3.904 | 1.09E−07 |
| THC2673918 | | | hs\|3q13.31 | −0.243 | 0.059 | 0.361 | 0.307 | 0.447 | 0.348 | 3.888 | 7.97E−04 |
| LRRC6 | NM_012472 | Hs.591865 | hs\|8q24.22 | 0.074 | 0.091 | 0.637 | 0.709 | 0.544 | 0.610 | 3.883 | 3.50E−05 |
| FAM90A1 | NM_018088 | Hs.196086 | hs\|12p13.31 | 0.255 | 0.358 | 0.242 | 0.304 | 0.597 | 0.843 | 3.870 | 1.08E−04 |
| BX538272 | BX538272 | Hs.567380 | hs\|1p31.1 | 0.081 | −0.008 | 0.188 | 0.252 | 0.399 | 0.669 | 3.867 | 2.41E−04 |
| BRWD1 | NM_018963 | Hs.654740 | hs\|21q22.2 | −0.051 | 0.304 | 0.021 | 0.122 | 0.560 | 0.612 | 3.831 | 2.58E−04 |
| CROP | NM_016424 | Hs.130293 | hs\|17q21.33 | −0.052 | 0.078 | 0.140 | 0.312 | 0.404 | 0.531 | 3.825 | 2.76E−06 |
| BI771054 | BI771054 | Hs.341729 | hs\|3p22.2 | 0.078 | 0.423 | 0.101 | 0.305 | 0.463 | 0.660 | 3.811 | 6.44E−04 |
| C2orf63 | NM_152385 | Hs.468590 | hs\|2p16.1 | −0.203 | 0.069 | −0.133 | 0.008 | 0.221 | 0.378 | 3.810 | 2.29E−04 |
| THC2679528 | | | hs\|9p12 | −0.314 | 0.067 | 0.285 | 0.289 | 0.264 | 0.234 | 3.804 | 3.51E−05 |
| CAMK2N1 | NM_018584 | Hs.197922 | hs\|1p36.12 | 0.000 | 0.117 | 0.725 | 0.210 | 0.470 | 0.580 | 3.803 | 3.46E−10 |
| RELN | NM_005045 | Hs.655654 | hs\|7q22.1 | 0.000 | 0.000 | 0.148 | 0.155 | 0.321 | 0.580 | 3.800 | 6.98E−06 |
| ANKRD12 | NM_015208 | Hs.464585 | hs\|18p11.22 | −0.027 | 0.086 | 0.176 | 0.281 | 0.341 | 0.379 | 3.789 | 2.02E−04 |
| ZBTB1 | NM_014950 | Hs.655536 | hs\|14q23.3 | 0.086 | 0.161 | 0.162 | 0.278 | 0.510 | 0.663 | 3.781 | 2.68E−04 |
| BU928689 | | | hs\|8q21.3 | −0.157 | 0.157 | −0.123 | −0.006 | 0.192 | 0.420 | 3.780 | 3.82E−04 |
| XRCC4 | NM_022550 | Hs.567359 | hs\|5q14.2 | 0.049 | 0.136 | 0.280 | 0.229 | 0.565 | 0.625 | 3.773 | 9.41E−05 |
| GEN1 | NM_182625 | Hs.467793 | hs\|2p24.2 | −0.027 | 0.052 | 0.047 | 0.212 | 0.586 | 0.577 | 3.766 | 7.46E−05 |
| IL1RAPL1 | NM_014271 | Hs.658912 | hs\|Xp21.2 | 0.000 | 0.043 | 0.308 | 0.773 | 0.565 | 0.576 | 3.763 | 2.34E−08 |
| ZNF493 | NM_175910 | Hs.656558 | hs\|19p12 | 0.013 | 0.091 | −0.018 | −0.089 | 0.276 | 0.588 | 3.759 | 6.14E−04 |
| AK026718 | AK026718 | Hs.125352 | hs\|5q23.2 | 0.142 | 0.128 | 0.237 | 0.354 | 0.555 | 0.715 | 3.756 | 1.11E−05 |
| TSPAN5 | AK055659 | Hs.591706 | hs\|4q23 | 0.035 | 0.320 | 0.521 | 0.531 | 0.463 | 0.390 | 3.720 | 6.68E−05 |
| AK127804 | AK127804 | Hs.438858 | hs\|9p24.2 | −0.204 | 0.040 | 0.174 | 0.389 | 0.285 | 0.365 | 3.709 | 2.12E−04 |
| DCLRE1C | BC022254 | Hs.656065 | hs\|10p13 | −0.025 | 0.145 | 0.157 | 0.321 | 0.522 | 0.545 | 3.708 | 2.67E−04 |
| RIMS4 | NM_182970 | Hs.517065 | hs\|20q13.12 | −0.216 | 0.019 | 0.402 | 0.466 | 0.341 | 0.312 | 3.701 | 3.03E−04 |
| BC009228 | BC009228 | Hs.633824 | hs\|1q24.1 | −0.076 | 0.390 | 0.023 | 0.174 | 0.304 | 0.493 | 3.691 | 7.04E−05 |
| AA861995 | AA861995 | Hs.153521 | hs\|1p13.3 | −0.693 | −0.340 | −0.462 | −0.699 | −0.244 | −0.105 | 3.685 | 5.47E−04 |
| AMY1C | NM_001008219 | Hs.655232 | hs\|1p21.1 | 0.154 | 0.345 | 0.418 | 0.563 | 0.655 | 0.721 | 3.683 | 6.03E−06 |
| STK31 | NM_032944 | Hs.309767 | hs\|7p15.3 | 0.137 | 0.220 | 0.289 | 0.286 | 0.582 | 0.702 | 3.678 | 5.89E−05 |
| TPRG1 | NM_198485 | Hs.338851 | hs\|3q28 | −0.302 | 0.000 | 0.210 | 0.340 | −0.002 | 0.264 | 3.673 | 5.40E−07 |
| GCC2 | NM_181453 | Hs.436505 | hs\|2q12.3 | 0.097 | 0.276 | 0.069 | 0.087 | 0.257 | 0.507 | 3.673 | 1.26E−04 |
| BC062758 | BC062758 | Hs.571424 | hs\|8q21.11 | −0.275 | −0.025 | 0.146 | −0.123 | 0.286 | 0.290 | 3.667 | 2.94E−08 |
| ZBBX | NM_024687 | Hs.478143 | hs\|3q26.1 | 0.106 | 0.330 | 0.691 | 0.607 | 0.617 | 0.701 | 3.663 | 1.88E−04 |
| TMEM67 | NM_153704 | Hs.116240 | hs\|8q22.1 | −0.236 | −0.134 | 0.033 | 0.202 | 0.074 | 0.327 | 3.657 | 5.56E−04 |
| FLJ32679 | NM_001012452 | Hs.510812 | | −0.102 | 0.046 | 0.087 | 0.201 | 0.338 | 0.461 | 3.656 | 4.42E−08 |
| CA2 | NM_000067 | Hs.155097 | hs\|8q21.2 | −0.566 | −0.372 | −0.388 | −0.473 | −0.143 | −0.005 | 3.635 | 3.35E−07 |
| C1orf63 | AK027318 | Hs.259412 | hs\|1p36.11 | −0.151 | 0.131 | 0.012 | 0.134 | 0.248 | 0.409 | 3.631 | 1.21E−05 |
| FGF12 | NM_004113 | Hs.584758 | hs\|3q28 | −0.151 | −0.089 | 0.044 | 0.009 | 0.321 | 0.406 | 3.615 | 5.55E−05 |
| C10orf118 | NM_018017 | Hs.159066 | hs\|10q25.3 | −0.072 | 0.143 | −0.128 | −0.145 | 0.273 | 0.484 | 3.612 | 2.78E−04 |
| THC2491396 | CB133932 | Hs.558671 | hs\|14q31.3 | −0.061 | −0.156 | −0.097 | 0.033 | 0.309 | 0.377 | 3.601 | 3.37E−04 |
| THC2606490 | | | hs\|3q13.31 | −0.253 | 0.084 | 0.298 | 0.282 | 0.334 | 0.253 | 3.599 | 4.21E−05 |
| ZNF708 | NM_021269 | Hs.466296 | hs\|19p12 | −0.009 | 0.143 | −0.089 | 0.042 | 0.344 | 0.552 | 3.588 | 4.78E−05 |
| CCNA1 | NM_003914 | Hs.417050 | hs\|13q13.3 | −0.589 | −0.032 | −0.220 | −0.350 | −0.057 | −0.035 | 3.580 | 2.16E−04 |
| ROCK2 | NM_004850 | Hs.591600 | hs\|2p25.1 | −0.111 | −0.126 | −0.149 | −0.123 | 0.338 | 0.427 | 3.579 | 6.17E−04 |
| NEFH | NM_021076 | Hs.198760 | hs\|22q12.2 | −0.507 | −0.104 | −0.262 | −0.035 | −0.032 | 0.049 | 3.572 | 6.61E−04 |
| CEP110 | NM_007018 | Hs.653263 | hs\|9q33.2 | −0.186 | −0.060 | 0.074 | 0.160 | 0.327 | 0.367 | 3.570 | 4.18E−05 |
| THC2642866 | | | hs\|12p13.2 | −0.266 | 0.055 | −0.194 | −0.051 | 0.192 | 0.291 | 3.568 | 9.35E−04 |
| THC2697162 | | | hs\|3q13.31 | −0.076 | 0.203 | 0.528 | 0.452 | 0.475 | 0.366 | 3.551 | 1.15E−05 |
| ZFP2 | NM_030613 | Hs.654533 | hs\|5q35.3 | −0.099 | −0.100 | 0.155 | 0.156 | 0.075 | 0.457 | 3.528 | 7.79E−04 |
| IPMK | NM_152230 | Hs.499690 | hs\|10q21.1 | −0.113 | 0.043 | 0.058 | 0.041 | 0.349 | 0.451 | 3.524 | 7.24E−04 |
| AV707343 | AV707343 | Hs.595279 | hs\|3q28 | −0.215 | −0.122 | 0.074 | 0.002 | 0.300 | 0.331 | 3.522 | 6.79E−04 |
| THC2701431 | | | hs\|1p13.3 | −0.223 | 0.118 | −0.036 | −0.274 | 0.194 | 0.342 | 3.516 | 1.14E−04 |
| SDCBP | AK128645 | Hs.200804 | hs\|8q12.1 | −0.079 | 0.123 | 0.062 | 0.070 | 0.373 | 0.470 | 3.511 | 1.76E−04 |
| ZNF813 | NM_001004301 | Hs.433293 | hs\|19q13.41 | 0.111 | −0.016 | 0.120 | 0.068 | 0.340 | 0.443 | 3.505 | 5.46E−04 |
| ODF3L1 | NM_175881 | Hs.144348 | hs\|15q24.2 | 0.000 | 0.000 | 0.204 | 0.743 | 0.614 | 0.393 | 3.505 | 8.61E−04 |
| WBSCR19 | NM_175064 | Hs.645483 | hs\|7p13 | 0.106 | 0.304 | 0.271 | 0.412 | 0.452 | 0.795 | 3.495 | 2.76E−04 |
| CTGLF4 | NM_133446 | Hs.656384 | hs\|10q11.21 | 0.003 | 0.208 | 0.348 | 0.482 | 0.540 | 0.467 | 3.492 | 3.25E−06 |
| ATM | NM_000051 | Hs.367437 | hs\|11q22.3 | −0.153 | 0.301 | −0.096 | 0.036 | 0.214 | 0.389 | 3.479 | 7.46E−07 |
| CB850583 | CB850583 | Hs.625122 | hs\|9p24.2 | −0.147 | 0.082 | 0.174 | 0.406 | 0.361 | 0.395 | 3.479 | 3.80E−04 |
| NBEA | NM_015678 | Hs.491172 | hs\|13q13.3 | −0.001 | −0.065 | 0.054 | 0.055 | 0.385 | 0.537 | 3.477 | 2.76E−04 |
| ITLN1 | NM_017625 | Hs.50813 | hs\|1q23.3 | −0.004 | 0.000 | 0.066 | 0.386 | 0.436 | 0.544 | 3.473 | 2.29E−07 |
| THC2750782 | | | hs\|19p13.11 | 0.011 | 0.109 | 0.048 | 0.122 | 0.303 | 0.597 | 3.471 | 6.13E−05 |
| IQCG | NM_032263 | Hs.591675 | hs\|3q29 | −0.082 | −0.083 | 0.197 | 0.362 | 0.285 | 0.248 | 3.466 | 3.91E−04 |
| ARID4A | NM_002892 | Hs.161000 | hs\|14q23.1 | −0.181 | −0.011 | −0.068 | −0.030 | 0.211 | 0.359 | 3.462 | 1.23E−04 |
| FANCF | NM_022725 | Hs.632151 | hs\|11p14.3 | −0.426 | −0.203 | −0.493 | −0.046 | 0.008 | 0.114 | 3.460 | 6.58E−04 |
| C7orf53 | NM_182597 | Hs.396189 | hs\|7q31.1 | 0.170 | 0.187 | 0.176 | 0.259 | 0.432 | 0.707 | 3.455 | 7.95E−06 |
| THC2551948 | | | | −0.080 | 0.069 | 0.273 | 0.366 | 0.489 | 0.440 | 3.431 | 2.00E−04 |
| ZDHHC21 | NM_178566 | Hs.649522 | hs\|9p22.3 | 0.080 | −0.068 | 0.035 | 0.108 | 0.534 | 0.567 | 3.428 | 1.42E−04 |
| THC2691405 | | | hs\|15q21.1 | −0.048 | 0.178 | 0.095 | 0.105 | 0.350 | 0.486 | 3.419 | 1.20E−04 |
| C8orf46 | NM_152765 | Hs.268869 | hs\|8q13.1 | 0.000 | 0.084 | 0.180 | 0.495 | 0.196 | 0.534 | 3.418 | 5.92E−07 |
| AIFM2 | NM_032797 | Hs.655377 | hs\|10q22.1 | 0.080 | 0.314 | 0.123 | 0.234 | 0.449 | 0.622 | 3.416 | 4.89E−04 |
| DNAJC21 | NM_194283 | Hs.131887 | hs\|5p13.2 | −0.153 | −0.441 | −0.160 | −0.064 | 0.283 | 0.359 | 3.412 | 4.94E−06 |
| AK096154 | AK096154 | Hs.594968 | hs\|5q22.3 | 0.131 | 0.106 | 0.252 | 0.284 | 0.453 | 0.566 | 3.395 | 1.44E−04 |
| PLGLB1 | NM_001032392 | Hs.652174 | hs\|2p11.2 | −0.121 | 0.057 | 0.147 | 0.243 | 0.238 | 0.419 | 3.392 | 2.47E−07 |
| RNF6 | NM_005977 | Hs.136885 | hs\|13q12.13 | −0.022 | −0.036 | −0.004 | −0.004 | 0.284 | 0.486 | 3.379 | 6.04E−05 |
| CAV1 | NM_001753 | Hs.74034 | hs\|7q31.2 | 0.208 | 0.105 | 0.708 | 0.844 | 0.745 | 0.734 | 3.374 | 3.85E−04 |
| EFCAB7 | NM_032437 | Hs.652324 | hs\|1p31.3 | 0.057 | 0.268 | 0.218 | 0.257 | 0.484 | 0.585 | 3.373 | 7.53E−05 |
| THC2735960 | | | hs\|9q31.2 | 0.078 | 0.000 | −0.065 | −0.036 | 0.482 | 0.525 | 3.366 | 1.48E−04 |
| TEX14 | NM_198393 | Hs.390221 | hs\|17q22 | 0.387 | 0.299 | 0.315 | 0.255 | 0.518 | 0.843 | 3.364 | 3.43E−04 |
| IFI16 | NM_005531 | Hs.380250 | hs\|1q23.1 | 0.000 | 0.146 | 0.014 | 0.000 | 0.172 | 0.546 | 3.364 | 3.70E−04 |
| ABCC2 | NM_000392 | Hs.368243 | hs\|10q24.2 | −0.001 | 0.139 | 0.513 | 0.552 | 0.421 | 0.526 | 3.361 | 5.14E−12 |
| ZNF429 | NM_001001415 | Hs.656558 | hs\|19p12 | −0.290 | 0.002 | −0.215 | −0.195 | 0.041 | 0.237 | 3.358 | 7.10E−04 |
| CHURC1 | NM_145165 | Hs.325531 | hs\|14q23.3 | −0.197 | −0.269 | −0.133 | −0.096 | 0.195 | 0.329 | 3.352 | 5.34E−04 |
| IFT80 | NM_020800 | Hs.478095 | hs\|3q26.1 | −0.028 | 0.081 | 0.156 | 0.277 | 0.413 | 0.495 | 3.335 | 1.87E−05 |
| ENST00000315707 | BC113564 | Hs.121692 | hs\|17p13.1 | −0.047 | −0.009 | 0.207 | 0.362 | 0.297 | 0.476 | 3.334 | 4.28E−04 |
| N4BP2L2 | U50529 | Hs.507680 | hs\|13q13.1 | −0.094 | 0.161 | −0.029 | 0.103 | 0.409 | 0.486 | 3.324 | 7.58E−12 |
| ZFP37 | NM_003408 | Hs.150406 | hs\|9q32 | −0.029 | 0.040 | 0.154 | 0.123 | 0.409 | 0.492 | 3.319 | 3.89E−09 |
| AREG | NM_001657 | Hs.270833 | hs\|4q13.3 | 0.268 | 0.548 | 0.555 | 0.596 | 0.739 | 0.835 | 3.316 | 4.65E−04 |
| C1orf118 | AK075118 | Hs.632414 | hs\|1p31.1 | 0.019 | 0.091 | 0.197 | 0.322 | 0.572 | 0.540 | 3.314 | 7.35E−10 |
| ENST00000290943 | | | hs\|9p13.3 | −0.002 | 0.231 | 0.340 | 0.470 | 0.547 | 0.504 | 3.313 | 2.86E−04 |
| GIGYF2 | BC012484 | | hs\|2q37.1 | −0.029 | 0.164 | 0.019 | 0.130 | 0.388 | 0.491 | 3.303 | 3.57E−04 |
| SUV39H2 | NM_024670 | Hs.554883 | hs\|10p13 | −0.051 | −0.340 | −0.238 | −0.171 | 0.174 | 0.276 | 3.271 | 1.13E−10 |
| CPNE8 | NM_153634 | Hs.40910 | hs\|12q12 | −0.187 | −0.081 | −0.013 | −0.115 | 0.254 | 0.334 | 3.265 | 1.57E−04 |
| ZNF25 | NM_145011 | Hs.499429 | hs\|10p11.21 | 0.099 | 0.152 | 0.334 | 0.399 | 0.327 | 0.670 | 3.264 | 7.44E−04 |
| THC2695576 | BF735554 | | hs\|3q13.31 | −0.104 | 0.043 | 0.306 | 0.361 | 0.371 | 0.477 | 3.263 | 5.31E−05 |
| SRI | NM_003130 | Hs.489040 | hs\|7q21.12 | 0.322 | 0.264 | 0.784 | 0.835 | 0.865 | 0.726 | 3.261 | 2.00E−04 |
| EFHB | NM_144715 | Hs.670883 | hs\|3p24.3 | −0.158 | 0.000 | 0.276 | 0.251 | 0.400 | 0.356 | 3.260 | 6.17E−04 |
| SEL1L | NM_005065 | Hs.181300 | hs\|14q31.1 | 0.116 | −0.023 | 0.256 | 0.200 | 0.401 | 0.629 | 3.255 | 3.13E−04 |
| CEP350 | NM_014810 | Hs.413045 | hs\|1q25.2 | 0.084 | 0.163 | 0.202 | 0.288 | 0.497 | 0.596 | 3.247 | 6.38E−05 |
| THAP2 | NM_031435 | Hs.245798 | hs\|12q21.1 | 0.053 | 0.159 | 0.275 | 0.220 | 0.476 | 0.652 | 3.241 | 6.67E−04 |
| THAP6 | NM_144721 | Hs.479971 | hs\|4q21.1 | 0.086 | 0.090 | −0.037 | 0.038 | 0.345 | 0.598 | 3.237 | 8.56E−04 |
| ZNF582 | NM_144690 | Hs.244391 | hs\|19q13.43 | 0.107 | −0.140 | −0.091 | 0.024 | 0.280 | 0.405 | 3.228 | 5.33E−11 |
| ABHD13 | NM_032859 | Hs.183528 | hs\|13q33.3 | −0.007 | 0.000 | −0.059 | 0.036 | 0.191 | 0.500 | 3.222 | 1.47E−05 |
| GOLGB1 | NM_004487 | Hs.213389 | hs\|3q13.33 | 0.055 | 0.231 | 0.076 | 0.142 | 0.376 | 0.561 | 3.218 | 1.95E−04 |
| ZNF571 | NM_016536 | Hs.590944 | hs\|19q13.12 | 0.037 | 0.120 | 0.059 | 0.056 | 0.293 | 0.544 | 3.216 | 3.57E−09 |
| ASPM | NM_018136 | Hs.121028 | hs\|1q31.3 | −0.014 | 0.037 | 0.084 | 0.183 | 0.484 | 0.494 | 3.215 | 1.59E−06 |
| LOC100129397 | AK095841 | Hs.683848 | hs\|15q21.1 | 0.238 | 0.000 | 0.525 | 0.589 | 0.522 | 0.536 | 3.213 | 2.24E−04 |
| MTHFD2L | NM_001004346 | Hs.479954 | hs\|4q13.3 | 0.004 | 0.000 | 0.132 | 0.078 | 0.501 | 0.511 | 3.209 | 7.50E−04 |
| LOC729806 | XM_001131376 | Hs.635482 | hs\|1q44 | 0.061 | 0.138 | 0.280 | 0.330 | 0.441 | 0.567 | 3.208 | 5.72E−07 |
| SUSD4 | NM_017982 | Hs.497841 | hs\|1q41 | 0.477 | 0.630 | 0.667 | 1.038 | 0.947 | 0.981 | 3.192 | 2.93E−05 |
| ZNF224 | | | hs\|19q13.31 | 0.026 | 0.158 | 0.194 | 0.229 | 0.432 | 0.528 | 3.180 | 3.19E−04 |
| RB1CC1 | NM_014781 | Hs.196102 | hs\|8q11.23 | −0.129 | 0.006 | −0.102 | −0.071 | 0.232 | 0.404 | 3.170 | 7.37E−06 |
| THC2659095 | | | hs\|9q12 | 0.030 | −0.165 | 0.525 | 0.503 | 0.464 | 0.435 | 3.160 | 5.50E−04 |
| SLC27A2 | NM_003645 | Hs.11729 | hs\|15q21.2 | −0.364 | −0.339 | −0.180 | −0.371 | 0.074 | 0.135 | 3.158 | 7.59E−04 |
| RPGR | NM_000328 | Hs.61438 | hs\|Xp11.4 | 0.076 | 0.084 | 0.310 | 0.465 | 0.553 | 0.576 | 3.146 | 4.81E−06 |
| AF237700 | AF237700 | | hs\|2p11.2 | 0.020 | 0.273 | 0.135 | 0.212 | 0.376 | 0.516 | 3.135 | 6.03E−05 |
| AVIL | NM_006576 | Hs.584854 | hs\|12q14.1 | 0.123 | 0.152 | 0.373 | 0.397 | 0.477 | 0.619 | 3.132 | 1.32E−04 |
| JMJD1C | NM_004241 | Hs.413416 | hs\|10q21.2 | −0.037 | 0.052 | 0.023 | 0.105 | 0.310 | 0.458 | 3.131 | 9.69E−05 |
| KIF27 | NM_017576 | Hs.546403 | hs\|9q21.32 | −0.008 | 0.165 | 0.159 | 0.274 | 0.449 | 0.488 | 3.127 | 1.36E−06 |
| ACE2 | NM_021804 | Hs.178098 | hs\|Xp22.2 | −0.372 | −0.285 | −0.187 | −0.215 | 0.028 | 0.123 | 3.126 | 4.62E−08 |
| C10orf28 | NM_014472 | Hs.419800 | hs\|10q24.2 | 0.040 | 0.017 | 0.047 | 0.019 | 0.270 | 0.352 | 3.119 | 6.87E−04 |
| AK124263 | AK124263 | Hs.649522 | hs\|9p22.3 | 0.097 | 0.170 | 0.200 | 0.238 | 0.520 | 0.591 | 3.118 | 5.02E−04 |
| ZNF181 | NM_001029997 | Hs.659191 | hs\|19q13.11 | 0.043 | 0.160 | 0.120 | 0.158 | 0.316 | 0.542 | 3.118 | 2.46E−05 |
| PIK3C2A | NM_002645 | Hs.175343 | hs\|11p15.1 | −0.015 | 0.006 | −0.062 | −0.082 | 0.242 | 0.478 | 3.113 | 2.33E−04 |
| ZNF449 | NM_152695 | Hs.28780 | hs\|Xq26.3 | 0.107 | 0.320 | 0.062 | 0.129 | 0.432 | 0.592 | 3.112 | 7.26E−05 |
| hCG_23177 | | | hs\|1p34.2 | −0.049 | 0.064 | 0.262 | 0.242 | 0.329 | 0.445 | 3.111 | 8.59E−05 |
| CSPP1 | NM_024790 | Hs.370147 | hs\|8q13.2 | −0.111 | 0.045 | 0.058 | 0.137 | 0.308 | 0.381 | 3.107 | 7.06E−05 |
| THC2635591 | | | hs\|12q14.3 | 0.206 | 0.049 | 0.117 | 0.175 | 0.496 | 0.607 | 3.100 | 7.21E−11 |
| ZNF721 | NM_133474 | Hs.428360 | hs\|4p16.3 | −0.053 | 0.147 | −0.175 | −0.146 | 0.322 | 0.437 | 3.092 | 6.82E−04 |
| KIAA1466 | AB040899 | Hs.147710 | hs\|7q33 | −0.008 | 0.035 | 0.124 | 0.307 | 0.419 | 0.482 | 3.091 | 1.85E−08 |
| INTU | NM_015693 | Hs.391481 | hs\|4q28.1 | −0.220 | −0.023 | −0.052 | 0.098 | 0.164 | 0.271 | 3.090 | 2.93E−04 |
| KIAA1009 | NM_014895 | Hs.485865 | hs\|6q14.3 | −0.043 | 0.034 | −0.026 | 0.012 | 0.244 | 0.458 | 3.080 | 5.72E−11 |
| THC2647962 | | | hs\|2p22.2 | 0.122 | 0.140 | 0.316 | 0.406 | 0.540 | 0.611 | 3.079 | 5.24E−05 |
| THC2652887 | | | hs\|15q21.3 | 0.015 | 0.183 | 0.255 | 0.343 | 0.456 | 0.503 | 3.077 | 4.41E−05 |
| GOLGA4 | NM_002078 | Hs.344151 | hs\|3p22.2 | −0.061 | 0.168 | −0.109 | −0.104 | 0.330 | 0.439 | 3.069 | 2.47E−04 |
| SLU7 | NM_006425 | Hs.435342 | hs\|5q33.3 | 0.084 | 0.150 | 0.165 | 0.192 | 0.424 | 0.543 | 3.064 | 1.07E−04 |
| BX090520 | BX090520 | Hs.574305 | hs\|16q12.2 | 0.202 | 0.372 | 0.587 | 0.666 | 0.764 | 0.571 | 3.063 | 4.24E−04 |
| ZNF658B | NM_033160 | Hs.522147 | hs\|9p12 | 0.104 | 0.130 | 0.213 | 0.277 | 0.509 | 0.590 | 3.062 | 2.57E−09 |
| IFT81 | NM_014055 | Hs.528382 | hs\|12q24.11 | −0.015 | 0.123 | 0.215 | 0.317 | 0.437 | 0.470 | 3.054 | 7.38E−07 |
| DMXL1 | NM_005509 | Hs.181042 | hs\|5q23.1 | 0.153 | 0.268 | 0.226 | 0.351 | 0.516 | 0.636 | 3.043 | 2.68E−06 |
| WDR33 | AK002156 | Hs.620490 | hs\|2q14.3 | −0.149 | −0.116 | −0.129 | −0.088 | 0.079 | 0.336 | 3.035 | 3.54E−05 |
| VCPIP1 | AF088033 | Hs.632066 | hs\|8q13.1 | 0.020 | 0.035 | 0.115 | 0.148 | 0.412 | 0.497 | 3.034 | 4.35E−04 |
| SFRS12 | NM_139168 | Hs.519347 | hs\|5q12.3 | 0.051 | 0.113 | 0.134 | 0.185 | 0.417 | 0.540 | 3.033 | 5.16E−04 |
| XR_018765 | XR_018765 | Hs.647996 | hs\|4q32.3 | −0.044 | −0.028 | −0.056 | −0.048 | 0.399 | 0.437 | 3.032 | 1.01E−07 |
| THC2612020 | | | hs\|Xq22.3 | −0.016 | 0.197 | −0.084 | 0.006 | 0.373 | 0.465 | 3.025 | 5.70E−12 |
| RBP4 | NM_006744 | Hs.50223 | hs\|10q23.33 | −0.002 | 0.000 | 0.322 | 0.409 | 0.492 | 0.436 | 3.014 | 7.78E−04 |
| RECK | NM_021111 | Hs.388918 | hs\|9p13.3 | −0.067 | 0.252 | 0.316 | 0.430 | 0.442 | 0.504 | 3.000 | 5.28E−05 |
| ZNF84 | NM_003428 | Hs.654730 | hs\|12q24.33 | −0.007 | 0.066 | 0.060 | 0.071 | 0.297 | 0.470 | 3.000 | 2.26E−05 |
| ZNF14 | NM_021030 | Hs.659932 | hs\|19p13.11 | 0.064 | 0.120 | 0.089 | 0.143 | 0.443 | 0.542 | 2.999 | 2.38E−05 |
| TUG1 | NR_002323 | Hs.554829 | hs\|22q12.2 | 0.250 | 0.159 | 0.429 | 0.590 | 0.640 | 0.726 | 2.996 | 8.71E−06 |
| AK022299 | AK022299 | Hs.565253 | hs\|19q12 | 0.033 | 0.257 | 0.009 | 0.067 | 0.485 | 0.503 | 2.994 | 5.02E−05 |
| ZNF471 | AB037817 | Hs.590979 | hs\|19q13.43 | 0.088 | 0.178 | 0.112 | 0.001 | 0.333 | 0.565 | 2.994 | 4.04E−04 |
| ZNF397OS | AK001503 | Hs.464896 | hs\|18q12.2 | −0.360 | −0.350 | 0.003 | 0.067 | 0.106 | 0.033 | 2.985 | 7.28E−05 |
| THC2646608 | | | hs\|18q23 | −0.090 | 0.008 | 0.176 | 0.216 | 0.224 | 0.409 | 2.983 | 6.84E−04 |
| AK098220 | AK098220 | Hs.664334 | hs\|5q13.2 | 0.096 | 0.339 | 0.310 | 0.419 | 0.588 | 0.570 | 2.980 | 3.92E−04 |
| THC2620401 | AV696077 | Hs.645617 | hs\|5q22.3 | 0.152 | −0.050 | −0.005 | 0.053 | 0.354 | 0.469 | 2.975 | 8.91E−13 |
| ENST00000342314 | XM_001126928 | Hs.568189 | hs\|15q13.3 | −0.107 | 0.028 | 0.072 | 0.183 | 0.294 | 0.366 | 2.975 | 1.59E−05 |
| F13B | NM_001994 | Hs.435782 | hs\|1q31.3 | 0.000 | −0.053 | 0.007 | 0.364 | 0.447 | 0.433 | 2.974 | 1.70E−05 |
| THC2769342 | | | hs\|5q23.2 | 0.112 | 0.058 | −0.023 | 0.101 | 0.419 | 0.529 | 2.961 | 4.02E−08 |
| ZNF789 | AK131429 | Hs.440384 | hs\|7q22.1 | 0.156 | 0.225 | 0.294 | 0.320 | 0.589 | 0.626 | 2.957 | 2.67E−04 |
| FANCM | NM_020937 | Hs.509229 | hs\|14q21.3 | −0.102 | −0.054 | −0.079 | 0.013 | 0.356 | 0.338 | 2.955 | 3.05E−05 |
| C17orf67 | BC041467 | Hs.658949 | hs\|17q22 | 0.008 | 0.184 | 0.266 | 0.397 | 0.369 | 0.476 | 2.944 | 2.06E−04 |
| FAM80B | AB033064 | Hs.504670 | hs\|12p13.31 | 0.114 | 0.157 | 0.166 | 0.190 | 0.469 | 0.578 | 2.942 | 8.33E−04 |
| FAM91A1 | NM_144963 | Hs.459174 | hs\|8q24.13 | −0.045 | 0.001 | 0.110 | 0.156 | 0.384 | 0.422 | 2.940 | 9.35E−04 |
| ZC3H6 | NM_198581 | Hs.190477 | hs\|2q13 | 0.080 | 0.173 | 0.477 | 0.329 | 0.407 | 0.637 | 2.932 | 6.48E−04 |
| IQGAP2 | NM_006633 | Hs.291030 | hs\|5q13.3 | −0.124 | −0.001 | −0.124 | −0.153 | 0.141 | 0.342 | 2.922 | 6.94E−04 |
| APC2 | NM_005883 | Hs.446376 | hs\|19p13.3 | 0.172 | 0.255 | 0.558 | 0.632 | 0.645 | 0.616 | 2.919 | 3.69E−13 |
| HERC2P2 | NM_199045 | Hs.531509 | hs\|15q13.1 | −0.073 | 0.247 | 0.100 | 0.163 | 0.286 | 0.404 | 2.909 | 5.52E−05 |
| TTC9 | D86980 | Hs.79170 | hs\|14q24.2 | −0.047 | −0.041 | 0.239 | 0.581 | 0.330 | 0.416 | 2.905 | 1.33E−08 |
| LZTFL1 | NM_020347 | Hs.30824 | hs\|3p21.31 | 0.093 | 0.142 | 0.340 | 0.432 | 0.417 | 0.556 | 2.903 | 3.75E−05 |
| ACOX1 | NM_004035 | Hs.464137 | hs\|17q25.1 | −0.644 | −0.021 | −0.644 | −0.513 | −0.278 | −0.182 | 2.901 | 1.52E−11 |
| SPDYA | NM_001008779 | Hs.511956 | hs\|2p23.2 | 0.130 | 0.211 | 0.171 | 0.196 | 0.391 | 0.591 | 2.896 | 2.35E−04 |
| BAZ2B | NM_013450 | Hs.470369 | hs\|2q24.2 | −0.015 | 0.088 | 0.016 | 0.044 | 0.252 | 0.447 | 2.892 | 2.59E−04 |
| OXTR | NM_000916 | Hs.2820 | hs\|3p25.3 | −0.021 | 0.125 | −0.025 | −0.004 | 0.392 | 0.440 | 2.888 | 2.91E−11 |
| MXRA8 | NM_032348 | Hs.558570 | hs\|1p36.33 | −0.116 | −0.100 | 0.361 | 0.513 | 0.320 | 0.301 | 2.887 | 3.01E−04 |
| ZBTB41 | NM_194314 | Hs.529439 | hs\|1q31.3 | 0.139 | 0.169 | 0.269 | 0.299 | 0.437 | 0.600 | 2.886 | 1.65E−06 |
| BX329117 | BX329117 | Hs.499925 | hs\|10q21.3 | 0.107 | −0.010 | −0.096 | −0.045 | 0.339 | 0.431 | 2.885 | 6.16E−04 |
| UNC13A | NM_001080421 | Hs.164502 | hs\|19p13.11 | −0.077 | 0.115 | 0.279 | 0.385 | 0.332 | 0.383 | 2.880 | 1.56E−04 |
| LOC220594 | NM_145809 | Hs.234573 | hs\|17p11.2 | −0.083 | 0.119 | 0.018 | 0.123 | 0.239 | 0.375 | 2.876 | 1.79E−06 |
| BF575152 | BF575152 | Hs.403246 | hs\|20p12.1 | 0.001 | 0.162 | 0.059 | 0.125 | 0.290 | 0.475 | 2.875 | 1.71E−05 |
| ENST00000356354 | AK127179 | | hs\|14q32.13 | −0.298 | −0.148 | 0.168 | 0.398 | −0.011 | 0.157 | 2.859 | 2.11E−04 |
| LOC149134 | AK022825 | Hs.677168 | hs\|1q44 | 0.006 | 0.128 | 0.387 | 0.559 | 0.448 | 0.461 | 2.854 | 2.98E−04 |
| CCDC132 | NM_017667 | Hs.222282 | hs\|7q21.3 | 0.163 | 0.191 | 0.210 | 0.238 | 0.507 | 0.620 | 2.853 | 6.18E−04 |
| TIGD7 | NM_033208 | Hs.653195 | hs\|16p13.3 | 0.085 | 0.165 | 0.073 | 0.080 | 0.296 | 0.540 | 2.851 | 2.59E−04 |
| PNN | NM_002687 | Hs.409965 | hs\|14q21.1 | −0.107 | −0.050 | −0.098 | −0.029 | 0.247 | 0.348 | 2.851 | 2.38E−04 |
| ABCC8 | NM_000352 | Hs.54470 | hs\|11p15.1 | −0.124 | 0.074 | 0.394 | 0.350 | 0.319 | 0.204 | 2.851 | 1.64E−04 |
| SH3GL2 | NM_003026 | Hs.75149 | hs\|9p22.2 | −0.272 | −0.177 | 0.090 | −0.122 | 0.103 | 0.182 | 2.839 | 4.41E−12 |
| AY358681 | AY358681 | Hs.661469 | hs\|11q24.2 | 0.029 | 0.401 | 0.000 | 0.220 | 0.358 | 0.482 | 2.837 | 7.37E−06 |
| LOC653071 | BC068588 | Hs.626311 | | 0.172 | 0.000 | 0.131 | 0.176 | 0.396 | 0.581 | 2.831 | 7.29E−09 |
| ENST00000355232 | AK160375 | Hs.645346 | hs\|10q11.22 | 0.040 | 0.133 | 0.351 | 0.459 | 0.484 | 0.319 | 2.828 | 5.32E−06 |
| SEC62 | NM_003262 | Hs.592561 | hs\|3q26.2 | 0.148 | 0.009 | 0.168 | 0.231 | 0.584 | 0.598 | 2.818 | 4.14E−11 |
| AK022030 | AK022030 | Hs.288178 | hs\|1q31.2 | 0.022 | 0.099 | 0.136 | 0.179 | 0.367 | 0.470 | 2.809 | 1.14E−04 |
| ENST00000378250 | AK090824 | Hs.653118 | hs\|12q23.1 | −0.464 | 0.010 | −0.267 | −0.154 | −0.078 | −0.071 | 2.806 | 9.73E−04 |
| FLJ37035 | AK094354 | Hs.652548 | hs\|10q26.13 | −0.094 | −0.042 | 0.468 | 0.295 | 0.415 | 0.353 | 2.800 | 6.34E−04 |
| HFM1 | NM_001017975 | Hs.454818 | hs\|1p22.2 | −0.183 | −0.237 | 0.091 | 0.198 | 0.436 | 0.264 | 2.800 | 9.78E−04 |
| SBNO1 | AK074256 | Hs.577403 | hs\|12q24.31 | −0.050 | 0.027 | −0.122 | −0.100 | 0.295 | 0.383 | 2.798 | 2.19E−04 |
| LNX1 | NM_032622 | Hs.655269 | hs\|4q12 | −0.863 | −0.656 | −0.908 | −0.762 | −0.625 | −0.418 | 2.789 | 2.59E−07 |
| BC006271 | BC006271 | | hs\|17q21.31 | 0.032 | 0.135 | 0.238 | 0.166 | 0.336 | 0.479 | 2.783 | 5.20E−04 |
| IFT74 | NM_025103 | Hs.145402 | hs\|9p21.2 | −0.133 | 0.033 | 0.058 | 0.070 | 0.218 | 0.312 | 2.780 | 8.07E−04 |
| SIX4 | NM_017420 | Hs.97849 | hs\|14q23.1 | −0.235 | 0.005 | −0.295 | −0.324 | 0.072 | 0.207 | 2.780 | 8.94E−04 |
| PLCL1 | NM_006226 | Hs.153322 | hs\|2q33.1 | 0.123 | 0.592 | 0.469 | 0.396 | 0.470 | 0.565 | 2.769 | 5.05E−04 |
| ATRX | BC002521 | Hs.533526 | hs\|Xq21.1 | 0.009 | 0.188 | 0.036 | 0.036 | 0.389 | 0.450 | 2.763 | 7.82E−04 |
| SOCS4 | NM_199421 | Hs.532610 | hs\|14q22.3 | −0.042 | −0.107 | −0.100 | −0.120 | 0.307 | 0.340 | 2.762 | 2.88E−06 |
| C14orf45 | NM_025057 | Hs.644621 | hs\|14q24.3 | −0.123 | 0.005 | 0.296 | 0.359 | 0.122 | 0.284 | 2.758 | 3.54E−04 |
| CEP170 | NM_014812 | Hs.533635 | hs\|1q43 | 0.076 | 0.152 | 0.172 | 0.216 | 0.380 | 0.515 | 2.748 | 2.60E−04 |
| THC2657781 | | | hs\|8q21.13 | 0.189 | 0.134 | 0.089 | 0.319 | 0.464 | 0.559 | 2.743 | 8.29E−04 |
| TMEM27 | NM_020665 | Hs.129614 | hs\|Xp22.2 | −0.473 | −0.416 | −0.168 | −0.203 | −0.181 | 0.008 | 2.735 | 6.60E−04 |
| HIST2H2AA4 | NM_003516 | Hs.530461 | hs\|1q21.2 | 0.312 | 0.440 | 0.503 | 0.486 | 0.573 | 0.894 | 2.735 | 7.15E−04 |
| ROCK1 | NM_005406 | Hs.306307 | hs\|18q11.1 | −0.074 | −0.003 | 0.068 | 0.097 | 0.228 | 0.323 | 2.735 | 8.17E−04 |
| DNAH5 | NM_001369 | Hs.212360 | hs\|5p15.2 | 0.000 | 0.000 | 0.563 | 0.408 | 0.414 | 0.165 | 2.733 | 1.42E−12 |
| LOC439949 | AY007155 | Hs.590987 | hs\|10p15.1 | −0.126 | −0.066 | −0.031 | −0.017 | 0.198 | 0.310 | 2.732 | 1.05E−04 |
| AK123861 | AK123861 | Hs.658919 | hs\|3q25.1 | 0.094 | 0.172 | 0.125 | 0.149 | 0.222 | 0.589 | 2.727 | 7.57E−04 |
| C1orf25 | NM_030934 | Hs.591488 | hs\|1q25.3 | 0.034 | 0.273 | −0.042 | 0.049 | 0.412 | 0.470 | 2.725 | 2.87E−10 |
| LCOR | NM_032440 | Hs.500695 | hs\|10q24.1 | 0.048 | 0.013 | −0.053 | 0.060 | 0.307 | 0.399 | 2.719 | 1.52E−04 |
| OXR1 | NM_181354 | Hs.148778 | hs\|8q23.1 | −0.125 | −0.082 | 0.063 | 0.069 | 0.163 | 0.237 | 2.715 | 7.41E−06 |
| IBSP | NM_004967 | Hs.518726 | hs\|4q22.1 | −0.401 | −0.222 | −0.430 | −0.083 | −0.019 | 0.034 | 2.715 | 5.50E−05 |
| TROVE2 | NM_004600 | Hs.288178 | hs\|1q31.2 | 0.028 | 0.127 | 0.131 | 0.183 | 0.370 | 0.462 | 2.713 | 2.38E−04 |
| CD1D | NM_014034 | Hs.292316 | hs\|6q22.31 | 0.334 | 0.133 | 0.367 | 0.346 | 0.504 | 0.786 | 2.707 | 3.93E−04 |
| BE612504 | BE612504 | Hs.618649 | hs\|6q25.3 | 0.009 | 0.105 | 0.205 | 0.318 | −0.097 | 0.441 | 2.705 | 9.73E−13 |
| TRPM7 | NM_017672 | Hs.512894 | hs\|15q21.2 | 0.052 | 0.100 | 0.086 | 0.139 | 0.354 | 0.484 | 2.704 | 2.66E−04 |
| NHLRC3 | AL833329 | Hs.507783 | hs\|13q13.3 | −0.222 | −0.213 | −0.125 | 0.084 | 0.055 | 0.210 | 2.701 | 1.85E−04 |
| THC2548755 | BE091362 | Hs.533222 | hs\|5q12.1 | 0.002 | 0.048 | 0.275 | 0.366 | 0.432 | 0.425 | 2.697 | 3.93E−06 |
| GPATCH2 | NM_018040 | Hs.420757 | hs\|1q41 | −0.012 | 0.068 | 0.183 | 0.252 | 0.336 | 0.418 | 2.695 | 6.85E−07 |
| THC2660448 | | | hs\|1q21.1 | 0.327 | 0.334 | 0.403 | 0.492 | 0.485 | 0.757 | 2.693 | 1.12E−04 |
| ZNF12 | NM_016265 | Hs.431471 | hs\|7p22.1 | 0.109 | 0.120 | 0.158 | 0.187 | 0.390 | 0.536 | 2.693 | 8.24E−06 |
| CCDC144B | NM_182568 | Hs.531547 | hs\|17p11.2 | −0.052 | 0.133 | 0.084 | 0.153 | 0.213 | 0.353 | 2.689 | 4.88E−06 |
| ANXA10 | NM_007193 | Hs.188401 | hs\|4q32.3 | 0.000 | 0.014 | −0.030 | 0.373 | 0.435 | 0.228 | 2.683 | 7.23E−05 |
| KIAA1109 | BC108274 | Hs.408142 | hs\|4q27 | −0.052 | 0.129 | −0.031 | 0.008 | 0.201 | 0.376 | 2.678 | 5.00E−05 |
| LRRC58 | AK056809 | Hs.518084 | hs\|3q13.33 | 0.094 | −0.065 | 0.104 | 0.157 | 0.352 | 0.440 | 2.676 | 4.45E−04 |
| PRDM13 | NM_021620 | Hs.287386 | hs\|6q16.3 | −0.147 | −0.166 | −0.200 | −0.426 | 0.106 | 0.267 | 2.671 | 2.66E−04 |
| SPINK2 | NM_021114 | Hs.98243 | hs\|4q12 | −0.502 | −0.310 | −0.256 | −0.333 | −0.108 | −0.075 | 2.671 | 1.04E−04 |
| CR617033 | CR617033 | Hs.624529 | hs\|2p22.3 | 0.042 | 0.206 | 0.284 | 0.361 | 0.473 | 0.532 | 2.671 | 8.27E−05 |
| THC2651395 | | | hs\|16q24.3 | 0.253 | 0.344 | 0.358 | 0.541 | 0.555 | 0.679 | 2.669 | 5.92E−05 |
| KIF18A | NM_031217 | Hs.301052 | hs\|11p14.1 | −0.145 | −0.092 | −0.103 | −0.129 | 0.317 | 0.281 | 2.667 | 9.49E−04 |
| RAB3GAP2 | NM_012414 | Hs.654849 | hs\|1q41 | 0.070 | 0.138 | 0.133 | 0.110 | 0.403 | 0.498 | 2.665 | 2.08E−04 |
| PLCB1 | NM_182734 | Hs.431173 | hs\|20p12.3 | 0.110 | 0.182 | 0.342 | 0.433 | 0.433 | 0.536 | 2.662 | 3.50E−07 |
| TBK1 | NM_013254 | Hs.505874 | hs\|12q14.2 | 0.066 | 0.094 | 0.066 | 0.095 | 0.404 | 0.491 | 2.660 | 3.80E−04 |
| ZNF10 | NM_015394 | Hs.507355 | hs\|12q24.33 | 0.037 | 0.006 | 0.208 | 0.296 | 0.289 | 0.462 | 2.660 | 1.45E−05 |
| THAP10 | NM_020147 | Hs.591123 | hs\|15q23 | 0.225 | 0.213 | 0.315 | 0.332 | 0.524 | 0.651 | 2.659 | 1.39E−04 |
| ZNF808 | CR749856 | | hs\|19q13.41 | 0.036 | 0.012 | 0.065 | 0.018 | 0.266 | 0.460 | 2.655 | 2.51E−08 |
| BC040982 | BC040982 | Hs.656958 | hs\|18q12.3 | −0.197 | −0.219 | 0.000 | 0.084 | 0.248 | 0.227 | 2.654 | 6.10E−04 |
| DYNC2H1 | NM_001080463 | Hs.503721 | hs\|11q22.3 | −0.136 | −0.064 | 0.028 | −0.100 | 0.266 | 0.287 | 2.650 | 8.22E−04 |
| DIS3 | NM_014953 | Hs.643464 | hs\|13q22.1 | −0.028 | −0.025 | 0.001 | 0.045 | 0.345 | 0.398 | 2.648 | 5.69E−05 |
| ZNF546 | NM_178544 | Hs.693657 | hs\|19q13.2 | −0.022 | 0.141 | −0.030 | 0.079 | 0.226 | 0.401 | 2.647 | 4.23E−04 |
| ZNF254 | NM_203282 | Hs.434406 | hs\|19p12 | −0.004 | 0.053 | −0.003 | 0.029 | 0.289 | 0.418 | 2.644 | 4.39E−05 |
| ZNF559 | NM_032497 | Hs.655107 | hs\|19p13.2 | 0.044 | 0.055 | 0.160 | 0.185 | 0.390 | 0.430 | 2.643 | 7.73E−04 |
| DZIP3 | NM_014648 | Hs.409210 | hs\|3q13.13 | −0.038 | 0.074 | −0.013 | 0.025 | 0.255 | 0.342 | 2.637 | 6.76E−04 |
| BLZF1 | U79751 | Hs.130746 | hs\|1q24.2 | 0.131 | 0.195 | 0.131 | 0.092 | 0.399 | 0.556 | 2.625 | 2.36E−05 |
| THC2642409 | | | hs\|19q13.31 | 0.023 | 0.237 | 0.018 | 0.020 | 0.350 | 0.441 | 2.624 | 1.79E−07 |
| THC2688475 | | | hs\|1q44 | 0.084 | 0.093 | 0.347 | 0.620 | 0.502 | 0.411 | 2.619 | 1.18E−10 |
| CCDC148 | NM_138803 | Hs.668597 | hs\|2q24.1 | 0.224 | 0.625 | 0.409 | 0.536 | 0.605 | 0.658 | 2.615 | 1.34E−04 |
| ZNF655 | NM_001009956 | Hs.521064 | hs\|7q22.1 | 0.220 | 0.146 | 0.231 | 0.305 | 0.469 | 0.617 | 2.612 | 1.30E−04 |
| RABGAP1L | NM_014857 | Hs.585378 | hs\|1q25.1 | −0.225 | −0.129 | −0.010 | −0.006 | 0.086 | 0.084 | 2.611 | 9.51E−05 |
| SETBP1 | NM_013239 | Hs.124942 | hs\|Yp11.32 | 0.323 | 0.166 | 0.731 | 0.783 | 0.739 | 0.695 | 2.610 | 4.77E−04 |
| KRIT1 | NM_194455 | Hs.531987 | hs\|7q21.2 | 0.150 | 0.198 | 0.145 | 0.184 | 0.452 | 0.566 | 2.609 | 2.78E−04 |
| ZFYVE16 | NM_014733 | Hs.482660 | hs\|5q14.1 | 0.159 | 0.292 | 0.269 | 0.260 | 0.436 | 0.575 | 2.607 | 6.56E−04 |
| ZNF765 | NM_001008401 | Hs.433293 | hs\|19q13.41 | 0.068 | 0.069 | 0.093 | 0.059 | 0.321 | 0.478 | 2.604 | 4.62E−04 |
| ZMYM6 | NM_007167 | Hs.533986 | hs\|1p34.3 | 0.027 | 0.143 | 0.179 | 0.193 | 0.378 | 0.443 | 2.602 | 3.01E−04 |
| FBXO15 | NM_152676 | Hs.664011 | hs\|18q22.3 | −0.191 | 0.044 | 0.129 | 0.187 | 0.172 | 0.232 | 2.599 | 9.29E−05 |
| MYO1B | NM_012223 | Hs.439620 | hs\|2q32.3 | −0.020 | −0.007 | −0.077 | −0.027 | 0.196 | 0.423 | 2.594 | 4.73E−04 |
| FGFR1OP2 | NM_015633 | Hs.591162 | hs\|12p11.23 | 0.048 | 0.074 | 0.121 | 0.152 | 0.386 | 0.467 | 2.592 | 1.51E−04 |
| THC2610657 | | | hs\|7q31.33 | −0.011 | 0.070 | 0.070 | 0.083 | 0.353 | 0.408 | 2.590 | 3.35E−04 |
| ENST00000305570 | XR_015683 | Hs.650957 | hs\|21q11.2 | −0.030 | 0.021 | −0.032 | −0.009 | 0.272 | 0.383 | 2.588 | 5.91E−04 |
| ZNF567 | NM_152603 | Hs.412517 | hs\|19q13.12 | 0.046 | 0.048 | 0.006 | −0.020 | 0.342 | 0.423 | 2.586 | 8.99E−05 |
| ZNF479 | AF277624 | Hs.616660 | hs\|7p11.2 | 0.030 | 0.000 | 0.051 | 0.006 | 0.174 | 0.442 | 2.584 | 8.09E−05 |
| ANGPT2 | NM_001147 | Hs.583870 | hs\|8p23.1 | −0.185 | 0.084 | −0.013 | 0.191 | 0.145 | 0.227 | 2.582 | 1.26E−04 |
| ENST00000397141 | XR_015756 | | hs\|19p12 | 0.002 | 0.038 | 0.015 | 0.040 | 0.286 | 0.409 | 2.582 | 6.03E−04 |
| DMTF1 | NM_021145 | Hs.654981 | hs\|7q21.12 | 0.130 | 0.120 | 0.214 | 0.273 | 0.450 | 0.510 | 2.580 | 3.56E−04 |
| BX393727 | BX393727 | Hs.440088 | hs\|5q22.3 | 0.094 | 0.087 | 0.209 | 0.268 | 0.447 | 0.506 | 2.580 | 2.42E−04 |
| H2AFJ | NM_177925 | Hs.524280 | hs\|12p12.3 | −0.121 | 0.124 | 0.025 | −0.023 | 0.182 | 0.290 | 2.579 | 4.35E−04 |
| LOC346887 | BC040619 | Hs.127286 | hs\|8q23.1 | −0.179 | 0.084 | 0.073 | 0.111 | 0.191 | 0.294 | 2.578 | 7.01E−05 |
| ZNF141 | NM_003441 | Hs.654355 | hs\|4p16.3 | −0.014 | −0.054 | 0.021 | 0.019 | 0.195 | 0.253 | 2.575 | 9.55E−04 |
| SDCCAG8 | NM_006642 | Hs.591530 | hs\|1q43 | −0.020 | 0.178 | 0.164 | 0.148 | 0.367 | 0.456 | 2.572 | 1.73E−06 |
| ANKRD26 | NM_014915 | Hs.361041 | hs\|10p12.1 | −0.083 | 0.128 | 0.187 | 0.188 | 0.348 | 0.346 | 2.571 | 4.97E−04 |
| SMCHD1 | AK126324 | Hs.8118 | hs\|18p11.32 | −0.002 | −0.017 | 0.059 | 0.106 | 0.371 | 0.406 | 2.569 | 2.57E−04 |
| RWDD2B | NM_016940 | Hs.34136 | hs\|21q21.3 | −0.257 | −0.143 | 0.156 | 0.087 | 0.093 | 0.153 | 2.565 | 6.28E−04 |
| AW365443 | AW365443 | Hs.568356 | hs\|12p11.21 | −0.166 | 0.000 | −0.172 | −0.295 | 0.000 | 0.243 | 2.564 | 5.15E−04 |
| THC2606573 | AW974708 | Hs.657348 | hs\|3p24.1 | 0.103 | −0.061 | 0.031 | 0.122 | 0.228 | 0.336 | 2.561 | 2.49E−04 |
| PER2 | NM_022817 | Hs.58756 | hs\|2q37.3 | −0.007 | −0.016 | 0.152 | 0.303 | 0.449 | 0.402 | 2.561 | 1.85E−04 |
| MED28 | AF321617 | Hs.644788 | hs\|1q32.1 | 0.219 | 0.172 | 0.408 | 0.473 | 0.559 | 0.626 | 2.557 | 4.98E−12 |
| WBP4 | XR_016161 | Hs.648272 | hs\|22q13.31 | −0.096 | −0.109 | −0.058 | −0.066 | 0.172 | 0.257 | 2.556 | 6.35E−04 |
| THC2641587 | BQ719988 | Hs.660796 | hs\|5q13.2 | 0.222 | 0.352 | 0.513 | 0.597 | 0.708 | 0.594 | 2.556 | 8.54E−11 |
| FAM76B | NM_144664 | Hs.288304 | hs\|11q21 | 0.111 | 0.063 | 0.213 | 0.201 | 0.407 | 0.467 | 2.552 | 9.47E−04 |
| MREG | NM_018000 | Hs.281680 | hs\|2q35 | −0.345 | −0.068 | −0.414 | −0.354 | −0.032 | 0.060 | 2.550 | 1.57E−04 |
| WDR63 | NM_145172 | Hs.97933 | hs\|1p22.3 | 0.050 | 0.224 | 0.344 | 0.391 | 0.255 | 0.472 | 2.550 | 3.68E−05 |
| AF086375 | AF086375 | Hs.264606 | hs\|8q21.13 | 0.001 | −0.035 | 0.104 | 0.107 | 0.249 | 0.407 | 2.549 | 7.78E−04 |
| BE780682 | BE780682 | Hs.355684 | hs\|5p13.2 | −0.052 | −0.005 | −0.083 | 0.001 | 0.272 | 0.365 | 2.548 | 9.95E−05 |
| CD250950 | CD250950 | Hs.658688 | hs\|3q26.33 | −0.163 | −0.163 | −0.162 | −0.166 | 0.229 | 0.242 | 2.545 | 5.47E−09 |
| AK023131 | AK023131 | Hs.648372 | hs\|1q25.3 | −0.110 | −0.066 | 0.058 | 0.124 | 0.221 | 0.296 | 2.545 | 1.30E−05 |
| MALAT1 | NR_002819 | Hs.642877 | hs\|11q13.1 | 0.162 | 0.434 | 0.377 | 0.500 | 0.547 | 0.567 | 2.542 | 3.29E−08 |
| TUBB2B | NM_178012 | Hs.300701 | hs\|6p25.2 | −0.231 | −0.111 | 0.206 | 0.275 | 0.135 | 0.151 | 2.539 | 8.29E−04 |
| MKLN1 | NM_013255 | Hs.44693 | hs\|7q32.3 | −0.051 | 0.128 | 0.003 | 0.072 | 0.242 | 0.353 | 2.531 | 7.54E−06 |
| SETX | NM_015046 | Hs.460317 | hs\|9q34.13 | −0.023 | 0.010 | 0.059 | 0.094 | 0.348 | 0.369 | 2.529 | 7.60E−07 |
| ZBTB20 | BC010934 | Hs.693802 | hs\|3q13.31 | −0.108 | 0.092 | 0.321 | 0.256 | 0.331 | 0.161 | 2.524 | 9.25E−04 |
| DYX1C1 | NM_130810 | Hs.126403 | hs\|15q21.3 | 0.076 | 0.252 | 0.305 | 0.376 | 0.475 | 0.539 | 2.520 | 4.73E−05 |
| CLK1 | NM_004071 | Hs.433732 | hs\|2q33.1 | −0.118 | 0.089 | 0.030 | 0.066 | 0.185 | 0.301 | 2.515 | 5.36E−05 |
| HMCN1 | NM_031935 | Hs.58877 | hs\|1q31.1 | 0.002 | −0.011 | 0.061 | −0.124 | 0.327 | 0.401 | 2.511 | 3.53E−05 |
| ARL13B | NM_182896 | Hs.533086 | hs\|3q11.2 | −0.080 | −0.104 | 0.041 | 0.068 | 0.225 | 0.324 | 2.503 | 5.62E−04 |
| F2R | NM_001992 | Hs.482562 | hs\|5q13.3 | 0.136 | 0.191 | 0.140 | 0.177 | 0.367 | 0.535 | 2.502 | 2.45E−04 |
| EPRS | NM_004446 | Hs.497788 | hs\|1q41 | 0.098 | 0.133 | 0.148 | 0.232 | 0.477 | 0.506 | 2.502 | 3.16E−05 |
| FNDC3A | NM_017416 | Hs.675519 | hs\|Xq22.3 | −0.274 | −0.286 | −0.224 | −0.158 | −0.003 | 0.097 | 2.502 | 1.40E−06 |
| PCDHB14 | NM_018934 | Hs.658497 | hs\|5q31.3 | 0.141 | 0.165 | 0.237 | 0.380 | 0.293 | 0.510 | 2.498 | 6.46E−04 |
| HIST1H2BH | NM_003524 | Hs.247815 | hs\|6p22.1 | 0.139 | 0.247 | 0.182 | 0.153 | 0.366 | 0.536 | 2.497 | 1.15E−06 |
| SFRS18 | AL080186 | Hs.520287 | hs\|6q16.3 | −0.012 | −0.019 | −0.052 | 0.047 | 0.294 | 0.375 | 2.493 | 2.98E−04 |
| TSNAX | NM_005999 | Hs.96247 | hs\|1q42.2 | 0.088 | 0.031 | 0.095 | 0.078 | 0.402 | 0.448 | 2.493 | 6.95E−04 |
| DNAJC13 | NM_015268 | Hs.12707 | hs\|3q22.1 | −0.100 | −0.058 | 0.063 | 0.093 | 0.216 | 0.296 | 2.491 | 7.59E−05 |
| NOP5/NOP58 | NM_015934 | Hs.471104 | hs\|2q33.1 | −0.033 | −0.099 | −0.114 | −0.104 | 0.298 | 0.276 | 2.490 | 3.59E−05 |
| NIN | NM_182944 | Hs.310429 | hs\|14q22.1 | −0.001 | −0.019 | −0.001 | 0.039 | 0.257 | 0.374 | 2.490 | 6.34E−05 |
| ZC3H11A | NM_014827 | Hs.532399 | hs\|1q32.1 | 0.055 | 0.086 | 0.181 | 0.268 | 0.291 | 0.417 | 2.488 | 5.58E−04 |
| C21orf71 | AF086441 | Hs.597706 | hs\|21q21.3 | −0.019 | −0.085 | 0.114 | 0.205 | 0.187 | 0.376 | 2.482 | 6.85E−05 |
| BF984502 | BF984502 | Hs.445603 | hs\|2q31.1 | 0.056 | 0.186 | 0.293 | 0.461 | 0.422 | 0.431 | 2.477 | 3.59E−05 |
| BC034623 | BC034623 | Hs.568682 | hs\|1p12 | −0.252 | −0.130 | 0.000 | 0.188 | −0.015 | 0.141 | 2.472 | 3.24E−11 |
| BNIP3L | NM_004331 | Hs.131226 | hs\|8p21.2 | −0.336 | −0.314 | −0.235 | −0.181 | −0.090 | 0.056 | 2.470 | 3.86E−06 |
| AK021664 | AK021664 | Hs.653123 | hs\|15q21.1 | 0.197 | 0.236 | 0.282 | 0.369 | 0.529 | 0.576 | 2.469 | 8.83E−05 |
| DHX36 | NM_020865 | Hs.446270 | hs\|3q25.2 | −0.063 | 0.006 | 0.035 | 0.075 | 0.286 | 0.337 | 2.467 | 6.76E−04 |
| WDR5B | NM_019069 | Hs.567513 | hs\|3q21.1 | 0.071 | 0.233 | 0.077 | 0.131 | 0.392 | 0.463 | 2.464 | 2.38E−04 |
| H1F0 | NM_005318 | Hs.226117 | hs\|22q13.1 | −0.078 | 0.163 | 0.174 | 0.299 | 0.338 | 0.312 | 2.461 | 1.29E−04 |
| ZNF121 | NM_001008727 | Hs.501537 | hs\|19p13.2 | 0.053 | −0.122 | 0.016 | 0.041 | 0.294 | 0.407 | 2.460 | 4.10E−11 |
| STXBP3 | NM_007269 | Hs.530436 | hs\|1p13.3 | 0.016 | 0.093 | 0.080 | 0.082 | 0.331 | 0.391 | 2.456 | 4.10E−04 |
| CCDC88A | NM_019858 | Hs.631654 | hs\|12p13.31 | −0.134 | −0.076 | −0.071 | −0.026 | 0.188 | 0.256 | 2.454 | 6.92E−04 |
| ARNTL | NM_001178 | Hs.65734 | hs\|11p15.2 | −0.120 | 0.137 | 0.061 | 0.097 | 0.226 | 0.269 | 2.453 | 5.86E−05 |
| SLC30A5 | BX537394 | Hs.631975 | hs\|5q13.2 | 0.147 | 0.170 | 0.211 | 0.217 | 0.410 | 0.536 | 2.449 | 5.93E−05 |
| THC2550620 | | | hs\|17q23.1 | −0.215 | 0.045 | 0.096 | 0.162 | 0.167 | 0.173 | 2.448 | 2.18E−05 |
| DENND4C | NM_017925 | Hs.249591 | hs\|9p22.1 | −0.055 | 0.047 | 0.035 | 0.100 | 0.247 | 0.285 | 2.447 | 1.95E−06 |
| THC2694186 | | | hs\|3p24.3 | −0.050 | 0.009 | 0.167 | 0.187 | 0.307 | 0.322 | 2.442 | 4.47E−04 |
| BM690036 | BM690036 | Hs.121667 | hs\|10q25.2 | −0.007 | −0.029 | 0.052 | 0.169 | 0.232 | 0.379 | 2.441 | 7.34E−07 |
| ICA1L | NM_138468 | Hs.516629 | hs\|2q33.1 | −0.019 | 0.062 | 0.234 | 0.217 | 0.291 | 0.367 | 2.431 | 4.88E−09 |
| ZNF28 | NM_006969 | Hs.554778 | hs\|19q13.41 | 0.108 | 0.069 | 0.058 | −0.009 | 0.278 | 0.451 | 2.429 | 4.31E−04 |
| TXNDC10 | NM_019022 | Hs.440534 | hs\|18q22.1 | −0.013 | −0.044 | 0.030 | 0.008 | 0.269 | 0.290 | 2.428 | 4.83E−04 |
| BBS10 | NM_024685 | Hs.96322 | hs\|12q21.2 | 0.190 | 0.168 | 0.262 | 0.401 | 0.565 | 0.575 | 2.425 | 4.54E−05 |
| KIF2A | NM_004520 | Hs.558351 | hs\|5q12.1 | −0.065 | 0.058 | 0.097 | 0.136 | 0.305 | 0.319 | 2.422 | 7.31E−04 |
| THC2654993 | | | hs\|8q12.1 | 0.118 | −0.012 | 0.071 | 0.044 | 0.245 | 0.327 | 2.421 | 7.73E−04 |
| TGM1 | NM_000359 | Hs.508950 | hs\|14q12 | −0.037 | −0.245 | −0.301 | −0.348 | 0.121 | 0.049 | 2.421 | 7.02E−04 |
| SCN2A | NM_021007 | Hs.93485 | hs\|2q24.3 | −0.304 | −0.001 | −0.022 | −0.077 | 0.013 | 0.082 | 2.418 | 7.22E−04 |
| ATG4C | AK027773 | Hs.7353 | hs\|1p31.3 | −0.030 | 0.041 | −0.019 | 0.010 | 0.237 | 0.353 | 2.417 | 3.46E−04 |
| MBTD1 | AL133577 | Hs.656803 | hs\|17q21.33 | −0.104 | −0.125 | −0.015 | 0.019 | 0.206 | 0.279 | 2.414 | 3.65E−04 |
| ENST00000377525 | BC119676 | Hs.567050 | hs\|9q12 | −0.036 | 0.292 | 0.149 | 0.227 | 0.273 | 0.348 | 2.411 | 1.60E−05 |
| C21orf91 | NM_017447 | Hs.293811 | hs\|21q21.1 | 0.168 | −0.027 | 0.040 | 0.066 | 0.289 | 0.400 | 2.409 | 9.64E−04 |
| ANKRD32 | NM_032290 | Hs.657315 | hs\|5q15 | 0.108 | 0.183 | 0.144 | 0.168 | 0.566 | 0.518 | 2.408 | 2.52E−04 |
| ENST00000381298 | AB074172 | Hs.532082 | hs\|5q11.2 | 0.216 | 0.199 | 0.273 | 0.316 | 0.432 | 0.598 | 2.407 | 7.12E−05 |
| SR140 | NM_001080415 | Hs.596572 | hs\|3q23 | −0.141 | −0.156 | −0.090 | −0.096 | 0.338 | 0.250 | 2.407 | 5.28E−04 |
| KGFLP1 | AY098593 | Hs.439341 | hs\|9p11.2 | −0.140 | −0.070 | 0.014 | −0.059 | 0.157 | 0.419 | 2.406 | 8.43E−04 |
| UBLCP1 | NM_145049 | Hs.591733 | hs\|5q33.3 | 0.082 | 0.101 | 0.137 | 0.202 | 0.337 | 0.463 | 2.404 | 2.38E−05 |
| NPDC1 | NM_015392 | Hs.105547 | hs\|9q34.3 | −0.054 | 0.275 | 0.207 | 0.246 | 0.371 | 0.425 | 2.403 | 8.12E−04 |
| BX641014 | BX641014 | Hs.648609 | hs\|9p11.2 | −0.144 | 0.151 | −0.275 | −0.216 | 0.347 | 0.547 | 2.403 | 6.15E−04 |
| ZNF75A | NM_153028 | Hs.513292 | hs\|16p13.3 | −0.063 | 0.036 | 0.131 | 0.116 | 0.243 | 0.393 | 2.402 | 4.13E−04 |
| SENP7 | NM_020654 | Hs.529551 | hs\|3q12.3 | 0.017 | 0.032 | 0.094 | 0.064 | 0.216 | 0.409 | 2.400 | 2.86E−05 |
| C3orf63 | NM_015224 | Hs.116877 | hs\|3p14.3 | 0.011 | 0.051 | 0.081 | 0.129 | 0.295 | 0.370 | 2.397 | 6.66E−04 |
| PDE5A | NM_001083 | Hs.647971 | hs\|4q27 | −0.527 | −0.423 | −0.280 | −0.218 | −0.257 | −0.147 | 2.395 | 1.54E−04 |
| LOC644192 | AK000872 | Hs.58690 | hs\|15q26.2 | 0.056 | 0.154 | 0.259 | 0.405 | 0.425 | 0.259 | 2.393 | 2.86E−05 |
| THC2693401 | | | hs\|11q22.3 | 0.033 | 0.129 | 0.102 | 0.175 | 0.304 | 0.411 | 2.391 | 1.42E−04 |
| XR_018202 | XR_018202 | Hs.567832 | hs\|Xq13.3 | −0.028 | 0.113 | 0.020 | 0.094 | 0.311 | 0.350 | 2.390 | 4.49E−04 |
| ATF7IP2 | NM_024997 | Hs.513343 | hs\|16p13.13 | −0.092 | 0.154 | −0.038 | 0.046 | 0.181 | 0.285 | 2.383 | 1.73E−04 |
| ZBTB10 | NM_023929 | Hs.591868 | hs\|8q21.13 | −0.236 | −0.054 | −0.131 | −0.176 | 0.086 | 0.141 | 2.382 | 9.24E−04 |
| IQCH | NM_022784 | Hs.657894 | hs\|15q23 | −0.056 | 0.068 | 0.230 | 0.291 | 0.402 | 0.320 | 2.382 | 2.75E−04 |
| ZNF624 | NM_020787 | Hs.128078 | hs\|17p11.2 | 0.015 | 0.094 | 0.108 | 0.194 | 0.330 | 0.394 | 2.382 | 1.96E−04 |
| C14orf115 | NM_018228 | Hs.578167 | hs\|14q24.3 | −0.367 | −0.033 | −0.147 | −0.102 | −0.034 | 0.010 | 2.381 | 9.05E−06 |
| ZNF347 | NM_032584 | Hs.467239 | hs\|19q13.41 | 0.013 | −0.072 | 0.027 | 0.019 | 0.247 | 0.369 | 2.381 | 1.18E−04 |
| HES1 | NM_005524 | Hs.250666 | hs\|3q29 | 0.140 | 0.630 | 0.323 | 0.358 | 0.434 | 0.534 | 2.380 | 9.79E−04 |
| AV714556 | AV714556 | Hs.459174 | hs\|8q24.13 | 0.005 | 0.040 | 0.109 | 0.151 | 0.377 | 0.382 | 2.380 | 1.19E−04 |
| ZNF345 | NM_003419 | Hs.362324 | hs\|19q13.12 | 0.246 | 0.155 | 0.196 | 0.290 | 0.503 | 0.571 | 2.378 | 2.78E−11 |
| THC2676741 | | | hs\|7q32.2 | 0.073 | 0.181 | 0.214 | 0.304 | 0.390 | 0.449 | 2.378 | 3.36E−05 |
| AI206757 | AI206757 | Hs.227777 | hs\|6q12 | 0.241 | 0.680 | 0.330 | 0.379 | 0.539 | 0.617 | 2.378 | 8.99E−04 |
| RND3 | NM_005168 | Hs.6838 | hs\|2q23.3 | 0.223 | 0.075 | 0.212 | 0.219 | 0.357 | 0.581 | 2.368 | 1.32E−04 |
| UCP3 | NM_003356 | Hs.101337 | hs\|11q13.4 | 0.042 | 0.154 | 0.246 | 0.276 | 0.325 | 0.422 | 2.365 | 2.22E−04 |
| FAM29A | NM_017645 | Hs.533468 | hs\|9p22.1 | −0.089 | −0.109 | −0.064 | −0.044 | 0.258 | 0.274 | 2.365 | 4.73E−04 |
| BX648207 | BX648207 | Hs.23554 | hs\|12q12 | 0.079 | 0.098 | 0.087 | 0.106 | 0.445 | 0.451 | 2.364 | 1.03E−04 |
| ABCA5 | NM_018672 | Hs.421474 | hs\|17q24.3 | 0.131 | 0.111 | 0.184 | 0.242 | 0.466 | 0.507 | 2.363 | 3.60E−05 |
| ENST00000369158 | BC015544 | | hs\|1q21.2 | 0.201 | 0.089 | 0.145 | 0.248 | 0.350 | 0.429 | 2.362 | 1.54E−04 |
| MGEA5 | AF307332 | Hs.500842 | hs\|10q24.32 | 0.053 | 0.060 | 0.135 | 0.219 | 0.431 | 0.419 | 2.362 | 1.04E−04 |
| VAMP4 | AK056124 | Hs.6651 | hs\|1q24.3 | 0.095 | 0.121 | 0.210 | 0.303 | 0.341 | 0.468 | 2.361 | 3.02E−05 |
| CCDC11 | NM_145020 | Hs.658630 | hs\|18q21.1 | 0.000 | −0.071 | 0.000 | 0.397 | 0.296 | 0.342 | 2.360 | 4.36E−04 |
| OSBPL8 | NM_020841 | Hs.430849 | hs\|12q21.2 | 0.110 | 0.077 | 0.090 | 0.151 | 0.353 | 0.447 | 2.360 | 3.35E−05 |
| THC2641484 | | | hs\|22q11.1 | −0.372 | −0.170 | 0.000 | 0.000 | 0.119 | −0.177 | 2.360 | 8.47E−04 |
| SCYL1BP1 | NM_152281 | Hs.183702 | hs\|1q24.2 | 0.038 | 0.081 | 0.219 | 0.236 | 0.345 | 0.477 | 2.358 | 6.04E−04 |
| UTP15 | NM_032175 | Hs.406703 | hs\|5q13.2 | 0.055 | −0.041 | −0.047 | −0.064 | 0.309 | 0.326 | 2.352 | 5.25E−05 |
| RRAD | NM_004165 | Hs.1027 | hs\|16q22.1 | 0.039 | 0.199 | 0.179 | 0.239 | 0.388 | 0.411 | 2.351 | 2.65E−04 |
| ZNF675 | NM_138330 | Hs.264345 | hs\|19p12 | −0.004 | 0.027 | 0.010 | 0.031 | 0.284 | 0.368 | 2.349 | 2.72E−04 |
| AK309617 | | | hs\|9q22.31 | −0.075 | −0.058 | −0.001 | 0.009 | 0.218 | 0.295 | 2.346 | 2.88E−04 |
| RP5-1022P6.2 | NM_019593 | Hs.636359 | hs\|20p12.3 | 0.065 | 0.053 | 0.076 | 0.198 | 0.485 | 0.435 | 2.343 | 1.35E−04 |
| LOC442590 | NM_175064 | Hs.645483 | hs\|7p13 | 0.055 | 0.198 | 0.271 | 0.541 | 0.370 | 0.427 | 2.343 | 2.92E−04 |
| CR622342 | AK057480 | Hs.527105 | hs\|4q21.22 | −0.106 | 0.094 | 0.034 | 0.107 | 0.215 | 0.264 | 2.342 | 3.42E−04 |
| ZNF227 | NM_182490 | Hs.371335 | hs\|19q13.31 | 0.009 | 0.072 | 0.005 | 0.021 | 0.280 | 0.378 | 2.340 | 8.06E−11 |
| LOC440295 | NM_198181 | Hs.660597 | | −0.073 | 0.076 | −0.022 | 0.022 | 0.168 | 0.296 | 2.339 | 5.68E−05 |
| MIA3 | NM_198551 | Hs.118474 | hs\|1q41 | 0.100 | 0.107 | 0.182 | 0.242 | 0.392 | 0.468 | 2.337 | 3.83E−05 |
| KRR1 | NM_007043 | Hs.645517 | hs\|12q21.2 | 0.112 | 0.059 | 0.059 | 0.096 | 0.423 | 0.427 | 2.334 | 1.98E−04 |
| TIA1 | NM_022173 | Hs.516075 | hs\|2p14 | −0.102 | −0.075 | 0.073 | 0.115 | 0.221 | 0.265 | 2.326 | 1.27E−04 |
| CCT6AP1 | AK092180 | | hs\|7q11.21 | 0.065 | 0.084 | 0.163 | 0.207 | 0.350 | 0.431 | 2.325 | 4.76E−05 |
| ZDHHC11 | NM_024786 | Hs.659832 | hs\|5p15.33 | −0.019 | 0.163 | 0.119 | 0.222 | 0.248 | 0.347 | 2.323 | 6.15E−04 |
| CCDC150 | NM_001080539 | Hs.132519 | hs\|2q33.1 | −0.069 | −0.096 | 0.049 | 0.216 | 0.211 | 0.187 | 2.323 | 9.23E−04 |
| POLQ | NM_199420 | Hs.241517 | hs\|3q13.33 | −0.085 | −0.103 | 0.036 | 0.179 | 0.346 | 0.264 | 2.323 | 5.57E−04 |
| VPS37A | AL834189 | Hs.343873 | hs\|8p22 | 0.006 | −0.003 | 0.026 | 0.030 | 0.233 | 0.337 | 2.323 | 2.55E−04 |
| LARP2 | NM_004208 | Hs.424932 | hs\|Xq25 | 0.141 | 0.159 | 0.334 | 0.356 | 0.434 | 0.530 | 2.320 | 6.11E−04 |
| ZNF177 | NM_003451 | Hs.172979 | hs\|19p13.2 | 0.038 | 0.314 | 0.082 | 0.082 | 0.230 | 0.442 | 2.315 | 3.05E−04 |
| C11orf47 | NM_173589 | Hs.377188 | hs\|11p15.4 | −0.299 | −0.043 | 0.000 | 0.053 | 0.110 | −0.021 | 2.314 | 6.77E−04 |
| THC2730719 | | | hs\|12p13.2 | 0.124 | −0.043 | −0.080 | −0.022 | 0.292 | 0.321 | 2.313 | 4.71E−12 |
| AGGF1 | NM_018046 | Hs.634849 | hs\|5q13.3 | 0.113 | 0.137 | 0.210 | 0.252 | 0.344 | 0.471 | 2.312 | 2.96E−05 |
| BIVM | NM_017693 | Hs.288809 | hs\|13q33.1 | −0.141 | −0.116 | −0.065 | 0.090 | 0.137 | 0.222 | 2.308 | 1.35E−09 |
| DQ786252 | DQ786252 | Hs.645142 | hs\|10q26.11 | −0.094 | −0.054 | −0.111 | −0.069 | 0.196 | 0.267 | 2.306 | 9.51E−04 |
| ATG2B | NM_018036 | Hs.168241 | hs\|14q32.2 | −0.063 | −0.043 | −0.037 | 0.065 | 0.149 | 0.299 | 2.303 | 2.11E−05 |
| CRYGS | NM_017541 | Hs.376209 | hs\|3q27.3 | 0.026 | 0.102 | 0.193 | 0.330 | 0.278 | 0.388 | 2.303 | 5.18E−04 |
| CR617865 | BQ018421 | Hs.525163 | hs\|13q34 | −0.074 | −0.014 | 0.028 | 0.095 | 0.266 | 0.288 | 2.302 | 7.94E−05 |
| CRH | NM_000756 | Hs.75294 | hs\|8q13.1 | 0.000 | 0.027 | 0.000 | 0.312 | 0.234 | 0.366 | 2.300 | 3.82E−04 |
| ZFAND1 | NM_024699 | Hs.655453 | hs\|8q21.13 | −0.038 | −0.053 | 0.066 | 0.126 | 0.272 | 0.324 | 2.299 | 1.76E−04 |
| TMEM68 | NM_152417 | Hs.420076 | hs\|8q12.1 | −0.095 | 0.002 | −0.023 | 0.000 | 0.166 | 0.266 | 2.296 | 4.58E−04 |
| SNX2 | NM_003100 | Hs.134822 | hs\|5q23.2 | 0.086 | 0.161 | 0.126 | 0.166 | 0.343 | 0.451 | 2.295 | 2.67E−04 |
| FUSIP1 | NM_054016 | Hs.3530 | hs\|1p36.11 | 0.004 | −0.084 | 0.022 | 0.051 | 0.325 | 0.364 | 2.294 | 3.16E−06 |
| MORC4 | NM_024657 | Hs.496544 | hs\|Xq22.3 | 0.099 | 0.076 | 0.110 | 0.228 | 0.330 | 0.431 | 2.294 | 1.97E−05 |
| AK091904 | AK091904 | Hs.202577 | hs\|3q13.31 | 0.018 | 0.205 | 0.269 | 0.214 | 0.389 | 0.446 | 2.293 | 7.75E−04 |
| JHDM1D | NM_030647 | Hs.308710 | hs\|7q34 | 0.015 | 0.194 | 0.082 | 0.110 | 0.252 | 0.374 | 2.289 | 7.52E−05 |
| DPY19L4 | NM_181787 | Hs.567828 | hs\|8q22.1 | −0.130 | −0.104 | 0.073 | 0.124 | 0.194 | 0.229 | 2.286 | 2.55E−05 |
| BU173515 | BU173515 | Hs.655113 | hs\|11p15.1 | 0.000 | 0.012 | 0.062 | 0.053 | 0.200 | 0.359 | 2.285 | 1.36E−06 |
| NAT12 | | | hs\|7p12.1 | −0.140 | −0.109 | −0.145 | −0.143 | 0.141 | 0.218 | 2.284 | 6.58E−05 |
| TMTC3 | NM_181783 | Hs.331268 | hs\|12q21.32 | 0.031 | 0.040 | 0.013 | 0.018 | 0.286 | 0.376 | 2.284 | 2.76E−06 |
| LACTB2 | NM_016027 | Hs.118554 | hs\|8q13.3 | −0.053 | 0.018 | 0.093 | 0.092 | 0.285 | 0.305 | 2.278 | 3.68E−04 |
| C1orf181 | NM_017953 | Hs.5111 | hs\|1p22.3 | 0.087 | 0.119 | 0.146 | 0.198 | 0.347 | 0.423 | 2.274 | 7.66E−04 |
| FLJ11292 | AK023417 | Hs.694230 | hs\|5q14.3 | −0.004 | 0.000 | 0.091 | 0.119 | 0.299 | 0.352 | 2.272 | 3.19E−05 |
| ZEB1 | NM_030751 | Hs.124503 | hs\|10p11.22 | −0.128 | −0.095 | −0.099 | −0.037 | 0.131 | 0.227 | 2.271 | 2.91E−05 |
| UFM1 | NM_016617 | Hs.693686 | hs\|13q13.3 | 0.005 | −0.110 | 0.007 | 0.035 | 0.245 | 0.359 | 2.271 | 1.01E−07 |
| LOC728927 | XM_001128828 | Hs.670568 | hs\|7q11.21 | 0.024 | −0.172 | 0.029 | 0.025 | 0.312 | 0.371 | 2.270 | 7.70E−06 |
| C1orf58 | NM_144695 | Hs.552608 | hs\|1q41 | 0.082 | 0.072 | 0.211 | 0.241 | 0.371 | 0.437 | 2.268 | 5.16E−04 |
| TAF2 | NM_003184 | Hs.122752 | hs\|8q24.12 | −0.006 | 0.005 | 0.030 | 0.067 | 0.282 | 0.355 | 2.267 | 1.29E−04 |
| ZNF30 | NM_194325 | Hs.657402 | hs\|19q13.11 | 0.070 | 0.048 | 0.087 | 0.097 | 0.305 | 0.419 | 2.265 | 8.68E−05 |
| HIST1H2BL | NM_003519 | Hs.137594 | hs\|6p22.1 | 0.127 | 0.199 | 0.167 | 0.133 | 0.342 | 0.513 | 2.261 | 2.02E−04 |
| CSNK1G3 | NM_004384 | Hs.129206 | hs\|5q23.2 | 0.108 | 0.091 | 0.164 | 0.213 | 0.375 | 0.463 | 2.260 | 3.34E−05 |
| PREPL | NM_006036 | Hs.444349 | hs\|2p21 | 0.031 | 0.017 | 0.042 | 0.040 | 0.265 | 0.325 | 2.258 | 6.98E−04 |
| ACADM | NM_000016 | Hs.445040 | hs\|1p31.1 | −0.076 | −0.024 | 0.023 | −0.033 | 0.183 | 0.276 | 2.250 | 6.88E−06 |
| FLJ39653 | AK093550 | Hs.445315 | hs\|4p15.32 | −0.008 | 0.144 | 0.141 | 0.265 | 0.309 | 0.350 | 2.249 | 5.03E−04 |
| MATR3 | NM_199189 | Hs.268939 | hs\|5q31.2 | −0.041 | −0.033 | −0.070 | −0.068 | 0.231 | 0.319 | 2.247 | 8.31E−05 |
| KIAA1012 | NM_014939 | Hs.202001 | hs\|18q12.1 | −0.015 | 0.047 | 0.111 | 0.117 | 0.265 | 0.336 | 2.245 | 1.27E−07 |
| SYF2 | NM_015484 | Hs.20013 | hs\|1p36.11 | 0.135 | 0.173 | 0.266 | 0.345 | 0.383 | 0.478 | 2.244 | 2.37E−04 |
| FAM71A | NM_153606 | Hs.129293 | hs\|1q32.3 | −0.348 | −0.050 | −0.172 | −0.003 | −0.031 | 0.016 | 2.243 | 3.20E−04 |
| RCOR3 | NM_018254 | Hs.356399 | hs\|1q32.3 | 0.100 | 0.142 | 0.255 | 0.285 | 0.420 | 0.452 | 2.242 | 2.57E−04 |
| HIST1H3F | BC062305 | Hs.70937 | hs\|6p22.1 | 0.156 | 0.209 | 0.154 | 0.150 | 0.310 | 0.506 | 2.241 | 2.89E−04 |
| SLC43A2 | NM_152346 | Hs.160550 | hs\|17p13.3 | 0.007 | 0.171 | −0.052 | −0.013 | 0.343 | 0.357 | 2.240 | 2.71E−05 |
| TSEN54 | AK094466 | Hs.655875 | hs\|17q25.1 | −0.098 | 0.051 | 0.134 | 0.195 | 0.329 | 0.260 | 2.239 | 9.66E−04 |
| FLJ13611 | NM_024941 | Hs.591760 | hs\|5q12.3 | 0.172 | 0.069 | 0.163 | 0.225 | 0.304 | 0.389 | 2.237 | 1.25E−04 |
| TWF1 | NM_002822 | Hs.189075 | hs\|12q12 | 0.026 | 0.004 | 0.018 | 0.021 | 0.296 | 0.368 | 2.233 | 4.11E−05 |
| PTAR1 | AL832683 | Hs.494100 | hs\|9q21.11 | 0.123 | 0.081 | 0.070 | 0.124 | 0.379 | 0.429 | 2.227 | 9.47E−06 |
| SMAD2 | NM_001003652 | Hs.12253 | hs\|18q21.1 | −0.005 | −0.063 | 0.145 | 0.227 | 0.296 | 0.278 | 2.226 | 4.30E−04 |
| THC2610890 | | | hs\|3p14.1 | 0.105 | 0.128 | 0.243 | 0.310 | 0.493 | 0.451 | 2.225 | 1.64E−04 |
| ARHGAP12 | NM_018287 | Hs.499264 | hs\|10p11.22 | −0.019 | 0.055 | 0.050 | 0.069 | 0.234 | 0.328 | 2.225 | 4.44E−04 |
| LOC727834 | XM_926013 | | | −0.170 | 0.070 | −0.058 | −0.001 | 0.087 | 0.177 | 2.223 | 2.62E−07 |
| KLC1 | AK092888 | Hs.20107 | hs\|14q32.33 | −0.293 | −0.178 | −0.032 | 0.074 | 0.055 | 0.052 | 2.222 | 1.88E−05 |
| UHRF1BP1L | NM_015054 | Hs.620701 | hs\|12q23.1 | 0.037 | 0.021 | 0.093 | 0.144 | 0.238 | 0.247 | 2.221 | 2.93E−04 |
| IL6ST | NM_002184 | Hs.532082 | hs\|5q11.2 | 0.221 | 0.163 | 0.220 | 0.303 | 0.379 | 0.550 | 2.221 | 1.52E−04 |
| RGS5 | NM_003617 | Hs.24950 | hs\|1q23.3 | 0.421 | 0.562 | 0.647 | 0.740 | 0.716 | 0.767 | 2.220 | 1.38E−04 |
| PRKAA2 | BC043195 | Hs.437039 | hs\|1p32.2 | 0.113 | 0.017 | 0.080 | 0.155 | 0.279 | 0.340 | 2.220 | 4.19E−05 |
| CF143262 | CF143262 | Hs.252387 | hs\|22q13.31 | 0.272 | 0.045 | 0.140 | 0.210 | 0.304 | 0.425 | 2.219 | 1.79E−04 |
| WRN | NM_000553 | Hs.632050 | hs\|8p12 | 0.191 | 0.194 | 0.325 | 0.450 | 0.538 | 0.536 | 2.217 | 9.80E−04 |
| THC2585464 | | | hs\|11q23.3 | −0.162 | −0.137 | −0.099 | −0.020 | 0.066 | 0.182 | 2.211 | 1.01E−05 |
| DPY19L2P4 | AK098759 | Hs.406964 | hs\|7q21.13 | 0.232 | 0.340 | 0.416 | 0.579 | 0.613 | 0.576 | 2.210 | 4.52E−04 |
| ZNF700 | NM_144566 | Hs.528486 | hs\|19p13.2 | 0.015 | 0.058 | 0.083 | 0.157 | 0.281 | 0.374 | 2.208 | 2.92E−04 |
| AK026668 | AK026668 | Hs.693653 | hs\|1q23.3 | 0.230 | 0.205 | 0.238 | 0.251 | 0.539 | 0.581 | 2.208 | 1.95E−04 |
| TRIM36 | NM_018700 | Hs.519514 | hs\|5q22.3 | 0.198 | 0.027 | 0.050 | 0.053 | 0.286 | 0.371 | 2.207 | 4.74E−04 |
| ZNF146 | NM_007145 | Hs.643436 | hs\|19q13.12 | −0.060 | −0.021 | −0.052 | −0.040 | 0.209 | 0.301 | 2.205 | 1.57E−04 |
| ZFP1 | NM_152904 | Hs.431045 | hs\|17p11.2 | 0.038 | 0.088 | 0.098 | 0.145 | 0.375 | 0.377 | 2.203 | 2.11E−04 |
| AK091744 | AK091744 | Hs.622771 | hs\|10q23.2 | −0.118 | 0.099 | 0.184 | 0.215 | 0.223 | 0.098 | 2.203 | 9.19E−07 |
| FLJ13305 | BX648834 | Hs.440466 | hs\|2p15 | −0.170 | −0.179 | −0.002 | 0.089 | 0.169 | 0.163 | 2.200 | 8.12E−04 |
| GOLGA1 | AK021820 | Hs.133469 | hs\|9q33.3 | 0.068 | 0.043 | 0.067 | 0.287 | 0.254 | 0.400 | 2.199 | 9.81E−04 |
| CAND1 | AK027783 | Hs.546407 | hs\|12q14.3 | −0.150 | −0.075 | 0.009 | 0.214 | 0.170 | 0.140 | 2.199 | 2.56E−04 |
| ZNF283 | AK098175 | Hs.652513 | hs\|19q13.31 | 0.044 | 0.069 | −0.013 | −0.040 | 0.252 | 0.337 | 2.199 | 1.87E−04 |
| ZZZ3 | NM_015534 | Hs.480506 | hs\|1p31.1 | 0.084 | 0.120 | 0.085 | 0.112 | 0.388 | 0.454 | 2.195 | 7.21E−04 |
| FAM44A | NM_148894 | Hs.444517 | hs\|4p15.33 | −0.164 | 0.209 | −0.265 | −0.172 | 0.140 | 0.176 | 2.187 | 1.46E−04 |
| ZBTB38 | NM_001080412 | Hs.518301 | hs\|3q23 | −0.003 | −0.102 | −0.018 | −0.008 | 0.117 | 0.181 | 2.187 | 5.26E−04 |
| ZNF492 | BC110575 | Hs.232108 | hs\|19p12 | −0.004 | 0.008 | −0.021 | 0.022 | 0.247 | 0.335 | 2.183 | 1.38E−04 |
| THC2658030 | BX089071 | Hs.664834 | hs\|1p34.2 | −0.057 | −0.002 | 0.138 | 0.212 | 0.259 | 0.228 | 2.177 | 3.95E−04 |
| MBNL1 | NM_021038 | Hs.478000 | hs\|3q25.2 | −0.054 | −0.012 | 0.073 | 0.108 | 0.255 | 0.284 | 2.177 | 7.04E−05 |
| BC027922 | BC027922 | Hs.288995 | hs\|19q13.43 | 0.239 | 0.082 | 0.126 | 0.210 | 0.284 | 0.434 | 2.176 | 6.04E−04 |
| AK130891 | AK130891 | Hs.656546 | hs\|8q24.13 | −0.231 | 0.013 | 0.090 | 0.103 | 0.223 | 0.045 | 2.176 | 1.27E−04 |
| THUMPD1 | NM_017736 | Hs.460232 | hs\|16p12.2 | 0.045 | 0.003 | −0.021 | −0.076 | 0.324 | 0.339 | 2.175 | 5.93E−05 |
| SUZ12P | CR597846 | Hs.628886 | hs\|17q11.2 | −0.200 | −0.134 | −0.081 | 0.016 | 0.070 | 0.130 | 2.174 | 1.11E−05 |
| BC037740 | BC037740 | Hs.597434 | hs\|17q11.2 | 0.033 | 0.061 | 0.093 | 0.116 | 0.289 | 0.369 | 2.172 | 1.68E−04 |
| EVI1 | NM_005241 | Hs.656395 | hs\|3q26.2 | 0.001 | −0.012 | 0.112 | 0.196 | 0.328 | 0.338 | 2.172 | 2.97E−04 |
| CAMTA1 | | | hs\|Xq25 | −0.062 | −0.002 | 0.170 | 0.182 | 0.196 | 0.275 | 2.172 | 9.65E−05 |
| ACTA1 | AK095258 | Hs.16622 | hs\|Xq28 | 0.025 | 0.057 | 0.117 | 0.111 | 0.251 | 0.363 | 2.168 | 2.29E−04 |
| CTGLF5 | NM_133446 | Hs.656384 | hs\|10q11.21 | −0.032 | 0.112 | 0.212 | 0.315 | 0.296 | 0.240 | 2.166 | 1.59E−04 |
| SMC5 | AB011166 | Hs.534189 | hs\|9q21.11 | 0.030 | 0.047 | 0.033 | 0.068 | 0.383 | 0.366 | 2.165 | 5.06E−05 |
| PIH1D2 | NM_138789 | Hs.420662 | hs\|11q23.1 | 0.022 | 0.145 | 0.200 | 0.189 | 0.369 | 0.357 | 2.163 | 2.38E−04 |
| SNX16 | NM_022133 | Hs.492121 | hs\|8q21.13 | −0.053 | 0.114 | 0.046 | 0.068 | 0.199 | 0.282 | 2.162 | 7.09E−05 |
| ZNF578 | AK095562 | Hs.676961 | hs\|19q13.41 | 0.057 | −0.022 | 0.031 | 0.001 | 0.201 | 0.366 | 2.161 | 2.93E−04 |
| THC2611971 | | | hs\|7p21.1 | 0.065 | 0.064 | 0.154 | 0.184 | 0.349 | 0.400 | 2.161 | 2.53E−04 |
| PNPLA8 | NM_015723 | Hs.617340 | hs\|7q31.1 | 0.122 | 0.125 | 0.123 | 0.070 | 0.224 | 0.477 | 2.160 | 7.98E−04 |
| ZNF826 | NM_001039884 | Hs.631635 | hs\|19p12 | 0.000 | 0.023 | 0.011 | 0.037 | 0.244 | 0.344 | 2.155 | 6.88E−10 |
| HIST1H2BK | NM_080593 | Hs.437275 | hs\|6p22.1 | 0.370 | 0.364 | 0.432 | 0.401 | 0.524 | 0.706 | 2.154 | 3.46E−04 |
| FAM27E3 | BC032035 | Hs.31240 | hs\|9q12 | 0.004 | 0.298 | 0.148 | 0.217 | 0.277 | 0.337 | 2.154 | 6.39E−07 |
| C1QTNF3 | NM_181435 | Hs.171929 | hs\|5p13.3 | −0.045 | 0.084 | 0.184 | 0.240 | 0.304 | 0.266 | 2.152 | 9.71E−05 |
| FBN2 | X62009 | Hs.519294 | hs\|5q23.3 | −0.351 | −0.137 | −0.032 | 0.000 | −0.021 | −0.019 | 2.151 | 3.48E−06 |
| APC | NM_000038 | Hs.158932 | hs\|5q22.2 | 0.110 | 0.130 | 0.114 | 0.128 | 0.377 | 0.446 | 2.148 | 2.61E−04 |
| TTC14 | NM_133462 | Hs.43213 | hs\|3q26.33 | 0.038 | −0.022 | −0.009 | 0.030 | 0.264 | 0.295 | 2.148 | 4.46E−04 |
| SPAG1 | NM_003114 | Hs.591866 | hs\|8q22.2 | 0.065 | −0.014 | 0.028 | 0.072 | 0.221 | 0.340 | 2.148 | 4.28E−04 |
| TWISTNB | NM_001002926 | Hs.353035 | hs\|7p15.3 | 0.210 | 0.122 | 0.149 | 0.146 | 0.444 | 0.487 | 2.148 | 3.26E−04 |
| LOC100132006 | AK092846 | Hs.593666 | hs\|16p13.2 | −0.050 | 0.105 | 0.089 | 0.164 | 0.223 | 0.176 | 2.146 | 3.28E−04 |
| DUXAP10 | AK056135 | Hs.536395 | hs\|22q11.1 | −0.130 | 0.035 | −0.070 | 0.077 | 0.152 | 0.201 | 2.141 | 5.07E−04 |
| LMLN | AL832783 | Hs.518540 | hs\|3q29 | −0.065 | 0.171 | 0.214 | 0.228 | 0.308 | 0.399 | 2.140 | 2.42E−04 |
| HLTF | NM_003071 | Hs.3068 | hs\|3q24 | −0.070 | −0.098 | −0.038 | 0.018 | 0.224 | 0.264 | 2.138 | 2.76E−04 |
| LOC100132439 | | | hs\|9q12 | 0.011 | 0.329 | 0.179 | 0.242 | 0.315 | 0.341 | 2.135 | 2.23E−05 |
| TRAM1 | NM_014294 | Hs.491988 | hs\|8q13.3 | −0.063 | −0.007 | 0.036 | 0.060 | 0.186 | 0.284 | 2.133 | 1.10E−04 |
| JMJD1A | NM_018433 | Hs.557425 | hs\|2p11.2 | −0.259 | −0.212 | −0.141 | −0.074 | −0.092 | 0.051 | 2.132 | 9.80E−05 |
| CCDC144A | BC133019 | Hs.531547 | hs\|17p11.2 | −0.086 | 0.070 | 0.043 | 0.114 | 0.186 | 0.235 | 2.131 | 2.56E−05 |
| RP5-1000E10.4 | CR936771 | Hs.632428 | hs\|1p13.2 | 0.040 | 0.039 | 0.048 | 0.068 | 0.247 | 0.366 | 2.130 | 3.06E−05 |
| THC2495785 | CN284574 | Hs.533222 | hs\|5q12.1 | 0.019 | 0.002 | 0.211 | 0.289 | 0.346 | 0.333 | 2.129 | 6.09E−04 |
| ZNF714 | NM_182515 | Hs.466291 | hs\|19p12 | 0.022 | 0.002 | 0.006 | 0.017 | 0.223 | 0.334 | 2.128 | 1.48E−06 |
| PJA2 | NM_014819 | Hs.483036 | hs\|5q21.3 | 0.167 | 0.171 | 0.280 | 0.342 | 0.375 | 0.495 | 2.125 | 2.14E−04 |
| ENST00000344759 | NM_001001675 | Hs.444446 | hs\|19q13.41 | −0.007 | 0.015 | −0.010 | −0.155 | 0.203 | 0.323 | 2.124 | 4.33E−05 |
| ARHGAP18 | NM_033515 | Hs.486458 | hs\|6q22.33 | 0.226 | 0.193 | 0.231 | 0.225 | 0.451 | 0.507 | 2.123 | 6.60E−04 |
| AF131777 | AF131777 | Hs.655994 | hs\|13q34 | −0.182 | 0.039 | 0.049 | 0.091 | 0.168 | 0.145 | 2.123 | 5.81E−04 |
| BX093444 | BC047720 | Hs.345877 | hs\|18q21.1 | −0.053 | 0.145 | 0.091 | 0.106 | 0.221 | 0.269 | 2.121 | 1.65E−04 |
| C14orf118 | AB032978 | | hs\|14q24.3 | −0.252 | −0.195 | −0.227 | −0.121 | −0.054 | 0.075 | 2.121 | 1.15E−04 |
| PHF20L1 | NM_032205 | Hs.304362 | hs\|8q24.22 | −0.055 | 0.092 | 0.187 | 0.240 | 0.256 | 0.222 | 2.120 | 2.95E−06 |
| DENND1A | NM_024820 | Hs.655834 | hs\|9q33.2 | −0.119 | −0.026 | 0.228 | 0.208 | 0.251 | 0.066 | 2.119 | 2.59E−04 |
| ZC3H11A | NM_014827 | Hs.532399 | hs\|1q32.1 | 0.073 | 0.091 | 0.203 | 0.304 | 0.305 | 0.412 | 2.118 | 4.54E−04 |
| ZC3H8 | NM_032494 | Hs.418416 | hs\|2q13 | −0.014 | −0.008 | −0.011 | 0.031 | 0.225 | 0.312 | 2.114 | 2.95E−06 |
| ZC3H12C | AB096241 | Hs.376289 | hs\|11q22.3 | −0.072 | −0.147 | 0.264 | 0.256 | 0.367 | 0.284 | 2.111 | 1.94E−04 |
| BC035156 | BC035156 | Hs.658127 | hs\|8q22.3 | −0.094 | −0.025 | 0.160 | 0.229 | 0.223 | 0.159 | 2.111 | 3.84E−04 |
| ENST00000354519 | NR_003246 | Hs.534573 | hs\|15q25.2 | −0.204 | −0.037 | −0.048 | 0.071 | 0.096 | 0.114 | 2.110 | 1.66E−04 |
| LOC728613 | BC014604 | Hs.379186 | hs\|5p15.33 | −0.002 | 0.062 | 0.226 | 0.273 | 0.308 | 0.279 | 2.110 | 2.90E−04 |
| LOC150759 | AK057596 | Hs.651675 | hs\|2q11.2 | 0.040 | 0.154 | 0.262 | 0.352 | 0.314 | 0.364 | 2.108 | 2.56E−04 |
| CYP2C9 | NM_000771 | Hs.282624 | hs\|10q23.33 | −0.122 | 0.157 | 0.063 | 0.138 | 0.200 | 0.226 | 2.108 | 3.08E−05 |
| AL137400 | BC002572 | Hs.161181 | hs\|10p13 | 0.064 | 0.148 | 0.273 | 0.309 | 0.298 | 0.402 | 2.107 | 6.57E−04 |
| FOXO1 | NM_002015 | Hs.370666 | hs\|13q14.11 | −0.041 | 0.147 | 0.095 | 0.114 | 0.236 | 0.283 | 2.106 | 6.95E−04 |
| GPBP1 | AL161991 | Hs.444279 | hs\|5q11.2 | 0.153 | 0.153 | 0.161 | 0.161 | 0.394 | 0.470 | 2.106 | 3.50E−04 |
| MDM2 | NM_002392 | Hs.567303 | hs\|12q15 | −0.237 | −0.142 | −0.263 | −0.309 | −0.031 | 0.089 | 2.106 | 8.80E−04 |
| R78584 | BU928961 | Hs.656847 | hs\|3q13.13 | −0.161 | −0.173 | −0.145 | −0.167 | −0.017 | 0.162 | 2.105 | 1.02E−05 |
| EG328448 | EG328448 | Hs.651090 | hs\|16p11.2 | −0.050 | 0.268 | 0.044 | 0.153 | 0.225 | 0.272 | 2.101 | 4.07E−04 |
| IFT57 | NM_018010 | Hs.412196 | hs\|3q13.12 | −0.021 | −0.028 | 0.197 | 0.279 | 0.372 | 0.300 | 2.100 | 1.12E−04 |
| ZNF354A | NM_005649 | Hs.484324 | hs\|5q35.3 | 0.043 | 0.064 | 0.048 | 0.114 | 0.298 | 0.368 | 2.099 | 5.68E−04 |
| LOC342892 | CR627133 | Hs.406307 | hs\|19q13.12 | 0.050 | 0.113 | 0.089 | 0.074 | 0.290 | 0.372 | 2.097 | 2.26E−04 |
| CLUAP1 | NM_024793 | Hs.155995 | hs\|16p13.3 | −0.113 | −0.016 | 0.052 | 0.109 | 0.269 | 0.208 | 2.095 | 1.67E−05 |
| PDCD4 | NM_145341 | Hs.232543 | hs\|10q25.2 | 0.083 | 0.114 | 0.215 | 0.263 | 0.203 | 0.404 | 2.094 | 4.58E−04 |
| CR603951 | BC032409 | Hs.632886 | hs\|2q11.2 | 0.024 | 0.164 | 0.254 | 0.310 | 0.289 | 0.351 | 2.094 | 2.37E−06 |
| SRFBP1 | NM_152546 | Hs.107622 | hs\|5q23.1 | 0.069 | 0.045 | −0.009 | 0.004 | 0.277 | 0.337 | 2.094 | 1.89E−04 |
| CCDC131 | NM_144982 | Hs.527874 | hs\|12q21.1 | 0.091 | 0.150 | 0.123 | 0.247 | 0.329 | 0.412 | 2.092 | 1.13E−04 |
| MAP1B | AK055112 | Hs.637017 | hs\|5q13.2 | 0.239 | −0.161 | 0.207 | 0.173 | 0.331 | 0.453 | 2.087 | 7.31E−04 |
| SNX13 | NM_015132 | Hs.585343 | hs\|7p21.1 | 0.039 | 0.104 | 0.114 | 0.173 | 0.338 | 0.358 | 2.086 | 3.78E−04 |
| PSIP1 | NM_033222 | Hs.658434 | hs\|9p22.3 | −0.025 | 0.011 | 0.088 | 0.155 | 0.349 | 0.269 | 2.083 | 4.02E−04 |
| EPC2 | NM_015630 | Hs.23270 | hs\|2q23.1 | 0.061 | 0.135 | 0.118 | 0.170 | 0.371 | 0.380 | 2.083 | 2.09E−04 |
| ZNF468 | NM_199132 | Hs.467223 | hs\|19q13.41 | 0.055 | 0.020 | 0.076 | 0.057 | 0.220 | 0.368 | 2.079 | 2.74E−04 |
| TNNI2 | NM_003282 | Hs.523403 | hs\|11p15.5 | −0.159 | −0.042 | −0.148 | −0.300 | 0.164 | 0.167 | 2.077 | 2.66E−04 |
| THC2691276 | AI694364 | Hs.533936 | hs\|18q12.3 | −0.044 | 0.044 | 0.081 | 0.162 | 0.220 | 0.068 | 2.076 | 3.20E−04 |
| OSTbeta | NM_178859 | Hs.534533 | hs\|15q22.31 | 0.556 | 0.108 | 0.060 | 0.097 | 0.424 | 0.425 | 2.075 | 2.58E−04 |
| ZKSCAN1 | NM_003439 | Hs.615360 | hs\|7q22.1 | 0.186 | 0.183 | 0.237 | 0.283 | 0.401 | 0.472 | 2.073 | 8.09E−05 |
| PPM1D | NM_003620 | Hs.591184 | hs\|17q23.2 | 0.148 | 0.060 | 0.116 | 0.126 | 0.249 | 0.420 | 2.073 | 1.08E−04 |
| LRRC44 | BX647210 | Hs.644625 | hs\|1p31.1 | −0.177 | 0.022 | −0.386 | −0.156 | 0.139 | 0.139 | 2.068 | 4.16E−06 |
| CLIP1 | NM_004168 | Hs.440475 | hs\|5p15.33 | −0.075 | −0.061 | −0.035 | 0.038 | 0.120 | 0.241 | 2.068 | 1.46E−07 |
| BM687502 | | | hs\|3q21.3 | −0.146 | −0.058 | 0.049 | 0.098 | 0.095 | 0.170 | 2.067 | 7.42E−05 |
| NACA | NM_005594 | Hs.505735 | hs\|12q13.3 | 0.092 | 0.047 | 0.124 | 0.153 | 0.250 | 0.317 | 2.066 | 5.52E−04 |
| ZNF117 | NM_024498 | Hs.250693 | hs\|7q11.21 | −0.008 | 0.002 | −0.018 | −0.001 | 0.264 | 0.307 | 2.066 | 4.63E−04 |
| LYPD1 | NM_144586 | Hs.656644 | hs\|2q21.2 | −0.204 | −0.066 | −0.130 | −0.193 | 0.024 | 0.156 | 2.065 | 7.42E−04 |
| MEIG1 | NM_001080836 | Hs.257249 | hs\|10p13 | 0.026 | −0.025 | 0.049 | 0.154 | 0.453 | 0.341 | 2.064 | 4.49E−04 |
| MOSPD2 | NM_152581 | Hs.190043 | hs\|Xp22.2 | 0.224 | 0.136 | 0.190 | 0.278 | 0.283 | 0.385 | 2.059 | 7.73E−04 |
| ZFP62 | AK091550 | Hs.509227 | | 0.190 | 0.246 | 0.278 | 0.402 | 0.482 | 0.503 | 2.056 | 2.06E−04 |
| KIAA0265 | NM_014997 | Hs.520710 | hs\|7q32.2 | −0.215 | 0.094 | −0.191 | −0.179 | −0.013 | 0.100 | 2.056 | 2.24E−05 |
| ZMYM4 | NM_005095 | Hs.269211 | hs\|1p34.3 | −0.045 | −0.012 | 0.057 | 0.059 | 0.257 | 0.267 | 2.053 | 9.41E−04 |
| C1orf71 | NM_152609 | Hs.368353 | hs\|1q44 | 0.007 | 0.087 | 0.137 | 0.170 | 0.241 | 0.358 | 2.053 | 2.20E−04 |
| AK095738 | CR627188 | Hs.666645 | hs\|8q24.3 | 0.075 | 0.101 | 0.367 | 0.498 | 0.367 | 0.387 | 2.052 | 5.10E−11 |
| NPNT | NM_001033047 | Hs.518921 | hs\|4q24 | 0.080 | 0.233 | 0.291 | 0.333 | 0.394 | 0.445 | 2.051 | 2.18E−04 |
| PDIA2 | NM_006849 | Hs.66581 | hs\|16p13.3 | −0.206 | −0.038 | 0.078 | 0.182 | 0.107 | 0.094 | 2.051 | 2.11E−04 |
| TRIM24 | NM_015905 | Hs.490287 | hs\|7q34 | 0.105 | 0.111 | 0.132 | 0.187 | 0.317 | 0.413 | 2.049 | 1.40E−06 |
| HIST2H2BE | NM_003528 | Hs.2178 | hs\|1q21.2 | 0.295 | 0.360 | 0.404 | 0.494 | 0.555 | 0.603 | 2.047 | 3.34E−06 |
| MYST4 | NM_012330 | Hs.35758 | hs\|10q22.2 | 0.005 | 0.019 | 0.072 | 0.121 | 0.283 | 0.316 | 2.046 | 4.60E−05 |
| FAM18B2 | NM_145301 | Hs.659357 | hs\|17p12 | 0.010 | 0.082 | 0.096 | −0.009 | 0.240 | 0.321 | 2.045 | 3.46E−04 |
| VPS13B | NM_152564 | Hs.191540 | hs\|8q22.2 | 0.001 | 0.083 | 0.186 | 0.264 | 0.268 | 0.313 | 2.044 | 1.53E−05 |
| LOC389634 | AK074886 | Hs.434403 | hs\|12p13.31 | −0.065 | 0.070 | 0.253 | 0.282 | 0.245 | 0.087 | 2.042 | 3.34E−04 |
| ZNF43 | NM_003423 | Hs.534365 | hs\|19p12 | −0.008 | 0.006 | −0.033 | −0.014 | 0.214 | 0.302 | 2.042 | 4.00E−04 |
| PCMTD1 | NM_052937 | Hs.308480 | hs\|8q11.23 | 0.079 | 0.045 | 0.371 | 0.430 | 0.185 | 0.379 | 2.040 | 6.09E−04 |
| GOLIM4 | NM_014498 | Hs.143600 | hs\|3q26.2 | −0.084 | −0.094 | −0.053 | −0.086 | 0.133 | 0.179 | 2.037 | 4.58E−04 |
| THC2672083 | | | hs\|3p14.3 | −0.259 | −0.049 | −0.072 | −0.219 | −0.011 | 0.050 | 2.037 | 7.68E−04 |
| RAB18 | NM_021252 | Hs.406799 | hs\|10p12.1 | 0.060 | 0.002 | 0.072 | 0.047 | 0.254 | 0.367 | 2.037 | 8.80E−04 |
| KNTC1 | NM_014708 | Hs.300559 | hs\|12q24.31 | −0.042 | −0.036 | 0.144 | 0.221 | 0.408 | 0.268 | 2.035 | 5.22E−04 |
| ZNF197 | NM_006991 | Hs.157035 | hs\|3p21.32 | −0.040 | −0.002 | 0.011 | 0.029 | 0.184 | 0.250 | 2.035 | 3.39E−04 |
| PIK3CA | NM_006218 | Hs.85701 | hs\|3q26.32 | 0.032 | 0.112 | 0.163 | 0.171 | 0.285 | 0.403 | 2.035 | 2.20E−04 |
| ENST00000306453 | | | hs\|7q31.1 | −0.179 | −0.094 | −0.020 | −0.036 | 0.137 | 0.129 | 2.033 | 7.86E−05 |
| ZNF506 | AK074757 | Hs.659321 | hs\|19p12 | 0.002 | 0.042 | 0.138 | 0.240 | 0.233 | 0.310 | 2.033 | 4.10E−04 |
| SUGT1L1 | BC020814 | Hs.442781 | hs\|13q14.11 | −0.058 | 0.000 | 0.169 | 0.131 | 0.000 | 0.250 | 2.031 | 5.48E−04 |
| NR3C1 | NM_000176 | Hs.122926 | hs\|5q31.3 | 0.096 | 0.004 | 0.138 | 0.194 | 0.306 | 0.399 | 2.028 | 1.05E−05 |
| ZNF529 | NM_020951 | Hs.654960 | hs\|19q13.12 | 0.100 | 0.072 | 0.113 | 0.123 | 0.235 | 0.407 | 2.028 | 3.88E−05 |
| THC2508355 | | | hs\|9q33.3 | 0.102 | 0.094 | 0.174 | 0.232 | 0.351 | 0.403 | 2.025 | 1.15E−04 |
| EID1 | NM_014335 | Hs.255973 | hs\|15q21.1 | 0.022 | 0.117 | 0.156 | 0.227 | 0.309 | 0.329 | 2.025 | 1.05E−05 |
| CRYZ | NM_001889 | Hs.83114 | hs\|1p31.1 | −0.096 | −0.060 | 0.018 | 0.049 | 0.133 | 0.210 | 2.025 | 1.31E−05 |
| SLC30A7 | NM_133496 | Hs.533903 | hs\|1p21.2 | 0.122 | 0.031 | 0.121 | 0.172 | 0.237 | 0.387 | 2.024 | 5.12E−04 |
| ALG10B | | | hs\|12q12 | 0.163 | 0.121 | 0.283 | 0.409 | 0.472 | 0.468 | 2.024 | 1.14E−06 |
| MAP3K2 | NM_006609 | Hs.145605 | hs\|2q14.3 | 0.167 | 0.116 | 0.133 | 0.193 | 0.316 | 0.412 | 2.023 | 3.02E−04 |
| AW858928 | AW858928 | Hs.81848 | hs\|8q24.11 | −0.070 | −0.074 | −0.020 | −0.043 | 0.122 | 0.250 | 2.019 | 1.31E−04 |
| EHHADH | NM_001966 | Hs.429879 | hs\|3q27.2 | −0.197 | 0.015 | −0.166 | −0.125 | −0.026 | 0.109 | 2.019 | 1.15E−04 |
| SLC12A2 | NM_001046 | Hs.162585 | hs\|5q23.3 | 0.119 | 0.095 | 0.153 | 0.277 | 0.444 | 0.420 | 2.018 | 7.74E−05 |
| BF207040 | BF207040 | Hs.353024 | hs\|22q11.23 | 0.099 | 0.056 | 0.440 | 0.426 | 0.400 | 0.235 | 2.017 | 5.37E−04 |
| SS18 | NM_001007559 | Hs.404263 | hs\|18q11.2 | −0.004 | −0.059 | 0.028 | 0.044 | 0.170 | 0.220 | 2.016 | 5.63E−04 |
| C3orf62 | NM_198562 | Hs.403828 | hs\|3p21.31 | −0.003 | 0.185 | 0.146 | 0.157 | 0.206 | 0.308 | 2.013 | 8.11E−04 |
| ANKIB1 | AL137349 | Hs.83293 | hs\|7q21.2 | 0.078 | 0.110 | 0.153 | 0.176 | 0.298 | 0.362 | 2.011 | 7.72E−05 |
| ZNF507 | NM_014910 | Hs.205392 | hs\|19q13.11 | 0.062 | −0.008 | 0.151 | 0.225 | 0.327 | 0.364 | 2.006 | 2.35E−04 |
| LOC653080 | AK097091 | Hs.652798 | hs\|5q13.2 | 0.091 | 0.158 | 0.249 | 0.334 | 0.376 | 0.393 | 2.005 | 9.27E−11 |
| CCDC100 | NM_153223 | Hs.483209 | hs\|5q23.2 | 0.155 | 0.096 | 0.233 | 0.282 | 0.378 | 0.454 | 2.003 | 7.00E−05 |

TABLE 2
First list of the under-expressed genes

| | Genbank | | | 5 | 12 | 25 | 50 | 100 | 200 | Fold | |
|---|---|---|---|---|---|---|---|---|---|---|---|
| Gene Symbol | Accession# | UniGeneID | Cytoband | nM | nM | nM | nM | nM | nM | Change | p.value |
| TPD52L1 | NM_001003395 | Hs.591347 | hs\|6q22.31 | 0.651 | 0.580 | −0.195 | −0.884 | −0.485 | −0.359 | −22.779 | 3.12E−04 |
| MFAP5 | NM_003480 | Hs.512842 | hs\|12p13.31 | 0.004 | −0.556 | −0.733 | −1.273 | −1.216 | −1.169 | −17.382 | 9.04E−05 |
| EHF | NM_012153 | Hs.502306 | hs\|11p13 | 1.175 | 1.108 | 1.152 | −0.130 | −0.030 | 0.067 | −16.013 | 1.82E−05 |
| NCF2 | NM_000433 | Hs.587558 | hs\|1q25.3 | −0.157 | −0.364 | −0.773 | −1.221 | −1.306 | −1.162 | −14.393 | 5.23E−06 |
| TRIM6 | NM_001003818 | Hs.125300 | hs\|11p15.4 | −0.431 | −0.359 | −0.712 | −1.189 | −1.188 | −1.069 | −13.030 | 2.24E−11 |
| PERLD1 | NM_033419 | Hs.462971 | hs\|17q12 | 0.905 | −0.125 | 0.129 | 0.017 | −0.188 | −0.152 | −11.387 | 2.95E−04 |
| ATXN1 | NM_000332 | Hs.434961 | hs\|6p22.3 | 0.292 | −0.061 | −0.395 | −0.802 | −0.731 | −0.483 | −10.898 | 8.07E−04 |
| INHBB | | | hs\|2q14.2 | 0.829 | 0.583 | 0.328 | −0.202 | −0.106 | −0.200 | −10.685 | 3.66E−06 |
| CR627122 | CR627122 | Hs.291319 | hs\|Xq26.2 | −0.297 | −0.407 | −0.848 | −1.286 | −1.328 | −1.116 | −10.322 | 1.10E−05 |
| JAM3 | NM_032801 | Hs.150718 | hs\|11q25 | −0.316 | −0.310 | −0.836 | −1.349 | −0.888 | −0.848 | −9.234 | 5.12E−05 |
| CXCL14 | NM_004887 | Hs.483444 | hs\|5q31.1 | 0.283 | −0.385 | −0.311 | −0.599 | −0.718 | −0.662 | −8.802 | 1.54E−04 |
| CR594735 | AK001432 | Hs.153408 | hs\|11p15.2 | −0.256 | −0.100 | −0.701 | −1.036 | −1.192 | −1.181 | −8.399 | 1.13E−07 |
| FLJ11235 | AK002097 | Hs.591264 | hs\|5q22.2 | 0.973 | 0.474 | 0.203 | 0.234 | −0.008 | 0.078 | −7.860 | 3.77E−07 |
| C15orf52 | NM_207380 | Hs.32433 | hs\|15q15.1 | 1.166 | 0.851 | 0.498 | 0.384 | 0.228 | 0.441 | −7.813 | 3.20E−04 |
| LIMCH1 | NM_014988 | Hs.335163 | hs\|4p13 | 0.669 | 0.610 | 0.158 | −0.316 | −0.197 | −0.061 | −7.337 | 6.57E−11 |
| LOH11CR2A | NM_198315 | Hs.152944 | hs\|11q24.1 | 0.677 | 0.255 | 0.000 | −0.151 | −0.078 | −0.185 | −7.274 | 9.65E−07 |
| BX281122 | AW014342 | Hs.665091 | hs\|6q22.31 | 0.745 | 0.377 | −0.064 | −0.450 | −0.101 | −0.065 | −7.023 | 9.80E−13 |
| GPR110 | NM_153840 | Hs.256897 | hs\|6p12.3 | 0.223 | 0.742 | 0.608 | 0.293 | 0.133 | 0.038 | −6.872 | 9.70E−05 |
| ARNT2 | NM_014862 | Hs.459070 | hs\|15q25.1 | 0.161 | 0.211 | 0.132 | 0.048 | −0.575 | −0.640 | −6.432 | 8.94E−04 |
| ATP6V0A4 | NM_020632 | Hs.98967 | hs\|7q34 | 0.741 | 0.106 | 0.477 | −0.063 | −0.054 | 0.096 | −6.236 | 8.61E−05 |
| PDGFRB | NM_002609 | Hs.509067 | hs\|5q33.1 | 0.861 | 0.308 | 0.148 | 0.027 | 0.078 | 0.538 | −6.068 | 3.71E−04 |
| ELA3B | NM_007352 | Hs.181289 | hs\|1p36.12 | 0.165 | −0.035 | −0.619 | −0.773 | −0.612 | −0.465 | −5.997 | 3.00E−05 |
| NEDD9 | NM_006403 | Hs.37982 | hs\|6p24.1 | 0.053 | −0.005 | −0.194 | −0.571 | −0.836 | −0.721 | −5.995 | 7.39E−06 |
| MYH6 | NM_002471 | Hs.278432 | hs\|14q11.2 | 0.314 | −0.051 | 0.249 | 0.015 | −0.269 | −0.454 | −5.863 | 4.84E−10 |
| SLC35F2 | NM_017515 | Hs.524014 | hs\|11q22.3 | −0.349 | −0.234 | −0.644 | −1.274 | −1.096 | −0.848 | −5.586 | 7.46E−04 |
| HAS3 | NM_005329 | Hs.592069 | hs\|16q22.1 | 0.220 | 0.050 | −0.384 | −0.510 | −0.646 | −0.520 | −5.496 | 1.50E−12 |
| COLEC12 | NM_130386 | Hs.464422 | hs\|18p11.32 | −0.119 | −0.280 | −0.458 | −1.049 | −0.938 | −0.896 | −5.494 | 3.38E−04 |
| SLC3A2 | NM_002394 | Hs.502769 | hs\|11q12.3 | 0.109 | 0.021 | −0.385 | −0.614 | −0.696 | −0.616 | −5.418 | 2.61E−11 |
| AW993939 | AW993939 | Hs.520819 | hs\|7q36.3 | −0.080 | −0.038 | 0.006 | 0.013 | −0.714 | −0.752 | −5.378 | 2.72E−05 |
| RUNX2 | NM_004348 | Hs.535845 | hs\|6p12.3 | 0.489 | 0.000 | −0.282 | −0.349 | −0.226 | −0.151 | −5.217 | 9.80E−04 |
| SUSD3 | NM_145006 | Hs.88417 | hs\|9q22.31 | −0.157 | −0.288 | −0.531 | −0.856 | −0.891 | −0.886 | −5.131 | 2.21E−04 |
| PLAU | NM_002658 | Hs.77274 | hs\|10q22.2 | 0.351 | 0.650 | 0.291 | 0.083 | −0.502 | −0.345 | −4.969 | 9.69E−04 |
| SLC22A3 | NM_021977 | Hs.567337 | hs\|6q25.3 | −0.012 | −0.073 | −0.331 | −0.853 | −0.709 | −0.677 | −4.966 | 1.05E−04 |
| FCRL4 | NM_031282 | Hs.120260 | hs\|1q23.1 | 0.549 | −0.008 | 0.287 | 0.000 | −0.117 | −0.146 | −4.954 | 5.67E−04 |
| DOCK2 | NM_004946 | Hs.586174 | hs\|5q35.1 | 0.545 | 0.210 | −0.055 | −0.129 | −0.150 | −0.038 | −4.847 | 1.38E−12 |
| SOX3 | NM_005634 | Hs.157429 | hs\|Xq27.1 | −0.164 | −0.082 | −0.543 | −0.844 | −0.883 | −0.769 | −4.789 | 7.72E−05 |
| THC2616558 | | | hs\|16q13 | −0.598 | −0.635 | −0.776 | −1.120 | −1.367 | −1.271 | −4.716 | 1.86E−06 |
| RNASET2 | NM_003730 | Hs.529989 | hs\|6q27 | −0.254 | −0.475 | −0.595 | −0.752 | −0.963 | −0.926 | −4.696 | 5.13E−05 |
| LOC100130360 | BX538057 | Hs.408455 | hs\|6p22.3 | 0.306 | 0.232 | −0.375 | −0.764 | −0.353 | −0.012 | −4.559 | 2.99E−04 |
| IL1R2 | NM_004633 | Hs.25333 | hs\|2q11.2 | 0.894 | 0.879 | 0.742 | 0.066 | 0.356 | 0.386 | −4.454 | 6.06E−04 |
| MGAT5B | NM_144677 | Hs.144531 | hs\|17q25.2 | 0.230 | 0.275 | 0.049 | −0.193 | −0.411 | −0.409 | −4.358 | 5.87E−05 |
| TCF7L1 | NM_031283 | Hs.516297 | hs\|2p11.2 | 0.047 | −0.199 | −0.093 | −0.179 | −0.451 | −0.592 | −4.356 | 8.41E−04 |
| AF222857 | AF222857 | Hs.673626 | hs\|15q21.1 | 0.333 | 0.066 | 0.000 | −0.080 | −0.195 | −0.318 | −4.342 | 1.09E−04 |
| AHNAK | NM_001620 | Hs.502756 | hs\|11q12.3 | 0.084 | −0.067 | −0.082 | −0.168 | −0.178 | −0.273 | −4.220 | 2.36E−07 |
| HOXB8 | NM_024016 | Hs.514292 | hs\|17q21.32 | 0.531 | 0.625 | 0.489 | −0.147 | −0.087 | 0.008 | −4.212 | 1.48E−04 |
| S100A16 | NM_080388 | Hs.515714 | hs\|1q21.3 | −0.036 | 0.022 | −0.317 | −0.736 | −0.649 | −0.560 | −4.132 | 1.34E−04 |
| INSIG1 | NM_198336 | Hs.520819 | hs\|7q36.3 | −0.200 | −0.042 | −0.104 | −0.176 | −0.711 | −0.718 | −4.127 | 1.49E−05 |
| DCDC2 | NM_016356 | Hs.660365 | hs\|6p22.2 | −0.102 | 0.022 | −0.225 | −0.687 | −0.777 | −0.644 | −4.045 | 3.41E−04 |
| LEMD1 | NM_001001552 | Hs.655520 | hs\|1q32.1 | −0.214 | −0.377 | −0.470 | −0.668 | −1.005 | −0.810 | −3.945 | 1.72E−05 |
| HEG1 | BQ184357 | Hs.619929 | hs\|3q21.2 | 0.222 | 0.015 | 0.056 | −0.051 | −0.378 | −0.373 | −3.936 | 3.21E−04 |
| AFAP1 | NM_021638 | Hs.529369 | hs\|4p16.1 | 0.075 | −0.063 | −0.168 | −0.171 | −0.461 | −0.520 | −3.929 | 3.82E−04 |
| PAQR9 | NM_198504 | Hs.656111 | hs\|3q23 | 0.355 | −0.208 | 0.199 | 0.106 | −0.132 | −0.236 | −3.904 | 4.69E−04 |
| LOC165186 | | | hs\|2p23.2 | −0.319 | −0.583 | −0.746 | −0.856 | −0.887 | −0.663 | −3.893 | 2.47E−07 |
| GUCA2B | NM_007102 | Hs.32966 | hs\|1p34.2 | 0.527 | 0.123 | 0.111 | −0.341 | −0.055 | −0.055 | −3.838 | 2.81E−06 |
| C9orf61 | NM_004816 | Hs.118003 | hs\|9q21.11 | 0.535 | 0.278 | 0.302 | −0.145 | 0.051 | −0.074 | −3.813 | 1.58E−04 |
| BX415272 | BX415272 | Hs.681876 | hs\|11p13 | −0.284 | −0.495 | −0.905 | −0.947 | −0.849 | −0.763 | −3.780 | 5.47E−04 |
| RRAGD | NM_021244 | Hs.485938 | hs\|6q15 | −0.250 | −0.196 | −0.510 | −0.903 | −0.822 | −0.537 | −3.768 | 5.43E−04 |
| FHL2 | NM_201555 | Hs.443687 | hs\|2q12.2 | −0.092 | −0.272 | −0.451 | −0.554 | −0.591 | −0.666 | −3.750 | 3.25E−06 |
| MBNL3 | NM_133486 | Hs.105134 | hs\|Xq26.2 | −0.279 | −0.556 | −0.762 | −1.012 | −0.845 | −0.694 | −3.699 | 1.26E−08 |
| HIST1H1A | NM_005325 | Hs.150206 | hs\|6p22.1 | −0.034 | −0.100 | −0.241 | −0.445 | −0.258 | −0.608 | −3.697 | 6.29E−05 |
| CPVL | NM_031311 | Hs.233389 | hs\|7p15.1 | 0.274 | 0.183 | −0.002 | −0.142 | −0.473 | −0.294 | −3.696 | 2.99E−05 |
| SLC16A2 | NM_006517 | Hs.75317 | hs\|Xq13.2 | −0.294 | −0.174 | −0.737 | −0.808 | −0.870 | −0.451 | −3.630 | 4.60E−04 |
| SCD5 | NM_001037582 | Hs.379191 | hs\|4q21.22 | −0.113 | −0.392 | −0.489 | −0.541 | −0.657 | −0.667 | −3.582 | 4.40E−04 |
| SLC34A2 | NM_006424 | Hs.479372 | hs\|4p15.2 | −0.024 | −0.159 | −0.385 | −0.537 | −0.571 | −0.492 | −3.578 | 1.40E−10 |
| CHST1 | NM_003654 | Hs.104576 | hs\|11p11.2 | 0.446 | −0.034 | 0.090 | 0.170 | −0.082 | −0.098 | −3.574 | 5.31E−04 |
| CDC14B | AF064105 | | hs\|9q22.33 | 0.558 | 0.000 | 0.598 | 0.511 | 0.366 | 0.008 | −3.550 | 1.76E−04 |
| EGLN3 | NM_022073 | Hs.135507 | hs\|14q13.1 | 0.559 | 0.243 | 0.121 | −0.245 | 0.070 | 0.009 | −3.544 | 2.71E−05 |
| ZFPM2 | NM_012082 | Hs.431009 | hs\|8q23.1 | 0.527 | 0.091 | 0.000 | 0.000 | −0.020 | −0.031 | −3.518 | 8.46E−08 |
| DLC1 | NM_182643 | Hs.134296 | hs\|8p22 | 0.086 | −0.152 | −0.255 | −0.416 | −0.417 | −0.516 | −3.516 | 3.41E−06 |
| ZNF649 | NM_023074 | Hs.567573 | hs\|19q13.33 | 0.061 | −0.199 | −0.440 | −0.658 | −0.478 | −0.328 | −3.451 | 5.84E−07 |
| BM129308 | BM129308 | Hs.653792 | hs\|8p21.2 | −0.432 | −0.331 | −0.564 | −1.111 | −0.968 | −0.944 | −3.440 | 6.61E−04 |
| BC043218 | BC043218 | Hs.220558 | hs\|22q13.1 | 0.505 | 0.164 | 0.202 | 0.069 | 0.016 | −0.041 | −3.432 | 6.24E−05 |
| MIRN155 | NR_001458 | Hs.662258 | hs\|21q21.3 | 0.163 | 0.000 | 0.138 | 0.000 | −0.121 | −0.371 | −3.421 | 3.75E−04 |
| TJP2 | NM_201629 | Hs.50382 | hs\|9q21.11 | 0.068 | 0.112 | −0.344 | −0.500 | −0.510 | −0.418 | −3.418 | 9.88E−11 |
| FOXD1 | NM_004472 | Hs.519385 | hs\|5q13.2 | −0.098 | −0.199 | −0.374 | −0.540 | −0.674 | −0.627 | −3.413 | 1.65E−06 |
| L1CAM | NM_000425 | Hs.522818 | hs\|Xq28 | 0.280 | −0.136 | 0.310 | 0.170 | −0.348 | −0.235 | −3.370 | 8.14E−04 |
| LOC388630 | XM_371250 | Hs.576171 | hs\|1p33 | −0.329 | −0.470 | −0.730 | −0.798 | −0.866 | −0.854 | −3.355 | 7.64E−05 |
| SMOC2 | NM_022138 | Hs.487200 | hs\|6q27 | −0.211 | −0.219 | −0.355 | −0.613 | −0.731 | −0.731 | −3.317 | 1.29E−07 |
| BCMO1 | NM_017429 | Hs.212172 | hs\|16q23.2 | 0.269 | 0.099 | −0.180 | −0.247 | −0.252 | −0.138 | −3.297 | 1.09E−06 |
| LDLR | NM_000527 | Hs.213289 | hs\|19p13.2 | −0.068 | 0.013 | −0.177 | −0.274 | −0.506 | −0.517 | −3.286 | 1.93E−06 |
| DLG3 | NM_021120 | Hs.522680 | hs\|Xq13.1 | 0.125 | 0.076 | 0.010 | −0.042 | −0.361 | −0.415 | −3.279 | 4.78E−04 |
| BC033829 | BC033829 | Hs.371240 | hs\|6q25.1 | −0.287 | −0.493 | −0.618 | −0.953 | −0.774 | −0.723 | −3.262 | 5.23E−04 |
| GSTM1 | NM_146421 | Hs.301961 | hs\|1p13.3 | 0.109 | 0.069 | −0.139 | −0.370 | −0.443 | −0.361 | −3.240 | 2.08E−07 |
| MARCKS | NM_002356 | Hs.519909 | hs\|6q22.1 | 0.630 | 0.538 | 0.427 | 0.216 | 0.125 | 0.187 | −3.198 | 4.47E−05 |
| LOC553137 | AK124400 | Hs.652438 | hs\|6q21 | −0.324 | −0.198 | −0.414 | −0.736 | −0.953 | −0.828 | −3.193 | 7.04E−04 |
| SRrp35 | NM_080743 | Hs.254414 | hs\|6q15 | −0.141 | −0.171 | −0.572 | −0.642 | −0.706 | −0.642 | −3.175 | 1.58E−12 |
| SEPTIN6 | NM_145802 | Hs.496666 | hs\|Xq24 | 0.285 | 0.210 | −0.059 | −0.224 | −0.250 | −0.212 | −3.161 | 7.28E−07 |
| SPNS2 | BC041772 | Hs.567664 | hs\|17p13.2 | 0.468 | 0.428 | 0.238 | 0.066 | −0.163 | −0.031 | −3.151 | 6.45E−05 |
| C5orf13 | NM_004772 | Hs.36053 | hs\|5q22.1 | 0.275 | 0.296 | 0.243 | 0.246 | −0.138 | −0.179 | −3.126 | 5.71E−04 |
| CDKN2A | NM_058197 | Hs.512599 | hs\|9p21.3 | 0.194 | 0.019 | −0.032 | −0.226 | −0.270 | −0.119 | −3.120 | 3.01E−04 |
| TOM1L2 | NM_001082968 | Hs.462379 | hs\|17p11.2 | 0.074 | −0.127 | −0.235 | −0.296 | −0.574 | −0.419 | −3.109 | 1.56E−04 |
| FZD4 | NM_012193 | Hs.591968 | hs\|11q14.2 | −0.176 | −0.136 | −0.478 | −0.677 | −0.667 | −0.517 | −3.092 | 1.62E−04 |
| GSTM3 | NM_000849 | Hs.2006 | hs\|1p13.3 | 0.114 | 0.085 | −0.141 | −0.353 | −0.399 | −0.324 | −3.050 | 9.13E−13 |
| TMEM16A | BC032907 | Hs.98470 | hs\|5p15.2 | 0.483 | 0.213 | 0.000 | −0.045 | 0.000 | 0.059 | −3.048 | 5.15E−06 |
| GSTM4 | NM_147148 | Hs.348387 | hs\|1p13.3 | 0.279 | 0.108 | 0.014 | −0.110 | −0.341 | −0.262 | −3.034 | 5.52E−05 |
| SLC44A1 | NM_080546 | Hs.573495 | hs\|9q31.1 | −0.125 | −0.131 | −0.317 | −0.516 | −0.505 | −0.530 | −3.029 | 2.98E−05 |
| DCLK1 | NM_004734 | Hs.507755 | hs\|13q13.3 | −0.626 | −0.615 | −0.840 | −1.107 | −1.094 | −1.002 | −3.024 | 1.34E−05 |
| MGC33846 | NM_175885 | Hs.448218 | hs\|11q14.1 | 0.031 | −0.267 | −0.338 | −0.480 | −0.500 | −0.433 | −3.023 | 2.39E−04 |
| NKX3-1 | NM_006167 | Hs.55999 | hs\|8p21.2 | −0.050 | −0.021 | −0.303 | −0.599 | −0.515 | −0.444 | −3.020 | 6.14E−05 |
| PKP2 | NM_004572 | Hs.164384 | hs\|12p11.21 | 0.290 | 0.008 | −0.138 | −0.174 | −0.219 | −0.155 | −3.012 | 1.03E−04 |
| ENST00000343505 | BC020940 | Hs.652741 | hs\|6q23.3 | 0.216 | 0.101 | −0.021 | −0.165 | −0.188 | −0.262 | −3.010 | 9.85E−06 |
| THC2616992 | | | hs\|13q13.1 | −0.217 | −0.209 | −0.687 | −0.802 | −0.692 | −0.404 | −3.007 | 7.44E−05 |
| SH3RF1 | NM_020870 | Hs.301804 | hs\|4q32.3 | 0.422 | 0.448 | 0.206 | 0.014 | −0.069 | −0.049 | −2.966 | 1.18E−04 |
| LGI2 | NM_017688 | Hs.632677 | hs\|9q32 | −0.196 | −0.255 | −0.353 | −0.565 | −0.700 | −0.668 | −2.954 | 5.53E−05 |
| GALM | NM_138801 | Hs.435012 | hs\|2p22.1 | −0.062 | −0.177 | −0.302 | −0.312 | −0.499 | −0.526 | −2.928 | 3.91E−05 |
| MAP6 | NM_033063 | Hs.585540 | hs\|11q13.5 | 0.510 | 0.020 | 0.502 | 0.432 | 0.094 | 0.042 | −2.891 | 2.88E−07 |
| ROR1 | NM_005012 | Hs.654491 | hs\|1p31.3 | 0.132 | 0.066 | −0.328 | −0.418 | −0.319 | −0.090 | −2.888 | 1.41E−04 |
| FRMD4A | NM_018027 | Hs.330463 | hs\|10p13 | −0.095 | −0.218 | −0.379 | −0.351 | −0.626 | −0.556 | −2.886 | 7.52E−04 |
| MAGEA10 | NM_001011543 | Hs.18048 | hs\|Xq28 | 0.000 | −0.069 | 0.000 | −0.081 | −0.404 | −0.457 | −2.865 | 3.62E−04 |
| UBASH3B | NM_032873 | Hs.444075 | hs\|11q24.1 | 0.031 | −0.070 | −0.005 | 0.004 | −0.317 | −0.463 | −2.858 | 6.30E−05 |
| BI832578 | BI832578 | Hs.669408 | hs\|1p22.2 | 0.071 | −0.033 | −0.311 | −0.320 | −0.307 | −0.385 | −2.854 | 2.03E−05 |
| SPON1 | NM_006108 | Hs.654637 | hs\|11p15.2 | 0.342 | 0.288 | 0.224 | 0.166 | −0.067 | −0.116 | −2.849 | 2.62E−04 |
| THC2533833 | AA873311 | Hs.693594 | hs\|6q25.3 | 0.048 | −0.259 | −0.248 | −0.313 | −0.286 | −0.410 | −2.838 | 5.51E−04 |
| OGFRL1 | NM_024576 | Hs.656091 | hs\|6q13 | 0.111 | −0.187 | −0.258 | −0.319 | −0.366 | −0.256 | −2.826 | 9.63E−05 |
| BU943730 | BU943730 | Hs.636188 | hs\|12p13.1 | 0.037 | −0.080 | −0.013 | −0.021 | −0.211 | −0.367 | −2.815 | 2.38E−04 |
| LOC388610 | NM_001013642 | Hs.355747 | hs\|1p36.11 | 0.387 | 0.291 | 0.084 | −0.046 | −0.067 | 0.032 | −2.806 | 5.72E−04 |
| SDC1 | NM_001006946 | Hs.224607 | hs\|2p24.1 | 0.398 | 0.092 | 0.294 | 0.159 | 0.042 | −0.049 | −2.802 | 5.22E−04 |
| BF312639 | BF312639 | Hs.655654 | hs\|7q22.1 | 0.221 | −0.034 | −0.236 | −0.210 | −0.227 | −0.048 | −2.795 | 3.73E−05 |
| FAM83A | NM_032899 | Hs.379821 | hs\|8q24.13 | 0.321 | 0.031 | −0.167 | −0.140 | −0.096 | −0.125 | −2.791 | 1.96E−04 |
| RCAN3 | NM_013441 | Hs.656799 | hs\|1p36.11 | 0.253 | 0.051 | −0.157 | −0.166 | −0.222 | −0.191 | −2.781 | 5.42E−05 |
| THC2636507 | | | hs\|11q23.1 | 0.382 | 0.000 | 0.000 | −0.039 | −0.106 | −0.057 | −2.749 | 2.35E−05 |
| ELK3 | NM_005230 | Hs.591015 | hs\|12q23.1 | −0.013 | −0.030 | −0.151 | −0.273 | −0.562 | −0.456 | −2.731 | 6.50E−04 |
| TMEM171 | NM_173490 | Hs.162246 | hs\|5q13.2 | −0.059 | −0.282 | −0.207 | −0.334 | −0.447 | −0.473 | −2.722 | 1.41E−04 |
| ENST00000370624 | AK092806 | Hs.407054 | hs\|1p22.3 | 0.085 | −0.171 | −0.283 | −0.392 | −0.345 | −0.152 | −2.704 | 6.74E−04 |
| LOC440900 | AK096065 | Hs.592185 | hs\|7p21.1 | 0.131 | −0.035 | −0.188 | −0.254 | −0.297 | −0.110 | −2.695 | 9.07E−06 |
| RAB7B | NM_177403 | Hs.534612 | | 0.464 | 0.162 | 0.330 | 0.139 | 0.019 | 0.036 | −2.689 | 9.42E−04 |
| ABHD2 | NM_007011 | Hs.122337 | hs\|15q26.1 | 0.352 | −0.011 | 0.191 | 0.185 | 0.014 | −0.081 | −2.685 | 4.95E−05 |
| PDE6A | NM_000440 | Hs.567314 | hs\|5q33.1 | −0.245 | −0.309 | −0.756 | −0.818 | −0.736 | −0.534 | −2.665 | 4.56E−04 |
| OR2H2 | NM_007160 | Hs.529493 | hs\|6p22.1 | 0.260 | −0.038 | 0.097 | −0.009 | −0.097 | −0.163 | −2.664 | 8.27E−06 |
| GJC1 | NM_005497 | Hs.659160 | hs\|17q21.31 | −0.795 | −0.801 | −0.866 | −1.177 | −1.249 | −1.195 | −2.660 | 2.46E−05 |
| AY007156 | AY007156 | Hs.593067 | hs\|20p11.23 | 0.251 | 0.130 | 0.031 | −0.104 | −0.202 | −0.139 | −2.659 | 2.71E−04 |
| IL6R | NM_000565 | Hs.591492 | hs\|1q21.3 | −0.301 | −0.394 | −0.523 | −0.599 | −0.656 | −0.557 | −2.657 | 7.80E−04 |
| HUS1B | NM_148959 | Hs.669039 | hs\|6p25.3 | 0.203 | −0.028 | −0.188 | −0.353 | −0.218 | −0.192 | −2.646 | 5.83E−04 |
| SOCS1 | NM_003745 | Hs.50640 | hs\|16p13.13 | 0.433 | 0.211 | 0.122 | 0.044 | 0.001 | 0.011 | −2.645 | 6.51E−06 |
| DB111455 | DB111455 | Hs.660706 | hs\|11q21 | 0.422 | 0.105 | 0.353 | 0.339 | 0.131 | 0.000 | −2.643 | 7.28E−05 |
| AK026194 | AK026194 | Hs.593067 | hs\|20p11.23 | 0.211 | 0.120 | −0.009 | −0.128 | −0.226 | −0.160 | −2.640 | 3.76E−05 |
| WDR40B | NM_178470 | Hs.120403 | hs\|Xq25 | 0.269 | 0.148 | −0.080 | −0.143 | 0.000 | −0.174 | −2.607 | 5.28E−11 |
| THC2707284 | | | hs\|11q13.4 | −0.044 | 0.010 | −0.350 | −0.437 | −0.439 | −0.460 | −2.605 | 5.74E−05 |
| HSU79275 | U79275 | Hs.598507 | hs\|12q13.11 | −0.038 | −0.169 | −0.314 | −0.398 | −0.476 | −0.453 | −2.600 | 6.05E−07 |
| TMEM2 | NM_013390 | Hs.494146 | hs\|9q21.13 | 0.453 | 0.112 | 0.115 | 0.067 | 0.043 | 0.002 | −2.600 | 4.51E−05 |
| PADI3 | NM_016233 | Hs.149195 | hs\|1p36.13 | 0.291 | 0.351 | 0.148 | −0.043 | −0.115 | −0.114 | −2.571 | 1.20E−06 |
| MMAB | NM_052845 | Hs.12106 | hs\|12q24.11 | −0.039 | −0.121 | −0.071 | −0.129 | −0.363 | −0.447 | −2.556 | 6.81E−07 |
| ME1 | NM_002395 | Hs.21160 | hs\|6q14.2 | 0.157 | 0.008 | −0.102 | −0.177 | −0.230 | −0.197 | −2.552 | 1.40E−05 |
| MPP1 | NM_002436 | Hs.496984 | hs\|Xq28 | 0.116 | 0.031 | −0.142 | −0.140 | −0.194 | −0.291 | −2.550 | 3.81E−05 |
| ACSS2 | NM_018677 | Hs.517034 | hs\|20q11.22 | 0.224 | 0.096 | −0.008 | −0.070 | −0.203 | −0.182 | −2.547 | 6.69E−06 |
| ZBTB24 | NM_014797 | Hs.409876 | hs\|6q21 | −0.017 | −0.411 | −0.286 | −0.386 | −0.440 | −0.420 | −2.531 | 5.31E−04 |
| DHCR7 | NM_001360 | Hs.503134 | hs\|11q13.4 | −0.118 | −0.136 | −0.201 | −0.256 | −0.533 | −0.521 | −2.527 | 6.45E−04 |
| ACAT2 | NM_005891 | Hs.571037 | hs\|6q25.3 | −0.032 | −0.077 | −0.078 | −0.155 | −0.324 | −0.425 | −2.520 | 1.82E−04 |
| G6PD | NM_000402 | Hs.461047 | hs\|Xq28 | 0.218 | 0.143 | −0.061 | −0.048 | −0.044 | −0.106 | −2.504 | 3.02E−04 |
| SEPP1 | NM_005410 | Hs.275775 | hs\|5p12 | 0.455 | 0.347 | 0.318 | 0.188 | 0.120 | 0.368 | −2.502 | 2.44E−04 |
| BARX2 | NM_005317 | Hs.465511 | hs\|19p13.3 | −0.052 | −0.186 | −0.344 | −0.424 | −0.541 | −0.278 | −2.499 | 4.82E−11 |
| PGC | NM_002630 | Hs.1867 | hs\|6p21.1 | 0.431 | 0.419 | 0.293 | 0.141 | 0.031 | 0.128 | −2.497 | 4.47E−04 |
| ZBED1 | NM_004729 | Hs.131452 | hs\|Yp11.31 | 0.169 | −0.092 | −0.136 | −0.195 | −0.263 | −0.327 | −2.494 | 5.73E−04 |
| TMCC3 | NM_020698 | Hs.370410 | hs\|12q22 | 0.018 | −0.061 | −0.084 | −0.203 | −0.331 | −0.379 | −2.492 | 5.20E−04 |
| THC2732364 | | | hs\|6q27 | 0.167 | −0.016 | 0.114 | −0.022 | −0.107 | −0.229 | −2.490 | 9.24E−04 |
| CHRFAM7A | BX395274 | Hs.663861 | hs\|15q13.3 | −0.067 | −0.209 | −0.269 | −0.376 | −0.568 | −0.463 | −2.484 | 3.17E−04 |
| LBH | NM_030915 | Hs.567598 | hs\|2p23.1 | −0.192 | −0.162 | −0.404 | −0.550 | −0.713 | −0.587 | −2.483 | 2.85E−04 |
| RAPGEF3 | NM_006105 | Hs.8578 | hs\|12q13.11 | −0.086 | −0.027 | −0.314 | −0.404 | −0.446 | −0.451 | −2.477 | 6.66E−05 |
| SLC18A1 | NM_003053 | Hs.158322 | hs\|8p21.3 | 0.322 | 0.362 | 0.000 | −0.186 | −0.029 | −0.006 | −2.473 | 3.97E−04 |
| PRKD1 | NM_002742 | Hs.508999 | hs\|14q12 | −0.289 | −0.218 | −0.411 | −0.718 | −0.711 | −0.524 | −2.468 | 9.84E−04 |
| THC2634493 | | | hs\|13q13.3 | −0.505 | −0.624 | −0.719 | −0.998 | −0.864 | −0.774 | −2.459 | 2.05E−04 |
| F3 | NM_001993 | Hs.62192 | hs\|1p21.3 | 0.109 | −0.160 | −0.138 | −0.223 | −0.263 | −0.282 | −2.459 | 3.00E−06 |
| MGC50722 | NM_203348 | Hs.530383 | | 0.314 | −0.081 | −0.015 | 0.032 | −0.086 | −0.104 | −2.454 | 3.02E−04 |
| B4GALT4 | NM_212543 | Hs.13225 | hs\|3q13.32 | 0.266 | 0.166 | −0.083 | −0.211 | −0.123 | −0.079 | −2.453 | 4.47E−11 |
| SLC12A8 | NM_024628 | Hs.658514 | hs\|3q21.2 | −0.057 | −0.025 | −0.264 | −0.535 | −0.445 | −0.402 | −2.448 | 4.13E−04 |
| DMRT3 | NM_021240 | Hs.189174 | hs\|9p24.3 | −0.564 | −0.356 | −0.432 | −0.608 | −0.747 | −0.723 | −2.446 | 8.20E−04 |
| TLE6 | BC007329 | Hs.334507 | hs\|19p13.3 | 0.046 | 0.006 | −0.230 | −0.162 | −0.321 | −0.342 | −2.440 | 8.41E−04 |
| MICAL2 | NM_014632 | Hs.501928 | hs\|11p15.3 | 0.010 | −0.062 | −0.041 | −0.050 | −0.269 | −0.306 | −2.424 | 9.30E−04 |
| ADRA1B | NM_000679 | Hs.368632 | hs\|5q33.3 | −0.137 | −0.314 | −0.471 | −0.582 | −0.515 | −0.521 | −2.420 | 6.83E−11 |
| NDRG2 | NM_201535 | Hs.525205 | hs\|14q11.2 | −0.048 | −0.082 | −0.053 | −0.206 | −0.340 | −0.431 | −2.417 | 2.08E−04 |
| CSF2RA | NM_172247 | Hs.520937 | hs\|Yp11.32 | 0.109 | −0.040 | −0.104 | −0.177 | −0.256 | −0.240 | −2.411 | 7.43E−06 |
| BG695979 | BG695979 | Hs.594262 | hs\|1p36.11 | 0.390 | 0.109 | 0.004 | −0.045 | −0.172 | −0.076 | −2.407 | 9.86E−04 |
| LOC100133991 | AK097219 | Hs.668927 | hs\|17q21.31 | 0.177 | −0.096 | −0.090 | 0.000 | −0.185 | −0.249 | −2.404 | 5.67E−04 |
| LOC389895 | | | hs\|Xq27.1 | −0.127 | −0.221 | −0.330 | −0.525 | −0.440 | −0.510 | −2.395 | 6.01E−05 |
| RNF8 | NM_003958 | Hs.485278 | hs\|6p21.2 | 0.013 | −0.090 | −0.200 | −0.264 | −0.394 | −0.359 | −2.377 | 3.79E−05 |
| CTSC | NM_024996 | Hs.518355 | hs\|3q25.32 | 0.172 | 0.038 | −0.014 | −0.035 | −0.151 | −0.207 | −2.375 | 7.15E−06 |
| RAB3B | NM_002867 | Hs.123072 | hs\|1p32.3 | −0.118 | −0.350 | −0.420 | −0.411 | −0.524 | −0.607 | −2.369 | 8.43E−04 |
| EPB41L2 | NM_001431 | Hs.486470 | hs\|6q23.1 | 0.169 | 0.196 | −0.213 | −0.282 | −0.167 | −0.205 | −2.368 | 4.09E−04 |
| HAVCR1 | NM_012206 | Hs.129711 | hs\|5q33.3 | 0.178 | 0.038 | 0.097 | −0.122 | −0.265 | −0.196 | −2.367 | 9.77E−04 |
| MGC3032 | AK096306 | Hs.568945 | hs\|11q13.1 | 0.182 | 0.096 | −0.030 | −0.087 | −0.148 | −0.049 | −2.366 | 3.59E−04 |
| AKAP7 | NM_016377 | Hs.486483 | hs\|6q23.2 | 0.158 | 0.004 | −0.144 | −0.206 | −0.188 | −0.216 | −2.364 | 1.69E−04 |
| CDKAL1 | NM_017774 | Hs.657604 | hs\|6p22.3 | 0.164 | 0.202 | 0.005 | −0.088 | −0.160 | −0.165 | −2.360 | 8.77E−04 |
| SLC2A1 | NM_003245 | Hs.2022 | hs\|20p13 | 0.009 | −0.197 | −0.328 | −0.343 | −0.367 | −0.363 | −2.360 | 1.10E−07 |
| PALLD | NM_016081 | Hs.151220 | hs\|4q32.3 | 0.052 | −0.173 | −0.179 | −0.268 | −0.315 | −0.320 | −2.352 | 3.13E−05 |
| BTBD9 | NM_152733 | Hs.654635 | hs\|6p21.2 | 0.174 | −0.078 | −0.292 | −0.202 | −0.192 | −0.042 | −2.347 | 5.78E−04 |
| C6orf64 | NM_018322 | Hs.58382 | hs\|6p21.2 | 0.176 | 0.077 | 0.019 | −0.009 | −0.131 | −0.194 | −2.341 | 2.05E−05 |
| FGFR4 | NM_213647 | Hs.165950 | hs\|5q35.2 | −0.123 | −0.195 | −0.291 | −0.302 | −0.419 | −0.500 | −2.335 | 1.78E−04 |
| RBMY1E | NM_001006118 | Hs.536001 | hs\|Yq11.223 | 0.279 | −0.127 | 0.195 | 0.109 | −0.018 | −0.089 | −2.332 | 4.12E−04 |
| RERE | NM_012102 | Hs.463041 | hs\|1p36.23 | −0.024 | −0.206 | −0.330 | −0.378 | −0.426 | −0.208 | −2.332 | 1.31E−10 |
| TINAGL1 | NM_022164 | Hs.199368 | hs\|1p35.2 | 0.372 | 0.293 | 0.286 | 0.116 | −0.092 | −0.138 | −2.327 | 1.33E−06 |
| THC2722891 | | | hs\|6q23.2 | 0.552 | 0.402 | 0.369 | 0.211 | 0.203 | 0.192 | −2.323 | 6.39E−04 |
| KIF13A | NM_022113 | Hs.189915 | hs\|6p22.3 | 0.010 | −0.157 | −0.244 | −0.331 | −0.310 | −0.354 | −2.314 | 1.93E−06 |
| LOC440335 | BC022385 | Hs.390599 | hs\|16p13.3 | 0.306 | −0.129 | 0.000 | −0.035 | −0.073 | −0.058 | −2.312 | 3.37E−04 |
| B3GALT5 | NM_033173 | Hs.655094 | hs\|21q22.2 | −0.112 | 0.340 | 0.379 | 0.367 | 0.091 | −0.023 | −2.311 | 1.27E−04 |
| GNB5 | BC011671 | Hs.155090 | hs\|15q21.2 | 0.110 | 0.123 | −0.151 | −0.210 | −0.239 | −0.199 | −2.276 | 1.05E−05 |
| GALNT6 | NM_007210 | Hs.505575 | hs\|12q13.13 | 0.166 | 0.051 | 0.028 | −0.147 | −0.137 | −0.191 | −2.276 | 3.94E−04 |
| THC2608967 | | | hs\|15q25.2 | 0.370 | 0.045 | 0.295 | 0.200 | 0.092 | 0.012 | −2.276 | 2.50E−04 |
| HFE | NM_139009 | Hs.233325 | hs\|6p22.1 | 0.188 | 0.074 | 0.051 | 0.014 | −0.148 | −0.169 | −2.274 | 9.29E−04 |
| GMDS | NM_001500 | Hs.144496 | hs\|6p25.3 | −0.025 | −0.080 | −0.257 | −0.294 | −0.375 | −0.381 | −2.266 | 2.04E−04 |
| THC2582296 | BU194531 | Hs.654439 | hs\|19q13.32 | 0.123 | 0.006 | −0.049 | −0.203 | −0.227 | 0.206 | −2.260 | 3.72E−05 |
| ADAMTS4 | BC030812 | | hs\|1q23.3 | 0.254 | 0.009 | 0.085 | 0.000 | −0.060 | −0.100 | −2.259 | 5.79E−05 |
| TBC1D1 | NM_015173 | Hs.176503 | hs\|4p14 | 0.052 | −0.136 | 0.060 | 0.049 | −0.134 | −0.302 | −2.259 | 3.75E−05 |
| POU5F1 | NM_002701 | Hs.249184 | hs\|6p21.33 | −0.240 | −0.136 | −0.225 | −0.364 | −0.514 | −0.457 | −2.252 | 6.40E−05 |
| DNMT3B | NM_175850 | Hs.655708 | hs\|20q11.21 | −0.093 | −0.015 | −0.126 | −0.166 | −0.284 | −0.445 | −2.246 | 2.68E−06 |
| CDH5 | NM_001795 | Hs.76206 | hs\|16q21 | 0.193 | 0.126 | 0.119 | −0.223 | −0.095 | −0.157 | −2.243 | 7.32E−11 |
| FLJ22222 | BC009297 | | hs\|17q25.3 | −0.097 | −0.156 | −0.180 | −0.257 | −0.316 | −0.483 | −2.243 | 1.37E−04 |
| KIAA1553 | NM_001080450 | Hs.418045 | hs\|6q21 | −0.073 | −0.238 | −0.202 | −0.223 | −0.275 | −0.424 | −2.240 | 5.64E−04 |
| XR_018059 | XR_018059 | Hs.648104 | hs\|7p13 | 0.066 | −0.050 | −0.050 | −0.116 | −0.136 | −0.301 | −2.239 | 2.50E−04 |
| MTA3 | AK127245 | Hs.435413 | hs\|2p21 | −0.018 | −0.121 | −0.165 | −0.145 | −0.342 | −0.367 | −2.236 | 5.94E−04 |
| RAB15 | NM_198686 | Hs.512492 | hs\|14q23.3 | −0.089 | −0.195 | −0.319 | −0.344 | −0.406 | −0.426 | −2.232 | 5.98E−05 |
| XIRP1 | NM_194293 | Hs.447868 | hs\|3p22.2 | 0.221 | −0.008 | 0.000 | −0.066 | −0.015 | −0.127 | −2.227 | 7.11E−04 |
| RAB11FIP4 | NM_032932 | Hs.406788 | hs\|17q11.2 | −0.067 | 0.036 | 0.171 | 0.189 | −0.179 | −0.173 | −2.220 | 6.39E−04 |
| LOC100132338 | AK074662 | Hs.473927 | hs\|21q22.3 | 0.250 | −0.058 | 0.062 | −0.016 | −0.082 | −0.095 | −2.215 | 1.67E−04 |
| HLA-DRB4 | NM_021983 | Hs.654405 | | 0.441 | 0.008 | 0.330 | 0.374 | 0.169 | 0.097 | −2.211 | 8.39E−05 |
| FZD5 | NM_003468 | Hs.17631 | hs\|2q33.3 | −0.018 | −0.080 | −0.178 | −0.284 | −0.321 | −0.361 | −2.199 | 1.41E−06 |
| PRAGMIN | AF075060 | Hs.657673 | hs\|8p23.1 | 0.112 | −0.031 | −0.056 | −0.195 | −0.227 | −0.007 | −2.198 | 1.39E−04 |
| UBE2J1 | NM_016021 | Hs.163776 | hs\|6q15 | −0.193 | −0.244 | −0.348 | −0.470 | −0.556 | −0.506 | −2.198 | 7.93E−05 |
| CHRDL2 | NM_015424 | Hs.432379 | hs\|11q13.4 | 0.230 | −0.089 | 0.053 | −0.021 | −0.020 | −0.112 | −2.197 | 3.50E−04 |
| C15orf50 | BC031958 | Hs.569502 | hs\|15q23 | 0.245 | −0.046 | 0.000 | 0.066 | −0.027 | −0.099 | −2.191 | 8.28E−04 |
| CLDN4 | NM_001305 | Hs.647036 | hs\|7q11.23 | 0.274 | 0.006 | 0.010 | −0.014 | −0.066 | −0.093 | −2.189 | 9.23E−05 |
| IGF2R | NM_000876 | Hs.487062 | hs\|6q25.3 | 0.054 | −0.023 | −0.152 | −0.205 | −0.317 | −0.285 | −2.187 | 3.94E−04 |
| GSTM2 | NM_000848 | Hs.279837 | hs\|1p13.3 | 0.292 | 0.098 | 0.083 | 0.015 | −0.125 | −0.092 | −2.182 | 1.99E−04 |
| RRAS2 | NM_012250 | Hs.502004 | hs\|11p15.2 | −0.040 | −0.098 | −0.293 | −0.349 | −0.366 | −0.319 | −2.173 | 2.08E−04 |
| AW268902 | AW268902 | Hs.29802 | hs\|4p15.31 | 0.133 | 0.054 | −0.124 | −0.157 | −0.192 | 0.087 | −2.169 | 2.76E−04 |
| HDDC2 | NM_016063 | Hs.32826 | hs\|6q22.31 | 0.183 | 0.054 | −0.123 | −0.224 | −0.150 | −0.158 | −2.167 | 8.89E−06 |
| CDC20B | NM_152623 | Hs.669184 | hs\|5q11.2 | 0.126 | −0.086 | 0.000 | 0.000 | −0.128 | −0.211 | −2.161 | 4.35E−04 |
| PFKL | NM_001002021 | Hs.255093 | hs\|21q22.3 | −0.043 | −0.124 | NA | −0.130 | −0.304 | −0.378 | −2.159 | 6.84E−05 |
| LRP8 | NM_033300 | Hs.576154 | hs\|1p32.3 | 0.148 | 0.181 | 0.122 | 0.076 | −0.019 | −0.186 | −2.156 | 7.10E−05 |
| STIM1 | NM_003156 | Hs.501735 | hs\|11p15.4 | 0.029 | −0.115 | −0.187 | −0.226 | −0.325 | −0.304 | −2.151 | 4.01E−05 |
| KLHL5 | NM_015990 | Hs.272251 | hs\|4p14 | 0.331 | 0.043 | 0.175 | 0.204 | 0.009 | −0.001 | −2.147 | 9.71E−04 |
| TP53INP2 | NM_021202 | Hs.516994 | hs\|20q11.22 | 0.008 | −0.018 | −0.179 | −0.300 | −0.457 | −0.324 | −2.145 | 4.67E−06 |
| FURIN | NM_002569 | Hs.513153 | hs\|15q26.1 | 0.038 | −0.058 | −0.151 | −0.045 | −0.320 | −0.309 | −2.144 | 6.16E−05 |
| TRIB3 | NM_021158 | Hs.516826 | hs\|20p13 | 0.120 | 0.020 | −0.111 | −0.099 | −0.183 | −0.214 | −2.139 | 3.92E−04 |
| ADCY4 | NM_139247 | Hs.443428 | hs\|14q12 | 0.213 | −0.045 | 0.066 | 0.026 | −0.069 | −0.117 | −2.137 | 2.06E−04 |
| KANK1 | NM_153186 | Hs.306764 | hs\|9p24.3 | 0.175 | −0.036 | 0.061 | 0.011 | −0.142 | −0.154 | −2.132 | 9.75E−04 |
| SH3KBP1 | NM_031892 | Hs.444770 | hs\|Xp22.12 | 0.040 | 0.010 | −0.281 | −0.363 | −0.236 | −0.303 | −2.132 | 7.91E−04 |
| S73202 | S73202 | | hs\|9q34.11 | 0.274 | 0.340 | 0.227 | −0.034 | −0.079 | −0.017 | −2.130 | 4.35E−05 |
| CCDC109B | NM_017918 | Hs.234149 | hs\|4q25 | 0.032 | −0.008 | −0.072 | −0.162 | −0.151 | −0.265 | −2.129 | 1.66E−04 |
| AGPAT4 | NM_020133 | Hs.353175 | hs\|6q26 | −0.069 | −0.083 | −0.190 | −0.248 | −0.431 | −0.397 | −2.129 | 7.92E−04 |
| ACSS1 | NM_032501 | Hs.529353 | hs\|20p11.21 | 0.181 | 0.013 | 0.000 | 0.000 | −0.078 | −0.147 | −2.125 | 9.18E−04 |
| AK094629 | AK094629 | Hs.594896 | hs\|6q26 | −0.136 | −0.153 | −0.476 | −0.626 | −0.461 | −0.281 | −2.123 | 4.79E−04 |
| LRRC31 | NM_024727 | Hs.411295 | hs\|3q26.2 | 0.552 | 0.383 | 0.432 | 0.173 | 0.289 | 0.226 | −2.118 | 6.26E−04 |
| AY090769 | AY090769 | Hs.275865 | hs\|6p21.32 | 0.062 | 0.007 | 0.004 | −0.113 | −0.153 | −0.263 | −2.117 | 2.63E−04 |
| NSDHL | NM_015922 | Hs.57698 | hs\|Xq28 | −0.011 | −0.057 | −0.128 | −0.135 | −0.291 | −0.335 | −2.110 | 1.24E−05 |
| SLC35B2 | NM_178148 | Hs.182885 | hs\|6p21.1 | 0.062 | 0.050 | −0.062 | −0.127 | −0.260 | −0.264 | −2.110 | 5.86E−04 |
| SVIL | NM_021738 | Hs.499209 | hs\|10p11.23 | 0.203 | 0.164 | 0.105 | 0.029 | −0.160 | −0.120 | −2.104 | 4.70E−08 |
| THC2569387 | AA832084 | Hs.159161 | hs\|17q25.3 | 0.108 | −0.110 | −0.009 | −0.045 | −0.111 | −0.228 | −2.089 | 9.93E−04 |
| ENST00000366623 | AJ311797 | Hs.547779 | hs\|1q42.2 | 0.238 | 0.056 | 0.129 | 0.070 | 0.043 | −0.087 | −2.089 | 3.18E−04 |
| RFFL | NM_057178 | Hs. 13680 | hs\|17q12 | 0.134 | 0.041 | −0.040 | −0.178 | −0.156 | −0.098 | −2.081 | 3.08E−04 |
| THC2512502 | AW589254 | Hs.653501 | hs\|Xp11.23 | 0.092 | −0.184 | −0.131 | −0.104 | −0.162 | −0.229 | −2.081 | 3.34E−04 |
| NQO2 | NM_000904 | Hs.533050 | hs\|6p25.2 | −0.018 | −0.077 | −0.048 | −0.083 | −0.215 | −0.337 | −2.080 | 3.42E−05 |
| RHOF | NM_019034 | Hs.524804 | hs\|12q24.31 | −0.096 | −0.126 | −0.239 | −0.294 | −0.321 | −0.413 | −2.074 | 4.10E−04 |
| MLL3 | NM_170606 | Hs.647120 | hs\|7q36.1 | 0.058 | −0.024 | −0.086 | −0.100 | −0.274 | −0.263 | −2.074 | 3.56E−04 |
| AL522622 | AL522622 | Hs.432121 | hs\|19p13.13 | −0.062 | −0.002 | −0.021 | −0.097 | −0.205 | −0.298 | −2.072 | 3.04E−04 |
| FBXO9 | NM_033481 | Hs.216653 | hs\|6p12.1 | 0.046 | −0.007 | −0.092 | −0.164 | −0.222 | −0.168 | −2.070 | 1.82E−04 |
| MSN | NM_002444 | Hs.87752 | hs\|Xq11.1 | 0.102 | 0.019 | −0.072 | −0.083 | −0.149 | −0.212 | −2.061 | 1.08E−04 |
| BY798802 | BY798802 | Hs.598990 | hs\|15q15.1 | 0.265 | 0.068 | −0.002 | −0.012 | −0.046 | −0.068 | −2.061 | 1.86E−05 |
| CSPG5 | NM_006574 | Hs.45127 | hs\|3p21.31 | −0.168 | −0.299 | −0.433 | −0.479 | −0.476 | −0.377 | −2.055 | 9.65E−04 |
| SHROOM2 | NM_001649 | Hs.567236 | hs\|Xp22.2 | 0.092 | 0.049 | −0.116 | −0.116 | −0.164 | −0.220 | −2.053 | 3.43E−04 |
| C11orf51 | NM_014042 | Hs.38044 | hs\|11q13.4 | −0.017 | −0.093 | −0.148 | −0.254 | −0.236 | −0.329 | −2.051 | 5.86E−04 |
| ENST00000399048 | BC110641 | Hs.572477 | hs\|17p11.2 | 0.273 | 0.277 | 0.242 | −0.058 | 0.044 | −0.040 | −2.050 | 8.29E−04 |
| SUV39H1 | NM_003173 | Hs.522639 | hs\|Xp11.23 | 0.049 | −0.116 | −0.013 | 0.015 | −0.159 | −0.274 | −2.049 | 4.17E−04 |
| CR599788 | CF124646 | Hs.650678 | hs\|17q25.3 | 0.327 | 0.155 | −0.059 | −0.025 | 0.017 | 0.032 | −2.045 | 6.49E−04 |
| SFTPA1 | XM_934590 | Hs.523084 | hs\|10q22.3 | 0.000 | −0.061 | 0.000 | −0.175 | −0.178 | −0.327 | −2.041 | 7.31E−04 |
| DYNLT1 | NM_006519 | Hs.445999 | hs\|6q25.3 | −0.023 | −0.128 | −0.130 | −0.216 | −0.276 | −0.333 | −2.041 | 8.72E−06 |
| ABCD1 | NM_000033 | Hs.159546 | hs\|Xq28 | 0.084 | 0.038 | −0.076 | −0.093 | −0.225 | −0.226 | −2.040 | 6.92E−04 |
| GPNMB | BC011595 | Hs.190495 | hs\|7p15.3 | 0.738 | 0.489 | 0.312 | 0.366 | −0.055 | 0.388 | −2.040 | 5.90E−04 |
| BOK | NM_032515 | Hs.293753 | hs\|2q37.3 | 0.058 | −0.063 | −0.076 | −0.129 | −0.196 | −0.254 | −2.039 | 9.11E−07 |
| TNIP2 | NM_024309 | Hs.368551 | hs\|4p16.3 | −0.096 | −0.028 | −0.082 | −0.120 | −0.221 | −0.265 | −2.038 | 1.41E−04 |
| KIFC1 | NM_002263 | Hs.436912 | hs\|6p21.32 | −0.067 | −0.101 | −0.160 | −0.153 | −0.261 | −0.402 | −2.037 | 1.33E−04 |
| PRPS1L1 | NM_175886 | Hs.169284 | hs\|7p21.1 | −0.041 | −0.099 | −0.238 | −0.290 | −0.326 | −0.346 | −2.032 | 4.51E−06 |
| COQ3 | NM_017421 | Hs.653253 | hs\|6q16.3 | −0.077 | −0.114 | −0.216 | −0.363 | −0.297 | −0.385 | −2.031 | 2.23E−04 |
| WASF2 | NM_006990 | Hs.590909 | hs\|1p36.11 | 0.164 | 0.072 | 0.029 | 0.077 | −0.099 | −0.143 | −2.030 | 1.01E−05 |
| BC040577 | BC040577 | Hs.563191 | hs\|4q34.1 | 0.191 | 0.155 | −0.145 | −0.129 | −0.116 | 0.149 | −2.029 | 6.87E−05 |
| LYRM4 | NM_020408 | Hs.387755 | hs\|6p25.1 | 0.047 | −0.025 | −0.018 | −0.008 | −0.164 | −0.216 | −2.024 | 3.17E−04 |
| CYorf16 | NR_001553 | Hs.638604 | hs\|Yp11.2 | 0.187 | −0.022 | 0.000 | −0.135 | −0.118 | −0.110 | −2.022 | 4.03E−04 |
| SPNS3 | NM_182538 | Hs.657543 | hs\|17p13.2 | 0.183 | 0.041 | 0.039 | −0.077 | −0.084 | −0.123 | −2.019 | 4.45E−04 |
| EVL | NM_016337 | Hs.125867 | hs\|14q32.2 | −0.116 | −0.176 | −0.163 | −0.149 | −0.325 | −0.368 | −2.012 | 3.96E−05 |
| HSPA12A | NM_025015 | Hs.654682 | hs\|10q25.3 | −0.062 | −0.173 | −0.242 | −0.428 | −0.314 | −0.366 | −2.011 | 5.29E−04 |
| APH1A | NM_016022 | Hs.108408 | hs\|1q21.2 | 0.148 | 0.049 | 0.118 | −0.016 | −0.062 | −0.155 | −2.010 | 3.81E−04 |
| NR2F6 | NM_005234 | Hs.466148 | hs\|19p13.11 | −0.025 | −0.077 | −0.140 | −0.144 | −0.317 | −0.327 | −2.006 | 5.14E−04 |
| CHRNA7 | NM_000746 | Hs.511772 | hs\|15q13.3 | −0.042 | −0.141 | −0.175 | −0.248 | −0.296 | −0.276 | −2.005 | 4.62E−05 |
| TAS2R7 | NM_023919 | Hs.533754 | hs\|12p13.2 | 0.223 | −0.034 | 0.385 | 0.432 | −0.027 | −0.066 | −2.004 | 4.06E−04 |

TABLE 3
Second list of the over-expressed genes

| | Genbank | | | 5 | 12 | 25 | 50 | 100 | 200 | Fold | |
|---|---|---|---|---|---|---|---|---|---|---|---|
| Gene Symbol | Accession# | UniGeneID | Cytoband | nM | nM | nM | nM | nM | nM | Change | p.value |
| TFPI2 | AK129833 | Hs.438231 | hs\|7q21.3 | 0.152 | 0.549 | 1.032 | 1.370 | 1.482 | 1.565 | 25.892 | 2.88E−09 |
| PCDH7 | NM_032456 | Hs.570785 | hs\|4p15.1 | −0.038 | 0.000 | 0.728 | 0.931 | 0.728 | 0.770 | 13.584 | 1.16E−05 |
| SMAD9 | BM802662 | Hs.586812 | hs\|13q13.3 | −1.698 | −1.063 | −1.451 | −1.366 | −1.015 | −0.788 | 8.127 | 1.71E−07 |
| AK090762 | AK090762 | Hs.531632 | hs\|19p12 | 0.128 | 0.146 | 0.468 | 0.479 | 0.674 | 0.981 | 7.646 | 4.79E−05 |
| RAB39B | NM_171998 | Hs.632832 | hs\|Xq28 | −0.264 | 0.000 | −0.694 | −0.226 | 0.293 | 0.516 | 6.031 | 4.98E−11 |
| BF831953 | | | hs\|7q31.1 | −0.111 | 0.000 | 0.231 | 0.448 | 0.658 | 0.189 | 5.987 | 9.33E−05 |
| AL050204 | AL050204 | Hs.28540 | hs\|11q22.3 | 0.110 | 0.043 | 0.221 | 0.351 | 0.694 | 0.883 | 5.922 | 6.79E−05 |
| VCX | NM_013452 | Hs.567503 | hs\|Xp22.31 | 0.559 | 0.486 | 0.538 | 0.372 | 0.883 | 1.203 | 5.235 | 8.85E−05 |
| ITGA2 | NM_002203 | Hs.482077 | hs\|5q11.2 | −1.460 | −0.725 | −1.429 | −1.566 | −0.809 | −0.748 | 4.838 | 5.20E−04 |
| AKAP9 | NM_147171 | Hs.651221 | hs\|7q21.2 | 0.101 | 0.299 | 0.159 | 0.244 | 0.502 | 0.694 | 3.908 | 5.59E−05 |
| AUTS2 | NM_015570 | Hs.654801 | hs\|7q11.22 | −1.021 | −0.844 | −0.522 | −0.587 | −0.547 | −0.433 | 3.823 | 2.33E−05 |
| CEP152 | NM_014985 | Hs.597323 | hs\|15q21.1 | 0.066 | 0.166 | 0.311 | 0.527 | 0.642 | 0.592 | 3.617 | 2.86E−06 |
| SLITRK6 | NM_032229 | Hs.525105 | hs\|13q31.1 | −0.617 | −0.363 | −0.352 | −0.370 | −0.200 | −0.071 | 3.519 | 7.87E−04 |
| CCPG1 | NM_020739 | Hs.612814 | hs\|15q21.3 | 0.382 | 0.542 | 0.493 | 0.535 | 0.767 | 0.889 | 3.213 | 1.72E−04 |
| MANEAL | NM_152496 | Hs.534562 | hs\|1p34.3 | 0.371 | 0.580 | 0.700 | 0.753 | 0.940 | 0.895 | 2.884 | 9.37E−04 |
| THC2733296 | | | hs\|21q21.1 | 0.163 | 0.091 | 0.289 | 0.381 | 0.474 | 0.578 | 2.603 | 1.00E−07 |
| CD55 | NM_000574 | Hs.527653 | hs\|1q32.2 | 0.389 | 0.306 | 0.664 | 0.740 | 0.785 | 0.784 | 2.505 | 6.48E−04 |
| ANKRD18A | AB095935 | Hs.561966 | hs\|9p13.1 | 0.354 | 0.482 | 0.527 | 0.655 | 0.723 | 0.629 | 2.340 | 3.02E−04 |
| LAT2 | NM_032463 | Hs.647049 | hs\|7q11.23 | −0.058 | −0.077 | 0.005 | −0.021 | 0.200 | 0.291 | 2.234 | 4.54E−04 |
| BRCA2 | NM_000059 | Hs.34012 | hs\|13q13.1 | −0.101 | −0.078 | 0.010 | 0.131 | 0.299 | 0.244 | 2.211 | 3.92E−06 |
| LRP2BP | NM_018409 | Hs.558513 | hs\|4q35.1 | −0.236 | −0.103 | −0.048 | 0.090 | 0.008 | 0.118 | 2.199 | 3.45E−04 |
| LPHN2 | NM_012302 | Hs.24212 | hs\|1p31.1 | 0.002 | 0.170 | 0.306 | 0.362 | 0.333 | 0.310 | 2.147 | 1.95E−11 |
| ITGB8 | NM_002214 | Hs.592171 | hs\|7p15.3 | 0.744 | 0.834 | 0.877 | 0.871 | 1.028 | 1.062 | 2.079 | 3.14E−04 |

TABLE 4
Second list of the under-expressed genes

| | Genbank | | | 5 | 12 | 25 | 50 | 100 | 200 | Fold | |
|---|---|---|---|---|---|---|---|---|---|---|---|
| Gene Symbol | Accession# | UniGeneID | Cytoband | nM | nM | nM | nM | nM | nM | Change | p.value |
| CXCR4 | NM_001008540 | Hs.593413 | hs\|2q21.3 | −0.410 | −0.660 | −1.321 | −1.590 | −1.751 | −1.556 | −15.696 | 2.05E−11 |
| SLC16A10 | NM_018593 | Hs.591327 | hs\|6q21 | −0.491 | −0.724 | −1.217 | −1.186 | −1.354 | −1.417 | −8.417 | 1.11E−07 |
| PDE1A | AL110263 | Hs.191046 | hs\|2q32.1 | −0.383 | −0.617 | −1.020 | −1.000 | −1.256 | −1.250 | −7.360 | 3.52E−05 |
| MAL | NM_002371 | Hs.80395 | hs\|2q11.1 | 0.983 | 1.206 | 1.000 | 0.879 | 0.459 | 0.253 | −5.715 | 3.35E−08 |
| KRT80 | NM_182507 | Hs.140978 | hs\|12q13.13 | 0.311 | −0.060 | 0.113 | −0.055 | −0.243 | −0.340 | −4.483 | 2.16E−04 |
| FXYD2 | AY946020 | Hs.413137 | hs\|11q23.3 | 0.550 | 0.262 | 0.237 | 0.277 | −0.068 | −0.070 | −4.167 | 6.81E−04 |
| AK3L1 | NM_001002921 | Hs.10862 | hs\|1p31.3 | −0.564 | −0.781 | −0.994 | −1.137 | −1.012 | −0.829 | −4.112 | 7.08E−05 |
| LIN7A | NM_004664 | Hs.144333 | hs\|12q21.31 | −0.523 | −0.608 | −0.593 | −0.982 | −1.110 | −0.778 | −3.868 | 9.34E−07 |
| GPR177 | NM_024911 | Hs.647659 | hs\|1p31.3 | −0.630 | −0.673 | −0.891 | −1.274 | −1.210 | −1.100 | −3.808 | 9.54E−05 |
| TNF | NM_000594 | Hs.241570 | hs\|6p21.33 | 1.035 | 1.276 | 1.284 | 1.222 | 0.739 | 0.661 | −3.603 | 2.51E−04 |
| WNT2B | NM_004185 | Hs.258575 | hs\|1p13.2 | 1.015 | 0.358 | 0.964 | 0.953 | 0.482 | 0.420 | −3.571 | 8.91E−05 |
| CGNL1 | NM_032866 | Hs.148989 | hs\|15q21.3 | −0.727 | −0.723 | −1.256 | −1.397 | −1.270 | −1.070 | −3.530 | 2.40E−05 |
| RPS6KA2 | NM_021135 | Hs.655277 | hs\|6q27 | 0.621 | 0.375 | 0.470 | 0.387 | 0.201 | 0.102 | −3.299 | 3.04E−04 |
| SUNC1 | NM_152782 | Hs.406741 | hs\|7p12.3 | −0.522 | −0.710 | −0.790 | −0.947 | −0.966 | −0.657 | −2.997 | 9.40E−04 |
| DIAPH2 | NM_006729 | Hs.656813 | hs\|Xq21.33 | −0.038 | −0.158 | −0.416 | −0.584 | −0.510 | −0.436 | −2.969 | 1.90E−06 |
| AKAP12 | NM_144497 | Hs.371240 | hs\|6q25.1 | −0.434 | −0.483 | −0.678 | −0.942 | −0.905 | −0.790 | −2.958 | 2.95E−04 |
| NRG1 | NM_013959 | Hs.453951 | hs\|8p12 | 0.334 | 0.000 | 0.077 | −0.296 | −0.095 | −0.129 | −2.898 | 5.02E−04 |
| PDE4DIP | NM_022359 | Hs.654651 | hs\|1q21.1 | 0.515 | 0.107 | 0.124 | 0.018 | 0.045 | 0.032 | −2.870 | 2.32E−04 |
| IL1R1 | NM_000877 | Hs.693591 | hs\|2q12.1 | 0.626 | 0.638 | 0.618 | 0.389 | 0.156 | 0.206 | −2.638 | 1.16E−07 |
| LZTS1 | NM_139201 | Hs.434996 | hs\|12q24.11 | −0.455 | −0.913 | −0.711 | −0.811 | −0.857 | −0.924 | −2.603 | 2.04E−06 |
| SLC3A1 | NM_000341 | Hs.112916 | hs\|2p21 | −0.507 | −0.558 | −0.737 | −0.869 | −0.968 | −0.921 | −2.594 | 2.28E−05 |
| MGST1 | NM_145791 | Hs.389700 | hs\|12p12.3 | −0.583 | −0.555 | −0.678 | −0.977 | −0.968 | −0.864 | −2.335 | 4.51E−05 |
| ACOT9 | NM_001037171 | Hs.298885 | hs\|Xp22.11 | 0.083 | −0.126 | −0.097 | −0.118 | −0.230 | −0.272 | −2.205 | 1.95E−04 |
| SLC12A3 | NM_000339 | Hs.658965 | hs\|16q13 | −0.569 | −0.521 | −0.673 | −0.855 | −0.936 | −0.833 | −2.182 | 6.59E−04 |
| ASRGL1 | BC006267 | Hs.535326 | hs\|11q12.3 | −0.038 | −0.172 | −0.316 | −0.299 | −0.342 | −0.417 | −2.053 | 6.67E−04 |
| HRG | NM_000412 | Hs.1498 | hs\|3q27.3 | 0.313 | 0.016 | 0.179 | 0.045 | −0.018 | −0.019 | −2.053 | 6.77E−04 |

TABLE 5
List of 44 genes

| AccessNum | UniGeneID | Symbol | Gene Name | FC exp1 | FC exp2 |
|---|---|---|---|---|---|
| NM_152688 | Hs.519794 | KHDRBS2 | KH domain containing, RNA binding, signal transduction associated 2 | 5.79 | 11.91 |
| NM_015570 | Hs.21631 | AUTS2 | Autism susceptibility candidate 2 | 3.82 | 10.44 |
| NM_032456 | Hs.479439 | PCDH7 | Protocadherin 7 | 13.58 | 8.19 |
| NM_207362 | Hs.469398 | C2orf55 | Chromosome 2 open reading frame 55 | 4.76 | 7.86 |
| NM_001999 | Hs.519294 | FBN2 | Fibrillin 2 | 2.15 | 5.51 |
| NM_001040874 | Hs.530461 | HIST2H2AA4 | Histone cluster 2, H2aa4 | 2.73 | 5.28 |
| NM_001012361 | Hs.133331 | WDR31 | WD repeat domain 31 | 4.73 | 4.62 |
| NM_152676 | Hs.664011 | FBXO15 | F-box protein 15 | 2.60 | 3.48 |
| NM_031435 | Hs.245798 | THAP2 | THAP domain containing, apoptosis associated protein 2 | 3.24 | 3.46 |
| BF207040 | Hs.353024 | BF207040 | Transcribed locus | 2.02 | 3.12 |
| NM_080593 | Hs.437275 | HIST1H2BK | Histone cluster 1, H2bk | 2.15 | 3.02 |
| NM_001080421 | Hs.164502 | UNC13A | Unc-13 homolog A (C. elegans) | 2.88 | 2.84 |
| BC032035 | Hs.31240 | FAM27E3 | Family with sequence similarity 27, member E3 | 2.15 | 2.75 |
| NR_003713 | Hs.720393 | LOC728613 | Programmed cell death 6 pseudogene | 2.11 | 2.69 |
| BC119676 | Hs.567050 | FAM27E1 | Family with sequence similarity 27, member E1 | 2.41 | 2.49 |
| NM_015392 | Hs.719906 | NPDC1 | Neural proliferation, differentiation and control, 1 | 2.40 | 2.15 |
| NM_003519 | Hs.137594 | HIST1H2BL | Histone cluster 1, H2bl | 2.26 | 2.02 |
| NM_016021 | Hs.163776 | UBE2J1 | Ubiquitin-conjugating enzyme E2, J1 (UBC6 homolog, yeast) | −2.20 | −2.06 |
| NM_201629 | Hs.50382 | TJP2 | Tight junction protein 2 (zona occludens 2) | −3.42 | −2.29 |
| NM_012206 | Hs.129711 | HAVCR1 | Hepatitis A virus cellular receptor 1 | −2.37 | −2.31 |
| NM_014797 | Hs.409876 | ZBTB24 | Zinc finger and BTB domain containing 24 | −2.53 | −2.39 |
| NM_017774 | Hs.657604 | CDKAL1 | CDK5 regulatory subunit associated protein 1-like 1 | −2.36 | −2.41 |
| NM_017421 | Hs.713623 | COQ3 | Coenzyme Q3 homolog, methyltransferase (S. cerevisiae) | −2.03 | −2.85 |
| NM_020698 | Hs.370410 | TMCC3 | Transmembrane and coiled-coil domain family 3 | −2.49 | −2.88 |
| NM_012082 | Hs.431009 | ZFPM2 | Zinc finger protein, multitype 2 | −3.52 | −3.35 |
| NM_000341 | Hs.112916 | SLC3A1 | Solute carrier family 3 (cystine, dibasic and neutral amino acid | −2.59 | −3.42 |
| | | | transporters, activator of cystine, dibasic and neutral amino acid | | |
| | | | transport), member 1 | | |
| NM_002394 | Hs.502769 | SLC3A2 | Solute carrier family 3 (activators of dibasic and neutral amino acid | −5.42 | −3.56 |
| | | | transport), member 2 | | |
| NM_014988 | Hs.335163 | LIMCH1 | LIM and calponin homology domains 1 | −7.34 | −3.71 |
| NM_001431 | Hs.486470 | EPB41L2 | Erythrocyte membrane protein band 4.1-like 2 | −2.37 | −4.33 |
| NM_212543 | Hs.13225 | B4GALT4 | UDP-Gal:betaGlcNAc beta 1,4-galactosyltransferase, polypeptide 4 | −2.45 | −4.64 |
| NM_144497 | Hs.371240 | AKAP12 | A kinase (PRKA) anchor protein 12 | −2.96 | −4.81 |
| NM_016356 | Hs.61345 | DCDC2 | Doublecortin domain containing 2 | −4.04 | −5.24 |
| AW014342 | Hs.665091 | BX281122 | Transcribed locus | −7.02 | −5.45 |
| NM_023074 | Hs.148322 | ZNF649 | Zinc finger protein 649 | −3.45 | −5.87 |
| NM_001003395 | Hs.591347 | TPD52L1 | Tumor protein D52-like 1 | −22.78 | −7.30 |
| NM_032801 | Hs.150718 | JAM3 | Junctional adhesion molecule 3 | −9.23 | −7.37 |
| NM_003730 | Hs.529989 | RNASET2 | Ribonuclease T2 | −4.70 | −7.43 |
| NM_000433 | Hs.587558 | NCF2 | Neutrophil cytosolic factor 2 | −14.39 | −7.68 |
| NM_182643 | Hs.134296 | DLC1 | Deleted in liver cancer 1 | −3.52 | −9.34 |
| NM_001008540 | Hs.593413 | CXCR4 | Chemokine (C-X-C motif) receptor 4 | −15.70 | −12.23 |
| CR594735 | Hs.153408 | CR594735 | hypothetical LOC100506305 (Homo sapiens) | −8.40 | −13.45 |
| NM_001003818 | Hs.729048 | TRIM6 | Tripartite motif-containing 6 | −13.03 | −16.24 |
| NM_133486 | Hs.105134 | MBNL3 | Muscleblind-like 3 (Drosophila) | −10.32 | −17.16 |
| NM_003480 | Hs.512842 | MFAP5 | Microfibrillar associated protein 5 | −17.38 | −91.67 |

TABLE 6
List of 17 genes.

| AccessNum | UniGeneID | Symbol | Gene Name | FC exp1 | FC exp2 | SEQ ID No |
|---|---|---|---|---|---|---|
| NM_152688 | Hs.519794 | KHDRBS2 | KH domain containing, RNA binding, signal | 5.79 | 11.91 | 1 |
| | | | transduction associated 2 | | | |
| NM_015570 | Hs.21631 | AUTS2 | Autism susceptibility candidate 2 | 3.82 | 10.44 | 2 |
| NM_032456 | Hs.479439 | PCDH7 | Protocadherin 7 | 13.58 | 8.19 | 3 |
| NM_207362 | Hs.469398 | C2orf55 | Chromosome 2 open reading frame 55 | 4.76 | 7.86 | 4 |
| NM_000341 | Hs.112916 | SLC3A1 | Solute carrier family 3 (cystine, dibasic and neutral | −2.59 | −3.42 | 5 |
| | | | amino acid transporters, activator of cystine, dibasic | | | |
| | | | and neutral amino acid transport), member 1 | | | |
| NM_144497 | Hs.371240 | AKAP12 | A kinase (PRKA) anchor protein 12 | −2.96 | −4.81 | 6 |
| NM_016356 | Hs.61345 | DCDC2 | Doublecortin domain containing 2 | −4.04 | −5.24 | 7 |
| NM_023074 | Hs.148322 | ZNF649 | Zinc finger protein 649 | −3.45 | −5.87 | 8 |
| NM_032801 | Hs.150718 | JAM3 | Junctional adhesion molecule 3 | −9.23 | −7.37 | 9 |
| NM_003730 | Hs.529989 | RNASET2 | Ribonuclease T2 | −4.70 | −7.43 | 10 |
| NM_000433 | Hs.587558 | NCF2 | Neutrophil cytosolic factor 2 | −14.39 | −7.68 | 11 |
| NM_182643 | Hs.134296 | DLC1 | Deleted in liver cancer 1 | −3.52 | −9.34 | 12 |
| NM_001008540 | Hs.593413 | CXCR4 | Chemokine (C-X-C motif) receptor 4 | −15.70 | −12.23 | 13 |
| CR594735 | Hs.153408 | CR594735 | hypothetical LOC100506305 (Homo sapiens) | −8.40 | −13.45 | 14 |
| NM_001003818 | Hs.729048 | TRIM6 | Tripartite motif-containing 6 | −13.03 | −16.24 | 15 |
| NM_133486 | Hs.105134 | MBNL3 | Muscleblind-like 3 (Drosophila) | −10.32 | −17.16 | 16 |
| NM_003480 | Hs.512842 | MFAP5 | Microfibrillar associated protein 5 | −17.38 | −91.67 | 17 |

1-15. (canceled)

16. An in vitro method for predicting or monitoring whether a patient affected by a cancer is responsive to a treatment with a molecule of the taxoid family, wherein the method comprises: 1) providing a biological sample from said subject; 2) determining in the biological sample the expression level of the genes JAM3, PCDH7, DCDC2, KHDRBS2, MFAP5, AUTS2, C2orf55, SLC3A1, AKAP12, ZNF649, RNASET2, NCF2, DLC1, CXCR4, CR594735, TRIM6, and MBNL3 thereby predicting or monitoring whether a patient affected by a prostate cancer is responsive to a treatment with a molecule of the taxoid family.

17. The method according to claim 16, wherein the method further comprises comparing the expression level of said genes to a reference expression level, the reference expression level being the expression level of the genes in cell-lines or patients sensitive to the treatment by the molecule of the taxoid family.

18. The method according to claim 17, wherein the over-expression of genes PCDH7, KHDRBS2, AUTS2, and C2orf55, and/or the under-expression of genes JAM3, DCDC2, MFAP5, SLC3A1, AKAP12, ZNF649, RNASET2, NCF2, DLC1, CXCR4, CR594735, TRIM6, and MBNL3 are indicative of a resistance to the treatment by the molecule of the taxoid family.

19. The method according to claim 16, wherein the molecule of the taxoid family is docetaxel, larotaxel, cabazitaxel (XRP6258), BMS-184476, BMS-188797, BMS-275183, ortataxel, RPR 109881A, RPR 116258, NBT-287, PG-paclitaxel, ABRAXANE®, Tesetaxel, IDN 5390, Taxoprexin, DHA-paclitaxel, and MAC-321.

20. The method according to claim 16, wherein the method further comprises determining the expression level of at least one gene selected from the group consisting of FBN2, HIST2H2AA4, WDR31, FBXO15, THAP2, BF207040, HIST1H2BK, UNC13A, FAM27E3, LOC728613, FAM27E1, NPDC1, HIST1H2BL, UBE2J1, TJP2, HAVCR1, ZBTB24, CDKAL1, COQ3, TMCC3, ZFPM2, SLC3A2, LIMCH1, EPB41L2, B4GALT4, BX281122 and TPD52L1.

21. The method according to claim 16, wherein the method further comprises determining the expression level of the genes FBN2, HIST2H2AA4, WDR31, FBXO15, THAP2, BF207040, HIST1H2BK, UNC13A, FAM27E3, LOC728613, FAM27E1, NPDC1, HIST1H2BL, UBE2J1, TJP2, HAVCR1, ZBTB24, CDKAL1, COQ3, TMCC3, ZFPM2, SLC3A2, LIMCH1, EPB41L2, B4GALT4, BX281122 and TPD52L1.

22. The method according to claim 16, wherein the method further comprises determining the expression level of at least one gene selected from the group consisting of the genes listed in Tables 1-4.

23. The method according to claim 16, wherein the biological sample is a cancer sample.

24. The method according to claim 16, wherein the cancer is selected from the group consisting of breast cancer, lung cancer, prostate cancer, gastric cancer and head and neck cancer.

25. A kit for predicting or monitoring whether a patient affected by a cancer is responsive to a treatment with a molecule of the taxoid family, wherein the kit comprises detection means selected from the group consisting of a pair of primers, a probe and an antibody specific to the genes JAM3, PCDH7, DCDC2, KHDRBS2, MFAP5, AUTS2, C2orf55, SLC3A1, AKAP12, ZNF649, RNASET2, NCF2, DLC1, CXCR4, CR594735, TRIM6, and MBNL3.

26. The kit according to claim 25, wherein the kit further comprises detection means for at least one gene selected from the group consisting of FBN2, HIST2H2AA4, WDR31, FBXO15, THAP2, BF207040, HIST1H2BK, UNC13A, FAM27E3, LOC728613, FAM27E1, NPDC1, HIST1H2BL, UBE2J1, TJP2, HAVCR1, ZBTB24, CDKAL1, COQ3, TMCC3, ZFPM2, SLC3A2, LIMCH1, EPB41L2, B4GALT4, BX281122 and TPD52L1.

27. A DNA chip for predicting or monitoring whether a patient affected by a cancer is responsive to a treatment with a molecule of the taxoid family, wherein the DNA chip comprises a solid support which carries nucleic acids that are specific to the genes JAM3, PCDH7, DCDC2, KHDRBS2, MFAP5, AUTS2, C2orf55, SLC3A1, AKAP12, ZNF649, RNASET2, NCF2, DLC1, CXCR4, CR594735, TRIM6, and MBNL3.

28. The DNA chip according to claim 27, wherein the DNA chip further comprises detection means for at least one gene selected from the group consisting of FBN2, HIST2H2AA4, WDR31, FBXO15, THAP2, BF207040, HIST1H2BK, UNC13A, FAM27E3, LOC728613, FAM27E1, NPDC1, HIST1H2BL, UBE2J1, TJP2, HAVCR1, ZBTB24, CDKAL1, COQ3, TMCC3, ZFPM2, SLC3A2, LIMCH1, EPB41L2, B4GALT4, BX281122 and TPD52L1.

29. A method for screening or identifying a compound suitable for improving the treatment of a cancer with a molecule of the taxoid family or for reducing the resistance development during the treatment of a cancer with a molecule of the taxoid family, comprising 1) providing a cell-line with the genes PCDH7, KHDRBS2, AUTS2, and C2orf55 being over-expressed and the genes JAM3, DCDC2, MFAP5, SLC3A1, AKAP12, ZNF649, RNASET2, NCF2, DLC1, CXCR4, CR594735, TRIM6, and MBNL3 being under-expressed; 2) contacting said cell-line with a test compound; 3) determining the expression level of said genes; and, 4) selecting the compound which decreases the expression level of one or several of the over-expressed genes and increases the expression level of one or several of the under-expressed genes.

30. The method according to claim 29, wherein the cell-line is a cancer cell-line.

31. A method for screening or identifying a compound suitable for improving the treatment of a cancer with a molecule of the taxoid family or for reducing the resistance development during the treatment of a cancer with the molecule of the taxoid family, comprising 1) providing a cell-line sensitive to the molecule of the taxoid family; 2) contacting said cell-line with a test compound and the molecule of the taxoid family; 3) determining the expression level of the genes JAM3, PCDH7, DCDC2, KHDRBS2, MFAP5, AUTS2, C2orf55, SLC3A1, AKAP12, ZNF649, RNASET2, NCF2, DLC1, CXCR4, CR594735, TRIM6, and MBNL3; and 4) selecting the compound which inhibits the appearance of an over-expression of the genes PCDH7, KHDRBS2, AUTS2, and C2orf55 and/or an under-expression of the genes JAM3, DCDC2, MFAP5, SLC3A1, AKAP12, ZNF649, RNASET2, NCF2, DLC1, CXCR4, CR594735, TRIM6, and MBNL3.

32. The method according to claim 31, wherein the cell-line is a cancer cell-line.


SEQUENCE LISTING (17 sequences)

SEQ ID NO: 1 - DNA, 2336 bp, Homo sapiens
ctggtgtgagccggggctctgcgcgagcgagggacgacggaagggacgggcaggtgtgggcgcggggccacgcagcccgacggcgggagtcgcaggtgctgggtgcatgggccagtgaggacgcacagagatccctcgccgcgcggaggaggagcagcgcgggagccaggcgctgccccaagaccctgcctgcgtccgagcgagcggaacctcgcgcttcgcccggggacaatccgaagtccgcgctatggaagaggagaaatatttgcctgagctgatggcagagaaagatagcctggatccatcttttgtgcatgcgtcgcgccttttggcagaagaaattgaaaagtttcaaggttctgatggaaaaaaggaagacgaagaaaagaagtatcttgatgtcatcagcaacaaaaacataaagctctcagaaagagtactgattcctgtcaagcagtatccaaagttcaattttgtggggaaattgcttggaccaagaggaaactccttgaagaggctacaggaagaaacaggtgctaaaatgtctatcctgggcaaaggatcaatgagagataaagctaaggaagaagaactaaggaagagtggggaagccaaatatgcccacttgagtgatgagcttcatgtattaattgaagtgtttgctccacctggggaagcttattcacgtatgagtcatgcattggaagagattaaaaaattcctggttcctgactacaatgatgaaattcgtcaggaacaactacgtgaattatcttacttaaatggctcagaggactctggtcgtggcagaggtattagaggcagagggatcagaatagctcccacagctccttcaaggggccgtgggggtgccattcctcctcccccaccacctggacgaggtgttctcacccctcggggaagcactgtaacccgtggagcgcttccagtgccacctgtagcaagaggtgtccctacccctcgagcccggggggcaccaacagtgccaggatacagggcacctcctcctccagcccatgaagcttatgaagaatatggttatgatgatggctacgggggtgaatatgatgaccagacctatgagacttatgataacagctatgcgacccaaacacaaagtgtgcctgaatactatgactacggtcatggagtaagtgaggatgcctatgacagctacgcaccagaagaatgggccacaacccgctctagcttgaaggcaccaccgcaaaggtcagccagagggggatacagggaacacccctatggtagatattgaaggtccttcccacctgtgacctcacctcaaagacaattcatagcctgtggtctccacataaacagcaacaagacaagtaatagtcctttttttgtttgtttgtttctattctagggataactgctcatgattactcccatatatttctagtatttccctctgtactgttggcgtgacattgacactagtattattttacaaaacagactgaaaaagacattagcaagcatagtctataaacctttcagtaggatgatattctcttggttgtttttcattgttgtgtgtaatcttttttttttctttttcttttttttttttttgaggaaaagcatgaatttgttaacagattttgagtctcaaccaactagcagaaactttgtttagggttgctaaaagactgtaatatgatttttgaactagctgataataaagttgtagataagatgttttaacctgtcttttaatatctgttagttagacaaagatgtttgatattaagtttgtaaatttaattttaatgctgttttaatggggttgaaaacaagcagctactgtatgtatgtagctaactgaatttgttcagtgttttaacctgtatttgttaaaaaagaaaaaaaaacacacataaagttccatgtgtaagcttctctaaataggaaaccacaatttgtcaaatatgtttgccataatttgtcaataaagctgaaaacttttgtaaaaattaaatttggaattacttgttttctagctttaaatgcctgctttatttcttcttcaagagatgcctaaaatgttttctatttaaaaattacaaaaatgaacccaagtctgttttaagatatttgtgtaatacttaaaaatgtattaataacttatggttgttaaataatttaaaagttaacaaactaaactgttacatgaatcatggttagtaaacactaatgtataacatgttaatggaaaaaaatggatttagaataataaatggattttaaactataaaaaaaaaaaaaaaaaaaaaaaaaaaaaaaaaaaa

SEQ ID NO: 2 - DNA, 6426 bp, Homo sapiens
gggagctgcgctcgcagtttcgccctctcttccgctaatgattgcattattatgctcccctctctggggggtctcgcccctcttgggtcgctccggagccccggcctcccctggctgcatttcttaaaaatttgggagcctgggagtgagttttctccgaggcgtgtgtgagaggcggcgggggtgttttcctgcgcgaggggcgggtgaagttcattgcccccacttttcccgcgacctttttcggacccgattttggatcgagttgaggggggcgcgggcgttttcggggggcggggggcgcggcggagaatggccgcggggagggctccccggagcctcccagtctcttgatcaaagcattccgctattctgatttattgcttgcttggtgagttatttttttttcctctaaaggagacctgtgtgttcagccattactttgctcggcgctgctcccaggcatctccgaccctcggtgctgtggggagccccacacttgggctcctcgcctctcgccctcgctccccgtccctcctcccctctctccgccccttcccccttttctttctcctctctttcttcccctctctcccttctttcggccgccgtctcccccgcgccctcctcggggcggagggaagccgtgaagggggagggagggctcggtgtcaatttttttttgtgtggctgcggccgtagcctgtggcgggcaagcggggagaccccggcgcagcagaaccatggatggcccgacgcggggccatggactccgcaaaaagcggcggtcgcggtcgcagcgagaccgggagaggcgctcccggggcgggctgggggccggcgcggccggcggcggcggggctggccggacccgggcgctctcactcgcctcgtcgtcgggctccgacaaggaagacaatgggaagcccccgtcctccgccccgtcccggcccagacccccgcggaggaagcggagagagtccacctcggcagaagaggacatcattgatggatttgccatgaccagctttgtcacttttgaagcgctggagaaagatgtagcacttaagcctcaggaacgtgtggagaaacgccagacgcccctgaccaagaagaaacgagaagcacttaccaatggcttgtcctttcattcaaagaagagcagactcagccacccacaccactacagctcagatcgagaaaatgaccgcaatctctgccagcaccttgggaagagaaagaaaatgccgaaggcactcagacagctcaagccaggacagaacagctgcagggacagtgacagtgaaagtgccagtggagaatccaagggcttccaccggagcagctctcgggaaaggctcagtgatagttcagctccttccagcttgggaacaggctacttctgtgacagtgacagtgaccaggaagagaaggcatcagatgccagctctgaaaaactcttcaacactgttattgtaaacaaagatccggagttaggtgttggcacgctaccagaacatgacagccaggatgcagggccgattgtccccaagatatcgggtctagagagaagccaggagaagagccaggactgttgcaaagagccaatctttgagcctgtggtgcttaaagacccctgccctcaggtcgcacagccaataccccagccgcagacggagccccaactccgagctccttctccggaccctgacttggtgcagcgcacagaggccccacctcaacccccacctctgagtacacagccaccacagggccctcctgaggcccagctccagcctgccccgcagcctcaggtgcagaggccacccaggccacagtcccccacccagctgctccatcagaacctcccacctgtgcaggcccacccctctgctcagagcctctcccagccattgtcagcctacaacagcagtagcttaagcctcaacagtttaagcagcagcagaagcagcactccagcgaagactcagcccgccccacctcacatctcccaccacccctctgcctccccgttccccctctccctgcccaaccacagccccctgcacagcttcacacccaccctccagccccccgcacactcacatcaccccaatatgtttgcccctcccactgctctgcctcctccaccaccactgacatcaggaagtctgcaggtggccggacacccggccgggagcacttactcagagcaagacatcttgcgacaggaactgaacactcgttttttggcctctcagagtgctgaccgcggggcttccctgggccctccgccctacctgcggaccgagttccatcagcaccagcaccagcaccagcacacccaccagcacacgcaccagcacaccttcacgccgttcccccacgccatcccacccaccgccatcatgccgacgccagcacctcccatgtttgacaaataccctacaaaagttgacccattctaccggcacagtctcttccattcctatcctcctgcagtgtcgggcatcccccctatgatcccacccactggcccttttggttcactacaaggagcatttcagccgaagacatccaaccctatcgatgtcgctgctcggcctgggacagtcccacacactttactccaaaaggacccgaggttgacagatcctttcagacctatgttaaggaaaccagggaagtggtgtgctatgcatgttcacatcgcctggcagatttaccaccaccaacagaaagtcaagaaacagatgcagtcagacccacataagctggactttggactgaaacctgagttcctgagccgccctccaggccccagtctttttggagccatccaccacccccatgacctggcacggccttcaactttgttctctgccgctggtgctgcacacccaactgggaccccttttgggccacctcctcatcacagcaacttcctcaaccctgctgcccacctagagccttttaatcggccgtctacattcacaggcctagcagcagttggtggcaatgccttcgggggacttggaaatccttccgttacacccaactcaatgttcggccacaaggatggccccagtgtgcagaactttagcaaccctcacgaaccctggaaccggctgcaccgaacgcctccgtcgttcccgacccctccgccctggctgaagccaggggagctggagcgcagcgcgtccgctgcagctcatgacagagatagagatgtagataaacgagactcatctgttagtaaagatgacaaagaaagggaaagcgtcgagaagagacactccagccacccttcaccagcacctgtcctcccggtgaatgccctgggacatacccgcagctccactgaacagatccgggctcatctgaacactgaggctcgggagaaggacaaacccaaagagagggagagagaccactcggaatcccgcaaggacctggccgccgacgagcacaaggcgaaagagggccacctgcccgagaaggacgggcacggccacgaggggcgcgccgcgggcgaagaggccaagcagctggcccgggtgccgtctccctacgtgcggaccccggtggtggagagtgccaggcccaacagcacctcgagccgggaggccgagccgcgcaagggtgagccggcctacgagaaccccaagaagagctccgaggtcaaggtgaaggaggagcggaaggaagaccatgacctgcctccagaggccccgcagacccaccgggcctcggagccgccgcctcccaactcctcgtccagcgtgcacccggggcccctggcctcgatgcccatgacggtgggggtgacgggcattcaccccatgaacagcatcagcagcctggacaggactcgcatgatgacccccttcatgggcatcagccccctcccgggcggagagcgcttcccgtacccttctttccactgggaccccatccgggaccccttgagggatccttaccgagaacttgacattcaccggagagacccgctgggcagggacttcctgctaaggaacgacccgctccaccggctctcgactccccggctgtacgaagccgaccgctccttcagggaccgggagcctcacgactacagccaccaccaccaccaccaccaccacccgctgtctgtggaccctcggcgggagcacgagcggggaggccacctggacgagcgggagcgcttgcacatgctcagagaagactacgagcacacgcggctccactccgtgcaccccgcctccctcgacggacacctcccccaccccagcctcatcaccccgggactccccagcatgcactatccccgcatcagccccaccgcgggcaaccagaacggactcctcaacaagacccctccgacagcagcgctgagcgcacctcccccgctcatctccacgctggggggccgcccggtctctcccagaaggacgactcctctgtccgcagagataagggagaggcccccttcccacacgctgaaggatatcgaggcccgataagccgagaacaggagcaagaacgaggaagaagaaaccctaggcagacaccaggccaggcttgagagacagaactcctgcatggctcacacagactgggggggaaagccccaccccttccccttgtaaaaaatgtatagactcagtgcacattttgaaatgttttgtatattatatgttgagatttttcagatcttttagcccagtcatatgttctcacgtctcctactttttgtttctcgtataaaactttttgatttgaaccaaaacagtgaagatgacaacacacaccaattggatgataattgtagcgggggcggtgggggggagaagtccacgccatccatcatgcaaaattctttcagatgaggtgggaaggccgtgtacatagttatgtaaaaagagattgcttcatgagctaatggttcatatatgcaaaagggtaagatgaaagctttactttgtacaaatgtaaatagataaagtaacataatacattaatacttcttaaaatgtgctatttgcaaacttacttaatatcagtgaacacagtcggctaaagctgtgttcccatatattgttatagacagctaaacccttcaactatgcaatgaatgttcgggcttttcacaaaagcccgcctaactcaaaggagccttttcaaatccatttacagcatacttaaggtcatattttccctgaacaagcgcttacgtgatatgactctgttttccttgcttgttttttttcaaacggagaaacatcctgttttgcaaattggaccccaggctggaacttagcatctgaagttgccgcttgtgggctctgggggaaagtgtagccccggagaggtaactgaggacatgagcaaccagtgccagggagggtgggatttgccagatgccaaaatcaggggacgggtggtggtgtctgtcagacacacacaggtcgccagtgacttcacacacacctcatgtgagaaccatgccttttttagtgtgtcctatttcatacctgtacacacttcctcgttttgtaatgagatttacttacacccaaacagatcctgaaagaaagcttcaagttttctcagatgatggatatgttttcactgtattcaataactgacggatgtaaggtgcacgtttcctgatgtgacgcactgtattccagctggtgatcaagtctgggaacagccgtaacaggtcaaccttgtggagccatcgcgagttagagggtgaaagatggcagaaaaaaaagtcttgtgtgtgagtgtgttttttgagtttgcatcaatcttaatgtctcttcataatacttttataatacattaagcctcttgtctacatatttggagagaatatgactttactagcagagaaatacaatatatcttgtctactggactgtaaaatatatgtatgaaataaaattagttccatttggtcttctagtatattaaagtgctatctgacgttgttatcctgtttttgcaaaaaaaaaaaaaaaaaaaagttaactacagaccattgtttctaataagcagagagatctattttagtagtaaactgaaggtttagttgtgagcttcagattttgtgaactccagatgttgtgcggtgtttttttttttttttaagacaacaactaaaaaaaatgcaaggaatatgtacactggaactgtagtggtagctttcagtattgtaaagagattgttctatacggacctttttgctgtttatcctgtatgtaataaagtcctttctagatcctatgtgaaaagaaaagtgaagcaactgaatcttcagcatgttctcatcggcggagccttcttgtgtaatgtaaactgtgccatgttattaaaaaatgtgaactaagcttccagctgcttgtttgtgtgaggtga

SEQ ID NO: 3 - DNA, 4928 bp, Homo sapiens
gcgggccgaatgcaggtgagaaaaggcacggactctgcggctgcgaacccaaacttgggcaccgcacggtgcgcactgctcagccttcgcccccgtgggcgaaaggctgctgcggtttcaggcggctgcttcgtgactaatgaccttgcgcagagttgttaagaaaaaagagaaacccgcgctctccggggtgagaagggactgactctgggcgtctctgaagatggctcgggcttctctttggcgcgccggggggaccctgacactgaccgctctgtgacgcgagtagtctcccctgcaccgtgcccgaagcgacgtgccgggggatttttcattctcgatctgttgactggctcccccgctgcatgagcagagtcggagttgagactggcttgttgctggccccagcgcctggtgcaggaagcgactcacgtttgtctgggtggccggagccggagcagagcctgggtttggagtgagtgcctggaacgtgaattggactcaactcgagtagcagcaaagaccagcgggctggcaggcgggggaggctgcaggctcattccccacctcttcccagccccactgcccgtctgccggagcggttctggccccttccgacagagcggggactagagccggggattctccgcccgctgaggggatgactctgggttgggggagcgccgaacccgcggcgcgcagtgtcccgtgaactgtgagtactgcgactgaacggcggcaggcgagcgggcgattagcacccattgcatgaattatgaaacaataactttcggaagaagcaggaggaaaaaaagaagcatctatcgctgccctcccacccccattcccggccaactctccacgccgcttttgccccctccctcccctccctctcgctccttcctttccgggagaggggagaggactcgggggagggcaggcggccggccccggaggaggggggcgccgagggggctgtggttagaaggagcagtagcagcagcagcaggagaagatgctgaggatgcggaccgcgggatgggcgcgcggctggtgcttgggctgctgcctcctcctgccgctctcgctcagcctggcggccgccaagcagctcctccggtaccggctggccgaggagggccccgccgacgtccgcatcggcaacgtggcttcagacctgggcatcgtgaccggatcgggtgaggtgactttcagcctggagtccggttccgagtacctgaagatcgacaacctcactggcgagctgagcacgagcgagcggcgcatcgaccgcgagaagctgccccagtgtcagatgatcttcgacgagaacgagtgcttcctggacttcgaggtgtcggtgatcgggccctcgcagagctgggtggacctgtttgagggtcaggtcatcgtgcttgacatcaacgacaacacgcccaccttcccgtcgcccgtgctcacgctcacggtggaggagaatcggccggtgggcacactttacctgctgcccacagccaccgaccgcgacttcggccgcaacggcatcgagcgctacgagctgctccaggagcccggaggcggcggcagcggcggcgagagccggcgcgccggggcggccgacagcgccccctaccccgggggcggcgggaacggcgcgagcggcggcggctcgggaggctccaagcggcggctggacgcatcagagggcggcggcggcaccaaccccggcggccgcagcagcgtgttcgagctgcaggtggcggacaccccggacggcgagaagcagccgcagctgatcgtgaagggggcgctggaccgcgagcagcgcgactcctacgagctgaccctgcgagtgcgcgacggcggcgacccgcctcgctcctcgcaggccatcctacgggtcctcatcaccgacgtgaacgacaacagcccccgcttcgagaagagcgtgtacgaggccgacttggctgagaacagcgccccggggacccccatcctgcaactgcgcgcagccgacttggacgtgggggtcaacgggcagatcgaatacgtgttcggggcggccaccgagtcggtgaggcggctgctgcgccttgacgagacgtccggctggctcagcgtcctgcaccggatcgaccgcgaggaggtgaaccagctgcgcttcacggtcatggcccgcgaccgcgggcagccccccaagaccgacaaggccaccgtggtccttaacatcaaagacgagaacgacaacgtgccgtccattgaaatccgcaagattgggcgcatccccctcaaggacggggtggccaacgtggccgaggacgttctggtcgacacccccatcgctctggtgcaggtgtccgaccgagaccaaggcgagaacggggtggtcacctgcaccgtggtgggcgacgtgcccttccagctcaagccagccagcgacaccgagggcgaccagaacaagaaaaagtacttcttgcacacctcgacccctctggactatgaggccacccgggagttcaacgtggtcatcgtggcggtggactcaggcagccccagcctctcgagcaacaactccctgattgtcaaggtgggagacaccaacgacaacccgcccatgttcggccagtcggtggtggaggtttacttccctgagaacaacatcccgggcgagagggtggccacggtgctggcgacagacgcagacagcggtaagaacgccgagatcgcctactcgctggactcctctgtgatggggatctttgccatcgatcccgattctggggacatcctggtcaataccgtgctggaccgcgagcagactgacaggtatgagtttaaagttaacgccaaagacaaaggcatccccgtgctgcagggcagcactacggtgattgtgcaggtggctgataaaaatgacaatgaccctaagtttatgcaggacgtcttcaccttttatgtgaaagaaaacttgcagcccaacagccctgtggggatggtcaccgtgatggatgctgacaaggggcggaatgcagagatgagcctgtacatagaggagaacaataacattttttctattgaaaatgacacggggaccatttactccacaatgtcttttgaccgggaacatcagaccacatacactttcagagtcaaggctgtggatgggggagatcctcccagatctgccacagctacagtctcgctttttgtgatggatgaaaatgacaatgctcccacagttacccttcccaaaaacatttcctacactttactgccaccttcgagtaatgtcaggacagtagtagctacagtgttggcaacagacagtgatgatggcatcaatgcagacctgaactacagcattgtgggaggaaatcccttcaagctgtttgaaattgatcccactagtggtgtggtttccttagtgggaaaactcacccaaaagcattatggcttgcacaggttggtggtgcaagtgaatgacagtgggcagccttcccagtccaccacgactctggtgcacgtgtttgtcaatgaaagtgtttctaatgcaactgcgattgactcccagatagctagaagtttgcacatcccactcacccaggatatagctggtgacccaagctatgaaattagcaaacagagactcagtattgtcattggcgtggttgctggcattatgacggtgattctaatcatcttaattgtagtgatggcaaggtactgcaggtccaaaaataaaaatggctatgaagccggcaaaaaagatcacgaagacttttttacaccccaacagcatgacaaatctaaaaagcctaaaaaggacaagaaaaacaaaaaatctaagcagcctctctacagcagcattgtcactgtggaggcttctaagccaaatggacagaggtatgatagtgtcaatgagaagctgtcagacagcccaagcatggggcgatacaggtccgttaatggtgggcccggcagtcctgacctggcaaggcattacaaatctagttccccattgcctactgttcagcttcatccccagtcaccaactgcaggaaaaaaacaccaggccgtacaagatctaccaccagccaacacatttgtgggagcaggagacaacatttcaattggatcagatcactgctctgagtacagctgtcaaaccaataacaagtacagcaaacaggtaagatgtatcccaaatatatttaaatatcccagggagggctaataactctgagacagattatcttctgtaaagttttgtcttctcgtttttaaagttattaaaattttaacagtaggaatttaatgttaatttttgcaccattaatttagcaaaggccatctacaagaggtataccactgtattttgcaaattagaattaaggttcactaaactactgaaagaagtattcagagtaggcagtgtttggcagttctctttgcacttgacatccctaattcatactacatttttttgtttccagaataaatatgtgtgtattacttagatggttagttttcacctgaaattattttcttctgtggcaactaagtggataattacatccagagaaaaagtttctaaagttactactggtgtctatgcaaattggattccatagaagttttagctatgttatttacaatgcaggattttacagataaatagatgctgattcatgcagatgtagtactaagtctgagatattgaaatactgactactttgatgaaactgctggtttaaaaatatttactgatatgcaaatgtcatgcaaaactccactttcaatcttttatatctataataatcgatacctataacaattagtgttctataatgatatgcaattaccatttgcattctttctgtttggtattaaaagtttgcaaacaaaaatacattgcatgagccatttat

SEQ ID NO: 4 - DNA, 3884 bp, Homo sapiens
ggagccgagcgaagccggcgcgcctgcagctgggagctgggagctgggagcgcgccgggcgcagggccgggccggccgcgggagggtagtgtcagcgcccgcgtccagctcttcggccgcagacccgggctgtctggaggccgcgcggccacctgctgcgtcgccccggctttgggctggggctagacgcgcaccgcggcgggagcagccccggctcgccgcaccccgcggaaggtgcccttctctgcgcgtctcgccgagcccgggccacgctcctgtcccagcgagcttgacatgatttccacaagggtgatggacattaagcttcgggaggctgcagaaggccttggggaggacagcacaggaaagaaaaaatctaaattcaaaacttttaagaagttttttgggaagaagaagagaaaagaatcgccgtcgtccacaggaagtagcacctggaaacaaagtcagacgaggaatgaggtcattgccatcgaatccgggccagtgggctacgactccgaggatgagctggaggagtcgaggggcacgctgggcagccgggccctttctcacgacagtattttcattcctgagtccggacaggacgctactcggcctgtgcgggtgttttcccaagaaaatgtgtgtgatcggattaaagctctgcagttaaaaatacagtgtaatgtgaaaatggggccaccacctcctccaggggggcttcctgccaagcggggagaggatgccggcatgagctctgaggacgacgggctgcccaggagccccccagagatgtctctgctgcacgacgtgggtcctggtaccaccataaaggtctctgtcgtgtctccagaccacgtgagcgacagcaccgtctctgcccggatctcagacaacagcctggcaccagtggctgacttcagttatcctgcagaatcctcctcctgcctggacaactctgcagctaagcacaagctccaggtcaagccccgcaaccagcggtcgagtaagatgaggcggctctcatcgcgcgctcagtctgaatccctgagcgacctgacgtgcaccccagaggaggaggaaaacgaggagaagccacttttggaagtcagcccagaagagcgccccagctctgggcagcaggacgtggcgccagacagaggccctgagcctgggccaccggcgcccttgccgccacccggaggggcccgtgccagacgcgcccgcctgcagcactcctccgcgctcacggccagcgtggaggaggggggcgtccccggggaggacccctcaagccgcccggccaccccggagctcgccgagcccgagtcggcccccactctccgcgtggagcccccgtccccgccggagggccccccgaatcccggtcccgacggcggaaagcaggatggggaggcgccccccgcaggcccgtgtgccccggccacggacaaggcggaggaggtggtctgtgctcccgaagacgtcgcgagcccgtttcccaccgccatccctgagggggacacgactccccccgagactgaccccgccgccacctcagaggcgccctctgctcgcgacgggccagaacgcagtgtcccgaaggaagcggagccgacgccgcccgtgctcccggatgaggagaaggggcccccagggccggcgcctgagcccgagagagaagcggagacggagcccgagagaggagcggggaccgagcccgagagaattgggaccgagccctccacggcgcccgccccgagcccgccggcgcccaagagctgcctgaaacaccggcccgcggccgccagcgagggccccgccgcgtccccgccgcttgccgctgcggagtctcccccggtggagcccggccccggttccctcgacgcagaggccgccgccccggagcgccccaaggccgagcgagccgaggcgccaccggcgggcgccgagagggcggcgccagagcggaaggcggagaggggcggtgccgagctgcgaggcgcgaagaagttctcggtgtcctcgtgccgagcgcggcctcgtccgggcgtctcccgcccgctggaacgggccagcggccgcctgcccctcgcgaggagcggcccggtctggaggagcgaggcggctcttgacgacctccagggtctccccgagccccagcacgcgaaacctggccctcggaagctggcggagcgcggccctcaggactcgggggacagggcggccagcccggccgggccgcgcaagagccctcaggaggcggccgccgcgcccggcacgagagagccctgcccagccgcccaggagccggccccgagtgaggacagaaaccccttccccgtcaagctccggtccacctccctctcgctcaaatacagggatggcgcctctcaggaggtgaagggtgtgaagaggtacagtgccgaggtccggttagaaaggtcgctgaccgtgctcccgaaggaggagaagtgtcccctcgggacggcccccgcccttcgaggcaccagggcccccagcgaccaaggaaaggggaaggcccggccccccgagccgctcagctccaagccgcccctgccccggaagccgcttctgcagagcttcacgctcccgcaccagcccgcgccccccgacgccggcccgggagagcgggaacccaggaaggagcccaggacggcggagaaaaggccgctgcgcaggggagctgaaaagagtctgccgcctgcagcaacagggcctggagctgatgggcagcctgcgccaccctggatcaccgtcactcggcagaagcggagggggaccttggaccagccacccaaccaggaagacaagcctggggcacggaccctgaagtctgaaccaggaaagcaagccaaggtgcccgagagaggccaggagcctgtgaagcaagctgactttgttcgcagcaagtctttcctgataacccctgtgaagcccgctgtggaccggaagcagggggcaaagctcaacttcaaggaggggctgcaaagaggaatctcattgtcccatcagaacttggctcagtctgcagtgatgatggagaaggaactgcatcagctgaagagagccagttatgccagtacagatcagccatcctggatggaacttgccagaaagaaatctcaagcttggagtgacatgccccagattataaaataggtgggcagtgtgctgataaagcatgtccactaccctgacaagccacttagttaaaaactgccagtgtattgtggcaaacaaactgtgaaacttaaagattgattttatcatcaagttatgacgaacttggggaagaggaaggaaccaggcaaaacaaagaggataaacacacagccatactcctgggccagagggcccagaaacaagaacaacattgaaaaagtccaggaaccagttcctgtgcccaaaggtacgaatgtgtcttttcgaattcccttgataaagaagcctcagacaaggaagaaaatagaaatgaaattggacacataaataggacacccagcaaaggccagaagagaagcccttctaggagaagggacatctgtgcttccagctgagatcagcccatgtctctggtcgctctcttgcctgtggatcctgtcttctttcatgtttactacgaggcatgacattcatggcaaaacagggatgccgtttccaagagcagctataaaacaatctgttttcctaaagacagccactctgagaaggagatgaaggaacacggcagggttcatcaattcatagactgtttccaaaaataatttttaaacttttccactaggcatctgtgtctttaaccactgcacaatgcattgcctgtttaataaaaggtttcaaacatgaaaaaaaaaaaaaaa

SEQ ID NO: 5 - DNA, 2353 bp, Homo sapiens
aagaccaagcattcagcaagccactcttccacctcccttactgcaggaaggcactccgaagacataagtcggtgagacatggctgaagataaaagcaagagagactccatcgagatgagtatgaagggatgccagacaaacaacgggtttgtccataatgaagacattctggagcagaccccggatccaggaagctcaacagacaacctgaagcacagcaccaggggcatccttggctcccaggagcccgacttcaagggcgtccagccctatgcggggatgcccaaggaggtgctgttccagttctctggccaggcccgctaccgcatacctcgggagatcctcttctggctcacagtggcttctgtgctggtgctcatcgcggccaccatagccatcattgccctctctccaaagtgcctagactggtggcaggaggggcccatgtaccagatctacccaaggtctttcaaggacagtaacaaggatgggaacggagatctgaaaggtattcaagataaactggactacatcacagctttaaatataaaaactgtttggattacttcattttataaatcgtcccttaaagatttcagatatggtgttgaagatttccgggaagttgatcccatttttggaacgatggaagattttgagaatctggttgcagccatacatgataaaggtttaaaattaatcatcgatttcataccaaaccacacgagtgataaacatatttggtttcaattgagtcggacacggacaggaaaatatactgattattatatctggcatgactgtacccatgaaaatggcaaaaccattccacccaacaactggttaagtgtgtatggaaactccagttggcactttgacgaagtgcgaaaccaatgttattttcatcagtttatgaaagagcaacctgatttaaatttccgcaatcctgatgttcaagaagaaataaaagaaattttacggttctggctcacaaagggtgttgatggttttagtttggatgctgttaaattcctcctagaagcaaagcacctgagagatgagatccaagtaaataagacccaaatcccggacacggtcacacaatactcggagctgtaccatgacttcaccaccacgcaggtgggaatgcacgacattgtccgcagcttccggcagaccatggaccaatacagcacggagcccggcagatacaggttcatggggactgaagcctatgcagagagtattgacaggaccgtgatgtactatggattgccatttatccaagaagctgattttcccttcaacaattacctcagcatgctagacactgtttctgggaacagcgtgtatgaggttatcacatcctggatggaaaacatgccagaaggaaaatggcctaactggatgattggtggaccagacagttcacggctgacttcgcgtttggggaatcagtatgtcaacgtgatgaacatgcttcttttcacactccctggaactcctataacttactatggagaagaaattggaatgggaaatattgtagccgcaaatctcaatgaaagctatgatattaatacccttcgctcaaagtcaccaatgcagtgggacaatagttcaaatgctggtttttctgaagctagtaacacctggttacctaccaattcagattaccacactgtgaatgttgatgtccaaaagactcagcccagatcggctttgaagttatatcaagatttaagtctacttcatgccaatgagctactcctcaacaggggctggttttgccatttgaggaatgacagccactatgttgtgtacacaagagagctggatggcatcgacagaatctttatcgtggttctgaattttggagaatcaacactgttaaatctacataatatgatttcgggccttcccgctaaaatgagaataaggttaagtaccaattctgccgacaaaggcagtaaagttgatacaagtggcatttttctggacaagggagagggactcatctttgaacacaacacgaagaatctccttcatcgccaaacagctttcagagatagatgctttgtttccaatcgagcatgctattccagtgtactgaacatactgtatacctcgtgttaggcacctttatgaagagatgaagacactggcatttcagtgggattgtaagcatttgtaatagcttcatgtacagcatgctgcttggtgaacaatcattaattcttcgatatttctgtagcttgaatgtaactgctttaagaaaggttctcaaatgttttgaaaaaaataaaatgtttaaaagtaaaaaaaaaaaaaaaaaaaaaaaaaaaaaaaaaaa

SEQ ID NO: 6 - DNA, 8246 bp, Homo sapiens
gtgcggcccgggcggggaagtttgcgctcccgaagtcctggagctcagcaagggaggggccagcgccagcccgcgtgtgggtggctgggtgggggcgtgggtgggggtccgcctataattatctggggaaatgcatccgcgctctgcttttcgctgcggcagctccgagggcacctccggttctcccccatcctccgggagtgtctgggcgctcagtccgctctgatcccgccgaaaccacctgcggttggcaggcaggagactaggcgtctgccggggagggcagggacccgctaagctgatctcctgtacagtagtgctacttaaaatatgctggggaccatcaccatcacagttggacagagagactctgaagatgtgagcaaaagagactccgataaagagatggctactaagtcagcggttgttcacgacatcacagatgatgggcaggaggagacacccgaaataatcgaacagattccttcttcagaaagcaatttagaagagctaacacaacccactgagtcccaggctaatgatattggatttaagaaggtgtttaagtttgttggctttaaattcactgtgaaaaaggataagacagagaagcctgacactgtccagctactcactgtgaagaaagatgaaggggagggagcagcaggggctggcgaccacaaggaccccagccttggggctggagaagcagcatccaaagaaagcgaacccaaacaatctacagagaaacccgaagagaccctgaagcgtgagcaaagccacgcagaaatttctcccccagccgaatctggccaagcagtggaggaatgcaaagaggaaggagaagagaaacaagaaaaagaacctagcaagtctgcagaatctccgactagtcccgtgaccagtgaaacaggatcaaccttcaaaaaattcttcactcaaggttgggccggctggcgcaaaaagaccagtttcaggaagccgaaggaggatgaagtggaagcttcagagaagaaaaaggaacaagagccagaaaaagtagacacagaagaagacggaaaggcagaggttgcctccgagaaactgaccgcctccgagcaagcccacccacaggagccggcagaaagtgcccacgagccccggttatcagctgaatatgagaaagttgagctgccctcagaggagcaagtcagtggctcgcagggaccttctgaagagaaacctgctccgttggcgacagaagtgtttgatgagaaaatagaagtccaccaagaagaggttgtggccgaagtccacgtcagcaccgtggaggagagaaccgaagagcagaaaacggaggtggaagaaacagcagggtctgtgccagctgaagaattggttgaaatggatgcagaacctcaggaagctgaacctgccaaggagctggtgaagctcaaagaaacgtgtgtttccggagaggaccctacacagggagctgacctcagtcctgatgagaaggtgctgtccaaaccccccgaaggcgttgtgagtgaggtggaaatgctgtcatcacaggagagaatgaaggtgcagggaagtccactaaagaagctttttaccagcactggcttaaaaaagctttctggaaagaaacagaaagggaaaagaggaggaggagacgaggaatcaggggagcacactcaggttccagccgattctccggacagccaggaggagcaaaagggcgagagctctgcctcatcccctgaggagcccgaggagatcacgtgtctggaaaagggcttagccgaggtgcagcaggatggggaagctgaagaaggagctacttccgatggagagaaaaaaagagaaggtgtcactccctgggcatcattcaaaaagatggtgacgcccaagaagcgtgttagacggccttcggaaagtgataaagaagatgagctggacaaggtcaagagcgctaccttgtcttccaccgagagcacagcctctgaaatgcaagaagaaatgaaagggagcgtggaagagccaaagccggaagaaccaaagcgcaaggtggatacctcagtatcttgggaagctttaatttgtgtgggatcatccaagaaaagagcaaggagagggtcctcttctgatgaggaagggggaccaaaagcaatgggaggagaccaccagaaagctgatgaggccggaaaagacaaagagacggggacagacgggatccttgctggttcccaagaacatgatccagggcagggaagttcctccccggagcaagctggaagccctaccgaaggggagggcgtttccacctgggagtcatttaaaaggttagtcacgccaagaaaaaaatcaaagtccaagctggaagagaaaagcgaagactccatagctgggtctggtgtagaacattccactccagacactgaacccggtaaagaagaatcctgggtctcaatcaagaagtttattcctggacgaaggaagaaaaggccagatgggaaacaagaacaagcccctgttgaagacgcagggccaacaggggccaacgaagatgactctgatgtcccggccgtggtccctctgtctgagtatgatgctgtagaaagggagaaaatggaggcacagcaagcccaaaaaagcgcagagcagcccgagcagaaggcagccactgaggtgtccaaggagctcagcgagagtcaggttcatatgatggcagcagctgtcgctgacgggacgagggcagctaccattattgaagaaaggtctccttcttggatatctgcttcagtgacagaacctcttgaacaagtagaagctgaagccgcactgttaactgaggaggtattggaaagagaagtaattgcagaagaagaaccccccacggttactgaacctctgccagagaacagagaggcccggggcgacacggtcgttagtgaggcggaattgacccccgaagctgtgacagctgcagaaactgcagggccattgggtgccgaagaaggaaccgaagcatctgctgctgaagagaccacagaaatggtgtcagcagtctcccagttaaccgactccccagacaccacagaggaggccactccggtgcaggaggtggaaggtggcgtacctgacatagaagagcaagagaggcggactcaagaggtcctccaggcagtggcagaaaaagtgaaagaggaatcccagctgcctggcaccggtgggccagaagatgtgcttcagcctgtgcagagagcagaggcagaaagaccagaagagcaggctgaagcgtcgggtctgaagaaagagacggatgtagtgttgaaagtagatgctcaggaggcaaaaactgagccttttacacaagggaaggtggtggggcagaccaccccagaaagctttgaaaaagctcctcaagtcacagagagcatagagtccagtgagcttgtaaccacttgtcaagccgaaaccttagctggggtaaaatcacaggagatggtgatggaacaggctatcccccctgactcggtggaaacccctacagacagtgagactgatggaagcacccccgtagccgactttgacgcaccaggcacaacccagaaagacgagattgtggaaatccatgaggagaatgaggtcgcatctggtacccagtcagggggcacagaagcagaggcagttcctgcacagaaagagaggcctccagcaccttccagttttgtgttccaggaagaaactaaagaacaatcaaagatggaagacactctagagcatacagataaagaggtgtcagtggaaactgtatccattctgtcaaagactgaggggactcaagaggctgaccagtatgctgatgagaaaaccaaagacgtaccatttttcgaaggacttgaggggtctatagacacaggcataacagtcagtcgggaaaaggtcactgaagttgcccttaaaggtgaagggacagaagaagctgaatgtaaaaaggatgatgctcttgaactgcagagtcacgctaagtctcctccatcccccgtggagagagagatggtagttcaagtcgaaagggagaaaacagaagcagagccaacccatgtgaatgaagagaagcttgagcacgaaacagctgttaccgtatctgaagaggtcagtaagcagctcctccagacagtgaatgtgcccatcatagatggggcaaaggaagtcagcagtttggaaggaagccctcctccctgcctaggtcaagaggaggcagtatgcaccaaaattcaagttcagagctctgaggcatcattcactctaacagcggctgcagaggaggaaaaggtcttaggagaaactgccaacattttagaaacaggtgaaacgttggagcctgcaggtgcacatttagttctggaagagaaatcctctgaaaaaaatgaagactttgccgctcatccaggggaagatgctgtgcccacagggcccgactgtcaggcaaaatcgacaccagtgatagtatctgctactaccaagaaaggcttaagttccgacctggaaggagagaaaaccacatcactgaagtggaagtcagatgaagtcgatgagcaggttgcttgccaggaggtcaaagtgagtgtagcaattgaggatttagagcctgaaaatgggattttggaacttgagaccaaaagcagtaaacttgtccaaaacatcatccagacagccgttgaccagtttgtacgtacagaagaaacagccaccgaaatgttgacgtctgagttacagacacaagctcacgtgataaaagctgacagccaggacgctggacaggaaacggagaaagaaggagaggaacctcaggcctctgcacaggatgaaacaccaattacttcagccaaagaggagtcagagtcaaccgcagtgggacaagcacattctgatatttccaaagacatgagtgaagcctcagaaaagaccatgactgttgaggtagaaggttccactgtaaatgatcagcagctggaagaggtcgtcctcccatctgaggaagagggaggtggagctggaacaaagtctgtgccagaagatgatggtcatgccttgttagcagaaagaatagagaagtcactagttgaaccgaaagaagatgaaaaaggtgatgatgttgatgaccctgaaaaccagaactcagccctggctgatactgatgcctcaggaggcttaaccaaagagtccccagatacaaatggaccaaaacaaaaagagaaggaggatgcccaggaagtagaattgcaggaaggaaaagtgcacagtgaatcagataaagcgatcacaccccaagcacaggaggagttacagaaacaagagagagaatctgcaaagtcagaacttacagaatcttaaaacatcatgcagttaaactcattgtctgtttggaagaccagaatgtgaagacaagtagtagaagaaaatgaatgctgctgctgagactgaagaccagtatttcagaactttgagaattggagagcaggcacatcaactgatctcatttctagagagcccctgacaatcctgaggcttcatcaggagctagagccatttaacatttcctctttccaagaccaacctacaattttcccttgataaccatataaattctgatttaaggtcctaaattcttaacctggaactggagttggcaatacctagttctgcttctgaaactggagtatcattctttacatatttatatgtatgttttaagtagtcctcctgtatctattgtatatttttttcttaatgtttaaggaaatgtgcaggatactacatgctttttgtatcacacagtatatgatggggcatgtgccatagtgcaggcttggggagctttaagcctcagttatataacccacgaaaaacagagcctcctagatgtaacattcctgatcaaggtacaattctttaaaattcactaatgattgaggtccatatttagtggtactctgaaattggtcactttcctattacacggagtgtgctaaaactaaaaagcattttgaaacatacagaatgttctattgtcattgggaaatttttctttctaacccagtggaggttagaaagaagttatattctggtagcaaattaactttacatcctttttcctacttgttatggttgtttggaccgataagtgtgcttaatcctgaggcaaagtagtgaatatgttttatatgttatgaagaaaagaattgttgtaagtttttgattctactcttatatgctggactgcattcacacatggcatgaaataagtcaggttctttacaaatggtattttgatagatactggattgtgtttgtgccatatttgtgccattcttttaagaacaatgttgcaacacattcatttggataagttgtgatttgacgactgatttaaataaaatatttgcttcacttagatttgctggttttattagataccaggaagcctgggacatatgtgtaccacattagaattctaaagataatgattgataagctagaactttctgatgtagtcattacatgaaaccccttgtcactggtttgtgtgttcagaggaagccatggccgagatagctttcctgaaataaaccagtagcttttcagattgacgttcttgctacaattgtaccatctggtaattcctgaaaatgtcaatttttttgtgttaatatttttggtttcaaacaataacaaatgtctctagaaagaaattttaagaaagcttaattaatagtaaaaatgcctttcctgaaataatcttggaaaattttttaaatgtcaaaatgatgagtcatgctaatacattgagggtttgtttttttgtttgtttgtttgtttgtttttgagacagagtttcgctcttgttgcccaggctggagtgcaatggcacgatctcagctcaccgcaacctccacctcccggattccagcgattctcctgcctcagcctacattaagggttttgtcagacaattgtcacacgaagaatagtgtcacttatctactcttgacacacagaactggcctggcatatagctttccagattttactcaaacttggtactccagtttgaaaatttaaattttgactgctgattagctggaaagcctagttttaatggaaagaaagtttgcttttaaaactgaaagtagtttctttttgctaacaaatctaacttcatacataattggccatattagtaaaacacctcatgatagcagtgtatatatagtcttgtttgtagttggaagtcatcttttaggagttattctcaaatatatataatagctacccatgcatcattattaaaatccccaaattcaaaaaacctctgatatatatatataatttttttttttttttttttttggccaactgagattgaaatccaagtgctggtttctagttctgaacatcaactaaagagttttggaaatgacagcaatttataacaagttcatattgacttcctctctatggcaggaagacattctgtgctgttttgaacagattaaagatttgtgtagtttgtgggaaattgacgtttttgtttaaattccacccgcgtttgtcttttcctaccacctgtggccaggtgctcgctggccatcacagttgcgattccatgagtagctgctttatgactgctttttgtactatctggatgtgcccagagttacttctgtacaagctctgtatctatgtccgttgagaacattattttaacaagaagaacaccaacagtagcatgaaatataatactgttttataattctaaagctgctgttaatttatgaagtacataataatctaatgtaaactgcagaagtcagagcaagtgcctacattttgttatttttggcattactacagagccatgtacaatagaaagcaatgcaagacttgtaaactctcaccacttcttgtaatatcaaatgttccccctcaggttattttgcttatggtacccatgagttgcctctctctgtacatagataaattgttccaatattttcctttgatgtttggaactacagatagtcaagggctggaaattttagttttcaatataagcttccagcttagcaattacctctagtcgaagacaatatttgattcctagttctgtttggggcaaattttcatttatctaaataaaatgcaatctaattaaatgccatggattttctttctgtaaaaaaaaaaaaaaaaaa

SEQ ID NO: 7 - DNA, 4708 bp, Homo sapiens
gtccaccttgattctgcccgtagcaggtgaacctggtgtggtgcgcgcactgcgggtcctgattggctgtgcacgccacggtgaaaaacttgagcgtctactccgagtcctgcaagctcctagtgtctcctctgggtggacgcctaggcgcacggccgttacttctctttcacctgtggattcgtcagtttctcgttcgtttcaggaggcgatcccgcgccctgggtgcggtgaggccgctgggacagcggaggtgcctcccgacgcgaagcagcgagctgaggcggcggccagcggggaagatgagcggcagcagcgccaggtccagccacctgtctcagcccgtcgtgaagagcgtgcttgtgtaccgcaacggggaccccttctacgcggggcgccgcgtcgtcatccatgagaagaaggtgtccagcttcgaagtcttcctgaaggaggtgaccggcggcgttcaggcaccctttggggccgtcaggaacatctacaccccgcggactggccaccgaatccggaagctagaccagatccagagcgggggcaattacgtggctggaggccaggaagccttcaagaaactcaattacttggacataggagaaatcaagaaaagaccaatggaagttgttaatacagaggtaaaaccagtaatccatagcaggatcaacgtgtcagctcgctttagaaaaccgcttcaggagccgtgcactatcttcttgattgcaaatggagacctcataaacccagcttctcgcctccttatccccagaaaaaccttgaatcagtgggatcatgtactacaaatggtcacagaaaaaatcactctgaggagcggggctgttcacaggctttatactttagaaggaaaacttgttgagagtggagcagagttggagaatgggcagttttatgtggctgttggcagagataagtttaagaaactgccttacagtgagttactttttgacaagtcaacgatgagaaggccttttggtcagaaagcttcttcactacctcctattgtaggatccagaaagtctaaagggagtggaaatgatcgccactctaagtcaacagttggatccagtgacaactcatctcctcagcccctgaagaggaaagggaaaaaagaagacgtgaattcagaaaaactgacgaaattgaaacaaaatgtaaaattaaagaattcacaagaaaccattccaaatagtgatgaaggcattttcaaagctggagcagagaggtctgaaacacggggggcagcagaagtccaagaagatgaagatactcaggttgaggttccagtcgatcagaggccagcagaaatagtagacgaggaagaagatggagagaaggcaaacaaggatgcagaacagaaagaagacttttcaggaatgaatggtgaccttgaagaggaaggaggtagggaggctacagatgcccctgagcaagtcgaggagattctggatcacagtgagcagcaggcacgccctgctcgtgtaaatggaggcaccgatgaggagaatggtgaggagctgcagcaggttaataatgagcttcaactggtcctagacaaggaaagaaagtctcaaggagctggcagtggacaagatgaggctgatgtagaccctcaaagaccaccaaggccagaagtaaaaattaccagtccagaagaaaatgaaaacaaccaacaaaacaaggactatgctgccgtggcttagaacatttttaaaaagagagtatatggatcgcaagaaaaatgaagggttatcatacttgaaagataagcacatagttattgctgaatataatgtgacactatggtcgaatactacctacgaattataacattagaagcctagtggaaagaccagataactttaaatggctactaaaggataattacttacttttattgcatgtgttttaaaagtcatatagaaatattaaataagacggacagaggagaatttgcactggaagacaattgccacttgtaaaggatgaaaaataggatcactcttattgtacgctttattataagtttagaaggcagtttattctaaataatttttctctaggaaggcgtagaattttaaagaactggtaataggaaagcatgtactattttcttaaagcaataaactcttgaatgaacagattgcgatttactttcagacataatttggagatggcagtagatcaaaatgtgtccatgacttgttaacatgcctttctttcttcctccttagccaaaatccacctttgaactacaaagacagagcaaggcgttcattttggtgggaggaagcattggttcagagtgttagtgactagtatcgccatgccgtcacttaaatgctttcaggcttgcatgcttgtggctccaatggcgcacactcaggaaggaattgtaaaggagcacccagttatattataaagcctggatgtatggtttgcagataatggaaatcctgtggattttcactgatccagtctatctttaccaatagtatctctctcttctcccttatgttattagagaacctgatattggctattccaaagattaaattattttcaaatagttttcaacaaaaataaaagtgtattaggaagaaaaaagtagactatatgaagagtttgtgactgctcaatttaacttgttttttgccttatttctattaagactgttttactatgtttttgccctagggatatcagcaaatatttatttttcctagcatgatatagttagaatttcaagcagatttctttgtaattagaaggcatctgatagaaattgtaaaactttagaagttattataatgaaaccaattcctgaatcacaacttcatggactgactaaattgatttatagttgccttgtgaggtatgtatggggaaaacataaaaacataattaaaacataatttcgtcctttttatgaattcttaagagatgttcttccataaatatagaaataatatatttttcttaaaggatatatttttaattatgtggaagttgtaagcttgaaattttaacttctagtgcttttctaaaattcacaattacaagtttaaaacattttttctcttccaggtttcatttggaataaagagttggcagttatataaagcacttaataatactatagaaaatagatgtgttttttcttaatatgattttggtattttccacagatcagaagtgtaaacagagagataatgtaacagtatttggaagagataatgacacaaggatatagtgatctgggagcatgaattagaaacagggtactatgtttctggtgtaaaatctaaattgtgctttctactagcgttactttttcaaatgggatgatataaagcactgtgggctataagcaacaatcttgggttggtaggcaaatagaccaattgatctttattattattattattattattattattattttttgagatgaagtcttgctcttgtcgcccaggctgggtgcagtggcatgatcttggcttactgcaacctctgcctcctgggttcaagcacctgagcctcccgagtagctgggattacaggcatctgccaccacacctggctaatttttgtattttttgtagagacagatgtttcaccatgttggccaggctggtctcaaactcctgacctcaggtgatccgcccgcctttgcatcccaaaatgctgggattacaggtgtgagccacagcacccggtcccagttggtcttttaatattaaatttgtgcactattactgggtctggagttaatgatttcaaatagtttctaaaaataactgattaaatgtaagaaattataactaattatcaaggtgattctcttttgtttcttcaatggagagtttctctttctcttttcctcctctaataaaaatatttctttttttcctgtccttttattcattatattatgaaacttaggttttaggaaacaccttaagcgtcattttaatgaagcacaacattgatattttacatgaataataacgtttggtattttaaatatacttcaagggggtaataagccacaataactgtaccaaaaaaacttaaaaatggggacatattcatcccttgtgtgtgcatattttaccacttgttgacaagatacaattgtgacctactttgaggggatgaataacgacttatcactttaagacactttgcaaacatgttactaaaaaaaggtgcctagccaaggaaaaaaccattcacttggaattaaaattgtggatgtcacttcagagatgactttaacataaatgctgaaagttcattcacctcacaaatgtggcagttttctctgtggagaatggtggtctccgtggcagactggctctccaaattaaatgaaactcattgacagacctgggaccttttatgtgagtggacagacaagatcttggtttggcttcatgtactgttttctagagagagtgctacgaaaaacttatgatgcttttatgtgataaccaatataatgctttaatatgttaattctttgtgatgctatatacatagtttttatagttttatgaaaccactttgttaaacttatctggattttaagacacttgtaaacaaatggtttgttagtaaaattggggcattaagagttttacagagcaaataaagaagattttgacaaaagtca

SEQ ID NO: 8 - DNA, 3234 bp, Homo sapiens
gaagagaagcccatgtcccatcgtcctagcgcggcagaattgactcgcgccctcgtccgccgccataggccagtgccggggtttaagggccaggaaaggaagcattcagggaatttaggtgtagccagaagaaaatcaggtcctggctccccagaagcaagagagttcaaatgaaggaaggaggaggttcctggatgtggatgtcatcatttctgggaacactcttaaatggagactcagatttcttagccaaaatttagggaggatccagaagaaaccaaagacgaagcatcccagttcttgggtatttcctgaaacagaagaaaatgacaaaggcccaggaatcactgaccctggaggatgtggctgtggacttcacctgggaggagtggcagttcctgagccctgctcagaaggacctgtaccgggatgtgatgttggagaactacagcaaccttgtgtcagtggggtatcaagccggcaaacctgatgccctcaccaagttggaacaaggagaaccactatggacactagaagatgaaatccacagtccagcccacccagaaattgagaaagctgatgatcatctgcagcagcccttgcaaaaccaaaaaatactgaagaggacgggacaacgctatgaacacggaagaactttgaaatcatatttaggtttaaccaaccagagcagaagatacaacagaaaggagcctgctgagtttaatggagatggagcttttctccatgataatcatgaacaaatgcctacggaaattgaattccctgaaagtagaaaacccatcagcaccaagtcacaattccttaaacatcagcaaacacacaacatagagaaagcccatgaatgcactgactgtgggaaagctttcctcaagaagtctcagctcactgagcataagagaattcatacaggaaagaaaccccacgtgtgtagcttgtgtgggaaagccttctacaagaagtacaggctcactgaacacgagagagctcacagaggagagaaaccccacgggtgtagcttgtgtgggaaagccttctacaagaggtacaggctcactgaacacgagagagctcacaaaggagagaaaccatacgggtgcagtgaatgtgggaaagccttccccaggaaatctgagcttactgaacatcaaaggattcacacgggaattaagccccatcaatgcagcgaatgtgggagagctttctccagaaaatcactactcgttgtacatcagcgaactcatacaggagagaagcctcatacatgcagtgaatgtggaaaaggcttcattcagaagggcaatctcaacatacatcaacgaactcacactggagagaaaccttatggatgcattgactgtggcaaggccttcagccagaagtcttgccttgtagcacatcagagatatcatacaggaaagactccctttgtatgtcctgaatgtgggcaaccctgttcacagaagtcaggactcattagacatcagaaaattcactcaggagagaaaccctataaatgcagtgactgtgggaaagccttccttacaaagacaatgctcattgtacatcacagaactcacacgggagagagaccctatggctgtgatgagtgtgagaaagcttacttctatatgtcttgccttgttaaacataagagaatacactcaagggagaaacggggggattcagtgaaggtggaaaatccttccacagcaagtcacagcttaagtcctagtgaacatgtgcaggggaaaagccctgttaatatggtaactgtggcaatggtggcagggcagtgtgagtttgcccacatcctgcattcatgataaacagtttgctgtttgatcatatagcctccagcggaatgctgagtttgtcatgtcccatgggcctttggctccctgcactaatatgtatagtagggtttacaagatatgaaatatattttacttttttatatcttataaacctcactacccctcccacaatattgtttttcatttactatcttgatcatagagtttggctggggaggggggcagttttagaggcttccacttggtgttcctcagaatgatatctcttactccgggggccaaggtaggggttagcttttgttctctttgtagtttagattgtatctcttgccttgttcaagttcacaaatctttttgtgtatacacatatgtacatgaaaatgatgttcatgctttttattattttacccttcattatttcattttttatagttctcatagctatgtctttcagttcactaatcttttttccacagtgtttaatctgtcattaatcccatccaaagtatgttttctctcagaaattgtaatttttaacctctaataatttgacatgggtttttccctgttatatctttcatatctttatttatcatggttttacttttaacctatttatgctttcatatttaaagtgggtgtttagtgggcaacatacagttgggccttccttttttatccagttaacaatctctgtctttttactggggtatttagatcatttacgttcaatgtattgatgtttttaggtttaaattactaacttactatttgttgtttattctatgtcttctttgttctccttttcttctttttctgctttcccttagagtagctgagtgtgtttatattacatattgtctcctttgttggcttattaactgtaaatccttgtcatttgattccttgagattttatagcatacatcattgtgagggattgcatatttatgtcctctccaaatttacatgttgaagtcctaatccctaagggatggcattaggaagtgtgacctttggaaggtaattacatcttgaaagtggaaccctcatgatgagattatgtgtcttatgagacaacacagaagagagtttgttttctctttctctgctctttgccatgtgaagacagaatgagatgaccatgcataaaccaggaaatggactctcactggacactagacctgccaacaccttgattgtgttttagagtccagttctagagactcctcagcctctggaacgtgagaaatgaatgtttgttaaggcagtcagtcagtctatggtgtattttttctagtagcctgaactgactgaaagaaaatcatgaacttatcattctaacttcaaggacattatatcacttactgtataataaaataatcttacagtaaaaaaaa

SEQ ID NO: 9 - DNA, 3675 bp, Homo sapiens
tagacctcagcttcctctgtcaccatggtgccggctcggctgggcccggcggtcgccatggtaactggggcgggtcgcagggtcctggcaggctgggcgcatgcgcgcggggactacaagccgcgccgcgctgccgctggcccctcagcaaccctcgacatggcgctgaggcggccaccgcgactccggctctgcgctcggctgcctgacttcttcctgctgctgcttttcaggggctgcctgataggggctgtaaatctcaaatccagcaatcgaaccccagtggtacaggaatttgaaagtgtggaactgtcttgcatcattacggattcgcagacaagtgaccccaggatcgagtggaagaaaattcaagatgaacaaaccacatatgtgttttttgacaacaaaattcagggagacttggcgggtcgtgcagaaatactggggaagacatccctgaagatctggaatgtgacacggagagactcagccctttatcgctgtgaggtcgttgctcgaaatgaccgcaaggaaattgatgagattgtgatcgagttaactgtgcaagtgaagccagtgacccctgtctgtagagtgccgaaggctgtaccagtaggcaagatggcaacactgcactgccaggagagtgagggccacccccggcctcactacagctggtatcgcaatgatgtaccactgcccacggattccagagccaatcccagatttcgcaattcttctttccacttaaactctgaaacaggcactttggtgttcactgctgttcacaaggacgactctgggcagtactactgcattgcttccaatgacgcaggctcagccaggtgtgaggagcaggagatggaagtctatgacctgaacattggcggaattattgggggggttctggttgtccttgctgtactggccctgatcacgttgggcatctgctgtgcatacagacgtggctacttcatcaacaataaacaggatggagaaagttacaagaacccagggaaaccagatggagttaactacatccgcactgacgaggagggcgacttcagacacaagtcatcgtttgtgatctgagacccgcggtgtggctgagagcgcacagagcgcacgtgcacatacctctgctagaaactcctgtcaaggcagcgagagctgatgcactcggacagagctagacactcattcagaagcttttcgttttggccaaagttgaccactactcttcttactctaacaagccacatgaatagaagaattttcctcaagatggacccggtaaatataaccacaaggaagcgaaactgggtgcgttcactgagttgggttcctaatctgtttctggcctgattcccgcatgagtattagggtgatcttaaagagtttgctcacgtaaacgcccgtgctgggccctgtgaagccagcatgttcaccactggtcgttcagcagccacgacagcaccatgtgagatggcgaggtggctggacagcaccagcagcgcatcccggcgggaacccagaaaaggcttcttacacagcagccttacttcatcggcccacagacaccaccgcagtttcttcttaaaggctctgctgatcggtgttgcagtgtccattgtggagaagctttttggatcagcattttgtaaaaacaaccaaaatcaggaaggtaaattggttgctggaagagggatcttgcctgaggaaccctgcttgtccaacagggtgtcaggatttaaggaaaaccttcgtcttaggctaagtctgaaatggtactgaaatatgcttttctatgggtcttgtttattttataaaattttacatctaaatttttgctaaggatgtattttgattattgaaaagaaaatttctatttaaactgtaaatatattgtcatacaatgttaaataacctatttttttaaaaaagttcaacttaaggtagaagttccaagctactagtgttaaattggaaaatatcaataattaagagtattttacccaaggaatcctctcatggaagtttactgtgatgttccttttctcacacaagttttagcctttttcacaagggaactcatactgtctacacatcagaccatagttgcttaggaaacctttaaaaattccagttaagcaatgttgaaatcagtttgcatctcttcaaaagaaacctctcaggttagctttgaactgcctcttcctgagatgactaggacagtctgtacccagaggccacccagaagccctcagatgtacatacacagatgccagtcagctcctggggttgcgccaggcgcccccgctctagctcactgttgcctcgctgtctgccaggaggccctgccatccttgggccctggcagtggctgtgtcccagtgagctttactcacgtggcccttgcttcatccagcacagctctcaggtgggcactgcagggacactggtgtcttccatgtagcgtcccagctttgggctcctgtaacagacctctttttggttatggatggctcacaaaatagggcccccaatgctattttttttttttaagtttgtttaattatttgttaagattgtctaaggccaaaggcaattgcgaaatcaagtctgtcaagtacaataacatttttaaaagaaaatggatcccactgttcctctttgccacagagaaagcacccagacgccacaggctctgtcgcatttcaaaacaaaccatgatggagtggcggccagtccagccttttaaagaacgtcaggtggagcagccaggtgaaaggcctggcggggaggaaagtgaaacgcctgaatcaaaagcagttttctaattttgactttaaatttttcatccgccggagacactgctcccatttgtggggggacattagcaacatcactcagaagcctgtgttcttcaagagcaggtgttctcagcctcacatgccctgccgtgctggactcaggactgaagtgctgtaaagcaaggagctgctgagaaggagcactccactgtgtgcctggagaatggctctcactactcaccttgtctttcagcttccagtgtcttgggttttttatactttgacagcttttttttaattgcatacatgagactgtgttgactttttttagttatgtgaaacactttgccgcaggccgcctggcagaggcaggaaatgctccagcagtggctcagtgctccctggtgtctgctgcatggcatcctggatgcttagcatgcaagttccctccatcattgccaccttggtagagagggatggctccccaccctcagcgttggggattcacgctccagcctccttcttggttgtcatagtgatagggtagccttattgccccctcttcttataccctaaaaccttctacactagtgccatgggaaccaggtctgaaaaagtagagagaagtgaaagtagagtctgggaagtagctgcctataactgagactagacggaaaaggaatactcgtgtattttaagatatgaatgtgactcaagactcgaggccgatacgaggctgtgattctgcctttggatggatgttgctgtacacagatgctacagacttgtactaacacaccgtaatttggcatttgtttaacctcatttataaaagcttcaaaaaaacccaaaaaaacccaaa

SEQ ID NO: 10 - DNA, 1250 bp, Homo sapiens
ctctgctccggtgcaggcccgcagggcgccctgggctgggagcaacgcgactgaccgtggtcgtgggcggacggcggctgcagcgtggaggagctggggtcgctgtgggtcgcgaagcagagcccgggacgtgcgcgcttggtgcacgatcctgaaggggagctccgaggggcccgggtcgccagggctgctgcggccattcccggagcccggcgcggggcccgcgagatactggtttaggccgtcccagggctccgggcgcacccggtggccgctgctgcagcggagggagcgcggcggcgcgggggctcggagacagcgtttctcccggaagtcttcctcgggcagcaggtgggaagtgggagccggagcggcagctggcagcgttctctccgcaggtcggcaccatgcgccctgcagccctgcgcggggccctgctgggctgcctctgcctggcgttgctttgcctgggcggtgcggacaagcgcctgcgtgacaaccatgagtggaaaaaactaattatggttcagcactggcctgagacagtatgcgagaaaattcaaaacgactgtagagaccctccggattactggacaatacatggactatggcccgataaaagtgaaggatgtaatagatcgtggcccttcaatttagaagagattaaggatcttttgccagaaatgagggcatactggcctgacgtaattcactcgtttcccaatcgcagccgcttctggaagcatgagtgggaaaagcatgggacctgcgccgcccaggtggatgcgctcaactcccagaagaagtactttggcagaagcctggaactctacagggagctggacctcaacagtgtgcttctaaaattggggataaaaccatccatcaattactaccaagttgcagattttaaagatgcccttgccagagtatatggagtgatacccaaaatccagtgccttccaccaagccaggatgaggaagtacagacaattggtcagatagaactgtgcctcactaagcaagaccagcagctgcaaaactgcaccgagccgggggagcagccgtcccccaagcaggaagtctggctggcaaatggggccgccgagagccggggtctgagagtctgtgaagatggcccagtcttctatcccccacctaaaaagaccaagcattgatgcccaagttttggaaatattctgttttaaaaagcaagagaaattcacaaactgcagctttctaaaaaacaa

SEQ ID NO: 11 - DNA, 2429 bp, Homo sapiens
acactccacccctactcgccctctctctctctgcttctttccttttctctctcatggtagggttatgagtcagttgccaaaaggtggggacatttcctgatgcatttgcaacactgagaagttatcttaagggaggctgggccccattctactcatctggcccagaaagtgaacaccttgggggccactaaggcagccctgctaggggagacgctccaacctgtcttctctctgtctcctggcagctctcttggcctcctagtttctacctaatcatgtccctggtggaggccatcagcctctggaatgaaggggtgctggcagcggacaagaaggactggaagggagccctggatgccttcagtgccgtccaggacccccactcccggatttgcttcaacattggctgcatgtacactatcctgaagaacatgactgaagcagagaaggcctttaccagaagcattaaccgagacaagcacttggcagtggcttacttccaacgagggatgctctactaccagacagagaaatatgatttggctatcaaagaccttaaagaagccttgattcagcttcgagggaaccagctgatagactataagatcctggggctccagttcaagctgtttgcctgtgaggtgttatataacattgctttcatgtatgccaagaaggaggaatggaaaaaagctgaagaacagttagcattggccacgagcatgaagtctgagcccagacattccaaaatcgacaaggcgatggagtgtgtctggaagcagaagctatatgagccagtggtgatccctgtgggcaagctgtttcgaccaaatgagagacaagtggctcagctggccaagaaggattacctaggcaaggcgacggtcgtggcatctgtggtggatcaagacagtttctctgggtttgcccctctgcaaccacaggcagctgagcctccacccagaccgaaaaccccagagatcttcagggctctggaaggggaggctcaccgtgtgctatttgggtttgtgcctgagacaaaagaagagctccaggtcatgccagggaacattgtctttgtcttgaagaagggcaatgataactgggccacggtcatgttcaacgggcagaaggggcttgttccctgcaactaccttgaaccagttgagctgcggatccaccctcagcagcagccccaggaggaaagctctccgcagtccgacatcccagctcctcctagttccaaagcccctggaagaccccagctgtcaccaggccagaaacaaaaagaagagcctaaggaagtgaagctcagtgttcccatgccctacacactcaaggtgcactacaagtacacggtagtcatgaagactcagcccgggctcccctacagccaggtccgggacatggtgtctaagaaactggagctccggctggaacacactaagctgagctatcggcctcgggacagcaatgagctggtgcccctttcagaagacagcatgaaggatgcctggggccaggtgaaaaactactgcctgactctgtggtgtgagaacacagtgggtgaccaaggctttccagatgaacccaaggaaagtgaaaaagctgatgctaataaccagacaacagaacctcagcttaagaaaggcagccaagtggaggcactcttcagttatgaggctacccaaccagaggacctggagtttcaggaaggggatataatcctggtgttatcaaaggtgaatgaagaatggctggaaggggagtgcaaagggaaggtgggcattttccccaaagtttttgttgaagactgcgcaactacagatttggaaagcactcggagagaagtctaggatgtttcacaaactacaaagctgaagaaaatgaagccctattacttgtttgtaagatttagcacccttctgctgtatactgtactgagacattacagtttggaagtgttaactatttattccctgttaaaatttaacctactagacaatgatgtgagtacccaggatgatttcctggggcacagtgggtgaggagatggggacaggtgaatggaggagttaggggagaggaaaagtggatggaagtgtctggaaagggcacgagagagtcttccaggtactgatcctgtttcttgctctgagtgctagctagccagctgtgttcacactgtaaacattcatcaagctgtacatttggtgcacttttctgtgtcataccacaataaaaaaaaacctagcatcttacaaaaacaagacacccaagtccaggcccaaggagtaagtacaaatattcctgtttctgaaccattactgtaattggctcttaaggcttgaagtaaccttataggttactcataaggcatatacaaataaacttgtttgttttcttttttcaaaaaaaaaaaaaaaaaa

SEQ ID NO: 12 - DNA, 7479 bp, Homo sapiens
tcctcagaggcaggaaatactttccaggcaatctgggcctggttgtggaggccccttttgcaaaacctcagtctgaatttagtagacagaagtcactaggaatgccttgacaggatcctgccttagctaaggctccctccagctgcagagggtgtttttgttagactcacacactgcgtgaaactgctcagaatagagccatgatctcaaccacgaaatgggaacttagattttggagaaactaacggggacggacttctttcctagcctgagtgttgagcagtgtcatgccttggcgtttcagctcctcgttgtctaggtggtgaaatgacagaactcattcgcttctttgattggtgattttgaaataatctttcatcaagttccatctcctttaccctcatatggaatatatctctctgtctgttgttaaactacgatgacatgtctgtagctatcagaaagagaagctgggaagaacatgtgacccactggatgggacagccttttaattctgatgatcgtaacacagcatgtcatcatggactagtagctgacagcttgcaggcaagtatggaaaaagatgcaactctaaatgtggaccgcaaagagaagtgtgtttcactacctgactgctgtcatggatcagagctgagagattttcctgggaggccaatgggtcatctttcaaaggatgtggacgaaaatgacagccatgaaggtgaagatcagtttctttctctggaagccagcacagaaacactagtgcatgtttctgatgaggataacaatgctgatttatgccttacagatgataaacaggttttaaatacccaagggcagaaaacatcaggccaacatatgatccaaggagcaggctccttagaaaaggcactgcccatcatacaaagtaaccaagtttcttctaactcctggggaatagctggtgaaactgaattagcactggtaaaagaaagtggggagagaaaagttactgactctataagtaaaagcctggagctttgcaatgaaataagcttaagtgaaataaaagatgcacccaaagtaaatgcagtggatactttgaacgtgaaagatattgcacctgagaaacaattgcttaactctgctgtaattgctcagcaacgaaggaaacctgacccccctaaagatgaaaatgaaagaagcacctgcaatgtagtacaaaatgagttcttggatactccttgcacaaacagaggactgccattattaaaaacagattttggaagctgccttctgcagcctccttcctgccccaatggaatgtcagctgaaaatggcctggagaagagtggtttttcacaacatcaaaacaaaagtccaccaaaggtcaaggcagaagatggcatgcagtgtttacaattaaaggagaccctggccacccaggaacccacagataaccaagtcagacttcgtaagagaaaggaaataagagaagatcgagatagggcgcggctggactccatggtgctgctgattatgaaactggaccagcttgatcaggacatagaaaatgccctcagcaccagctcctctccatcaggcacaccaacaaacctgcggcggcacgttcctgatctggaatcaggatctgaaagtggagcagataccatttcagtaaatcagacacgagtaaatttgtcttctgacactgagtccacggacctcccatcttccactccagtagccaattctggaaccaaacccaagactacggctattcaaggtatttcagagaaggaaaaggctgaaattgaagccaaggaagcttgtgattggctacgggcaactggtttcccccagtatgcacagctttatgaagatttcctgttccccatcgatatttccttggtcaagagagagcatgattttttggacagagatgccattgaggctctatgcaggcgtctaaatactttaaacaaatgtgcggtgatgaagctagaaattagtcctcatcggaaacgaagtgacgattcagacgaggatgagccttgtgccatcagtggcaaatggactttccaaagggacagcaagaggtggtcccggcttgaagagtttgatgtcttttctccaaaacaagacctggtccctgggtccccagacgactcccacccgaaggacggccccagccccggaggcacgctgatggacctcagcgagcgccaggaggtgtcttccgtccgcagcctcagcagcactggcagcctccccagccacgcgccccccagcgaggatgctgccaccccccggactaactccgtcatcagcgtttgctcctccagcaacttggcaggcaatgacgactctttcggcagcctgccctctcccaaggaactgtccagcttcagcttcagcatgaaaggccacgaaaaaactgccaagtccaagacgcgcagtctgctgaaacggatggagagcctgaagctcaagagctcccatcacagcaagcacaaagcgccctcaaagctggggttgatcatcagcgggcccatcttgcaagaggggatggatgaggagaagctgaagcagctcaactgcgtggagatctccgccctcaatggcaaccgcatcaacgtccccatggtacgaaagaggagcgtttccaactccacgcagaccagcagcagcagcagccagtcggagaccagcagcgcggtcagcacgcccagccctgttacgaggacccggagcctcagtgcgtgcaacaagcgggtgggcatgtacttagagggcttcgatcctttcaatcagtcaacatttaacaacgtggtggagcagaactttaagaaccgcgagagctacccagaggacacggtgttctacatccctgaagatcacaagcctggcactttccccaaagctctcaccaatggcagtttctccccctcggggaataacggctctgtgaactggaggacgggaagcttccacggccctggccacatcagcctcaggagggaaaacagtagcgacagccccaaggaactgaagagacgcaattcttccagctccatgagcagccgcctgagcatctacgacaacgtgccgggctccatcctctactccagttcaggggacctggcggatctggagaacgaggacatcttccccgagctggacgacatcctctaccacgtgaaggggatgcagcggatagtcaatcagtggtcggagaagttttctgatgagggagattcggactcagccctggactcggtctctccctgcccgtcctctccaaaacagatacacctggatgtggacaacgaccgaaccacacccagcgacctggacagcacaggcaactccctgaatgaaccggaagagccctccgagatcccggaaagaagggattctggggttggggcttccctaaccaggtccaacaggcaccgactgagatggcacagtttccagagctcacatcggccaagcctcaactctgtatcactacagattaactgccagtctgtggcccagatgaacctgctgcagaaatactcactcctaaagctaacggccctgctggagaaatacacaccttctaacaagcatggttttagctgggccgtgcccaagttcatgaagaggatcaaggttccagactacaaggaccggagtgtgtttggggtcccactgacggtcaacgtgcagcgcacaggacaaccgttgcctcagagcatccagcaggccatgcgatacctccggaaccattgtttggatcaggttgggctcttcagaaaatcgggggtcaagtcccggattcaggctctgcgccagatgaatgaaggtgccatagactgtgtcaactacgaaggacagtctgcttatgacgtggcagacatgctgaagcagtattttcgagatcttcctgagccactaatgacgaacaaactctcggaaacctttctacagatctaccaatatgtgcccaaggaccagcgcctgcaggccatcaaggctgccatcatgctgctgcctgacgagaaccgggaggttctgcagaccctgctttatttcctgagcgatgtcacagcagccgtaaaagaaaaccagatgaccccaaccaacctggccgtgtgcttagcgccttccctcttccatctcaacaccctgaagagagagaattcctctcccagggtaatgcaaagaaaacaaagtttgggcaaaccagatcagaaagatttgaatgaaaacctagctgccactcaagggctggcccatatgatcgccgagtgcaagaagcttttccaggttcccgaggaaatgagccgatgtcgtaattcctataccgaacaagagctgaagcccctcactctggaagcactcgggcacctgggtaatgatgactcagctgactaccaacacttcctccaggactgtgtggatggcctgtttaaagaagtcaaagagaagtttaaaggctgggtcagctactccacttcggagcaggctgagctgtcctataagaaggtgagcgaaggaccccctctgaggctttggaggtcagtcattgaagtccctgctgtgccagaggaaatcttaaagcgcctacttaaagaacagcacctctgggatgtagacctgttggattcaaaagtgatcgaaattctggacagccaaactgaaatttaccagtatgtccaaaacagtatggcacctcatcctgctcgagactacgttgttttaagaacctggaggactaatttacccaaaggagcctgtgcccttttactaacctctgtggatcacgatcgcgcacctgtggtgggtgtgagggttaatgtgctcttgtccaggtatttgattgaaccctgtgggccaggaaaatccaaactcacctacatgtgcagagttgacttaaggggccacatgccagaatggtacacaaaatcttttggacatttgtgtgcagctgaagttgtaaagatccgggattccttcagtaaccagaacactgaaaccaaagacaccaaatctaggtgatcactgaagcaacgcaaccgcttccaccaccatggtgtttgtttctagaacttttgccagtccttgaagaatgggttctgtgtctaatcctgaaacaaagaaaactacaagctggagtgtaggaattgactatagcaatttgatacatttttaaagctgcttcctgtttgttgagggtctgtattcatagaccttgactggaatatgtaagactgtgcaaaaaaaaaaaaaaaatttatgtgtattcttattcaaattgcttctgagaagcaaaactctttaaatacattatggaagataatgaagatacttcattctcttgtgatatcagtgtatgcgtacctgtgtcgctttatttgcagtgtgttgagggactggtgtatccactggaatagttggtactcttggatgtgttttctcaccaagatgagcaaagaaaggtttgcacagaggagtgtgaatgtgtgtttgttgctggctgaatggcaatagatgtctaaggtggattcagtgtctggcacactgagacacctccaagaaggagattgatgcatcaggttcagtttaacctggaatatctgactacccctgaatccacccagaaagggggcccaacacccttgtccatttatgggtattttttttcgaagttattaagcatattccttttccacgaacctcttctgtactttgattgtaataggttggctcttacacccattccaaatgcagtttatttttagacccgattgcaaatagtgatgtagttttaaccagtatggattagttcagggatgaactgctccctctagccttactggctctgatccacagggttttgttttgttttgttttgttttttgtttaagtcgagatataaaaactgaacacgataacacttactcttaaatcaagcatcaacactttttccctgttagaattctttgcatttttgtgtttgtaacagaaacgccttaagacactatgtttgggaatataggaaactatgtgtgtcccaaggaaatccctgtaaatttaactcacctacaaaaggctttttccccgcctttggttgttaacggcattcctgaaagccacatgtgtttattcattgggcttgttcttatcagcaaataggttttctggttttatgactttttgtcttattttatttttcctacatttctttttttttttttttcctttagaatgccctggaaatatatttaagtggtaatgaaaaatagtaatcatagtaaaacgcaacaagaagaaaaccaacccaaaccagtgaagttttttagaacctttagaagggtggtctttattcaggttttactgtaatggtaaggattgactcaagagacagtattagtaaatttattgtgtatggatcaaaagtgaataatgtatgaatgagagctgtaagaaggatttttattttgttataatttagttaccattttcagtgttatttcaaaggttctttgaagaattttggggcagggcatcagattagagttttaaaatttgagtattttggatatcagtgttcctcatgaagatatacatggatattcaattttgatggcttccagatttgtaagattgtatgttgtatataccattctattaagaaacatgtccactgtgctttcaaacatagataaagcatgataaagattattatttaagatatacttgtatttatacctcagatattcttttgggttttgtacctcaaggcttttttcttcttattgtaaatacactttacgtgaatacagtctaagtgaagaaaataaataaaaggaagaggtttataacttgctctatatctgtacagattataatcaataagtgcactattattaaatgtttaaagtaagggaaaagtctgggctgccttccttaatattgcatctcactcccacccttaaaaccacagattgcaaagcatagcattttagcatcaactacaatcaaaagagcgatttgctgaaggaaaaatcggactgcaaatcattccaaggccaaactgcaactgagccacccactcccaaacaggaaaccctggtgaaggttcaggaagcacggagattctctccaacaaaggtccagttaggaaacgacgctgagaggatgacgacaacgtgcaacagcagaaagatgcttgcaagcagagtcagggtcaccagtgaatgccacaaaagttctctttcccactgtttaatttgacaagagaagaatttgaaggatatgaacattttcaagaactctgctgaggtcacttagagcgccatcacaacttatttgtgtgactaattgcctagattgtaagctctttgagggcagggcttgtctcttacacatctttataatcccctgcagcggctttcagtattttgtacttgtaggcacctaataaatttattatttgctatactg

SEQ ID NO: 13 - DNA, 1912 bp, Homo sapiens
ttttttttcttccctctagtgggcggggcagaggagttagccaagatgtgactttgaaaccctcagcgtctcagtgcccttttgttctaaacaaagaattttgtaattggttctaccaaagaaggatataatgaagtcactatgggaaaagatggggaggagagttgtaggattctacattaattctcttgtgcccttagcccactacttcagaatttcctgaagaaagcaagcctgaattggttttttaaattgctttaaaaattttttttaactgggttaatgcttgctgaattggaagtgaatgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 14 - DNA, 1548 bp, Homo sapiens
tctcctgctgcattgtgaagaaactgcttgcttccctctcaccctctgcagtttcctgaggcctccccagccatgcggaacggccagatgcagtgcctcatgcctgtaatcccagcactttgggaggctgagggaggattgcttgagcctgcttccatatcgtaaactgccttggaaccaattaccactaccagggagacaaactattgcttagaggatgctgacaggagcagcatgccaaaattggaagaaggagaaagtttaagctctcctcactatgagttttcaagtataaaagactttttcttccacgattttgagaacaactgaggactcttgtgaccaggacaacagggaagcttgcagcaagatagctccaggttggattcatgcttcgcaccccaagggctgccagccagagaggaggagaagcaatcactcctgcagtttctgaacactacacagacgccaggtagcttcttcaggagaacagccctctgaggaggcaggaagaggaggcttatctttcagcagccggagctgctgagatctctgggcagattaagctctctctaatggatgggctccagcctggcacattcagtggagagggatccactcatccatcatcaacataatatggtcctccctgcacttcacagtgtcctcttgctattgaaaaggcttttttgccttctcaagtttctttgtcaacagtctacaggaagaagctcaggccgccaccggcagaggtgaatgcaagctcacgttttatttctgactgcttaatcattgcctcgatcactgctcaagctctgcctttgtttccaaaggttacctgtgggaaaacttctttttctatgctgaaattaatagggaggcaaagatgagtccactgataagcagagccttaaaactcacatagagaaacaactttgctggagtgtgtgtgagtgaaccactaaggaatcagatagtgtgatggcagttatcattgcaggttaagacatttctacaaatatttcgacatctccatatactcactcctttcccccctgagtggagagactcagctaccaagagaggaagctcaaaaaaaacagaagcttcaaacaaacaaccaaccaaaaaaaaaaaaaaactgtgggttcatcagaggtggtagggaagacaaaaacatgggaggagcagctgggcacggtggctcaagcctgtaatcccagcactttgcgaggctgaggtgggtggatcacctgaggtcaggagttcgagaccagcctggccaacatggtgaaaccccatctctactaaaaatacaaaaaattagccgggcgttgtggtacgcacctgtaatcccagctactccggaggctgaggcagcagaatcgcttgaacccaggaggcagaggttgcaacgagccaagatcgcaccattgcactccagcctgggcaacaagagcgaaactccatctaaaaaaaacaaacaagcaaacaaacaaaaaacacgggaggagcaataaaaaggaatccatgctttgattttttttt

SEQ ID NO: 15 - DNA, 3430 bp, Homo sapiens
ccagaaggagttgggagatgagccttccgcaaactcctgacctgtgggtccgtccgttcaacggccaaaggctggcggaggagggatcccctgcctttctcggaacggaacggagcagagtcgtgcgtggttgagtttagataaaagccgagtgagcgcgctctgttccttaagattagtttaaggtgccttggattgctctgaagagctttgaccacctgatattgcttacatctggaacttcttggcttctcattccccagatgtgcgggtcagagaggattctacaggcaggaaacatcttagaaatcagggttgggcaggcaggagccaggagagtagctacaatgacttcaccagtactggtggacatacgagaagaggtgacctgccctatctgcctggagctcctaacagaacccctgagcatagactgtggccacagcttctgccaagcctgcatcacaccaaatggcagggaatcagtgattggtcaagaaggggaaagaagctgccctgtgtgccagaccagctaccagccagggaacctgcggcctaatcggcatctggccaacatagtgaggcggctcagagaggtagtgttgggccctgggaagcagctgaaagcagttctttgtgcagaccatggagaaaaactgcagctcttctgtcaggaggatgggaaggtcatttgctggctttgtgagcggtctcaggagcaccgtggtcaccacacgttcctcgtggaggaggttgcccaggagtaccaggagaagtttcaggagtctctaaagaagctgaagaacgaggagcaggaagctgagaagctaacagcttttatcagagagaagaaaacatcctggaagaatcagatggagcctgagagatgcaggatccagacagagtttaatcagctgcgaaatatcctagacagagtggagcaacgggagctgaaaaagctggaacaggaagagaagaaggggctacgaattatagaagaggctgagaatgatctggtccaccagacccagtcgctgcgagagctcatctcggatctggagcgtcgatgtcaggggtcaacaatggagctgctgcaggatgtgagtgatgtcacagaaaggagtgagttctggaccctgaggaagccagaagctctccctacaaagctgagaagtatgttccgagccccagatctgaaaaggatgctgcgagtgtgtagagagctgacagatgtccaaagctactgggttgacgtgaccctgaatccacacacagctaatttaaatcttgtcctggctaaaaaccggagacaagtgaggtttgtgggagctaaagtatctggaccttcctgtctggaaaagcattatgactgtagtgtcctgggctcccagcacttctcctctggtaagcattactgggaggtagatgtggccaagaagactgcctggatcctgggggtatgcagcaattcactgggacctacattctctttcaaccattttgctcaaaatcacagtgcttactccaggtatcagcctcagagtggatactgggtgattgggttacagcataaccatgaatatagggcctatgaggattcttccccttccctgcttctctccatgacagtgccccctcgccgtgttggggttttcttagattatgaggctggtactgtctccttttataatgtcacaaaccatggcttccccatctacactttctctaaatattactttcccactactctttgtccatattttaatccttgcaactgtgtaattcctatgaccctgcgtcgtccaagctcttgaatattcttctgttcccacccacttctgataagtaccctgaggcttatcagcatgtgattctcccttctgatcttctgtttttctgtgttctcaattcttttgttgttttttggtttttgaatcttttttgagatggaatctcgctctgtcgcccaggctggagtgcactggcgcaatctcggctcactgcaacctctgcctcctgggttcaagcgaacctcctgcctcagcatcccaagtagctgggattacaggcacccaccaccatgcccaactaatttttgtatttttatagagatagggtttcaccgtgttggccaggctgatctcgaactcctgaccgcaagtgatccacccgcctcggtctcccaaagtgctgggattacagatgtgagccgccgcgcccagacagttctttcgttttaaacagttactcagtactaggatgcacccagtggtgagagtaagcatctttgactgatgacaggtcttgaggtggatagggggcgctttcagtattttgccattaagcatagtatttgatgcaggttttttttgattgatgcaggggatcaaatttaggaagttctgatctgttaatttactacaagtttttgtataaaatgaaaactcgtattctacctatgtcttttctgtaaattattgagacaattgtgtatcatttttgttctgttaatgtggcttagtacattgatttacttcaatttgttaccacaacttgctgaaatacaccattattatttgttgtatgcaatactggatttattttgataacgtattgtttagattttgttctcatctatgttaatgaaagaaattggcctgtatttttacattcttgtaatatctttgccaggttctataaaatgcaataataagcaaataaattactttgtttttgtataagtatgtataggattgagctctaaaaccaaaccattataatgataatttgggtagcttcaaactcaaattgaagagagtcttcactacaatcttcactacaacctttggccttcctctctacatttaagagtagcaatagaacaataaaaaaaaatacgtctttcaaattaattgcagtaaaaacaaaagagtagtggggagagaaaaacaatccaatacatatttttctctcagaatagaaagaaaacacaaagagaaaatatgaacaatataaagcacttaaaggaagaggatgatttcttccagcttttccacactctctttatgcgcaggaacaaacagctgttgagtgagctgctatgactttggtagagaggtgcaatatgttttttgaaccagctataaaagaaattaagaggtacatgtgagggtagaaacaaatatcaaggatgagaatggcactggctgtcccatatggtatccaggagccaccaatgtctgttgagtacttcatacatgcctagtctgaattgagatgtgctcaatgtataaaatatataccagatttcaaatacctacttcaaaaaataatgtaaaatatctcactaataacttttcctttgtgtattgcaatgataatatgttggctctattggattaaataaagtatgttattcattttacctgtt

SEQ ID NO: 16 - DNA, 11471 bp, Homo sapiens
aattcatttttaatcctttaatagtccacagtaatattgtcctaaagagggtacattggattttaattttgctttcaatatgacggctgtcaatgttgccctgattcgtgataccaagtggctgactttagaagtctgtagagaatttcagagaggaacttgctctcgagctgatgcagattgcaagtttgcccatccaccaagagtttgccatgtggaaaatggtcgtgtggtggcctgttttgattctctaaagggtcggtgtacccgagagaactgcaagtaccttcaccctcctccacacttaaaaacgcagctggagattaatgggcggaacaatctgattcaacagaagactgccgcagccatgttcgcccagcagatgcagcttatgctccaaaacgctcaaatgtcatcacttggttcttttcctatgactccatcaattccagctaatcctcccatggctttcaatccttacataccacatcctgggatgggcctcgttcctgcagaacttgtaccaaatacacctgttctgattcctggaaacccacctcttgcaatgccaggagctgttggcccaaaactgatgcgttcagataaactggaggtttgccgagaatttcagcgtggaaattgtacccgtggggagaatgattgccgctatgctcaccctactgatgcttccatgattgaagcgagtgataatactgtgacaatctgcatggattacatcaaaggtcgatgctcgcgggagaaatgcaagtactttcatcctcctgcacacttgcaagccagactcaaggcagctcatcatcagatgaaccattcagctgcctctgccatggctctgactaacctgcagctcccacagccggcatttatccctgcagggccaatactgtgcatggcacccgcttcaaatattgtgcccatgatgcacggtgctacacctaccactgtgtctgcagcaacaacacctgccaccagcgttccgttcgctgcaccaactacaggcaatcagataccccaattatcaatagatgaactgaatagcagcatgtttgtttcacagatgtagaagttctcagtagaacactgaaattctgaacagcagagttatggagtatcagaatctttccatggaaacctccatatggcctttctatatatattctcgtatgtcttattctaccaacacaacaataagcgtgttgcagtcaatgtattaagcaaagcaaacctgccagccagcaaattcaaataaaaaataaagcattaaaaatcaatggagatgttaaaacaacacaaatagaaaactagtaactaccatccatcctatttgaattatcaagcagaacatgaccataaaatttggtaacttgttacattactctttgtgattttctaataaccatgctaagtgtatttccacagtgagcttttggcttactatatacattcttggtggataaattgttcatctgtttttgaagtgttaccttactattttgtttacaagatagtctattgggttgattcaggatgtaacaaatatattcagtaccatttcttgtgttgtattgtgttgtgctgtgttaggtttttacatactgtagtgttttgctgtatatgtgtggtgtttgatttcaactaaagtgttattagtggggaacagaagtatatgtgcttaagaacatgacaggttcatgcaaatatgctctctttctttagaatatttctgtaggtttcttgggactgacatttaaaacgcctcacttttgaatgtgcacaaaacctgctcattaacatgcatgtgtataatttgtacctgcagatctgatgttgcataatacaatcaaattactagattttttaaagagagaaataattacctgcacaaagcagagaacttcataaaacattaacccctaattcactcttcttaaatagcttggcaaataagactttacctttaaatgaatttctcagcatttatactaaaaattatgtaaagtgctcattagattttttgtgtgtgtggcttgagaatcccatctcctaaattgagtgtctaaaactgagccatttgtcatcttcagctgagaaactggtacttgggagcttaaaaatatgctaattacaagttataaatcaaacggagagatgggggcatggagatagtttttacgtactggaggaaagtgtgtaaaaccatggcaatgtcaccttttacacaaatgccattttccaaatgcaaatggctcatgctctttagactactctttgaataacaagtaagatgcaatctagcaaaagtcagtcagggtgaaagagaattggttgcaaatgaggacttccctccccaaatggacagtcttctctgttgatcacagagggagcctgagtacaggcttggagaaatggctaggacagggaacagggaagcacttacaattattccttgatttattcaaaagaactgggaaagatggttgtagttgtctttagcttcggttcaactgagtttcgttttgttaaacagttcagtgaaggagaaagcacctgtgatatatggcaagtgtccccctgcccaaactttaacatcagaccctctcacatcataaccaagcctcttttatgcttgtttggattcgtttgcctttatatatgtttgaaaatgacaatagaaagggttttatcaccctttgaatgagacagagtggctatactccacagagctcagctgagttcacattactaatatttatgaaagaattttacaaccaaataattcctgtggtgtgtgacatatctcatccactctactcatttattaatgttattatacagtggtggaaacacatgacattttacatcaagtaatagatttaatattttctagtaaataggcgctcttccaccagaaagtataaaaccatcatatacatatatatatatatatataatggtttttctccttcatgaaaatgacaagtaaaaagcaaatctgattgaggataccttaagctggctagaagtgtttgaatcacagaacaatggaattagcaaaatcctttttatagttgatggaactgaatccaggggcatatgacttgcccaaggctaccttagccattgtgggacccagaaatatctcctgcttcccattgttcctccttttcacattgtttttgcttcagaacatttgatgctctcttccaaagcagacccatttggctcagaatataagagagcaaaattagctttctgggaattgatactctgttgatcttaattatccatccaaatcctgatcaacagtgctttctaaacagccttgcttgtattgagtcattgtcagctgttgacaatgttgcgtggaattagagatcaagagttgtcctgacctttacctatggactctttgaaccagttaatttctatttctgaaatcttaacattgctatagaagaaagtagtgtatgcctttttgaaattcatcatgtatggaactgagttttgtatgatccgtgccagtgcctctccagaagggcagatctgattacatgaatccaggagggatgcacctgagctagtctggatatattccttaaaggagacaagaacttttttgtgcaaaatgaacttttgtgtagtttgacatctgcatgcagtgaactgtctcacttaactttttcaactttgggcaaagatatacataaaccatagataatagacctacagtacagctgttttcatgttgcgttaagcactggaagacattaacaatgacttaccttaaagtaaaaggttccgtgaggggtatttagggccactgtatttttggtgccacaattttctacattgttggcattttaaaagttgttttctaaatgaacttaaagaaagaaagctcaaagccctaaagttttgatcaaattcaatagagttttcagaaaaaaatacaattttaagatagcactctttaaactagatgtgcaatgacttattcaaattatctttattcaagaaaagagaaaagaagaaagataagtcattagactataaatttccatttaaaaagacaagtcacaaaactaatcagtgactctaaacaaaaatttactggatgaattgtatatataaaattctaatcacttatgattcttagttaacattttataagctttgtattttacaggggcctctatttatccaaatttattgtttttgtccatttttctcagccttttgcaaaaaatatgcaagagtgctgaaacttggataaaacgggtcatgtatacagagaaaataatctaaaagttatctttaagagggttttagtagtgtcaactttttaaaaaagtactatgtgaccataacatacgtatattttaaagagggattaagattaaattaaaataaatggattcataaaattgctatttaaatacagtatttacttttgtaattgaagcatgattccacaaccagctgtggcattttatttcacttttctagatcagaggctttaatttgtagttaatcatgatgtggaaagtcatcttctctctactccatcaatttttttcatccgtttttgaacatgctgaagcactttgatcttgccataagtgttgtcctataccactaggtggtgttcactaatgggagggcctcagctcaccggctctccaatttgggaaaaagcaagtacctcctccctaccatcagcagaaactgaatgaaattaatctttgcaacttgaatctgttttaagtagcattaccaataagcatagtggtatctgaataaggaatgacatttacattacattttattaaataagagagaaagttaatgtgctacttgaaatgttatttggttctttgtccccctcattgcagagtatagataaggatgctcttaggtaaaatgcagccacaagttttccgagttcctttagtctgtagggtaggctatgaggtacagaatgattagaaactgaaatcctcattatttttctctcaaatacatttcacatacattgaaacagaaataggcaaaatttatgagggaaatctgatttcccaattctgatgaatagatatactaagacagtagccttgtttaaccagtgcctgtagaaggcactagaataaatagcatgtccaagagtataacaactttttttgtaaatttagttactagcctaatcaaactgtaaatataactcactaattggtaccatattttcatttttcttattgttcatctttaaggtttcattttattgcttttttgtagctctaagtaactaatcaatttaatttcaaaggaaagttaaagaaagtttaagcttttactgcatggattctgcccgcctagctaggttctgaactgacactgaagtatgacaagatacagattatagtccaaaactatcctttacagttcacaatttaagaacaaatttaactttatatacaaataataattgttatctaattatatttagttgttgctttggcaacaaaattacaacatggctacctttagacatttgtgtatgtgaacattttacttgtaaactataggtgatttgtgacagtatgagcctgtagaaaatacagctcaaaactatgaaaacagacatggttgagttcttttttaatcccaaaggcccatatataaaagctgaatttgtgtccttgtgtgactttttgtgaaaaagtttataacacatggattctagaatcaggctttatagtttatagaatttgttgtgtggatagggttattccacagtatttagacttcaaaacgcaatcattgcatttttaatacattacctaactttttttataacttaattcaaaattaccccattcaagtggcatagctgtagctgcaaaaattctgttttctggaattacatgagcaaatatagctatatgaggattaatgtatgactaaagcctcttttgaaattaattcatatttttaaaactatacataaagatactgtggaatgaccccttaacagtgtttaagaaacatatgctgcagttaattgactgtctcagttagggtgcggatgtgtgagacacaccttttttgcacttatgtacatagtccatgaaagcaatccttggagctttttttaaatgtaagaagtctgtgataatagtggaaaagttagtaagagagagttatttctaaggctttgaaaaggagggtgtttttgctctaagacttaactaaagcataatgttgctttgctgttacggaggctttctagtttgctacaggtcaccaaaagagaaggctagtaacaggtaacaggtcagcctcagctaacagcactgaacaagagtagaataacttcatcttttaacaagatcattagcaaagattgtttgctttttaaattaatcccccctataaacacacacacacgtgtgcacacacacacacacacacacacaccgggcagtttctagaacaagttttcctttttcagtggttgtaagcagtgaaatacctcatgtattacaagatagatattaccgttatgatagaataagatttcatctttcacagaaagtgatcagctgatatatgtgtcagttaaaatgtttatcaattaataggtatttaagtgacaaattgttaatgttgaccattgttcctcctttaagatccagtaaccaaaaccaaatcggtaaccattgaaagatgatatttaacagttaaggatggccagcatagggccgttcattcccttcaacaaagggaaaaaaaatgctcctggcacaactttataactggcttcatttacattatacctgttcagtggaagctgttttttgacctacctttaccagattacaaagtagttaactgtgccacttcagcctcccactgttttgttttaactaaacagaagagaaccagccaaaatcttacgtaacacaggagctggaagacaatgtttcaatctgaccatctctgccaattttagctcccacgccacctatagatagccagttaactttctttttgcccccaggcaatgaggcctagaagaggtcaaggttagaaaggttaagcatttgatcactacaggtacttgtagtacctagagaaggaacccatcctggggttggtggagttggtattgctactcgttttgatagttctcatttacagagaaagaaaaataagccaaagccagaaagcaatccaagccttacagttgtccaatacctcagggccagttggctagaattttcttttccctcagcccataatcacttagagttagccatacaacaaacagcctgacaatctgaccagcatttccattgatataaaatgtaagcattccttcattctgtagttaggaactccaacttcagccttatctccaacataatttcccaatttaacctatgtactgatatggacatttctttgtgaatatgccagattcaagaatgtatttttctaaggtcaatagagatacgacattttaagtcaggaaaaatcacctataaattagtaggaataactgtagaagcatcacaaaagataccccatgaatagatggcatggtgtgcatgctgacagccacggggtcatactctggagtttcaacatagaggctgtgtttcaataaataagacaaattggggaattaagtaaaacaaatggatgtaactgaggcccccccccaaaaaaagccctctattactaacctcaagaaaatatttagtttgactaatttgtgcagtttccagctcttctacttgttcttgcctgtaagagaatcactaatactcaaaagctggattattgaagagaaattttatctttaaatagaatccatccacaattatggtatatgtaaaaaaaatgtatgactatgaaataagtcgcagctctttgcagatattgcaaaatccttgctaggcatctacatcatattagttttttaaaaaatacctctgtaacaagacctaaaagtaacctaactattaagcagtgcttttttctctgaaaacacacaacctaaagatttatctgtcataaaactgcatggttccagagctttatttctgtgtagagatgctatttgttagtgatggaaacaaattactttgttgctatgtcttttgtgtctagcagttgagagaatgatatgaccatcatatcactggattatgggaatgaatgcccttttcaaaataaacttatttattaatattttaatattattttaatattataatattaataatattttaatattattgttgctgctttaacagaaaatgagccatttgtccccatgctcccaaattacaatgatttaaattaaaaaacttttgaaggattttcctaaatatattcttttttaaaggactaactttgtgcattcatataacaacaatttggaataatgtgaaaaatggaggcacataatttgggtggaaaatggcgattaaaaatatatataagaaaattattagccatgtgtaacatatttaaataatgaccagaatatttcatgagtaccaactcttcccatagtaattttcctccaaaatgcaatagctcattccctcaactgcagacgctttgattttttttcaagttatagtgctgtagtgcttgtttatgtttaaaagtgcacattatgcagctcattttagtatgctacttctatgtttattttttgttcttctaataaaatgcataaacttcttggttatgtctttttttatgaatccatattcacacattaggagttcacttgaaggctattcatgctctagtatattaatcctaatatttagtaattctaaaatgaaatttaaatttctgatttggctcaatgttatatttcatacatagagtaaatttatttccaaatataccatcagtattttgaattatgtagataatatatccgttgtgttagacctattactcttataagattgaagttactgacacaagaaaaacacttctattttaataaaagaatacttaaaattttgagagaatgcttactagtttctcttgtgcctcagaaatcctgaggcatattctaagatgcattattagaagtttctctttcccagttattcatgtctatcctgcatccctagtgtaacatatgaaaccagtgttgtttatttcatactttccagatgggcatcaatcatttcataacaatgagatgcatgaatttttccattaaaatgttttctctcacttaaaattggttatcttcaacaaatggcacagtaataaaaggatgatcatttttgttctttaagaaagtaaatgtaatttagtaatactttattctgccttatttcaaaatgcttcaaaaatgcgaatctggaacttctctgcattgaccactgttctggagttgggctgatagaaaactgcggtgaagagagtttagatcagaggctatggcagagcaaagtattactgatgtgtctcttaacagtgaagagggtcggggaggaggttgggtccttgtcaagtgtcagctgccctatacatagttctcttgccccttgtgccctgttttactatttctaaggtggttcccatgatggcatttcttgataagcccactttgagccgtttaatgttttgtaggtgtttctgtgagtctgataataccagcactaaaaacagttcagtatatagacatttacaagaggtcttaggcctcttcagaattcagttaatgtctttctctttgaggctgctgaatgttaggtaagaattcagggcaagcattcccgaccatcatcaaattggagacttggagtttctattggctggaagaaagttgacccacaggcaaaaccagatgtaaaccaagtttgtcttgggtacttttcaaattcttttcacagcatccttttctcatccctcatggtggcctctggccctggaaaatcctcatgatagcaacttatcaggcctgtttcctctgatagatctggacttttccccttaagtgctttcctgttgggcttctcagactatggagaactgctcatagttgagatctaagagacaaattaccctatggtgttcaagaatggccgtatttagtatactgcctgtcctggaagtgttcacatttaaaaaggcatgactttccaatgggcaaatatcagacacctacatgcatagataagggaaatatgttttacttaagcaaacttctaggcattgaatctcagtgtttaggtgcagagatggaattcctataagcaatgcagtcattcaccacatcaggaaggccaaaaaatgaacactctcgtaattttgttattatctggaaagttctgatatgataatgaacctaaaatactgaatagatagtgaacatcctaaaattccactgaccagaatccattcccatggtcatcatctgtagagggcatgatacaatgacaggcctgaggtatgattataattctggagtaatttaaagcctatatttgttaaactttaacagatggacacacattctctgattttctacttggctctttcctgtactagaaatgcgcatttaactaaaccttcccagcttttctgttcagtgcctgacttacactactcccttaggcaggaggacagtgggtgtctggggaataaaagccaagagctccaaacagaggattttggatacaaagggcagccaccaggaaatagctttgtgagagggacagactatctgattcccatttgtacaaacatgtccaaagcaaaagcacagcattctaaagtggatgcagaaattagtatacaatggcagttctggccagtgcagcccagcatttttctgttgtgcatggaaagaacacagtgaaaaggcaggcagggcatccggtgcaaagtgcaacagacatagacacacaaatccagttttcctgatgagagagggagctatctcttcataagtgatgcaatgcaatgcaggcatgtacaagtacaaggaaagatttatttttaaaaaatcatagtgtaactggatgaataccagcaacagcacctgctaaagcaaacagcctgttcattaaaaactggacttgagggaatggagaaagaatctgaggagcctgcagtcagcatgcttgcagagaccttatggctacagaagacgatgattaacccatagaggttcaatgtagttttcaagaagagggtgggaaagcataagatggggactaagactttgcctttaaccttcatgacatgaagaattgaatgactctttgttggaaaaaatggcagttatgagataattggatccatgttaattatatatatttctgtatatatagtaagtggttattgatgtgcctttgttaaccatttcactatttaataaatgtgtgtgtagtttattcaaaaaaaaa

SEQ ID NO: 17 - DNA, 2900 bp, Homo sapiens
attccagcctcattgtaacacacattctacgcctagcctggctttcttgctctccctcatctcattgtttcagcggaggccaaatctgaagtcctttccagggagtggctctgttcatcttattcgccagccaaagtaggaacagcgtaagaggagagagacacattcagcagccaaaggactcggtggaaagagcagaacaccatagacaatatgtcgctcttgggacccaaggtgctgctgtttcttgctgcattcatcatcacctctgactggatacccctgggggtcaatagtcaacgaggagacgatgtgactcaagcgactccagaaacattcacagaagatcctaatctggtgaatgatcccgctacagatgaaacagttttggctgttttggctgatattgcaccttccacagatgacttggcctccctcagtgaaaaaaataccactgcagagtgctgggatgagaaatttacctgcacaaggctctactctgtgcatcggccggttaaacaatgcattcatcagttatgcttcaccagtttacgacgtatgtacatcgtcaacaaggagatctgctctcgtcttgtctgtaaggaacacgaagctatgaaagatgagctttgccgtcagatggctggtctgccccctaggagactccgtcgctccaattacttccgacttcctccctgtgaaaatgtggatttgcagagacccaatggtctgtgatcattgaaaaagaggaaagaagaaaaaatgtatgggtgagaggaaggaggatctccttcttctccaaccattgacagctaacccttagacagtatttcttaaaccaatccttttgcaatgtccagcttttacccctactctctactttttcacccaaactgataacatttatctcattttctagcacttaaaatacaaagtctatattattgcataattttgctgcttctcaatatcatagacacagtgaatagatgatgactatatggcttatatacaaacattctatgtacaatttcaagggagactaaactttaggctaataatctttactattgaatctgtctgatatagatcttagggttgaagaagctatctttgtctatttgggctaaccatagaatttcatttattttcctcacaatattttcctagaccaactccccatcattcacgtgttcctctttactcttactttaactattttgctggcttgcccgaaaatttgcctggcaagtcttccttataagacacatcatggtaagttttgtagtcctgtaagattctgcaacacagtcaagaattatacaatcctactagcaatatataaggacccaaaatgtcttctgctaagctcagaggctggggctaaagcatgaggactatgccagctatagaacttggactcataattcgctatccaatttttcatgcagttgtctagtcgggaagtaaggttggaaactaagtctcatttactgattcgtttatgggtagtaccgggatgaacccaccaccacaaagcaaattagacaacttaatgtgaaatcataccattggttgacgtttccttgagttgctacttcgttcatcttcacaacttaacaagtgcacggtcgaattattgtgcaagtggcttttggatatcctgattggggcctaagaagggcattcagacttgaattttaataggcagacagaaagtttgcctaatagttaatacgaaagagtgaaagaaacacaatattcagacaacccacattcttatcctggctctagcagtaaccacgtagccttggataagccattttccttcattaggtcctggtttaatttcctcatctttaaaatgagaaggttaaatttatcttagtactgctgggcgcagtggctcatgcctgtaatctgagcactttgggaagctgaggcgggtggatcacttgaggtcagaaatttgagacgagcctggccaacatggtgaaaccccatctctactaaaaatacaaaaattagctgggcgtggtggcacgtgcctgtaatcccagctactcgggaggctgaggcaggagaatcaattgaacctgggaggcagaggttgcagtgagccgagatggcgccattgcactccagcctgggtgacaaaagcaaaagtccatcttaagaaatatatatatatattatatatattcttagttctaagatttcctttaattctatgattctctggatttaaatgcattattcatatttcttgaagcttagatacagtctaattcatagcaaccatatctgctttatcctaggtgagggtagcagtccacaatggaatagaagaaaatcccattataacaaatgacaaattatatatcatgaatccttctgtctgactaactcaataactttctataaaagccaatggaattcaaataggagctaggagacaacaagttatatatgacagtggaggttgtattccttttatattgctgagaaaactagttaaatgatcagattcttgctgttaagaaacaatttcgtttaatgggatctgtacaactgattttaaaaaaatgctacaaaaagccccaaagcatataatctctactccttacagtctctagaattaaatgtactcatttagacaacatattaaatgcatattttagccactttagagaaacctcataggcacagagtttccaagattaattttaagaatatcttcacgaacttgaccctcctactccacattgcaacatttccatcagacagcatttcaattccagtattatgtatattgcaaattaaacattttaaaatatttttttccaatttatttctcaaaataaaatgtcttttgttctggtaaaaaaaaaaaaaaaaaa